(19) United States
(12) Patent Application Publication
(10) Pub. No.: US 2012/0129870 A1
(43) Pub. Date: May 24, 2012

(54) PIPERAZINYL DERIVATIVES USEFUL AS MODULATORS OF THE NEUROPEPTIDE Y2 RECEPTOR

(76) Inventors: Wenying Chai, San Diego, CA (US); Jill A. Jablonowski, San Diego, CA (US); Dale A. Rudolph, San Diego, CA (US); Chandravadan R. Shah, San Diego, CA (US); Devin M. Swanson, La Jolla, CA (US); Victoria D. Wong, San Diego, CA (US)

(21) Appl. No.: 12/809,023

(22) PCT Filed: Dec. 17, 2008
(86) PCT No.: PCT/US08/87261

§ 371 Date: Jun. 17, 2010

Related U.S. Application Data
(60) Provisional application No. 61/014,186, filed on Dec. 17, 2007.

(51) Int. Cl. A61K 31/496; C07D 295/135; A61K 31/495; C07D 405/12; C07D 401/06; C07D 417/06; C07D 403/06; A61K 31/506; C07D 413/14; C07D 405/06; A61P 25/22; A61P 25/24; A61P 9/00; A61P 19/10; A61P 5/00; A61P 25/32; A61P 3/04; A61P 15/08; A61P 25/00; A61P 29/00; C07D 409/06; C07D 401/14; C07D 295/15; C07D 413/06

(52) U.S. Cl. 514/252.19; 544/367; 514/254.03; 544/393; 514/255.03; 544/374; 514/254.1; 544/369; 514/254.02; 544/360; 514/253.01; 544/295; 514/252.14; 544/364; 514/253.1; 514/253.09; 544/370; 514/254.05; 514/254.04; 544/368

(57) ABSTRACT

The present invention is directed to piperidinyl and piperazinyl derivatives of formula (II) useful as inhibitors of the NPY Y2 receptor, pharmaceutical compositions comprising said compounds, processes for the preparation of said compounds and the use of said compounds for the treatment and/or prevention of disorders, diseases and conditions mediated by the NPY Y2 receptor.

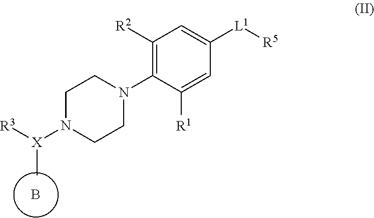

### FIELD OF THE INVENTION

The present invention is directed to piperazinyl derivatives useful as inhibitors of the NPY Y₂ receptor, pharmaceutical compositions comprising said compounds, processes for the preparation of said compounds and the use of said compounds for the treatment and/or prevention of disorders, diseases and conditions mediated by the NPY Y₂ receptor, including, but not limited to anxiolytic disorders, depression; pain, injured mammalian nerve tissue; conditions responsive to treatment with a neurotrophic factor; neurological disorders; bone loss; cardiovascular diseases; sleep-wake state disorders, substance abuse and addiction related disorders; obesity; and obesity-related disorders. The compounds of the present invention are further useful in modulating endocrine functions; particularly endocrine functions controlled by the pituitary and hypothalamic glands, and are therefore useful in the treatment of metabolic disorders, inovulation and infertility.

### BACKGROUND OF THE INVENTION

Regulation and function of the mammalian central nervous system is governed by a series of interdependent receptors, neurons, neurotransmitters, and proteins. The neurons play a vital role in this system, for when externally or internally stimulated, they react by releasing neurotransmitters that bind to specific proteins. Common examples of endogenous small molecule neurotransmitters such as acetylcholine, adrenaline, norepinephrine, dopamine, serotonin, glutamate, and gamma-aminobutyric acid are well known, as are the specific receptors that recognize these compounds as ligands (“The Biochemical Basis of Neuropharmacology”, Sixth Edition, Cooper, J. R.; Bloom, F. E.; Roth, R. H. Eds., Oxford University Press, New York, N.Y. 1991).

In addition to the endogenous small molecule neurotransmitters, there is increasing evidence that neuropeptides play an integral role in neuronal operations. Neuropeptides are now believed to be co-localized with perhaps more than one-half of the 100 billion neurons of the human central nervous system. In addition to being measured in humans, neuropeptides have been discovered in a number of animal species. In some instances, the composition of these peptides is remarkably homogenous among species. This finding suggests that the function of neuropeptides is vital and has been impervious to evolutionary changes. Furthermore, neuropeptides, unlike small molecule neurotransmitters, are typically synthesized by the neuronal ribosome. In some cases, the active neuropeptides are produced as part of a larger protein that is enzymatically processed to yield the active substance. Based upon these differences, compared to small molecule neurotransmitters, neuropeptide-based strategies may offer novel therapies for the treatment of CNS diseases and disorders. Specifically, agents that affect the binding of neuropeptides to their respective receptors or that ameliorate responses that are mediated by neuropeptides are potential therapies for diseases associated with neuropeptides.

There are a number of afflictions that are associated with the complex interdependent system of receptors and ligands within the central nervous system; these include neurodegenerative diseases, affective disorders such as anxiety, depression, pain and schizophrenia, and affective conditions that include a metabolic component, namely obesity. Such conditions, disorders, and diseases have been treated with small molecules and peptides that modulate neuronal responses to endogenous neurotransmitters.

One example of this class of neuropeptides is neuropeptide Y (NPY). NPY was first isolated from porcine brain (Tatemoto, K. et al. Nature 1982, 296, 659) and was shown to be structurally similar to other members of the pancreatic polypeptide (PP) family such as peptide YY (PYY), which is primarily synthesized by endocrine cells in the gut, and pancreatic polypeptide, which is synthesized by the pancreas. NPY is a single peptide protein that consists of thirty-six amino acids containing an amidated C-terminus. Like other members of the pancreatic polypeptide family, NPY has a distinctive conformation that consists of an N-terminal polyproline helical region and an amphiphilic alpha-helix joined by a characteristic PP-fold (Vladimir, S. et al. Biochemistry 1990, 20, 4509). Furthermore, NPY sequences from a number of animal species have been elucidated and all show a high degree of amino acid homology to the human protein (more than 94% in rat, dog, rabbit, pig, cow, sheep) (see Larhammar, D. in “The Biology of Neuropeptide Y and Related Peptides”, Colmers, W. F. and Wahlestedt, C. Eds., Humana Press, Totowa, N.J. 1993).

Endogenous receptor proteins that bind NPY and related peptides as ligands have been identified and distinguished, and several such proteins have been cloned and expressed. Six different receptor subtypes [Y1, Y2, Y3, Y4(PP), Y5, Y6 (formerly designated as a Y5 receptor)] are recognized based upon binding profile, pharmacology, and/or composition if identity is known (Wahlestedt, C. et al. Ann. N.Y. Acad. Sci. 1990, 611, 7; Larhammar, D. et al. J. Biol. Chem. 1992, 267, 10935; Wahlestedt, C. et al. Regul. Pept. 1986, 13, 307; Fuhlendorff, J. U. et al. Proc. Natl. Acad. Sci. U.S.A. 1990, 87, 182; Grundemar, L. et al. J. Pharmacol. Exp. Ther. 1991, 258, 633; Laburthe, M. et al. Endocrinology 1986, 118, 1910; Castan, I. et al. Endocrinology 1992, 131, 1970; Gerald, C. et al. Nature 1996, 382, 168; Weinberg, D. H. et al. J. Biol. Chem. 1996, 271, 16435; Gehlert, D. et al. Curr. Pharm. Des. 1995, 1, 295; Lundberg, J. M. et al. Trends Pharmacol. Sci. 1996, 17, 301). Most and perhaps all NPY receptor proteins belong to the family of so-called G-protein coupled receptors (GPCRs).

NPY itself is the archetypal substrate for the NPY receptors and its binding can elicit a variety of pharmacological and biological effects in vitro and in vivo. When administered to the brain of live animals (intracerebro-ventricularly (icy) or into the amygdala), NPY produced anxiolytic effects in established animal models of anxiety such as the elevated plus-maze, Vogel punished drinking, and Geller-Seifter's bar-pressing conflict paradigms (Heilig, M. et al. Psychopharmacology 1989, 98, 524; Heilig, M. et al. Regul. Pept. 1992, 41, 61; Heilig, M. et al. Neuropsychopharmacology 1993, 8, 357). Thus, compounds that mimic NPY are postulated to be useful for the treatment of anxiolytic disorders.

The immunoreactivity of NPY is notably decreased in the cerebrospinal fluid of patients with major depression and those of suicide victims (Widdowson, P. S. et al. J. Neurochem. 1992, 59, 73), and rats treated with tricyclic antidepressants displayed significant increases of NPY relative to a control group (Heilig, M. et al. Eur. J. Pharmacol. 1988, 147, 465). These findings suggest that an inadequate NPY response may play a role in some depressive illnesses, and that compounds that regulate the NPY-ergic system may be useful for the treatment of depression.

It is known that the anxiolytic properties of NPY are mediated through postsynaptic Y1 receptors, whereas presynaptic Y2 receptors negatively control the release of NPY and other cotransmitters (e.g. GABA). Consequently, antagonism of the Y2 receptor may lead to enhanced GABAergic and NPYergic effects and Y2 receptor antagonists should prove useful in the treatment of depression and anxiety.

NPY improved memory and performance scores in animal models of learning (Flood, J. F. et al. Brain Res. 1987, 421, 280) and therefore may serve as a cognition enhancer for the treatment of neurodegenerative diseases such as Alzheimer's Disease (AD) as well as AIDS-related and senile dementia.

Elevated plasma levels of NPY were present in animals and humans experiencing episodes of high sympathetic nerve activity such as surgery, newborn delivery, and hemorrhage (Morris, M. J. et. al. J. Auton. Nerv. Syst. 1986, 17, 143). Thus, chemical substances that alter the NPY-ergic system may be useful for alleviating migraine, pain, and the condition of stress.

NPY also mediates endocrine functions such as the release of luteinizing hormone (LH) in rodents (Kalra, S. P. et. al. Front. Neuroendrocrinol. 1992, 13, 1). Since LH is vital for mammalian ovulation, a compound that mimics the action of NPY could be useful for the treatment of infertility, particularly in women with so-called luteal phase defects.

NPY is a powerful stimulant of food intake; as little as one-billionth of a gram, when injected directly into the CNS, caused satiated rats to overeat (Clark, J. T. et al. Endocrinology 1984, 115, 427; Levine, A. S. et al. Peptides 1984, 5, 1025; Stanley, B. G. et al. Life Sci. 1984, 35, 2635; Stanley, B. G. et al. Proc. Nat. Acad. Sci. U.S.A. 1985, 82, 3940). Thus NPY is orexigenic in rodents but not anxiogenic when given intracerebroventricularly and so antagonism of neuropeptide receptors may be useful for the treatment of diabetes and eating disorders such as obesity, anorexia nervosa, and bulimia nervosa.

Recently, a key role of presynaptic hypothalamic Y2 receptor was suggested in central coordination of energy homeostasis and bone mass regulation (Herzog, H. et al. Drug News & Perspectives 2002, 15, 506-510). Studies analyzing Y2 receptor knockout mice have started to unravel some of the individual functions of this receptor subtype. Y2 receptor knockout mice showed a reduced body weight despite an increase in food intake, possibly due to the lack of the feedback inhibition of the postprandially released PYY₃₋₃₆ (Batterham, R. L. et al. Nature 2002, 418, 650-654). The Y2 receptor knockout mice also showed a significant increase in bone formation (Baldock, P. A. J. Clin. Invest. 2002, 109, 915-921). Specific deletion of the Y2 receptor in the hypothalamus in adult conditional Y2 receptor knockout mice also caused an increase in bone formation.

Studies have also indicates that NPY Y2 is involved in the neurobiological responses to ethanol and other drugs of abuse. Thiele and coworkers (Neuropeptides, 2004, 38(4), 235-243; Peptides 2004, 25(6), 975-983) described the low ethanol consumption of Y2 receptor knockout mice, as well as their increased voluntary water consumption. Therefore, modulators of NPY Y2 may allow for the treatment of alcohol and drug abuse.

Grouzmann and coworkers described a peptide-based ligand, T4-[NPY 33-36], which showed considerable affinity (IC₅₀=67 nM) for the NPY Y2 receptor (Grouzmann, E., et al. J. Biol. Chem. 1997, 272, 7699-7706). BIIE0246 also bound to the NYP Y2 receptor with significant affinity (IC₅₀=3.3 nM) (Doods, H., et al. Eur. J. Pharmacol. 1999, 384, R3-R5). However, the therapeutic potential for these compounds is limited due to their peptide-like composition and elevated molecular weight.

There remains however, a need for potent NPY Y2 modulators with desirable pharmaceutical properties.

### SUMMARY OF THE INVENTION

The present invention is directed to piperazinyl derivatives, compounds of formula (II)

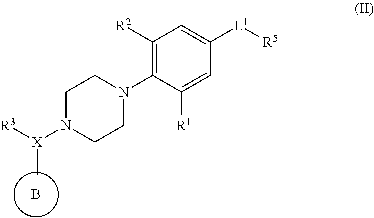

wherein

R¹ and R² are each independently selected from the group consisting of hydrogen, halogen, C₁₋₄alkyl, —C₁₋₄alkyl-OH, —C₁₋₄alkyl-O—C₁₋₄alkyl, —C₁₋₄alkoxy, —S—C₁₋₄alkyl, —SO—C₁₋₄alkyl, —SO₂—C₁₋₄alkyl, cyano, nitro, —NR^{A}R^{B}, —CH₂—NR^{A}R^{B}, —C(O)—NR^{A}R^{B} and —C(O)H; wherein R^{A} and R^{B} are each independently selected from the group consisting of hydrogen and C₁₋₄alkyl;

provided that at least one of R¹ or R² is other than hydrogen;

L¹ is selected from the group consisting of —NR^{J}—, —NR^{J}—C(O)—, —C(O)—NR^{J}—, —(CH₂)ₐ—C(O)—NR^{J}—, —(CH₂)ₐ—NR^{J}—C(O)— and —C(O)O—; wherein R^{J} is selected from the group consisting of hydrogen and C₁₋₄alkyl; and wherein a is an integer from 1 to 3;

R⁵ is selected from the group consisting of C₁₋₈alkyl, C₃₋₈cycloalkyl, aryl, heteroaryl, and heterocycloalkyl; wherein the C₁₋₈alkyl, C₃₋₈cycloalkyl, aryl, heteroaryl or heterocycloalkyl is optionally substituted with one or more substituents independently selected from the group consisting of halogen, C₁₋₄alkyl, halogenated C₁₋₄alkyl, C₁₋₄alkoxy, halogenated C₁₋₄alkoxy, hydroxy, cyano, nitro and NR^{K}R^{L}; wherein R^{K} and R^{L} are each independently selected from the group consisting of hydrogen and C₁₋₄alkyl;

X is selected from the group consisting of CH and CR¹⁰; wherein R¹⁰ is selected from the group consisting of —C₁₋₄alkyl;

R³ is selected from the group consisting of cyano, C₁₋₄alkyl, C₂₋₄alkenyl, C₃₋₈cycloalkyl, aryl, C₁₋₄aralkyl, and 5 to 6 membered heteroaryl; wherein the aryl or heteroaryl, whether alone or as part of a substituent group is optionally substituted with one or more substituents independently selected from the group consisting of halogen, hydroxy, C₁₋₄alkyl, halogenated C₁₋₄alkyl, C₁₋₄alkoxy, halogenated C₁₋₄alkoxy, cyano, nitro, NR^{E}R^{F} and —C(O)—NR^{E}R^{F}; wherein R^{E} and R^{F} are each independently selected from the group consisting of hydrogen and C₁₋₄alkyl;

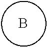

is selected from the group consisting of cycloalkyl, aryl, heteroaryl, heterocycloalkyl, —C(O)—C₁₋₄alkyl, —C(O)-aryl, and —C(O)-aryl; wherein the cycloalkyl, aryl, heteroaryl or heterocycloalkyl is optionally substituted with one or more substituents independently selected from the group consisting of halogen, C₁₋₈alkyl, halogenated C₁₋₄alkyl, C₁₋₄alkoxy, cyano, oxo, —C(O)OH, —C(O)O—C₁₋₄alkyl, —C(O)—NR^{C}R^{D}, —C(O)—NR^{E}—NR^{C}R^{D}, C₃₋₈cycloalkyl, aryl, heteroaryl and heterocycloalkyl;

wherein R^{C} and R^{D} are each independently selected from the group consisting of hydrogen and C₁₋₄alkyl; alternatively, R^{C} and R^{D} are taken together with the nitrogen atom to which they are bound to form a 4 to 8 membered saturated ring structure; and wherein R^{E} is selected from the group consisting of hydrogen and C₁₋₄alkyl;

provided that when R¹ is fluoro, R² is hydrogen, X is CH, R³ is phenyl, L^{1 is —CH}₂—C(O)—N(CH₃)— and R⁵ is ethyl, then

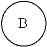

is not isopropyl-carbonyl;

provided further that when R¹ is fluoro, R² is hydrogen, X is CH, R³ is phenyl, L¹ is —NH—C(O)— and R⁵ is isopropyl, then

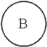

is not phenyl-carbonyl;

provided further that when R¹ is nitro or amino, R² is hydrogen, X is CH, R³ is phenyl or 4-fluoro-phenyl, L¹ is —C(O)O— and R⁵ is methyl; then

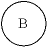

is other than phenyl or 4-fluoro-phenyl;

provided further that when R¹ fluoro, R² is hydrogen, X is CH, R³ is phenyl, L¹ is —NH—C(O)— and R⁵ is isopropyl, then

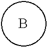

is other than 1-pyrrolidinyl;

and enantiomers and pharmaceutically acceptable salts thereof.

Illustrative of the invention is a pharmaceutical composition comprising a pharmaceutically acceptable carrier and any of the compounds described herein. An illustration of the invention is a pharmaceutical composition made by mixing any of the compounds described herein and a pharmaceutically acceptable carrier. Illustrating the invention is a process for making a pharmaceutical composition comprising mixing any of the compounds described herein and a pharmaceutically acceptable carrier.

Exemplifying the invention are methods of treating a disorder mediated by the neuropeptide Y₂ receptor (selected from the group consisting of anxiolytic disorders, depression; pain, injured mammalian nerve tissue; conditions responsive to treatment with a neurotrophic factor; neurological disorders; bone loss; cardiovascular diseases; sleep-wake state disorders, substance abuse and addiction related disorders; obesity; obesity-related disorders, disorders responsive to modulation of endocrine function, inovulation and infertility; comprising administering to a subject in need thereof a therapeutically effective amount of any of the compounds or pharmaceutical compositions described above.

Another example of the invention is the use of any of the compounds described herein in the preparation of a medicament for treating: (a) anxiolytic disorders, (b) depression; (c) pain, (d) injured mammalian nerve tissue; (d) conditions responsive to treatment with a neurotrophic factor; (e) neurological disorders; (f) bone loss; (g) cardiovascular diseases; (h) sleep-wake state disorders, (i) substance abuse and addiction related disorders; (j) obesity; (k) obesity-related disorders, (l) disorders responsive to modulation of endocrine function (more particularly, disorders responsive to modulation of the pituitary and/or hypothalamic gland); (m) inovulation; and (n) infertility; in a subject in need thereof.

### DETAILED DESCRIPTION OF THE INVENTION

The present invention is directed to compounds of formula (II)

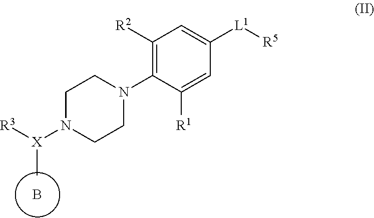

wherein R¹, R², L¹, R⁵, X, R³, and

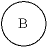

are as herein defined and enantiomers and pharmaceutically acceptable salts thereof. The compounds of the present invention are modulators of the NPY Y₂ receptor, useful in the treatment of disorders and conditions including, but not limited to anxiolytic disorders, depression; pain, injured mammalian nerve tissue; conditions responsive to treatment with a neurotrophic factor; neurological disorders; bone loss; cardiovascular diseases; sleep-wake state disorders, substance abuse and addiction related disorders; obesity; obesity-related disorders, disorders responsive to modulation of endocrine function, inovulation and infertility.

The compounds of formula (II) are preferably, useful for the treatment of disorders or conditions mediated by the NPY Y₂ receptor, selected from the group consisting of substance abuse (more preferably alcohol abuse), anxiolytic disorders (more preferably anxiety), bone loss, obesity and obesity-related disorders. More preferably, the compounds of formula (II) are useful in the treatment of anxiety and alcohol abuse.

In an embodiment of the present invention, R¹ and R² are each independently selected from the group consisting of hydrogen, halogen, C₁₋₄alkyl, —C₁₋₄alkyl-OH, —C₁₋₄alkoxy, —S—C₁₋₄alkyl, —SO—C₁₋₄alkyl, —SO₂—C₁₋₄alkyl, cyano, nitro and —NR^{A}R^{B}; wherein R^{A} and R^{B} are each independently selected from the group consisting of hydrogen and C₁₋₂alkyl; provided that at least one of R¹ or R² is other than hydrogen. In another embodiment of the present invention, R¹ and R² are each independently selected from the group consisting of hydrogen, halogen, C₁₋₄alkyl and cyano; provided that at least one of R¹ or R² is other than hydrogen.

In an embodiment of the present invention, R¹ is selected from the group consisting of fluoro, bromo, methyl and cyano. In another embodiment of the present invention, R¹ is selected from the group consisting of fluoro, bromo, methyl and cyano. In another embodiment of the present invention, R¹ is selected from the group consisting of fluoro, bromo and cyano. In another embodiment of the present invention, R¹ is cyano.

In an embodiment of the present invention, R² is selected from the group consisting of hydrogen and halogen. In another embodiment of the present invention, R² is selected from the group consisting of hydrogen and fluoro. In another embodiment of the present invention, R² is hydrogen.

In an embodiment of the present invention, L¹ is selected from the group consisting of —NR^{J}—, —NR^{J}—C(O)—, —(CH₂)ₐ—NR^{J}—C(O)—, —C(O)—NR^{J}— and —(CH₂)ₐ—C(O)—NR^{J}—; wherein R^{J} is selected from the group consisting of hydrogen and C₁₋₄alkyl; and wherein a is an integer from 1 to 3. In another embodiment of the present invention, L¹ is selected from the group consisting of —NR^{J}—, —NR^{S}—C(O)—, —(CH₂)ₐ—NR^{J}—C(O)—, —C(O)—NR^{J}— and —(CH₂)ₐ—C(O)—NR^{J}—; wherein R^{J} is selected from the group consisting of hydrogen and C₁₋₂alkyl; and wherein a is an integer from 1 to 2. In another embodiment of the present invention, L¹ is selected from the group consisting of —NH—, —NH—C(O)—, —CH₂—NH—C(O)—, —C(O)—NH— and —CH₂—C(O)—N(ethyl)-. In another embodiment of the present invention, L¹ is —NH—C(O)—.

In an embodiment of the present invention, R⁵ is selected from the group consisting of C₁₋₆alkyl, C₃₋₈cycloalkyl, aryl, heteroaryl, and heterocycloalkyl; wherein the C₁₋₆alkyl, C₃₋₈cycloalkyl, aryl, heteroaryl or heterocycloalkyl is optionally substituted with one or more substituents independently selected from the group consisting of halogen, C₁₋₄alkyl, fluorinated C₁₋₄alkyl, C₁₋₄alkoxy, fluorinated C₁₋₄alkoxy, hydroxy, cyano, nitro and NR^{K}R^{L}; wherein R^{K} and R^{L} are each independently selected from the group consisting of hydrogen and C₁₋₄alkyl. In another embodiment of the present invention, R⁵ is selected from the group consisting of C₁₋₆alkyl, C₃₋₈cycloalkyl, aryl, heteroaryl and heterocycloalkyl; wherein the C₃₋₈cycloalkyl, aryl, heteroaryl or heterocycloalkyl is optionally substituted with one to three substituents independently selected from the group consisting of halogen, C₁₋₄alkyl and NR^{K}R^{L}; wherein R^{K} and R^{L} are each independently selected from the group consisting of hydrogen and C₁₋₂alkyl.

In another embodiment of the present invention, R⁵ is selected from the group consisting of methyl, ethyl, 2-n-propyl, isopropyl, n-butyl, 3-n-pentyl, 1-(1-(R)-methyl-n-propyl), 1-(1-methyl-3,3,3-trifluoro-n-propyl), cyclopropyl, cyclobutyl, cylopentyl, cyclohexyl, 4,4-difluoro-cyclohexyl, 2-methyl-phenyl, 3-(S)-tetrahydrofuranyl, 3-(R)-tetrahydrofuranyl, 2-(3-methyl-pyridyl), 2-(6-methyl-pyridyl), 2-(1-methyl-imidazolyl), 2-(4-methyl-pyrimidinyl) and 4-(3,5-dimethyl-isoxazolyl). In another embodiment of the present invention, R⁵ is selected from the group consisting of methyl, ethyl, 2-n-propyl, isopropyl, n-butyl, 3-n-pentyl, 1-(1-(R)-methyl-n-propyl), 1-(1-methyl-3,3,3-trifluoro-n-propyl), dimethylamino-methyl-, cyclopropyl, cyclobutyl, cylopentyl, cyclohexyl, 4,4-difluoro-cyclohexyl, 2-methyl-phenyl, 3-tetrahydrofuranyl, 3-(S)-tetrahydrofuranyl, 3-(R)-tetrahydrofuranyl, 2-(3-methyl-pyridyl), 2-(6-methyl-pyridyl), 2-(1-methyl-imidazolyl), 2-(4-methyl-pyrimidinyl) and 4-(3,5-dimethyl-isoxazolyl). In another embodiment of the present invention, R⁵ is selected from the group consisting of isopropyl, 3-n-pentyl, 1-(1-(R)-methyl-n-propyl), 1-(1-methyl-3,3,3-trifluoro-n-propyl), cyclopropyl, cyclobutyl, 3-(S)-tetrahydrofuranyl and 3-(R)-tetrahydrofuranyl. In another embodiment of the present invention, R⁵ is selected from the group consisting of 3-n-pentyl, 1-(1-(R)-methyl-n-propyl), 1-(1-methyl-3,3,3-trifluoro-n-propyl) and 3-(R)-tetrahydrofuranyl. In another embodiment of the present invention, R⁵ is selected from the group consisting of 3-n-pentyl, 1-(1-(R)-methyl-n-propyl), 1-(1-methyl-3,3,3-trifluoro-n-propyl), cyclopropyl, 3-(R)-tetrahydrofuranyl and 4-(3,5-dimethyl-isoxazolyl). In another embodiment of the present invention, R⁵ is selected from the group consisting of 3-n-pentyl, cyclopropyl, and 4-(3,5-dimethyl-isoxazolyl). In another embodiment of the present invention, R⁵ is 3-n-pentyl.

In an embodiment of the present invention, X is selected from the group consisting of CH and CR¹⁰; wherein R¹⁰ is selected from the group consisting of —C₁₋₂alkyl. In another embodiment of the present invention, X is CH.

In an embodiment of the present invention, R³ is selected from the group consisting of cyano, C₁₋₄alkyl, C₃₋₈cycloalkyl, aryl and 5 to 6 membered heteroaryl; wherein the aryl or heteroaryl, whether alone or as part of a substituent group is optionally substituted with one or more substituents independently selected from the group consisting of halogen, hydroxy, C₁₋₄alkyl, halogenated C₁₋₄alkyl, C₁₋₄alkoxy, halogenated C₁₋₄alkoxy, cyano, nitro, NR^{E}R^{F} and —C(O)—NR^{E}R^{F}; wherein R^{E} and R^{F} are each independently selected from the group consisting of hydrogen and C₁₋₄alkyl. In another embodiment of the present invention, R³ is selected from the group consisting of cyano, C₃₋₈cycloalkyl, aryl and 5 to 6 membered heteroaryl; wherein the aryl is optionally substituted with a substituent selected from the group consisting of halogen, C₁₋₄alkoxy and cyano. In another embodiment of the present invention, R³ is selected from the group consisting of cyano, C₃₋₈cycloalkyl, aryl and 5 to 6 membered heteroaryl; wherein the aryl is optionally substituted with a substituent selected from the group consisting of halogen, C₁₋₄alkoxy, fluorinated C₁₋₄alkoxy and cyano.

In another embodiment of the present invention, R³ is selected from the group consisting of cyano, cyclopropyl, cyclohexyl, phenyl, (R)-phenyl, (S)-phenyl, 4-fluorophenyl, 3-fluorophenyl, 4-chlorophenyl, 4-methoxy-phenyl, 4-cyano-phenyl, 2-pyridyl and 2-oxazolyl. In another embodiment of the present invention, R³ is selected from the group consisting of cyano, cyclopropyl, cyclohexyl, phenyl, (R)-phenyl, (S)-phenyl, 4-fluorophenyl, 3-fluorophenyl, 4-chlorophenyl, 4-methoxy-phenyl, 4-cyano-phenyl, 4-trifluoromethoxyphenyl, 2-pyridyl and 2-oxazolyl. In another embodiment of the present invention, R³ is selected from the group consisting of cyano, cyclopropyl, cyclohexyl, phenyl, (R)-phenyl, (S)-phenyl, 4-fluorophenyl, 3-fluorophenyl, 4-chlorophenyl, 4-methoxy-phenyl, 4-cyano-phenyl, 4-trifluoromethoxyphenyl, 2-pyridyl, 3-pyridyl, 4-pyridyl and 2-oxazolyl. In another embodiment of the present invention, R³ is selected from the group consisting of phenyl, (R)-phenyl, (S)-phenyl, 4-fluorophenyl, 3-fluorophenyl, 4-methoxy-phenyl, 4-cyano-phenyl and 2-pyridyl. In another embodiment of the present invention, R³ is selected from the group consisting of phenyl, (R)-phenyl, (S)-phenyl, 4-fluorophenyl, 3-fluorophenyl, 4-methoxy-phenyl, 4-cyano-phenyl, 4-trifluoromethoxyphenyl, and 2-pyridyl. In another embodiment of the present invention, R³ is selected from the group consisting of phenyl, (S)-phenyl, 4-fluorophenyl, 3-fluorophenyl, 4-methoxy-phenyl and 4-cyano-phenyl. In another embodiment of the present invention, R³ is selected from the group consisting of phenyl, (R)-phenyl, (S)-phenyl, 4-fluorophenyl, 3-fluorophenyl, 4-methoxy-phenyl and 4-cyano-phenyl. In another embodiment of the present invention, R³ is selected from the group consisting of phenyl and 3-fluorophenyl. In another embodiment of the present invention, R³ is phenyl.

In an embodiment of the present invention,

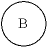

is selected from the group consisting of cycloalkyl, aryl, heteroaryl, heterocycloalkyl, —C(O)—C₁₋₄alkyl, —C(O)-aryl, and —C(O)-aryl; wherein the cycloalkyl, aryl, heteroaryl or heterocycloalkyl is optionally substituted with one or more substituents independently selected from the group consisting of halogen, C₁₋₆alkyl, fluorinated C₁₋₄alkyl, C₁₋₄alkoxy, cyano, C₃₋₈cycloalkyl, aryl, heteroaryl and heterocycloalkyl. In another embodiment of the present invention,

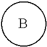

is selected from the group consisting of cycloalkyl, aryl, heteroaryl, heterocycloalkyl and —C(O)—C₁₋₄alkyl; wherein the cycloalkyl, aryl, heteroaryl and heterocycloalkyl is optionally substituted with one to two substituents independently selected from halogen, cyano, C₁₋₆alkyl, fluorinated C₁₋₄alkyl, C₁₋₄alkoxy, —C(O)O—C₁₋₄alkyl, phenyl and 5 to 6 membered heteroaryl.

In another embodiment of the present invention,

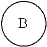

is selected from the group consisting of methylcarbonyl-, cyclopropyl, cyclobutyl, cyclopentyl, 1-(2,2-dichloro-3-methyl-cyclopropyl), phenyl, 4-fluorophenyl, 2-methoxy-phenyl, 2-cyano-phenyl, 3-tetrahydrofuranyl, 2-furyl, 2-pyridyl, 3-pyridyl, 2-thienyl, 2-thiazolyl, 2-pyrimidinyl, 2-(1-methyl-imidazolyl), 2-benzoxazolyl, 2-benzthiazolyl, 5-(3-methyl-isoxazolyl), 2-(4,5-dihydro-4-methoxycarbonyl-oxazolyl), 2-oxazolyl, 2-(5-methyl-[1,3,4]-oxadiazolyl), 2-(5-ethyl-[1,3,4]-oxadiazolyl), 2-(5-isopropyl-[1,3,4]-oxadiazolyl), 2-(5-(3-n-pentyl)-[1,3,4]-oxadiazolyl), 5-(3-methyl-[1,2,4]-oxadiazolyl), 5-(3-ethyl-[1,2,4]-oxadiazolyl), 5-(3-isopropyl-[1,2,4]-oxadiazolyl), 3-(5-isopropyl-[1,2,4]-oxadiazolyl), 3-(5-methyl-[1,2,4]-oxadiazolyl), 3-(5-fluoromethyl-[1,2,4]-oxadiazolyl), 3-(5-trifluoromethyl-[1,2,4]-oxadiazolyl), 3-(5-ethyl-[1,2,4]-oxadiazolyl), 3-(5-phenyl-[1,2,4]-oxadiazolyl), and 3-(5-(3-pyridyl)-[1,2,4]-oxadiazolyl).

In another embodiment of the present invention,

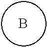

is selected from the group consisting of methylcarbonyl-, cyclopropyl, cyclobutyl, cyclopentyl, 1-(2,2-dichloro-3-methyl-cyclopropyl), phenyl, 4-fluorophenyl, 4-chlorophenyl, 2-methylphenyl, 2-methoxy-phenyl, 2-cyano-phenyl, 3-tetrahydrofuranyl, 2-furyl, 2-pyridyl, 3-pyridyl, 4-pyridyl, 2-thienyl, 2-thiazolyl, 2-pyrimidinyl, 2-(1-methyl-imidazolyl), 2-benzoxazolyl, 2-benzthiazolyl, 5-(3-methyl-isoxazolyl), 2-(4,5-dihydro-4-methoxycarbonyl-oxazolyl), 2-oxazolyl, 2-(5-methyl-[1,3,4]-oxadiazolyl), 2-(5-ethyl-[1,3,4]-oxadiazolyl), 2-(5-isopropyl-[1,3,4]-oxadiazolyl), 2-(5-(3-n-pentyl)[1,3,4]-oxadiazolyl), 5-(3-methyl-[1,2,4]-oxadiazolyl), 5-(3-ethyl-[1,2,4]-oxadiazolyl), 5-(3-isopropyl-[1,2,4]-oxadiazolyl), 3-(5-isopropyl-[1,2,4]-oxadiazolyl), 3-(5-methyl-[1,2,4]-oxadiazolyl), 3-(5-fluoromethyl-[1,2,4]-oxadiazolyl), 3-(5-trifluoromethyl-[1,2,4]-oxadiazolyl), 3-(5-ethyl-[1,2,4]-oxadiazolyl), 3-(5-phenyl-[1,2,4]-oxadiazolyl), and 3-(5-(3-pyridyl)-[1,2,4]-oxadiazolyl).

In another embodiment of the present invention,

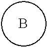

is selected from the group consisting of cyclopropyl, cyclobutyl, phenyl, 2-pyridyl, 3-pyridyl, 2-thiazolyl, 2-pyrimidinyl, 2-benzoxazolyl, 2-benzthiazolyl, 5-(3-methyl-isoxazolyl), 2-(4,5-dihydro-4-methoxycarbonyl-oxazolyl), 2-oxazolyl, 2-(5-methyl-[1,3,4]-oxadiazolyl), 2-(5-ethyl-[1,3,4]-oxadiazolyl), 2-(5-isopropyl-[1,3,4]-oxadiazolyl), 2-(5-(3-n-pentyl)[1,3,4]-oxadiazolyl), 5-(3-methyl-[1,2,4]-oxadiazolyl), 5-(3-ethyl-[1,2,4]-oxadiazolyl), 5-(3-isopropyl-[1,2,4]-oxadiazolyl), 3-(5-isopropyl-[1,2,4]-oxadiazolyl), 3-(5-methyl-[1,2,4]-oxadiazolyl), 3-(5-fluoromethyl-[1,2,4]-oxadiazolyl), 3-(5-ethyl-[1,2,4]-oxadiazolyl), 3-(5-phenyl-[1,2,4]-oxadiazolyl), and 3-(5-(3-pyridyl)[1,2,4]-oxadiazolyl). In another embodiment of the present invention,

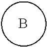

is selected from the group consisting of phenyl, 2-pyridyl, 3-pyridyl, 2-thiazolyl, 2-pyrimidinyl, 2-benzoxazolyl, 2-benzthiazolyl, 5-(3-methyl-isoxazolyl), 2-(4,5-dihydro-4-methoxycarbonyl-oxazolyl), 2-oxazolyl, 2-(5-methyl-[1,3,4]-oxadiazolyl), 2-(5-ethyl-[1,3,4]-oxadiazolyl), 2-(5-isopropyl-[1,3,4]-oxadiazolyl), 2-(5-(3-n-pentyl)-[1,3,4]-oxadiazolyl), 5-(3-methyl-[1,2,4]-oxadiazolyl), 5-(3-ethyl-[1,2,4]-oxadiazolyl), 5-(3-isopropyl-[1,2,4]-oxadiazolyl), 3-(5-isopropyl-[1,2,4]-oxadiazolyl), 3-(5-methyl-[1,2,4]-oxadiazolyl), 3-(5-fluoromethyl-[1,2,4]-oxadiazolyl), 3-(5-ethyl-[1,2,4]-oxadiazolyl), 3-(5-phenyl-[1,2,4]-oxadiazolyl), and 3-(5-(3-pyridyl)[1,2,4]-oxadiazolyl). In another embodiment of the present invention,

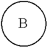

is selected from the group consisting of 2-pyridyl, 2-benzoxazolyl, 5-(3-methyl-isoxazolyl), 2-oxazolyl, 2-(5-(3-n-pentyl)-[1,3,4]-oxadiazolyl) and 3-(5-methyl-[1,2,4]-oxadiazolyl). In another embodiment of the present invention,

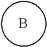

is selected from the group consisting of phenyl, 2-pyridyl, 3-pyridyl, 2-thiazolyl, 2-pyrimidinyl, 2-benzoxazolyl, 2-benzthiazolyl, 5-(3-methyl-isoxazolyl), 2-oxazolyl, 2-(5-methyl-[1,3,4]-oxadiazolyl), 2-(5-ethyl-[1,3,4]-oxadiazolyl), 2-(5-isopropyl-[1,3,4]-oxadiazolyl), 2-(5-(3-n-pentyl)-[1,3,4]-oxadiazolyl), 5-(3-methyl-[1,2,4]-oxadiazolyl), 5-(3-ethyl-[1,2,4]-oxadiazolyl), 3-(5-isopropyl-[1,2,4]-oxadiazolyl), 3-(5-methyl-[1,2,4]-oxadiazolyl), 345-fluoromethyl-[1,2,4]-oxadiazolyl), 3-(5-ethyl-[1,2,4]-oxadiazolyl), 3-(5-phenyl-[1,2,4]-oxadiazolyl), and 3-(5-(3-pyridyl)[1,2,4]-oxadiazolyl).

In another embodiment of the present invention,

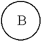

is selected from the group consisting of phenyl, 2-methyl-phenyl, 2-pyridyl, 3-pyridyl, 4-pyridyl, 2-thiazolyl, 2-pyrimidinyl, 2-benzoxazolyl, 2-benzthiazolyl, 5-(3-methyl-isoxazolyl), 2-oxazolyl, 2-(5-methyl-[1,3,4]-oxadiazolyl), 2-(5-ethyl-[1,3,4]-oxadiazolyl), 2-(5-isopropyl-[1,3,4]-oxadiazolyl), 2-(5-(3-n-pentyl)-[1,3,4]-oxadiazolyl), 5-(3-methyl-[1,2,4]-oxadiazolyl), 5-(3-ethyl-[1,2,4]-oxadiazolyl), 3-(5-isopropyl-[1,2,4]-oxadiazolyl), 3-(5-methyl-[1,2,4]-oxadiazolyl), 3-(5-fluoromethyl-[1,2,4]-oxadiazolyl), 3-(5-ethyl-[1,2,4]-oxadiazolyl), 3-(5-phenyl-[1,2,4]-oxadiazolyl), and 3-(5-(3-pyridyl)[1,2,4]-oxadiazolyl). In another embodiment of the present invention,

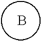

is selected from the group consisting of phenyl, 2-methyl-phenyl, 4-chlorophenyl, 3-pyridyl and 4-pyridyl.

Additional embodiments of the present invention, include those wherein the substituents selected for one or more of the variables defined herein (e.g. R¹, R², X, R³, L¹, R⁵, etc.) are independently selected to be any individual substituent or any subset of substituents selected from the complete list as defined herein. In another embodiment of the present invention is any single compound or subset of compounds selected from the representative compounds listed in Tables 1-2 below.

Representative compounds of formula (II) of the present invention are as listed in Tables 1 and 2 below. Unless otherwise noted, wherein a stereogenic center is present in the listed compound, the compound was prepared as a mixture of stereo-configurations. Where a stereogenic center is present, the (S) and (R) designations are intended to indicate that the exact stereo-configuration of the center has not been determined.

TABLE 1
Representative Compounds of Formula (II)

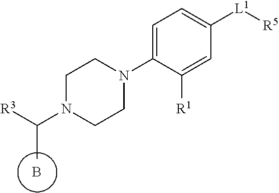

| Cmpd. No. | R¹ | L¹ | R⁵ | R³ | 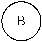 |
|---|---|---|---|---|---|
| 1 | F | —NH—C(O)— | 3-n-pentyl | phenyl | 5-(3-ethyl- |
| | | | | | [1,2,4]- |
| | | | | | oxadiazolyl |
| 2 | F | —NH—C(O)— | 3-n-pentyl | phenyl | 5-(3-methyl- |
| | | | | | [1,2,4]- |
| | | | | | oxadiazolyl |
| 4 | F | —NH—C(O)— | 3-n-pentyl | phenyl | phenyl |
| 12 | F | —NH—C(O)— | 3-n-pentyl | 4-fluoro- | 4-fluoro-phenyl |
| | | | | phenyl | |
| 13 | F | —NH—C(O)— | 3-n-pentyl | 4-chloro- | phenyl |
| | | | | phenyl | |
| 14 | F | —NH—C(O)— | 3-(S)- | phenyl | phenyl |
| | | | tetrahydro- | | |
| | | | furanyl | | |
| 20 | F | —NH—C(O)— | 3-n-pentyl | phenyl | 2-oxazolyl |
| 24 | F | —NH—C(O)— | 3-n-pentyl | phenyl | 3-pyridyl |
| 27 | F | —NH—C(O)— | 3-n-pentyl | phenyl | 2-thienyl |
| 33 | F | —NH—C(O)— | 3-n-pentyl | phenyl | 2-pyridyl |
| 39 | F | —NH—C(O)— | 3-n-pentyl | phenyl | 2-thiazolyl |
| 40 | F | —NH—C(O)— | 3-n-pentyl | phenyl | 2-pyrimidinyl |
| 46 | F | —C(O)—NH— | 3-n-pentyl | phenyl | 2-oxazolyl |
| 49 | F | —C(O)—NH— | cyclopentyl | phenyl | 2-oxazolyl |
| 51 | F | —CH₂—C(O)—N(ethyl)- | ethyl | phenyl | phenyl |
| 53 | F | —NH—C(O)— | 3-n-pentyl | phenyl | cyclopropyl |
| 54 | F | —NH—C(O)— | cyclopropyl | phenyl | 2-oxazolyl |
| 55 | F | —NH—C(O)— | 3-n-pentyl | 2-pyridyl | 2-pyridyl |
| 57 | F | —NH— | 2-(3-methyl- | phenyl | 2-oxazolyl |
| | | | pyridyl) | | |
| 58 | F | —NH— | 2-(1-methyl- | phenyl | 2-oxazolyl |
| | | | imidazolyl) | | |
| 59 | F | —NH—C(O)— | 3-n-pentyl | (R)-phenyl | 2-oxazolyl |
| 60 | F | —NH—C(O)— | 3-n-pentyl | (S)-phenyl | 2-oxazolyl |
| 61 | F | —NH— | 2-(4-methyl- | phenyl | 2-oxazolyl |
| | | | pyrimidinyl) | | |
| 62 | F | —NH— | 2-(6-methyl- | phenyl | 2-oxazolyl |
| | | | pyridyl) | | |
| 63 | F | —NH— | 2-methyl-phenyl | phenyl | 2-oxazolyl |
| 64 | F | —NH—C(O)— | 3-n-pentyl | phenyl | 2-(1-methyl- |
| | | | | | imidazolyl) |
| 66 | F | —NH—C(O)— | 3-n-pentyl | cyclopropyl | 2-oxazolyl |
| 72 | F | —NH—C(O)— | 2-n-propyl | phenyl | 2-oxazolyl |
| 73 | F | —NH—C(O)— | cyclobutyl | phenyl | 2-oxazolyl |
| 74 | methyl | —NH—C(O)— | 2-n-propyl | phenyl | 2-oxazolyl |
| 76 | F | —NH—C(O)— | cyclobutyl | phenyl | 2-oxazolyl |
| 78 | F | —NH—C(O)— | 3-n-pentyl | phenyl | cyclobutyl |
| 81 | methyl | —NH—C(O)— | cyclopropyl | phenyl | 2-oxazolyl |
| 82 | F | —NH—C(O)— | 3-n-pentyl | phenyl | 3-(5-isopropyl- |
| | | | | | [1,2,4]- |
| | | | | | oxadiazolyl) |
| 83 | F | —NH—C(O)— | 3-n-pentyl | phenyl | cyclopentyl |
| 84 | F | —NH—C(O)— | 3-n-pentyl | 4-methoxy- | cyclopropyl |
| | | | | phenyl | |
| 85 | methyl | —NH—C(O)— | 3-n-pentyl | phenyl | 2-oxazolyl |
| 86 | cyano | —NH—C(O)— | 3-n-pentyl | phenyl | 2-oxazolyl |
| 87 | cyano | —NH—C(O)— | isopropyl | phenyl | 2-oxazolyl |
| 90 | cyano | —NH—C(O)— | 3-(R)- | phenyl | 2-oxazolyl |
| | | | tetrahydro- | | |
| | | | furanyl | | |
| 91 | F | —NH—C(O)— | 1-(1-(R)-methyl- | phenyl | 2-oxazolyl |
| | | | n-propyl) | | |
| 92 | F | —NH—C(O)— | 3-(R)- | phenyl | 2-oxazolyl |
| | | | tetrahydro- | | |
| | | | furanyl | | |
| 93 | cyano | —NH—C(O)— | 1-(1-(R)-methyl- | phenyl | 2-oxazolyl |
| | | | n-propyl) | | |
| 95 | cyano | —NH—C(O)— | cyclobutyl | phenyl | 2-oxazolyl |
| 96 | cyano | —NH—C(O)— | 2-methyl-phenyl | phenyl | 2-oxazolyl |
| 98 | cyano | —NH—C(O)— | n-butyl | phenyl | 2-oxazolyl |
| 100 | F | —NH—C(O)— | n-butyl | phenyl | 2-oxazolyl |
| 101 | cyano | —NH—C(O)— | cyclopropyl | phenyl | 2-oxazolyl |
| 102 | F | —NH—C(O)— | 3-n-pentyl | 4-chloro- | cyclopropyl |
| | | | | phenyl | |
| 103 | cyano | —NH—C(O)— | methyl | phenyl | 2-oxazolyl |
| 104 | F | —NH—C(O)— | ethyl | phenyl | 2-oxazolyl |
| 106 | F | —NH—C(O)— | 3-n-pentyl | 4-fluoro- | cyclobutyl |
| | | | | phenyl | |
| 107 | F | —NH—C(O)— | cyclohexyl | phenyl | 2-oxazolyl |
| 108 | F | —NH—C(O)— | 4,4-difluoro- | phenyl | 2-oxazolyl |
| | | | cyclohexyl | | |
| 109 | cyano | —NH—C(O)— | 4,4-difluoro- | phenyl | 2-oxazolyl |
| | | | cyclohexyl | | |
| 110 | cyano | —NH—C(O)— | 1-(1-methyl- | phenyl | 2-oxazolyl |
| | | | 3,3,3-trifluoro-n- | | |
| | | | propyl) | | |
| 111 | F | —NH—C(O)— | 1-(1-methyl- | phenyl | 2-oxazolyl |
| | | | 3,3,3-trifluoro-n- | | |
| | | | propyl) | | |
| 112 | cyano | —NH—C(O)— | cyclohexyl | phenyl | 2-oxazolyl |
| 113 | F | —NH—C(O)— | 4-(3,5-dimethyl- | phenyl | 2-oxazolyl |
| | | | isoxazolyl) | | |
| 114 | F | —NH—C(O)— | 3-n-pentyl | phenyl | 3-tetrahydro- |
| | | | | | furanyl |
| 115 | F | —NH—C(O)— | 3-n-pentyl | phenyl | 1-(2,2-dichloro- |
| | | | | | 3-methyl- |
| | | | | | cyclopropyl) |
| 116 | F | —NH—C(O)— | 3-n-pentyl | 2-oxazolyl | cyclobutyl |
| 117 | F | —NH—C(O)— | 3-n-pentyl | 2-oxazolyl | cyclopentyl |
| 120 | F | —NH—C(O)— | 3-n-pentyl | 4-fluoro- | 2-oxazolyl |
| | | | | phenyl | |
| 122 | F | —NH—C(O)— | 3-n-pentyl | cyclohexyl | 2-oxazolyl |
| 126 | F | —NH—C(O)— | cyclopentyl | phenyl | 2-oxazolyl |
| 127 | F | —NH—C(O)— | 3-(R)- | 4-fluoro- | 2-oxazolyl |
| | | | tetrahydro- | phenyl | |
| | | | furanyl | | |
| 131 | cyano | —NH—C(O)— | 3-n-pentyl | 3-fluoro- | 2-oxazolyl |
| | | | | phenyl | |
| 132 | F | —NH—C(O)— | 3-n-pentyl | 3-fluoro- | 2-oxazolyl |
| | | | | phenyl | |
| 134 | F | —NH—C(O)— | 3-n-pentyl | 3-fluoro- | 2-(5-methyl- |
| | | | | phenyl | [1,3,4]- |
| | | | | | oxadiazolyl) |
| 135 | F | —NH—C(O)— | 3-n-pentyl | cyano | 2-furyl |
| 136 | F | —NH—C(O)— | 3-n-pentyl | cyano | 2-cyano-phenyl |
| 137 | F | —NH—C(O)— | 3-n-pentyl | cyano | 2-methoxy- |
| | | | | | phenyl |
| 138 | F | —NH—C(O)— | 3-n-pentyl | cyano | 2-pyridyl |
| 139 | cyano | —NH—C(O)— | 3-n-pentyl | 4-fluoro- | 2-oxazolyl |
| | | | | phenyl | |
| 140 | F | —NH—C(O)— | 3-n-pentyl | 4-methoxy- | 2-oxazolyl |
| | | | | phenyl | |
| 141 | cyano | —NH—C(O)— | 3-n-pentyl | 4-methoxy- | 2-oxazolyl |
| | | | | phenyl | |
| 145 | cyano | —NH—C(O)— | 3-n-pentyl | 4-cyano- | 2-oxazolyl |
| | | | | phenyl | |
| 146 | F | —NH—C(O)— | 3-n-pentyl | 4-cyano- | 2-oxazolyl |
| | | | | phenyl | |
| 153 | F | —NH—C(O)— | 3-n-pentyl | phenyl | 2-(5-ethyl- |
| | | | | | [1,3,4]- |
| | | | | | oxadiazolyl) |
| 155 | cyano | —NH—C(O)— | 3-n-pentyl | 2-pyridyl | 2-oxazolyl |
| 156 | F | —NH—C(O)— | 3-n-pentyl | phenyl | 2-oxazolyl |
| 157 | F | —NH—C(O)— | 3-n-pentyl | 4-chloro- | 2-oxazolyl |
| | | | | phenyl | |
| 160 | F | —NH—C(O)— | 3-n-pentyl | phenyl | 2-(5-isopropyl- |
| | | | | | [1,3,4]- |
| | | | | | oxadiazolyl) |
| 163 | F | —NH—C(O)— | 3-n-pentyl | phenyl | 2-(5-(3-n-pentyl)- |
| | | | | | [1,3,4]- |
| | | | | | oxadiazolyl) |
| 165 | F | —NH—C(O)— | 3-n-pentyl | phenyl | 2-benzoxazolyl |
| 166 | cyano | —NH—C(O)— | 3-n-pentyl | phenyl | 2-benzoxazolyl |
| 168 | F | —NH—C(O)— | 3-n-pentyl | phenyl | 2-benzthiazolyl |
| 169 | cyano | —NH—C(O)— | 3-n-pentyl | phenyl | 2-benzthiazolyl |
| 171 | F | —CH₂—NH—C(O)— | cyclopropyl | phenyl | 2-oxazolyl |
| 179 | F | —NH—C(O)— | 3-n-pentyl | phenyl | 2-(4,5-dihydro-4- |
| | | | | | methoxy- |
| | | | | | carbonyl- |
| | | | | | oxazolyl) |
| 230 | F | —NH—C(O)— | 3-n-pentyl | phenyl | 5-(3-methyl- |
| | | | | | isoxazolyl) |
| 231 | F | —NH—C(O)— | 3-n-pentyl | phenyl | 2-oxazolyl |
| 232 | F | —NH—C(O)— | 3-n-pentyl | phenyl | methyl-carbonyl- |
| 234 | F | —NH—C(O)— | 3-n-pentyl | phenyl | 3-(5-trifluoro- |
| | | | | | methyl-[1,2,4]- |
| | | | | | oxadiazolyl) |
| 235 | F | —NH—C(O)— | 3-n-pentyl | phenyl | 3-(5-fluoro- |
| | | | | | methyl-[1,2,4]- |
| | | | | | oxadiazolyl) |
| 236 | F | —NH—C(O)— | 3-n-pentyl | phenyl | 3-(5-ethyl- |
| | | | | | [1,2,4]- |
| | | | | | oxadiazolyl) |
| 237 | Br | —NH—C(O)— | 3-n-pentyl | phenyl | 2-oxazolyl |
| 238 | F | —NH—C(O)— | 3-n-pentyl | phenyl | 3-(5-(3-pyridyl)- |
| | | | | | [1,2,4]- |
| | | | | | oxadiazolyl) |
| 239 | F | —NH—C(O)— | 3-n-pentyl | phenyl | 3-(5-methyl- |
| | | | | | [1,2,4]- |
| | | | | | oxadiazolyl) |
| 240 | F | —NH—C(O)— | 3-n-pentyl | phenyl | 3-(5-phenyl- |
| | | | | | [1,2,4]- |
| | | | | | oxadiazolyl) |
| 241 | F | —NH—C(O)— | 3-n-pentyl | phenyl | 5-(3-isopropyl- |
| | | | | | [1,2,4]- |
| | | | | | oxadiazolyl) |
| 242 | F | —NH—C(O)— | 3-n-pentyl | phenyl | 5-(3-methyl- |
| | | | | | [1,2,4]- |
| | | | | | oxadiazolyl) |
| 280 | F | —NH—C(O)— | 3-tetrahydro- | 4-trifluoro- | 3-(5-methyl- |
| | | | furanyl | methoxy- | [1,2,4]- |
| | | | | phenyl | oxadiazolyl) |
| 281 | F | —NH—C(O)— | 3-n-pentyl | 4-trifluoro- | 3-(5-methyl- |
| | | | | methoxy- | [1,2,4]- |
| | | | | phenyl | oxadiazolyl) |
| 300 | cyano | —NH—C(O)— | dimethylamino- | phenyl | phenyl |
| | | | methyl- | | |
| 301 | cyano | —NH—C(O)— | 4-(3,5-dimethyl- | phenyl | 2-methyl-phenyl |
| | | | isoxazolyl) | | |
| 302 | cyano | —NH—C(O)— | 4-(3,5-dimethyl- | phenyl | phenyl |
| | | | isoxazolyl) | | |
| 304 | cyano | —NH—C(O)— | 3-n-pentyl | phenyl | phenyl |
| 305 | cyano | —NH—C(O)— | 3-n-pentyl | phenyl | 2-methyl-phenyl |
| 307 | cyano | —NH—C(O)— | 3-n-pentyl | 4-fluoro- | 4-fluoro-phenyl |
| | | | | phenyl | |
| 309 | cyano | —NH—C(O)— | 4-(3,5-dimethyl- | 4-fluoro- | 4-fluoro-phenyl |
| | | | isoxazolyl) | phenyl | |
| 311 | cyano | —NH—C(O)— | 3-n-pentyl | phenyl | 4-chloro-phenyl |
| 312 | cyano | —NH—C(O)— | 4-(3,5-dimethyl- | phenyl | 4-chloro-phenyl |
| | | | isoxazolyl) | | |
| 315 | cyano | —NH—C(O)— | 3-n-pentyl | phenyl | 4-pyridyl |
| 317 | cyano | —NH—C(O)— | 3-n-pentyl | phenyl | 3-pyridyl |
| 321 | cyano | —NH—C(O)— | cyclopropyl | phenyl | 3-pyridyl |
| 322 | cyano | —NH—C(O)— | cyclopropyl | phenyl | 4-pyridyl |

(Prophetic Examples)

| | | | | | |
|---|---|---|---|---|---|
| 330 | cyano | —NH—C(O)— | cyclopropyl | 4-pyridyl | 4-pyridyl |
| 331 | cyano | —NH—C(O)— | cyclopropyl | 3-pyridyl | 3-pyridyl |
| 332 | cyano | —NH—C(O)— | cyclopropyl | phenyl | 2-pyrimidinyl |
| 333 | cyano | —NH—C(O)— | cyclopropyl | 4-fluoro- | 4-pyridyl |
| | | | | phenyl | |
| 334 | cyano | —NH—C(O)— | cyclopropyl | 4-fluoro- | 3-pyridyl |
| | | | | phenyl | |
| 340 | cyano | —NH—C(O)— | 3-n-pentyl | 4-pyridyl | 4-pyridyl |
| 341 | cyano | —NH—C(O)— | 3-n-pentyl | 3-pyridyl | 3-pyridyl |
| 342 | cyano | —NH—C(O)— | 3-n-pentyl | phenyl | 2-pyrimidinyl |
| 343 | cyano | —NH—C(O)— | 3-n-pentyl | 4-fluoro- | 4-pyridyl |
| | | | | phenyl | |
| 344 | cyano | —NH—C(O)— | 3-n-pentyl | 4-fluoro- | 3-pyridyl |
| | | | | phenyl | |

Additional representative compounds of formula (II) are as listed in Table 2, below.

TABLE 2
Representative Compounds of Formula (II)

| Cmpd. | Structure |
|---|---|
| No. | |
| 243 | 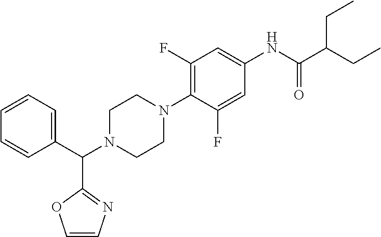 |

As used herein, “halogen” shall mean chlorine, bromine, fluorine and iodine.

As used herein, the term “alkyl” whether used alone or as part of a substituent group, include straight and branched chains. For example, alkyl radicals include methyl, ethyl, propyl, isopropyl, butyl, isobutyl, sec-butyl, t-butyl, pentyl and the like. Unless otherwise noted, “lower” when used with alkyl means a carbon chain composition of 1-4 carbon atoms.

As used herein, unless otherwise noted, the term “halogenated C₁₋₄alkyl” shall mean any C₁₋₄alkyl group as defined above substituted with at least one halogen atom, preferably substituted with a least one fluoro atom. Suitable examples include but are not limited to —CH₂F, —CF₃, —CCl₃, —CH₂—CF₃, —CH₂—CCl₃, —CF₂—CF₂—CF₂—CF₃, and the like. Similarly, the term “fluorinated C₁₋₄alkyl” shall mean any C₁₋₄alkyl group as defined above substituted with at least one fluoro atom. Suitable examples include but are not limited to —CH₂F, —CF₃, —CH₂—CF₃, —CF₂—CF₂—CF₂—CF₃, and the like.

As used herein, unless otherwise noted, the term “hydroxy substituted alkyl” shall mean alkyl group as defined above substituted with at least one hydroxy group. Preferably, the alkyl group is substituted with one hydroxy group. Preferably, the alkyl group is substituted with a hydroxy group at the terminal carbon. Suitable examples include, but are not limited to, —CH₂(OH), —CH₂—CH₂(OH), —CH₂—CH(OH)—CH₂, and the like.

As used herein, unless otherwise noted, “alkoxy” shall denote an oxygen ether radical of the above described straight or branched chain alkyl groups. For example, methoxy, ethoxy, n-propoxy, sec-butoxy, t-butoxy, n-hexyloxy and the like.

As used herein, unless otherwise noted, the term “halogenated C₁₋₄alkoxy” shall mean any oxygen ether radical as defined above substituted with at least one halogen atom, preferably substituted with a least one fluoro atom. Suitable examples include but are not limited to —OCH₂F, —OCF₃, —OCCl₃, —CH₂—CF₃, —OCH₂—CCl₃, —OCF₂—CF₂—CF₂—CF₃, and the like. Similarly, the term “fluorinated C₁₋₄alkOXY” shall mean any oxygen ether radical as defined above substituted with at least one fluoro atom. Suitable examples include but are not limited to —OCH₂F, —OCF₃, —OCH₂—CF₃, —OCF₂—CF₂—CF₂—CF₃, and the like.

As used herein, unless otherwise noted, “aryl” shall refer to carbocylic aromatic groups such as phenyl, naphthyl, and the like.

As used herein, unless otherwise noted, “aralkyl” shall mean any lower alkyl group substituted with an aryl group such as phenyl, naphthyl and the like. For example, benzyl, phenylethyl, phenylpropyl, naphthylmethyl, and the like.

As used herein, unless otherwise noted, the term “cycloalkyl” shall mean any stable 3-8 membered monocyclic, saturated ring system, for example cyclopropyl, cyclobutyl, cyclopentyl, cyclohexyl, cycloheptyl and cyclooctyl.

As used herein, unless otherwise noted, “heteroaryl” shall denote any five or six membered monocyclic aromatic ring structure containing at least one heteroatom selected from the group consisting of O, N and S, optionally containing one to three additional heteroatoms independently selected from the group consisting of O, N and S; or a nine or ten membered bicyclic aromatic ring structure containing at least one heteroatom selected from the group consisting of O, N and S, optionally containing one to four additional heteroatoms independently selected from the group consisting of O, N and S. Unless otherwise noted, the heteroaryl group may be attached at any heteroatom or carbon atom of the ring such that the result is a stable structure. Examples of suitable heteroaryl groups include, but are not limited to, pyrrolyl, furyl, thienyl, oxazolyl, imidazolyl, purazolyl, isoxazolyl, isothiazolyl, triazolyl, thiadiazolyl, pyridyl, pyridazinyl, pyrimidinyl, pyrazinyl, pyranyl, furazanyl, indolizinyl, indolyl, isoindolinyl, indazolyl, benzofuryl, benzothienyl, benzimidazolyl, benzthiazolyl, purinyl, quinolizinyl, quinolinyl, isoquinolinyl, isothiazolyl, cinnolinyl, phthalazinyl, quinazolinyl, quinoxalinyl, naphthyridinyl, pteridinyl, and the like. Further, the term “5 to 6 membered heteroaryl” shall mean monocyclic heteroaryl as herein defined, wherein the monocyclic ring structure contains 5 to 6 ring atoms.

As used herein, the term “heterocycloalkyl” shall denote any three to eight, preferably any five to seven, membered monocyclic, saturated or partially unsaturated ring structure containing at least one heteroatom selected from the group consisting of O, N and S, optionally containing one to three additional heteroatoms independently selected from the group consisting of O, N and S; or a nine to ten membered saturated, partially unsaturated or partially aromatic bicyclic ring system containing at least one heteroatom selected from the group consisting of O, N and S, optionally containing one to four additional heteroatoms independently selected from the group consisting of O, N and S. The heterocycloalkyl group may be attached at any heteroatom or carbon atom of the ring such that the result is a stable structure. Examples of suitable heteroaryl groups include, but are not limited to, pyrrolinyl, pyrrolidinyl, dioxalanyl, imidazolinyl, imidazolidinyl, pyrazolinyl, pyrazolidinyl, piperidinyl, dioxanyl, morpholinyl, dithianyl, thiomorpholinyl, piperazinyl, trithianyl, indolinyl, chromenyl, 3,4-methylenedioxyphenyl, 2,3-dihydrobenzofuryl, and the like.

As used herein, unless otherwise noted the term “nitrogen containing heteroaryl” shall mean any heteroaryl as defined above provided that the heteroaryl contains at least one N heteroatom. Similarly, the term “nitrogen containing heterocycloalkyl” shall mean any heterocycloalkyl as defined above provided that the heterocycloalkyl contains at least one N heteroatom.

When a particular group is “substituted” (e.g., alkyl, cycloalkyl, aryl, heteroaryl, heterocycloalkyl, etc.), that group may have one or more substituents, preferably from one to five substituents, more preferably from one to three substituents, most preferably from one to two substituents, independently selected from the list of substituents.

With reference to substituents, the term “independently” means that when more than one of such substituents is possible, such substituents may be the same or different from each other.

As used herein, the notation “*” shall denote the presence of a stereogenic center.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Preferably, wherein the compound is present as an enantiomer, the enantiomer is present at an enantiomeric excess of greater than or equal to about 80%, more preferably, at an enantiomeric excess of greater than or equal to about 90%, more preferably still, at an enantiomeric excess of greater than or equal to about 95%, more preferably still, at an enantiomeric excess of greater than or equal to about 98%, most preferably, at an enantiomeric excess of greater than or equal to about 99%. Similalry, wherein the compound is present as a diastereomer, the diastereomer is present at an diastereomeric excess of greater than or equal to about 80%, more preferably, at an diastereomeric excess of greater than or equal to about 90%, more preferably still, at an diastereomeric excess of greater than or equal to about 95%, more preferably still, at an diastereomeric excess of greater than or equal to about 98%, most preferably, at an diastereomeric excess of greater than or equal to about 99%.

Furthermore, some of the crystalline forms for the compounds of the present invention may exist as polymorphs and as such are intended to be included in the present invention. In addition, some of the compounds of the present invention may form solvates with water (i.e., hydrates) or common organic solvents, and such solvates are also intended to be encompassed within the scope of this invention.

Under standard nomenclature used throughout this disclosure, the terminal portion of the designated side chain is described first, followed by the adjacent functionality toward the point of attachment. Thus, for example, a “phenylC₁-C₆alkylaminocarbonylC₁-C₆alkyl” substituent refers to a group of the formula

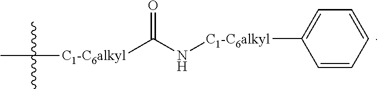

Abbreviations used in the specification, particularly the Schemes and Examples, are as follows:
ACN=Acetonitrile
BINAP=2,2′-Bis(diphenylphosphino)-1,1′-binaphthyl
BOC=t-Butoxycarbonyl
Burgess reagent=Methyl N-(triethylammoniumsulphonyl)carbamate
CBz=Benzyloxycarbonyl
n-BuOH=n-Butanol
^{t}BuOH=t-Butanol
n-BuLi=n-Butyl Lithium
DOC=N,N′-Dicyclohexylcarbodiimide
DCE=Dichloroethane
DCM=Dichloromethane
DIPEA or DIEA or =Diisopropylethylamine
Hunig's base
DMA=N,N-Dimethylacetamide
DMF=N,N-Dimethylformamide
EDC=1-(3-Dimethylaminopropyl)-3-ethylcarbodiimide hydrochloride
EDTA=Ethylene Diamine Tetraacetic Acid
Et₂O=Diethyl ether
EtOAc=Ethyl acetate
EtOH=Ethanol
HATU=O-(7-Azabenzotriazol-1-yl)-N,N,N″,N″-Tetramethyl Uronium Hexafluorophosphate
HEPES=4-(2-Hydroxyethyl)-1-Piperizine Ethane Sulfonic Acid
Hex=Hexanes
HOBT=1-Hydroxybenzotriazole
HPLC=High Pressure Liquid Chromatography
MeOH=Methanol
NaOAc=Sodium Acetate
NaBH(OAc)₃=Sodium triacetoxyborohydride
NMM=4-Methylmorpholine
NMP=N-methyl-2-pyrrolidinone
PBS=Phosphate Buffered Saline
Pd/C=Palladium on Carbon Catalyst
Pd₂Cl₂(PPh₃)₂=Palladium Bis(triphenylphosphine) chloride
Pd₂(dba)₃=Tris(dibenzylidene acetone)dipalladium(0)
PPh₃=Triphenylphosphine
PS-carbodiimide=Polystyrene bound N-benzyl-N′-cyclohexylcarbrodiimide
PyBop=Benzotriazol-1-yloxytris(pyrrolidino)phosphonium hexafluorophosphate
PyBrop=Bromortri(pyrrolidino)phosphonium hexafluorophosphate
t-BOC or Boc=Tert-Butoxycarbonyl
TEA=Triethylamine
TFA=Trifluoroacetic Acid
TFAA=Trifluoroacetic Anhydride
THF=Tetrahydrofuran
TMSCN=Trimethylsilyl cyanide
Tris HCl or Tris-Cl=Tris[hydroxymethyl]aminomethyl hydrochloride
X-Phos=2-Dicyclohexylphino-2′,4′,6′-triisopropylbiphenyl

As used herein, unless otherwise noted, the term “anxiolytic disorders” shall be defined to include anxiety and related disorders including generalized anxiety disorder, acute stress disorder, post traumatic stress disorder, obsessive-compulsive disorder, social phobia (also known as social anxiety disorder), specific phobia, panic disorder with or without agoraphobia, agoraphobia without a history of panic disorder, anxiety disorder due to general medical condition, substance abuse induced anxiety disorder and anxiety disorder not otherwise specified (as these conditions are described by their diagnostic criteria, as listed in the Diagnostic and Statistical Manual of Mental Disorders, 4^{th} Edition, Text Revision, American Psychiatric Association, 2000, incorporated herein by reference). Anxiolytic disorders shall further include stress disorders including but not limited to hemorrhagic stress, stress-induced psychotic episodes, psychosocial dwarfism, stress headaches, stress-induced immune systems disorders such as stress-induced fever, and stress-related sleep disorders. Preferably, the anxiety or related disorder is selected from the group consisting of generalized anxiety disorder, acute stress disorder, post traumatic stress disorder and obsessive-compulsive disorder. More preferably, the anxiety and related disorder is generalized anxiety disorder.

As used herein, unless otherwise noted, the term “depression” shall be defined to include major depressive disorder (including single episode and recurrent), unipolar depression, treatment-refractory depression, resistant depression, anxious depression, dysthymia (also referred to as dysthymic disorder) as well as bipolar or manic disorders. Further, the term “depression” shall encompass any major depressive disorder, dysthymic disorder and depressive disorder not otherwise specific as defined by their diagnostic criteria, as listed in the Diagnostic and Statistical Manual of Mental Disorders, 4^{th} Edition, Text Revision, American Psychiatric Association, 2000. Preferably, the depression is major depressive disorder, unipolar depression, treatment-refractory depression, resistant depression or anxious depression. More preferably, the depression is major depressive disorder.

As used herein, unless otherwise noted, the term “neurological disorders” include CNS disorders such as tinitus, spasticity, and neuropathic pain, supranuclear palsy, AIDS related dementias, multiinfarct dementia, neurodegenerative disorders such as Alzheimer's disease, Parkinson's disease, and Huntington's disease, head trauma, spinal cord trauma, ischemic neuronal damage, amyotrophic lateral sclerosis, and disorders of pain perception such as fibromyalgia and epilepsy.

As used herein, the term “pain” shall be defined to include acute, chronic, inflammatory and neuropathic pain (preferably diabetic neuropathy). Further, the pain may be centrally mediated, peripherally mediated, caused by structural tissue injury, caused by soft tissue injury or caused by progressive disease. Any centrally mediated, peripherally mediated, structural tissue injury, soft tissue injury or progressive disease related pain may be acute or chronic.

As used herein, unless otherwise noted, pain shall include inflammatory pain, centrally mediated pain, peripherally mediated pain, visceral pain, structural related pain, cancer pain, soft tissue injury related pain, progressive disease related pain, neuropathic pain, acute pain from acute injury, acute pain from trauma, acute pain from surgery, headache, dental pain, back pain (preferably lower back pain), chronic pain from neuropathic conditions and chronic pain from post-stroke conditions.

“Nerve tissue” as used herein refers to any vertebrate nerve tissue, particularly including mammalian cells of the central nervous system (CNS) and peripheral nervous system (PNS). More particularly, nerve tissue includes spinal cord neuronal structures, peripheral nervous system nerves, and even nerve cells of the brain. “Nerve tissue injury”, “injured mammalian nerve tissue”, or “CNS or PNS nerve tissue injury” include any damage to relevant nerve tissue irrespective of cause, e.g., injuries attributable to trauma including but not limited to nerve tissue lesions, traumatically-induced compression, tumors, hemorrhage, infectious processes, spinal stenosis, or impaired blood supply.

“Treating injured mammalian nerve tissue” includes, but is not limited, to the in vivo administration of compounds, compositions, and methods of the instant invention to restore action potential or nerve impulse conduction through a nerve tissue lesion. The term may also include such administration in an effort to reduce the damaging effects of any injury to mammalian nerve tissue, whether through restoration of action potential or nerve impulse conduction, by stimulating growth or proliferation of nervous tissue, by ameliorating unwanted conditions in the extracellular microenvironment near an injury, or otherwise.

As used herein, unless otherwise noted, the term “cardiovascular diseases” shall include, for example, cardiac arrhythmia, post-myocardial infarction, and heart failure.

As used herein, unless otherwise noted, the term “sleep-wake state disorders” shall include narcolepsy; sleep apnea disorders such as central sleep apnea, obstructive sleep apnea, and mixed sleep apnea; hypersomnia, including excessive daytime sleepiness (EDS), and, in particular, hypersomnia associated with narcolepsy or sleep apnea disorder; sleep/wake disturbances associated with attention deficit hyperactive disorder (ADHD); circadian rhythm abnormalities such as delayed sleep phase syndrome, advance sleep phase syndrome, non-24 hour sleep/wake disorder, jet lag, or shift-work disorder; parasomnia disorders such as somnambulism, pavor nocturnus, REM sleep behavior disorder, sleep bruxism, or sleep enuresis; sleep-related movement disorders such as sleep bruxism, restless legs syndrome, or periodic limb movement; insomnia, including extrinsic insomnia, psychophysiologic insomnia, drug-dependent insomnia, or alcohol-dependent insomnia; sleep/wake disturbances associated with mental disorders such as depression, anxiety, schizoprenia, or other psychotic disorders; sleep/wake disturbances associated with neurological disorders such as migraine, epilepsy, Parkinson's disease, or Alzheimer's disease; and sleep/wake disturbances associated with fibromyalgia, headaches, gastroesophageal reflux disease, coronary artery ischemia, cardiac arrhythmias, abnormal swallowing, choking, or laryngospasm.

As used herein, unless otherwise noted the term “substance” when referring to substances of abuse and/or addiction shall include any legal or illegal substance to which a subject or patient may develop an addiction. Suitable examples include, but are not limited to alcohol, amphetamines (such as, for example, 3,4-methylene-dioxy-N-methylamphetamine, also known as “MDMA” or “ecstacy”), cannabis, hallucinogens (such as, for example, cocaine), inhalants, heroine, ketamine, Ecstacy, nicotine, oxycontin/oxycodone, codeine, morphine, opiods, phencyclidine, narcotics, or sedatives, or combinations thereof.

As used herein, unless otherwise noted, the term “substance abuse and addiction related disorders” shall include misuse, addiction, and/or dependence disorders related to substances of abuse. “Substance abuse and addiction related disorders” shall further include cravings, symptoms of withdrawal, and the like, associated with the misuse, addiction and/or dependency to substances of abuse.

As used herein, the term “obesity” shall be defined as a body mass index (BMI) of greater than or equal to about 25, preferably a BMI of greater than or equal to about 30. (The body mass index and other definitions are according to the “NIH Clinical Guidelines on the Identification and Evaluation, and Treatment of Overweight and Obesity in Adults” (1998)) Thus as used herein, the term “obesity” shall include both overweight and clinically obese subjects/patients.

As used herein, unless otherwise noted, the term “obesity-related disorders” shall include anorexia nervosa, wasting, AIDS-related weight loss, bulimia, cachexia, lipid disorders including hyperlipidemia and hyperuricemia, insulin resistance, noninsulin dependent diabetes mellitus (NIDDM, or Type II diabetes), insulin dependent diabetes mellitus (IDDM or Type I diabetes), diabetes-related complications including microangiopathic lesions, ocular lesions, retinopathy, neuropathy, and renal lesions, cardiovascular disease including cardiac insufficiency, coronary insufficiency, and high blood pressure, atherosclerosis, atheromatous disease, stroke, hypertension, Syndrome X, gallbladder disease, osteoarthritis, sleep apnea, forms of cancer such as uterine, breast, colorectal, kidney, and gallbladder, high cholesterol levels, complications of pregnancy, menstrual irregularities, hirsutism, muscular dystrophy, infertility, and increased surgical risk.

Recently, Kuo et al. (Kuo L E, Kitlinska J B, Tilan J U, et al., Nat Med 2007) disclosed evidence which suggest that NPY acts directly in the periphery on fat tissue and mediates stress-induced obesity and metabolic syndrome. Thus, manipulation of NPY2 receptor within fat tissue offers a new way to remodel fat and treat obesity and metabolic syndrome. Additionally, NPY2 receptor antagonism has anti-angiogenic/adipogenic effects and improves glucose tolerance. NPY2 receptor antagonist are therefore useful in the treatment of obesity, obesity related disorders, impaired oral glucose tolerance, elevated glucose levels, diabetes mellitus and related glucose related disorders.

As used herein, unless otherwise noted, the term “disorders responsive to modulation of endocrine function (more particularly, disorders responsive to modulation of the pituitary and/or hypothalamic gland)” include, but are not limited to elevated glucose level, pre-diabetes, impaired oral glucose tolerance, poor glycemic control, Type II Diabetes Mellitus, Syndrome X (also known as metabolic syndrome), gestational diabetes, insulin resistance, hyperglycemia and loss of muscle mass as a results of hyperglycemia (cachexia), ifertility, inovulation, and the like. Further, the term “metabolic disorders” shall include disorders related to the metabolic system, including, but not limited to elevated glucose level, pre-diabetes, impaired oral glucose tolerance, poor glycemic control, Type II Diabetes Mellitus, Syndrome X (also known as metabolic syndrome), gestational diabetes, insulin resistance, hyperglycemia, and the like.

“Neurotrophic factor”, as used herein, refers to compounds that are capable of stimulating growth or proliferation of nervous tissue, including compounds of the instant invention and known neurotrophic factors described previously herein. Thus, the term “disorders responsive to treatment through administration of a neurotrophic factor” shall refer to any disorder which whose symptoms, pathways and/or progression may be treated and/or prevented through the use of a neurotropic factor agent.

As used herein, unless otherwise noted, the term “bone loss” refers to enhancement of bone growth or prevention of bone loss caused by conditions such as osteoporosis, osteomalacia, Paget's disease, disorders of bone homeostasis, and the like.

As used herein, unless otherwise noted, the term “infertility” shall include both male and female infertility. As used herein, unless otherwise noted, the term “inovulation” shall include inovulation regardless of underlying cause.

The term “subject” as used herein, refers to an animal, preferably a mammal, most preferably a human, who has been the object of treatment, observation or experiment. Preferably, the subject has experienced and/or exhibited at least one symptom of the disease or disorder to be treated and/or prevented.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

As used herein, the term “composition” is intended to encompass a product comprising the specified ingredients in the specified amounts, as well as any product which results, directly or indirectly, from combinations of the specified ingredients in the specified amounts.

One skilled in the art will recognize that, where not otherwise specified, the reaction step(s) is performed under suitable conditions, according to known methods, to provide the desired product.

One skilled in the art will recognize that, in the specification and claims as presented herein, wherein a reagent or reagent class/type (e.g. base, solvent, etc.) is recited in more than one step of a process, the individual reagents are independently selected for each reaction step and may be the same of different from each other. For example wherein two steps of a process recite an organic or inorganic base as a reagent, the organic or inorganic base selected for the first step may be the same or different than the organic or inorganic base of the second step.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

As used herein, unless otherwise noted, the term “leaving group” shall mean a charged or uncharged atom or group which departs during a substitution or displacement reaction. Suitable examples include, but are not limited to, Br, Cl, I, mesylate, tosylate, and the like.

As used herein, unless otherwise noted, the term “nitrogen protecting group” shall mean a group which may be attached to a nitrogen atom to protect said nitrogen atom from participating in a reaction and which may be readily removed following the reaction. Suitable nitrogen protecting groups include, but are not limited to carbamates—groups of the formula —C(O)O—R wherein R is for example methyl, ethyl, t-butyl, benzyl, phenylethyl, CH₂═CH—CH₂—, and the like; amides—groups of the formula —C(O)—R′ wherein R′ is for example methyl, phenyl, trifluoromethyl, and the like; N-sulfonyl derivatives—groups of the formula —SO₂—R″ wherein R″ is for example tolyl, phenyl, trifluoromethyl, 2,2,5,7,8-pentamethylchroman-6-yl-, 2,3,6-trimethyl-4-methoxybenzene, and the like. Other suitable nitrogen protecting groups may be measured in texts such as T. W. Greene & P. G. M. Wuts, Protective Groups in Organic Synthesis, John Wiley & Sons, 1991.

One skilled in the art will recognize that wherein a reaction step of the present invention may be carried out in a variety of solvents or solvent systems, said reaction step may also be carried out in a mixture of the suitable solvents or solvent systems.

Where the processes for the preparation of the compounds according to the invention give rise to mixture of stereoisomers, these isomers may be separated by conventional techniques such as preparative chromatography.

The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

The present invention includes within its scope prodrugs of the compounds of this invention. In general, such prodrugs will be functional derivatives of the compounds which are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs”, ed. H. Bundgaard, Elsevier, 1985.

For use in medicine, the salts of the compounds of this invention refer to non-toxic “pharmaceutically acceptable salts.” Other salts may, however, be useful in the preparation of compounds according to this invention or of their pharmaceutically acceptable salts. Suitable pharmaceutically acceptable salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. Thus, representative pharmaceutically acceptable salts include the following:

acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

Representative acids and bases which may be used in the preparation of pharmaceutically acceptable salts include the following:

acids including acetic acid, 2,2-dichloroactic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydrocy-ethanesulfonic acid, formic acid, fumaric acid, galactaric acid, gentisic acid, glucoheptonic acid, D-gluconic acid, D-glucoronic acid, L-glutamic acid, α-oxo-glutaric acid, glycolic acid, hipuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinc acid, nitric acid, oleic acid, orotic acid, oxalic acid, palmitric acid, pamoic acid, phosphoric acid, L-pyroglutamic acid, salicylic acid, 4-amino-salicylic acid, sebaic acid, stearic acid, succinic acid, sulfuric acid, tannic acid, (+)-L-tartaric acid, thiocyanic acid, p-toluenesulfonic acid and undecylenic acid; and

bases including ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

Compounds of formula (II) may be prepared as outlined in Scheme 1 below.

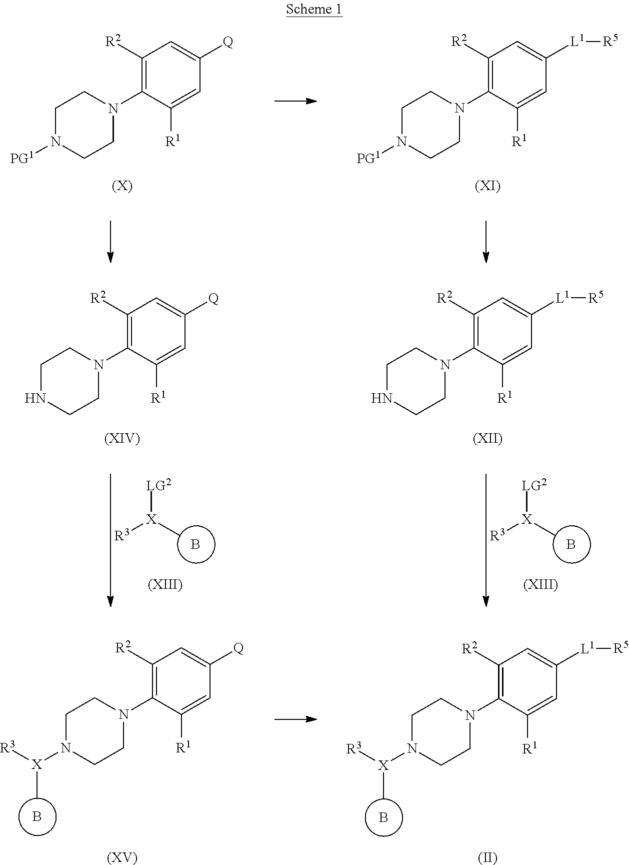

Accordingly, a suitably substituted compound of formula (X) is reacted to yield the corresponding compound of formula (XI). The compound of formula (XI) is de-protected according to known methods to yield the corresponding compound of formula (XII). The compound of formula (XII) is then reacted with a suitably substituted compound of formula (XIII), to yield the corresponding compound of formula (II).

Alternatively, a suitably substituted compound of formula (X) is de-protected according to known methods, to yield the corresponding compound of formula (XIV). The compound of formula (XIV) is reacted with a suitably substituted compound of formula (XIII), to yield the corresponding compound of formula (XV). The compound of formula (XV) is then reacted to yield the corresponding compound of formula (II). One skilled in the art will recognize that an unprotected version of the compound of formula (X) may alternatively used, thereby avoiding the de-protection step.

Schemes 2 through 9 below detail the processes by which the -L¹-R⁵ and the

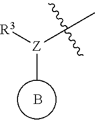

substituent groups may be attached to the piperazinyl-phenyl portion of the compound of formula (II) and unless otherwise noted, may be used in either order to yield the desired compound of formula (II).

The compound of formula (XIV) may be prepared according to the process outlined in Scheme 2 below.

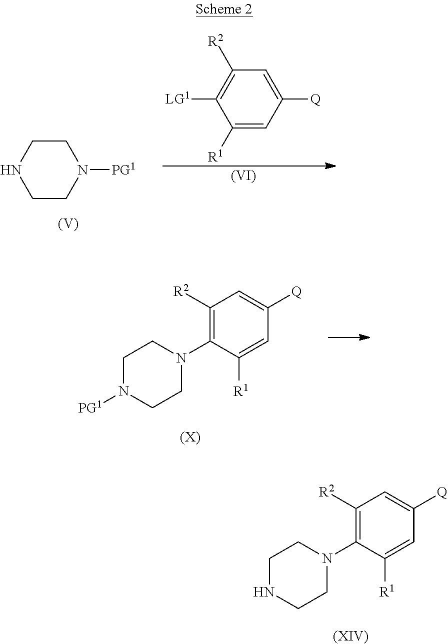

Accordingly, a suitably substituted compound of formula (V), wherein PG¹ is a suitably selected nitrogen protecting group such as BOC, CBz, benzyl, and the like, preferably BOC, a known compound or compound prepared by known methods, is reacted with a suitably substituted compound of formula (VI), wherein LG¹ is a suitably selected reactive group such as F, Cl, Br, triflate, and the like, preferably F; and wherein Q is a suitable reactive group such as Br, Cl, CN, —C(O)H, —C(O)OH, —C(O)O—C₁₋₄alkyl, —C₁₋₄alkyl-C(O)OH, —C₁₋₄alkyl-NH₂, NO₂, and the like; wherein the compound of formula (VI) is preferably present in an amount in the range of from about 1.0 to about 1.5 molar equivalents; in the presence of a base such as K₂CO₃, Na₂CO₃, KOH, Hunigs' base, and the like, preferably Hunig's base; neat or in an organic solvent such as THF, DMF, NMP, acetonitrile, and the like, preferably in acetonitrile, preferably at a temperature in the range of from about 50° C. to about 80° C.; to yield the corresponding compound of formula (X).

One skilled in the art will recognize that the compound of formula (X) may be further, optionally de-protected according to known methods, to yield the corresponding compound of formula (XIV). For example, wherein the compound of formula (X), PG¹ is BOC, the compound of formula (X) may be de-protected by reacting with a suitably selected acid such as HCl, TFA, and the like, in an organic solvent such as methanol, ethanol, diethyl ether, and the like.

In the synthesis of the compounds of formula (II), the

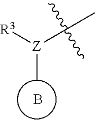

substituent group may be attached to the piperazinyl-phenyl portion according to the process outlined in Scheme 3 below. As an example, Scheme 3 below outlines the process for attaching the

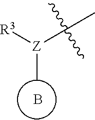

substituent group, by reacting with a suitably substituted compound of formula (XII). One skilled in the art will recognize that as described in Scheme 1 above, the

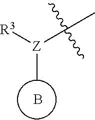

substituent group may alternatively, be reacted with a suitably substituted compound of formula (XIV), according to the process conditions as described below.

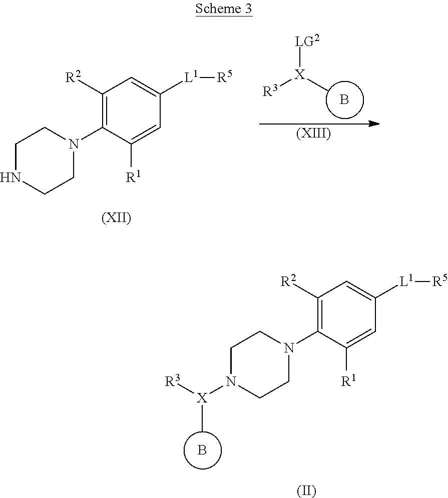

Accordingly, a suitably substituted compound of formula (XII) is reacted with a suitably substituted compound of formula (XIII), wherein LG² is a suitably selected leaving group such as iodide, bromide, chloride, tosylate, mesylate, and the like, wherein the compound of formula (XIII) is preferably present in an amount in the range of from about 1.0 to about 1.5 molar equivalents; in the presence of a base such as K₂CO₃, Na₂CO₃, NaH, and the like, preferably K₂CO₃; in an organic solvent such as THF, DMF, and the like, preferably DMF; preferably at a temperature between room temperature and reflux temperature, to yield the corresponding compound of formula (II).

Alternatively, the compound of formula (XII) is reacted with a suitably substituted compound of formula (XIII), wherein LG² is a carboxyl group, a known compound or compound prepared by known methods, wherein the compound of formula (XIII) is preferably present in an amount in the range of from about 1.0 to about 1.5 molar equivalents; in the presence of a suitably selected reducing agent such as NaBH(OAc)₃, NaBH₃CN, and the like; in an organic solvent such as DCM, DCE, MeOH, EtOH, and the like, to yield the corresponding compound of formula (II).

In the synthesis of the compounds of formula (II), the -L¹-R⁵ substituent group may be attached to the piperazinyl-phenyl portion according to the processes outlined in Scheme 4 through Scheme 9, below. Solely for the purpose of brevity, Scheme 4 through Scheme 9 below outline the process for attaching the -L¹-R⁵ substituent group, by reacting with a suitably substituted compound of formula (X) to yield the corresponding compound of formula (XI). One skilled in the art will recognize that as described in Scheme 1 above, the -L¹-R⁵ substituent group may alternatively, be reacted with a suitably substituted compound of formula (XV), according to the process conditions as described below, to yield the corresponding compound of formula (II).

Compounds of formula (XI) wherein -L¹-R⁵ is —NH—R⁵, and R^{J} is hydrogen, may be prepared as outlined in Scheme 4, below.

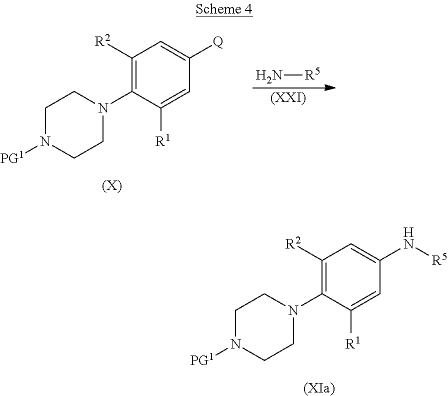

Compounds of formula (XI) wherein L¹-R⁵ is —NH—R⁵ may alternatively be prepared by activating a suitably substituted compound of formula (X) wherein Q is Br, by reacting with a suitably substituted compound of formula (XXI), in the presence of a coupling agent system such as, tri(dibenzylideneacetone)dipalladium (0), a phosphine ligand such as PPh₃, X-Phos and the like, and in the presence of a base such as sodium t-butoxide, K₂CO₃, K₃PO₄, and the like; in an organic solvent such as toluene, 1,4-dioxane, and the like, to form the corresponding compound of formula (XIa). Example 25 which follows herein, describes the preparation of a representative compound of formula (II) wherein L¹-R⁵ is —NH-(2-methylphenyl).

Compounds of formula (XI) wherein -L¹-R⁵ is —NH—C(O)—R⁵ may be prepared according to the process outlined in Scheme 5 below.

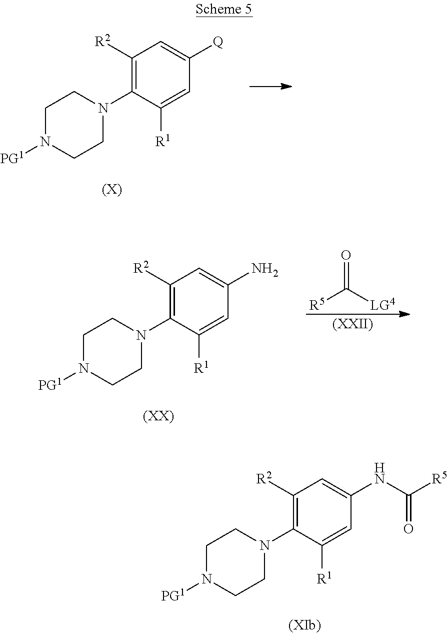

Accordingly, a suitably substituted compound of formula (X) herein Q is —NO₂, is reacted with a suitably selected reducing agent, such as SnCl₂, SnCl₂.2H₂O, and the like, in an organic solvent such as EtOH, EtOAc, and the like, or in a mixture of said organic solvents; at a temperature in the range of from about room temperature to about reflux temperature, to yield the corresponding compound of formula (XX). Alternatively, the compound of formula (X) wherein Q is —NO₂ is reacted with hydrogen over a palladium catalyst such as Pd/C, in an organic solvent such as EtOH, and the like, to yield the corresponding compound of formula (XX).

Accordingly, a suitably substituted compound of formula (XX) is reacted with a suitably substituted compound of formula (XXII), wherein LG⁴ is a suitably selected leaving group such as Cl, Br, and the like, preferably Cl, a known compound or compound prepared by known methods; in the presence of a tertiary amine base such as TEA, DIPEA, NMM, and the like, in an organic solvent such as DCM, DCE, THF, DMF, and the like; to yield the corresponding compound of formula (XIb). Alternatively, the compound of formula (XX) is reacted with a suitably substituted compound of formula (XXI), wherein LG³ is a suitably selected leaving group such as OH, a known compound or compound prepared by known methods, wherein the compound of formula (XXI) is preferably present in an amount in the range of from about 1.0 to about 1.5 molar equivalents; in the presence of a suitably selected coupling agent such as EDC, DCC, HATU, PyBoP, PyBroP, polymer-supported carbodiimide, and the like, optionally in the presence of a suitably selected ligand such as HOBt, a tertiary amine base (such as TEA, DIPEA, NMM, and the like), and the like; in an organic solvent such as DCM, DCE, THF, DMF, and the like; to yield the corresponding compound of formula (XIa).

One skilled in the art will recognize that compound of formula (XI), wherein L¹-R⁵ is selected from the group consisting of —NR^{J}—R⁵ and —NR^{J}—C(O)—R⁵ may be prepared from the corresponding compound of formula (XI) wherein L¹-R⁵ is —NH—R⁵ or —NH—C(O)—R⁵, respectively (prepared as described in for example Scheme 4 or Scheme 5 above), by reacting with a suitably substituted compound of the formula R^{J}-LG⁵, wherein LG⁵ is a suitable selected leaving group such as I, Br, Cl, and the like, preferably I, a known compound or compound prepared by known methods, in the presence of a base such as NaH, K₂CO₃, Na₂CO₃, and the like; in an organic solvent such as THF, DMF, and the like.

Compounds of formula (XI) wherein -L¹-R¹⁵ is —C(O)—NR^{J}—R⁵ may be prepared according to the process outlined in Scheme 6 below.

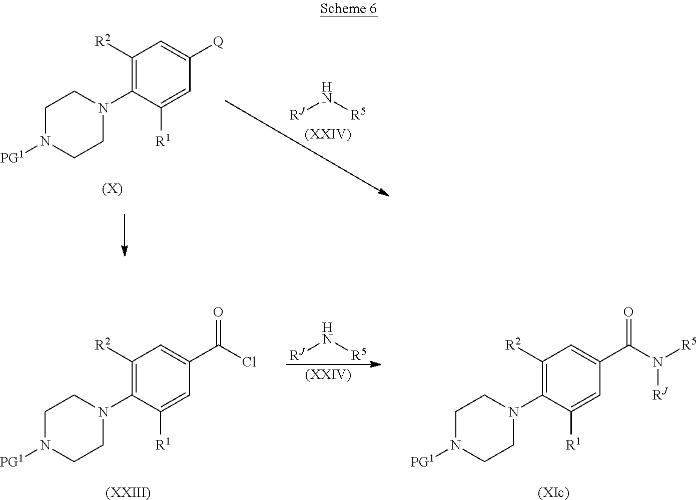

Accordingly, a suitably substituted compound of formula (X), wherein Q is —C(O)OH is reacted with a suitable source of chloride such as oxalyl chloride, and the like, in the presence of a catalyst such as DMF, DMA, and the like, in an organic solvent such as DCM, DCE, and the like, to yield the corresponding compound of formula (XXIII).

The compound of formula (XXIII) is reacted with a suitably substituted compound of formula (XXIV), a known compound or compound prepared by known methods; in the presence of a tertiary amine base such as TEA, DIPEA, NMM, and the like; in an organic solvent such as DCM, DCE, THF, DMF, and the like; to yield the corresponding compound of formula (XIc).

Alternatively a suitably substituted compound of formula (X), wherein Q is —C(O)OH is reacted with a suitably substituted compound of formula (XXIV), a known compound or compound prepared by known methods, in the presence of a suitably selected coupling agent such as EDC, DCC, HATU, PyBoP, PyBroP, polymer-supported carbodiimide, and the like, optionally in the presence of a suitably selected ligand such as HOBt, a tertiary amine base such as TEA, DIPEA, NMM, and the like; in an organic solvent such as DCM, DCE, THF, DMF, and the like; to yield the corresponding compound of formula (XIc).

Compounds of formula (XI) wherein -L¹-R⁵ is —C(O)O—R⁵ may be prepared according to the process outlined in Scheme 7 below.

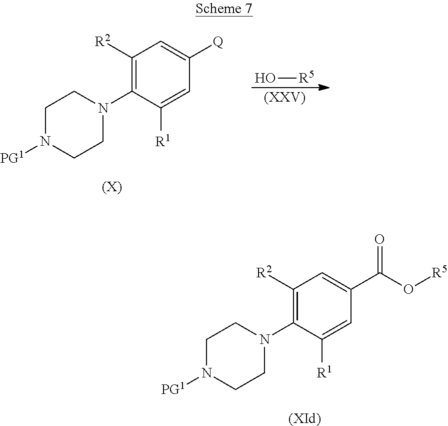

Accordingly, a suitably substituted compound of formula (X), wherein Q is —C(O)OH is reacted with a suitably substituted compound of formula (XV), a known compound or compound prepared by known methods, in the presence of an acid such as HCl, H₂SO₄, and the like; in an organic solvent such as methanol, ethanol, and the like, to yield the corresponding compound of formula (XId).

Compounds of formula (XI) wherein -L¹-R⁵ is —(CH₂)ₐ—NR^{J}—C(O)—R⁵ may be prepared according to the process outlined in Scheme 8 below.

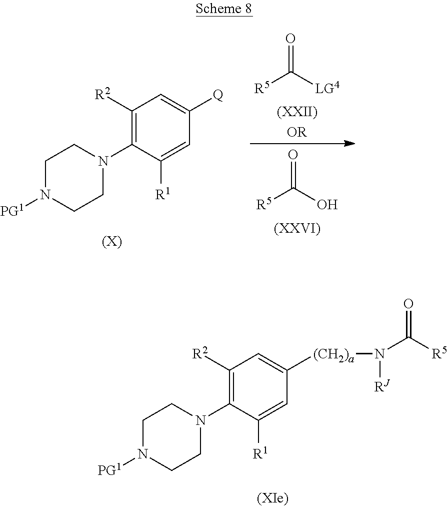

Accordingly, a suitably substituted compound of formula (X), wherein Q is —(CH₂)ₐ—NHR^{J} is reacted with a suitably substituted compound of formula (XXII) wherein LG⁴ is chloro (i.e. a suitably substituted acid chloride), a known compound or compound prepared by known methods; in the presence of a tertiary amine base such as TEA, DIPEA, NMM, and the like; in an organic solvent such as DCM, DCE, THF, DMF, and the like; to yield the corresponding compound of formula (XIe).

Alternatively, a suitably substituted compound of formula (X) wherein Q is —(CH₂)ₐ—NHR^{J} is reacted with a suitably appropriately substituted compound of formula (XXVI), a known compound or compound prepared by known methods; in the presence of a suitably selected coupling agent such as EDC, DCC, HATU, PyBoP, PyBroP, polymer-supported carbodiimide, and the like, optionally in the presence of a suitably selected ligand such as HOBt, a tertiary amine base such as TEA, DIPEA, NMM, and the like; in an organic solvent such as DCM, DCE, THF, DMF, and the like; to yield the corresponding compound of formula (XIe).

Compounds of formula (XI) wherein -L¹-R⁵ is —(CH₂)ₐ—C(O)—NR^{J}—R⁵ may be prepared according to the process outlined in Scheme 9 below.

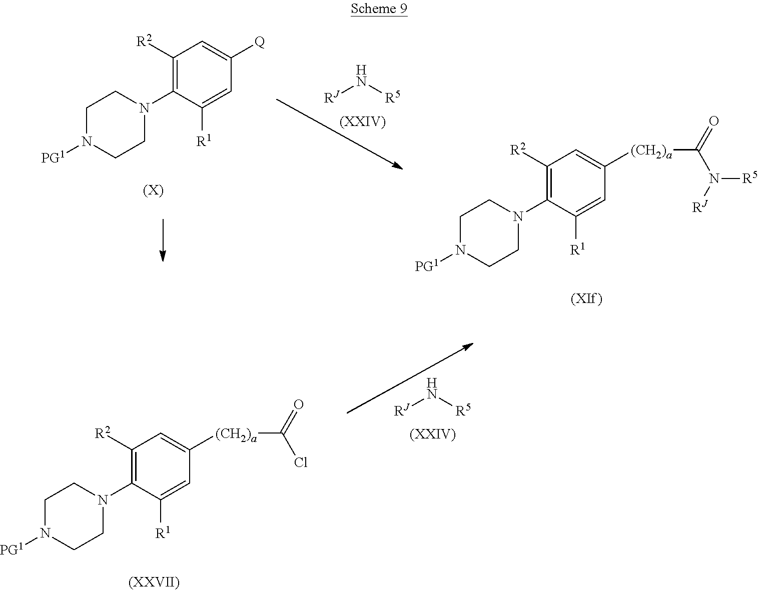

Accordingly, a suitably substituted compound of formula (X), wherein Q is —(CH₂)ₐ—C(O)OH is reacted with a suitably selected source of chlorine, such as oxalyl chloride, and the like; in the presence of a catalyst such as DMF, DMA, and the like; in an organic solvent such as DCM, DCE, and the like; to yield the corresponding compound of formula (XXVII).

The compound of formula (XXVII) is reacted with a suitably substituted compound of formula (XXIV), a known compound or compound prepared by known methods; in the presence of a tertiary amine base such as TEA, DIPEA, NMM, and the like; in an organic solvent such as DCM, DCE, THF, DMF, and the like; to yield the corresponding compound of formula (XIf).

Alternatively a suitably substituted compound of formula (X), wherein Q is —(CH₂)ₐ—C(O)OH is reacted with a suitably substituted compound of formula (XXIV), a known compound or compound prepared by known methods, in the presence of a suitably selected coupling agent such as EDC, DCC, HATU, PyBoP, PyBroP, polymer-supported carbodiimide, and the like, optionally in the presence of a suitably selected ligand such as HOBt, a tertiary amine base such as TEA, DIPEA, NMM, and the like; in an organic solvent such as DCM, DCE, THF, DMF, and the like; to yield the corresponding compound of formula (XIf).

One skilled in the art will recognize that compounds of formula (X) wherein Q is —(CH₂)ₐ—NHR^{J} or —(CH₂)ₐ—C(O)OH may be prepared for example, as described in Scheme 2 above, reacting a protected piperazine of formula (V) with a suitably substituted phenyl of formula (VI), wherein Q is —(CH₂)ₐ—NHR^{J} or —(CH₂)ₐ—C(O)OH, respectively. One skilled in the art will further recognize that compound of formula (X) wherein Q is —(CH₂)ₐ—NHR^{J} or —(CH₂)ₐ—C(O)OH may alternatively be prepared from the corresponding compound of formula (X) wherein Q is —C(O)H, according to known methods.

The present invention further comprises pharmaceutical compositions containing one or more compounds of formula (II) with a pharmaceutically acceptable carrier. Pharmaceutical compositions containing one or more of the compounds of the invention described herein as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate major site of absorption. For parenteral administration, the carrier will usually consist of sterile water and other ingredients may be added to increase solubility or preservation. Injectable suspensions or solutions may also be prepared utilizing aqueous carriers along with appropriate additives.

To prepare the pharmaceutical compositions of this invention, one or more compounds of the present invention as the active ingredient is intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, through other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, of from about 0.1-1000 mg or any range therein, and may be given at a dosage of from about 0.01-300 mg/kg/day, or any range therein, preferably from about 0.5-50 mg/kg/day, or any range therein. The dosages, however, may be varied depending upon the requirement of the patients, the severity of the condition being treated and the compound being employed. The use of either daily administration or post-periodic dosing may be employed.

Preferably these compositions are in unit dosage forms from such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from 0.1 to about 10,000 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The liquid forms in which the novel compositions of the present invention may be incorporated for administration orally or by injection include, aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

The method of treating disorders described in the present invention may also be carried out using a pharmaceutical composition comprising any of the compounds as defined herein and a pharmaceutically acceptable carrier. The pharmaceutical composition may contain between about 0.01 mg and 1000 mg of the compound, or any range therein; preferably about 10 to 500 mg of the compound, and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixers, emulsions, and suspensions. Forms useful for parenteral administration include sterile solutions, emulsions and suspensions.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitably flavored suspending or dispersing agents such as the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

To prepare a pharmaceutical composition of the present invention, a compound of formula (II) as the active ingredient is intimately admixed with a pharmaceutical carrier according to conventional pharmaceutical compounding techniques, which carrier may take a wide variety of forms depending of the form of preparation desired for administration (e.g. oral or parenteral). Suitable pharmaceutically acceptable carriers are well known in the art. Descriptions of some of these pharmaceutically acceptable carriers may be measured in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

Compounds of this invention may be administered in any of the foregoing compositions and according to dosage regimens established in the art whenever treatment of disorders mediated by the NPY Y2 receptor is required.

The daily dosage of the products may be varied over a wide range from 0.01 to 10,000 mg per adult human per day, or any range therein. For oral administration, the compositions are preferably provided in the form of tablets containing, 0.01, 0.05, 0.1, 0.5, 1.0, 2.5, 5.0, 10.0, 15.0, 25.0, 50.0, 100, 150, 200, 250, 500 and 1000 milligrams of the active ingredient for the symptomatic adjustment of the dosage to the patient to be treated. An effective amount of the drug is ordinarily supplied at a dosage level of from about 0.01 mg/kg to about 50 mg/kg of body weight per day, or any range therein. Preferably, the range is from about 0.5 to about 15.0 mg/kg of body weight per day, or any range therein. More preferably, from about 1.0 to about 10.0 mg/kg of body weight per day, or any range therein. More preferably, from about 1.0 to about 5.0 mg/kg of body weight per day, or any range therein. The compounds may be administered on a regimen of 1 to 4 times per day.

Optimal dosages to be administered may be readily determined by those skilled in the art, and will vary with the particular compound used, the mode of administration, the strength of the preparation, the mode of administration, and the advancement of the disease condition. In addition, factors associated with the particular patient being treated, including patient age, weight, diet and time of administration, will result in the need to adjust dosages.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trails including first-in-human, dose ranging and efficacy trials, in healthy patients and/or those suffering from a given disorder, may be completed according to methods well known in the clinical and medical arts.

The following Examples are set forth to aid in the understanding of the invention, and are not intended and should not be construed to limit in any way the invention set forth in the claims which follow thereafter. The examples which follow herein are only meant to suggest methods of practicing the invention. Ones skilled in the art may find other methods of practicing the invention, which are obvious to them. However, those methods are deemed to be within the scope of this invention.

In the Examples which follow, some synthesis products are listed as having been isolated as a residue. It will be understood by one of ordinary skill in the art that the term “residue” does not limit the physical state in which the product was isolated and may include, for example, a solid, an oil, a foam, a gum, a syrup, and the like. Unless otherwise noted, the materials used in the examples were obtained from readily available commercial sources or synthesized by standard methods known to those skilled in the art.

### Purification and Analytical Methods

Mass spectra were obtained on an Agilent series 1100 MSD using electrospray ionization (ESI) in either positive or negative modes as indicated. Calculated mass corresponds to the exact mass.

Thin-layer chromatography was performed using Merck silica gel 60 F₂₅₄ 2.5 cm×7.5 cm 250 μm or 5.0 cm×10.0 cm 250 μm pre-coated silica gel plates. Preparative thin-layer chromatography was performed using EM Science silica gel 60 F₂₅₄ 20 cm×20 cm 0.5 mm pre-coated plates with a 20 cm×4 cm concentrating zone.

NMR spectra were obtained on either a Bruker model DPX400 (400 MHz) or DPX500 (500 MHz) spectrometer. The format of the ¹H NMR data below is: chemical shift in ppm down field of the tetramethylsilane reference (multiplicity, coupling constant J in Hz, integration).

Normal phase flash column chromatography (FCC) was typically performed with RediSep® silica gel columns using either 2 M ammonia in methanol/dichloromethane or hexanes/ethyl acetate as eluents.

Chiral chromatography was performed using supercritical fluid chromatography (SFC)HPLC on a Chiralpak AD-H column (Chiral Technologies), eluting with isocratic 20% TEA/MeOH/CO₂ under 100 bar pressure at 25° C. Analytical: 4.6×250 mm column, 2 mL/min flow rate. Preparative: 21×250 mm column, 37.5 mL/min flow rate.

Preparative Reversed-Phase HPLC was performed on a Gilson® instrument under the following conditions: Column: YMC-Pack ODS-A, 5 μm, 75×30 mm; Flow rate: 25 mL/min; Detection: λ=220 & 254 nm; Gradient (acetonitrile/water, 0.05% trifluoroacetic acid): 15% acetonitrile/85% water to 99% acetonitrile/1% water ramp over 20 min; or on an Agilent® 1100 Series instrument under the following conditions: Column: Phenomenex Gemini, 5 μm, 100×30 mm; Flow rate: 30 mL/min; Detection: λ=220 & 254 nm; Gradient (acetonitrile/water, 20 mM NH₄OH): 5% acetonitrile/95% water to 99% acetonitrile/1% water ramp over 20 min.

### Synthetic Methods:

Unless otherwise stated, reaction solutions were stirred at room temperature. Chemical names were generated using ChemDraw Version 6.0.2 (CambridgeSoft, Cambridge, Mass.).

Representative Intermediates in the synthesis of the compounds of formula (II) of the present invention were prepared as described in Examples I-A through I-S which follow herein.

### Example I-A

### 1-(2-Fluoro-4-nitro-phenyl)-piperazine dihydrochloride

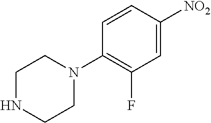

### Step A. 4-(2-Fluoro-4-nitro-phenyl)-piperazine-1-carboxylic acid tert-butyl ester

A mixture of piperazine-1-carboxylic acid tert-butyl ester (10.0 g, 53.7 mmol), 3,4-difluoronitrobenzene (6.0 mL, 54.2 mmol) and K₂CO₃ (22.0 g, 159 mmol) in DMF (60.00 mL) was heated to about 90-95° C. for 18 h. The resulting mixture was cooled to room temperature and diluted with ethyl acetate (700.0 mL) and water (200.0 mL). The organic phase was separated and washed with water (3×300 mL), dried (Na₂SO₄), filtered and concentrated to yield a yellow solid (17.00 g, 97%).

¹H NMR (CDCl₃): 8.01-7.96 (m, 1H), 7.95-7.88 (m, 1H), 6.90 (t, J=8.8, 1H), 3.65-3.55 (m, 4H), 3.28-3.20 (m, 4H), 1.48 (s, 9H).

### Step B. 1-(2-Fluoro-4-nitro-phenyl)-piperazine dihydrochloride

4-(2-fluoro-4-nitro-phenyl)piperazine-1-carboxylic acid tert-butyl ester (17.0 g, 52.25 mmol) prepared as in Step A above was dissolved into EtOH (150.0 mL) and 4M HCl in dioxane (50.0 mL) was then added. The resulting mixture was stirred for 6 h and then concentrated to dryness. The residue was co-evaporated with acetonitrile (3×100 mL) to yield the title compound.

### Example I-B

### 3-Fluoro-4-[4-(oxazol-2-yl-phenyl-methyl)-piperazin-1-yl]-phenylamine

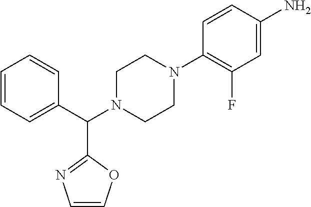

### Step A. 2-(Chloro-phenyl-methyl)-oxazole

To a solution of oxazol-2-yl-phenyl-methanol (3.94 g, 22.5 mmol) in toluene (50.0 ml) was slowly added thionyl chloride (2.0 mL, 28.1 mmol) at room temperature. The resulting solution was heated to 110° C. for 1.5 h and concentrated to yield a dark brown oil (4.4 g). Chromatography of the oil (SiO₂, DCM/Hexane) yielded the title compound.

MS (ESI) mass calculated for C₁₀H₈ClNO, 193.63; m/z measured, 194.3 [M+H]⁺

¹H NMR (CDCl₃): 7.65 (d, J=0.8, 1H), 7.60-7.55 (m, 2H), 7.43-7.42 (m, 3H), 6.10 (s, 1H).

### Step B. 1-(2-Fluoro-4-nitro-phenyl)-4-(oxazol-2-yl-phenyl-methyl)-piperazine

A mixture of 2-(chloro-phenyl-methyl)-oxazole (1.94 g, 10 mmol), 1-(2-fluoro-4-nitro-phenyl)-piperazine (2.25 g, 10 mmol), potassium carbonate (4.14 g, 30 mmol) and DMF (25.0 mL) was heated to 100° C. for 18 h. The resulting mixture was then cooled to room temperature, diluted with water (500 mL) and extracted with DCM (3×80 mL). The organic phase was dried (Na₂SO₄), filtered and concentrated to dryness to yield a reddish oil (2.52 g, 66%). Chromatography of the oil (SiO₂, 5% Acetone/DCM) yielded the title compound.

MS (ESI) mass calculated for C₂₀H₁₉FN₄O₃, 382.39; m/z measured, 383.5 [M+H]⁺

¹H NMR (CDCl₃): 7.96 (dd, J=9.0, 2.6, 1H), 7.87 ((dd, J=13.1, 2.6, 1H), 7.64 (d, J=0.7, 1H), 7.54-7.47 (m, 2H), 7.40-7.28 (m, 3H), 7.10 (d, J=0.8, 1H), 6.87 (t, J=8.8, 1H), 4.97 (s, 1H), 3.37-3.29 (m, 4H), 2.75-2.67 (m, 2H), 2.59-2.49 (m, 2H).

### Step C. 3-Fluoro-4-[4-(oxazol-2-yl-phenyl-methyl)-piperazin-1-yl]-phenylamine

1-(2-Fluoro-4-nitro-phenyl)-4-(oxazol-2-yl-phenyl-methyl)-piperazine (650 mg, 1.7 mmol) was dissolved into EtOH (20 mL) in a Parr bottle, Pd/C (10%, 35 mg) was added and the resulting mixture was shaken for 2.5 h on Parr Hydrogenation unit under 15 psi hydrogen pressure. The mixture was then filtered and concentrated to yield the title compound as a solid.

MS (ESI) mass calculated for C₂₀H₂₁FN₄O, 352.41; m/z measured, 353.2 [M+H]⁺

¹H NMR (CDCl₃): 7.63 (s, 1H), 7.55-7.50 (m, 2H), 7.38-7.27 (m, 3H), 7.08 (s, 1H), 6.78 (t, J=8.5, 1H), 6.43-6.34 (m, 2H), 4.76 (s, 1H), 3.50 (br s, 2H), 3.05-2.94 (m, 4H), 2.75-2.62 (m, 2H), 2.58-2.48 (m, 2H).

### Example I-C

### 1-(2-Methyl-4-nitro-phenyl)-piperazine

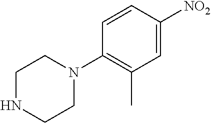

### Step A. 4-(2-Methyl-4-nitro-phenyl)-piperazine-1-carboxylic acid tert-butyl ester

The compound was prepared according to the process described in Example I-A, Step A. More particularly, a mixture of piperazine-1-carboxylic acid tert-butyl ester (8.82 g, 47.4 mmol), 1-fluoro-2-methyl-4-nitro-benzene (7.36 g, 47.4 mmol), potassium carbonate (19.7 g, 142.75 mmol) and DMF (95 mL) was used in the reaction to yield the product.

MS (ESI) mass calculated for C₁₆H₂₃N₃O₄, 321.38; m/z measured, 322.2 [M+H]⁺

¹H NMR (CDCl₃): 8.06-7.98 (m, 2H), 7.00-6.94 (m, 1H), 3.64-3.54 (m, 4H), 3.00-2.90 (m, 4H), 2.37 (s, 3H), 1.48 (s, 9H).

### Step B. 1-(2-Methyl-4-nitro-phenyl)-piperazine

4-(2-Methyl-4-nitro-phenyl)-piperazine-1-carboxylic acid tert-butyl ester (7.2 g, 22.40 mmol) prepared as in Step A above was dissolved into EtOH (150.0 mL) and 4M HCl in dioxane (40.0 mL) was added. The resulting mixture was stirred for 6 h and then concentrated to dryness. The resulting residue was co-evaporated with acetonitrile (3×100 mL) to yield the title compound as its corresponding dihydrochloride salt. The dihydrochloride salt was stirred in a mixture of DCM (150 mL) and sat. NaHCO₃ aq. solution (100.0 mL) for 3 h. Organic phase was separated and concentrated to yield the title compound.

MS (ESI) mass calculated for C₁₁H₁₅N₃O₂, 221.26; m/z measured, 222.2 [M+H]⁺

¹H NMR (CDCl₃): 8.04-7.98 (m, 2H), 7.00-6.95 (m, 1H), 3.18-2.86 (m, 8H), 2.35 (s, 3H).

### Example I-D

### 3-Methyl-4-[4-(oxazol-2-yl-phenyl-methyl)-piperazin-1-yl]-phenylamine

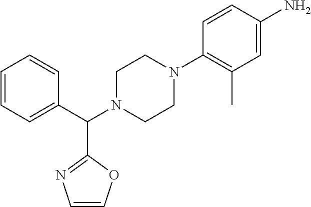

### Step A. 1-(2-Methyl-4-nitro-phenyl)-4-(oxazol-2-yl-phenyl-methyl)-piperazine

A mixture of 2-(chloro-phenyl-methyl)-oxazole (1.5 g, 7.75 mmol), 1-(2-methyl-4-nitro-phenyl)-piperazine (2.0 g, 7.75 mmol), Cs₂CO₃ (3.16 g, 9.70 mmol) in acetonitrile was heated to 50° C. for 18 h. The resulting mixture was cooled to room temperature and partitioned between water (100 mL) and DCM (3×100 mL). Organic phase was separated, dried (Na₂SO₄), filtered and concentrated to dryness to yield a residue. Chromatography of the residue (SiO₂, 0-2% acetone/DCM, gradient) yielded the title compound.

MS (ESI) mass calculated for C₂₁H₂₂N₄O₃, 378.42; m/z measured, 379.2 [M+H]⁺

¹H NMR (CDCl₃): 8.05-7.99 (m, 2H), 7.65 (br s, 1H), 7.55-7.49 (m, 2H), 7.40-7.29 (m, 3H), 7.10 (d, J=0.5, 1H), 6.99-6.95 (m, 1H), 4.79 (s, 1H), 3.08-3.04 (m, 4H), 2.78-2.67 (m, 2H), 2.58-2.48 (m, 2H), 2.31 (s, 3H).

### Step B. 3-Methyl-4-[4-(oxazol-2-yl-phenyl-methyl)-piperazin-1-yl]-phenylamine

1-(2-Methyl-4-nitro-phenyl)-4-(oxazol-2-yl-phenyl-methyl)-piperazine (2.2 g, 5.8 mmol) was dissolved into EtOH (50 mL) in a Parr bottle. Pd/C (10%, 150 mg) was added and then the hydrogenation was carried out atwas charged with 30 psi of hydrogen on Parr unit and shaken for 4 h. The resulting mixture was then filtered and concentrated to yield the title compound.

MS (ESI) mass calculated for C₂₁H₂₄N₄O, 348.24; m/z measured, 349.2 [M+H]⁺

¹H NMR (CDCl₃): 7.63 (br s, 1H), 7.57-7.51 (m, 2H), 7.38-7.31 (m, 3H), 7.08 (d, J=0.7, 1H), 6.89-6.84 (m, 1H), 6.57-6.47 (m, 2H), 4.73 (s, 1H), 3.01-2.96 (m, 2H), 2.89-2.84 (m, 4H), 2.80-2.75 (m, 2H), 2.64 (br s, 2H), 2.18 (s, 3H).

### Example I-E

### 5-Nitro-2-piperazin-1-yl-benzonitrile

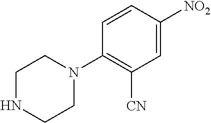

### Step A. 4-(2-Cyano-4-nitro-phenyl)-piperazine-1-carboxylic acid tert-butyl ester

The compound was prepared according to the procedure as described in Example I-A, Step A, reacting a mixture of piperazine-1-carboxylic acid tert-butyl ester (5.60 g, 30.1 mmol), 2-fluoro-5-nitro-benzonitrile (5.00 g, 30.1 mmol), potassium carbonate (12.5 g, 90.3 mmol) and DMF (60 mL) to yield the title compound.

MS (ESI) mass calculated for C₁₆H₂₀N₄O₄, 332.35 m/z measured, 333.4 [M+H]⁺

¹H NMR (CDCl₃): 8.44 (d, J=2.7, 1H), 8.29 (dd, J=9.3, 2.7, 1H), 6.98 (d, J=9.3, 1H), 3.70-3.62 (m, 4H), 3.51-3.42 (m, 4H), 1.48 (s, 9H).

### Step B: 5-Nitro-2-piperazin-1-yl-benzonitrile

4-(2-Cyano-4-nitro-phenyl)-piperazine-1-carboxylic acid tert-butyl ester (9.76 g, 29.4 mmol) was dissolved into MeOH (100.0 mL) and 4M HCl in dioxane (650.0 mL) was added. The resulting mixture was stirred for 6 h and then concentrated to dryness. The residue was taken into the mixture of DCM (250.0 mL) and sat. aq. NaHCO₃ (100.0 mL) and stirred for 2 h. The organic phase was separated, dried (Na₂SO₄) and concentrated to yield the title compound.

### Example I-F

### 5-Amino-2-[4-(oxazol-2-yl-phenyl-methyl)-piperazin-1-yl]-benzonitrile

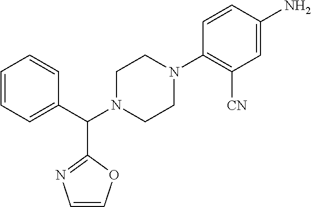

### Step A. 5-Nitro-2-[4-(oxazol-2-yl-phenyl-methyl)-piperazin-1-yl]-benzonitrile

A mixture of 2-(chloro-phenyl-methyl)-oxazole (2.5 g, 12.9 mmol), 5-nitro-2-piperazin-1-yl-benzonitrile (3.0 g, 12.9 mmol), Cs₂CO₃ (5.3 g, 16.2 mmol) in acetonitrile was heated to 50° C. for 18 h. The resulting mixture was cooled to room temperature and partitioned between water (100 mL) and DCM (3×100 mL). The organic phase was separated, dried (Na₂SO₄), filtered and concentrated to dryness to yield a residue. Chromatography of the residue (SiO₂, 0-3% acetone/DCM, gradient) yielded the title compound.

MS (ESI) mass calculated for C₂₁H₁₉N₅O₃, 389.41; m/z measured, 390.2 [M+H]⁺.

### Step B. 5-Amino-2-[4-(oxazol-2-yl-phenyl-methyl)-piperazin-1-yl]-benzonitrile

5-Nitro-2-[4-(oxazol-2-yl-phenyl-methyl)-piperazin-1-yl]-benzonitrile (2.27 g, 5.8 mmol) was dissolved into EtOH (50 mL) and Pd/C 10% (150 mg) was added. The resulting mixture was stirred over an atmosphere of hydrogen provided by hydrogen balloon for 18 h. The resulting mixture was filtered and concentrated to dryness to yield the title compound.

MS (ESI) mass calculated for C₂₁H₂₁N₅O, 359.43; m/z measured, 360.2 [M+H]⁺

¹H NMR (CDCl₃): 7.62 (s, 1H), 7.54-7.48 (m, 2H), 7.38-7.30 (m, 3H), 7.09-7.06 (m, 1H), 6.89-6.77 (m, 3H), 4.75 (s, 1H), 3.65 (br s, 2H), 3.12-3.04 (m, 4H), 2.74-2.64 (m, 2H), 2.59-2.51 (m, 2H).

### Example I-G

### Methanesulfonic acid (4-fluoro-phenyl)-oxazol-2-yl-methyl ester

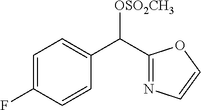

### Step A. (4-Fluoro-phenyl)-oxazol-2-yl-methanol

To a solution of oxazole (5.25 g, 76 mmol) in THF (150.0 mL) was added BH₃.THF (1M solution, 84 mL, 84 mmol) at room temperature. The resulting mixture was stirred for 1 h at room temperature and then cooled to −78° C. To the resulting mixture was then slowly added n-BuLi 1.6M in THF (52.5 mL, 84 mmol). The resulting mixture was stirred at −78° C. for 1 h. To the resulting mixture was then added 4-fluorobenzaldehyde and the mixture stirred for 4 h at −78° C. To the resulting mixture was then added 5% acetic acid in EtOH (100 mL) at −78° C. The resulting mixture was stirred for 18 h, then extracted it with ethyl ether (3×300 mL), dried (Na₂SO₄), filtered and concentrated to yield a residue. Chromatography of the residue (SiO₂, 0-3% acetone/DCM) yielded the title compound.

MS (ESI) mass calculated for C₁₀H₈FNO₂, 193.17; m/z measured, 176.4 [M−17]

¹H NMR (CDCl₃): 7.60 (s, 1H), 7.49-7.38 (m, 2H), 7.13-7.00 (m, 3H), 5.89 (s, 1H), 1.77 (br s, 1H).

### Step B. Methanesulfonic acid (4-fluoro-phenyl)-oxazol-2-yl-methyl ester

To a solution of (4-fluoro-phenyl)-oxazol-2-yl-methanol (0.58 g, 3.0 mmol) and TEA (0.48 g, 4.8 mmol) in DCM (10.0 mL) cooled to 0° C., was added methane sulfonyl chloride (0.39 g, 3.4 mmol). The ice bath was then removed and the resulting mixture was stirred at room temperature for 3 more h. The resulting mixture was then treated with water and the organic portion was separated, dried (Na₂SO₄), filtered and concentrated to dryness to yield the title compound.

### Example I-H

### Methanesulfonic acid (3-fluoro-phenyl)-oxazol-2-yl-methyl ester

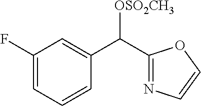

### Step A. (3-Fluoro-phenyl)-oxazol-2-yl-methanol

The title compound was prepared according to the process described in Example I-G, Step A with appropriate reagent substitutions.

MS (ESI) mass calculated for C₁₀H₈FNO₂, 193.17; m/z measured, 194.1 [M+H]⁺

¹H NMR (CDCl₃): 7.61 (d, J=0.8, 1H), 7.37-7.30 (m, 1H), 7.25-7.16 (m, 2H), 7.07 (d, J=0.7, 1H), 7.05-7.00 (m, 1H), 5.90 (br s, 1H), 4.31 (br s, 1H).

### Step B. Methanesulfonic acid (3-fluoro-phenyl)-oxazol-2-yl-methyl ester

The title compound was prepared according to the process described in Example I-G, Step B with appropriate reagent substitutions.

### Example I-I

### Methanesulfonic acid (4-cyano-phenyl)-oxazol-2-yl-methyl ester

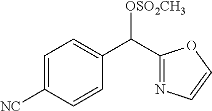

### Step A. 4-(Hydroxy-oxazol-2-yl-methyl)-benzonitrile

The title compound was prepared according to the process described in Example I-G, Step A with appropriate reagent substitutions.

MS (ESI) mass calculated for C₁₁H₈N₂O₂, 200.19; m/z measured, 201.2 [M+H]⁺

¹H NMR (CDCl₃): 7.70-7.59 (m, 5H), 7.1 (s, 1H), 5.96 (br s, 1H), 3.80 (br s, 1H).

### Step B. Methanesulfonic acid (4-cyano-phenyl)-oxazol-2-yl-methyl ester

The title compound was prepared according to the process described in Example I-G, Step B with appropriate reagent substitutions.

### Example I-J

### Methanesulfonic acid (4-methoxy-phenyl)-oxazol-2-yl-methyl ester

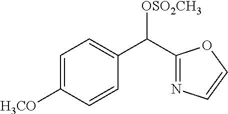

### Step A. (4-Methoxy-phenyl)-oxazol-2-yl-methanol

The title compound was prepared according to the process described in Example I-G, Step A with appropriate reagent substitutions.

MS (ESI) mass calculated for C₁₁H₁₁NO₃, 205.21; m/z measured, 206.2 [M+H]⁺

¹H NMR (CDCl₃): 7.60 (d, J=0.7, 1H), 7.38-7.34 (m, 2H), 7.08 (d, J=0.5, 1H), 6.93-6.88 (m, 2H), 5.84 (br s, 1H), 3.80 (s, 3H), 3.46 (br s, 1H).

### Step B. Methanesulfonic acid (4-methoxy-phenyl)-oxazol-2-yl-methyl ester

The title compound was prepared according to the process described in Example I-G, Step B with appropriate reagent substitutions.

### Example I-K

### Methanesulfonic acid (4-chloro-phenyl)-oxazol-2-yl-methyl ester

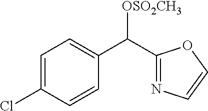

### Step A. (4-Chloro-phenyl)-oxazol-2-yl-methanol

The title compound was prepared according to the process described in Example I-G, Step A with appropriate reagent substitutions.

MS (ESI) mass calculated for C₁₀H₈ClNO₂, 209.63; m/z measured, 210.2 [M+H]⁺

¹H NMR (CDCl₃): 7.60 (d, J=0.8, 1H), 7.42-7.32 (m, 4H), 7.08 (s, 1H), 5.90 (s, 1H), 3.81 (s, 1H).

### STEP B. METHANESULFON IC ACID (4-CHLORO-PHENYL)-OXAZOL-2-YL-METHYL ESTER

The title compound was prepared according to the process described in Example I-G, Step B with appropriate reagent substitutions.

### Example I-L

### Methanesulfonic acid oxazol-2-yl-pyridin-2-yl-methyl ester

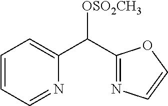

### Step A. Oxazol-2-yl-pyridin-2-yl-methanol

The title compound was prepared according to the process described in Example I-G, Step A with appropriate reagent substitutions.

MS (ESI) mass calculated for C₉H₈N₂O₂, 176.17; m/z measured, 177.2 [M+H]⁺

¹H NMR (CDCl₃): 8.62-8.56 (m, 1H), 7.75-7.68 (m, 1H), 7.61 (s, 1H), 7.40-7.34 (m, 1H), 7.30-7.25 (m, 1H), 7.08 (s, 1H), 5.96 (s, 1H).

### Step B. Methanesulfonic acid oxazol-2-yl-pyridin-2-yl-methyl ester

The title compound was prepared according to the process described in Example I-G, Step B with appropriate reagent substitutions.

### Example I-M

### 2-Ethyl-N-(3-fluoro-4-piperazin-1-yl-phenyl)-butyramide

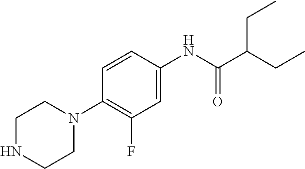

### Step A. 4-(4-Amino-2-fluoro-phenyl)-piperazine-1-carboxylic acid tert-butyl ester

4-(2-Fluoro-4-nitro-phenyl)-piperazine-1-carboxylic acid tert-butyl ester (4.0 g, 12.3 mmol), was dissolved into EtOH (200 mL) and Pd/C 10% (300 mg) was added carefully. The resulting mixture was stirred under hydrogen atmosphere provided by hydrogen balloon for 24 h. The catalyst was removed and the concentration of the filtrate yield the title compound.

MS (ESI) mass calculated for C₁₅H₂₂FN₃O₂, 295.35; m/z measured, 296.5 [M+H]⁺

¹H NMR (CDCl₃): 6.76 (t, J=9.2, 1H), 6.44-6.35 (m, 2H), 3.62-3.49 (m, 6H), 2.93-2.82 (m, 4H), 1.47 (s, 9H).

### Step B. 4-[4-(2-Ethyl-butyrylamino)-2-fluoro-phenyl]-piperazine-1-carboxylic acid tert-butyl ester

4-(4-Amino-2-fluoro-phenyl)-piperazine-1-carboxylic acid tert-butyl ester (4.54 g, 15.4 mmol) and DIPEA (2.95 mL, 16.9 mmol) were dissolved in DCM (90.0 mL) and cooled to 0° C. 2-Ethyl-butyryl chloride (2.28 mL, 16.2 mmol) was added slowly. The resulting mixture was then stirred at 0° C. for 1 h and at room temperature for 4 h. The resulting mixture was then washed with water, 1N NaOH solution and water. The organic phase was dried (Na₂SO₄), filtered and concentrated to yield a residue. Chromatography of the residue (SiO₂, 0-8% acetone/DCM) yielded the title compound.

MS (ESI) mass calculated for C₂₁H₃₂FN₃O₃, 393.50, m/z measured, 394.6 [M+H]⁺

¹H NMR (CDCl₃): 7.50 (dd, J=13.9, 2.4, 1H), 7.22 (s, 1H), 7.15-7.07 (m, 1H), 6.88 (t, J=9.0, 1H), 3.63-3.52 (m, 4H), 3.00-2.92 (m, 4H), 2.06-1.93 (m, 1H), 1.75-1.65 (m, 2H), 1.59-1.52 (m, 2H), 1.48 (s, 9H), 1.00-0.88 (m, 6H).

### Step C. 2-Ethyl-N-(3-fluoro-4-piperazin-1-yl-phenyl)-butyramide

To a solution of 4-[4-(2-Ethyl-butyrylamino)-2-fluoro-phenyl]-piperazine-1-carboxylic acid tert-butyl ester (4.26 g, 10.8 mmol) into EtOH (100.0 mL) was added 4M HCl in dioxane (25 mL). The resulting mixture was stirred for 3 h and then concentrated to dryness to yield the title compound as its corresponding HCl salt. The hydrochloride salt was treated with 2M NH₃ in MeOH (60 mL), diluted with DCM (200.0 mL), washed with water, dried (Na₂SO₄), filtered and concentrated to dryness to yield the title compound.

MS (ESI) mass calculated for C₁₆H₂₄FN₃O, 293.38, m/z measured, 294.4 [M+H]⁺

¹H NMR (CDCl₃): 7.45 ((dd, J=14.0, 2.4, 1H), 7.21 (s, 1H), 7.14-7.08 (m, 1H), 6.87 (t, J=9.0, 1H), 3.08-2.95 (m, 8H), 2.05-1.95 (m, 1H), 1.76-1.64 (m, 3H), 1.60-1.50 (m, 2H), 0.93 (t, J=7.4, 6H).

### Example I-N

### N-(3-Cyano-4-piperazin-1-yl-phenyl)-2-ethyl-butyramide

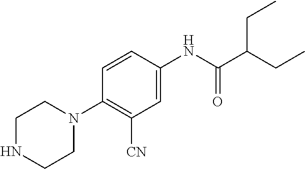

### Step A. 4-(4-Amino-2-cyano-phenyl)-piperazine-1-carboxylic acid tert-butyl ester

The title compound was prepared according to the process described in Example I-M, Step A with appropriate reagent substitutions.

MS (ESI) mass calculated for C₁₆H₂₂N₄O₂, 302.37, m/z measured, 303.4 [M+H]⁺

¹H NMR (CDCl₃): 6.90-6.85 (m, 2H), 6.84-6.80 (m, 1H), 3.64-3.55 (m, 4H), 3.00-2.93 (m, 4H), 1.48 (s, 9H).

### Step B. 4-[2-Cyano-4-(2-ethyl-butyrylamino)-phenyl]-piperazine-1-carboxylic acid tert-butyl ester

The title compound was prepared according to the process described in Example I-N, Step B with appropriate reagent substitutions.

MS (ESI) mass calculated for C₂₂H₃₂N₄O₃, 400.51, m/z measured, 345.3 [M−57+H]⁺

¹H NMR (CDCl₃): 7.80 (d, J=2.6, 1H), 7.70 (dd, J=8.9, 2.6, 1H), 7.19 (br s, 1H), 6.97 (d, J=8.9, 1H), 3.66-3.59 (m, 4H), 3.11-3.05 (m, 4H), 2.06-1.98 (m, 1H), 1.77-1.65 (m, 2H), 1.63-1.53 (m, 2H), 1.48 (s, 9H), 0.94 (t, J=7.4, 6H).

### Step C. N-(3-Cyano-4-piperazin-1-yl-phenyl)-2-ethyl-butyramide

The title compound was prepared according to the process described in Example I-M, Step C with appropriate reagent substitutions.

MS (ESI) mass calculated for C₁₇H₂₄N₄O, 300.40, m/z measured, 301.3 [M+H]⁺

¹H NMR (CDCl₃): 7.77 (d, J=2.6, 1H), 7.69 (dd, J=8.9, 2.6, 1H), 7.29 (br s, 1H), 6.97 (d, J=8.9, 1H), 3.14-3.04 (m, 8H), 2.00-1.98 (m, 1H), 1.75-1.65 (m, 3H), 1.61-1.52 (m, 2H), 0.94 (t, J=7.4, 6H).

### Example I-O

### (R)-tetrahydro-furan-3-carboxylic acid (3-fluoro-4-piperazin-1-yl-phenyl)-amide

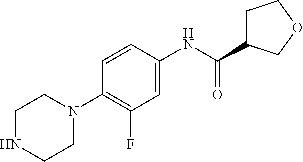

### Step A. 4-{2-Fluoro-4-[(R-tetrahydro-furan-3-carbonyl)-amino]-phenyl}-piperazine-1-carboxylic acid tert-butyl ester

A mixture of 4-(4-Amino-2-fluoro-phenyl)piperazine-1-carboxylic acid tert-butyl ester (0.7 g, 2.4 mmol), (R)-tetrahydro-furan-3-carboxylic acid (0.33 g, 2.9 mmol) and HATU (1.1 g, 2.9 mmol) was dissolved into DMF (15.0 mL) and stirred for 1 h at room temperature. The resulting mixture was diluted with ethyl acetate (200 mL) and washed with 1N NaOH (30 mL) and then with water (2×200 mL), dried (Na₂SO₄), filtered and concentrated to dryness to yield a residue. Chromatography of the residue (SiO₂, 0-5% acetone/DCM) yielded the title compound.

MS (ESI) mass calculated for C₂₀H₂₈FN₃O₄, 393.46, m/z measured, 394.6 [M+H]⁺

¹H NMR (CDCl₃): 7.50-7.40 (m, 2H), 7.11-7.06 (m, 1H), 6.86 (t, J=9.0, 1H), 4.09-3.98 (m, 2H), 3.97-3.90 (m, 1H), 3.88-3.80 (m, 1H), 3.62-3.54 (m, 4H), 3.08-2.92 (m, 5H), 2.30-2.20 (m, 2H), 1.48 (s, 9H).

### Step B. (R)-tetrahydro-furan-3-carboxylic acid (3-fluoro-4-piperazin-1-yl-phenyl)-amide

The title compound was prepared according to the process described in Example I-M, Step C with appropriate reagent substitutions.

### Example I-P

### Cyclopentanecarboxylic acid (3-fluoro-4-piperazin-1-yl-phenyl)-amide

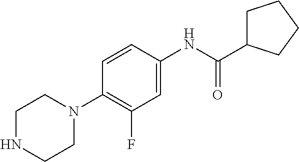

### Step A. 4-[4-(Cyclopentanecarbonyl-amino)-2-fluoro-phenyl]-piperazine-1-carboxylic acid tert-butyl ester

The title compound was prepared according to the process described in Example I-O, Step A with appropriate reagent substitutions. More particularly, 4-(4-amino-2-fluoro-phenyl)piperazine-1-carboxylic acid tert-butyl ester (1.2 g, 4.1 mmol), cyclopentanecarboxylic acid (0.58 g, 5.1 mmol) was reacted to yield the title compound.

MS (ESI) mass calculated for C₂₁H₃₀FN₃O₃, 391.49, m/z measured, 392.6 [M+H]⁺

¹H NMR (CDCl₃): 7.50-7.43 (m, 1H), 7.14 (br s, 1H), 7.11-7.06 (m, 1H), 6.85 (t, J=9.0, 1H), 3.62-3.54 (m, 4H), 3.02-2.93 (m, 4H), 2.68-2.61 (m, 1H), 1.99-1.83 (m, 4H), 1.82-1.71 (m, 2H), 1.68-1.57 (m, 2H), 1.48 (s, 9H).

### Step B. Cyclopentanecarboxylic acid (3-fluoro-4-piperazin-1-yl-phenyl)-amide

The title compound was prepared according to the process described in Example I-M, Step C with appropriate reagent substitutions.

### Example I-Q

### 2-[Chloro-(3-fluoro-phenyl)-methyl]-oxazole

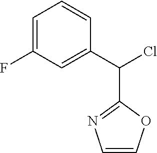

The title compound was prepared according to the process described in Example I-B, C with appropriate reagent substitutions, reacting (3-fluoro-phenyl)-oxazol-2-yl-methanol (1.60 g, 8.3 mmol) to yield the title compound.

### Example I-R

### Methanesulfonic acid benzooxazol-2-yl-phenyl-methyl ester

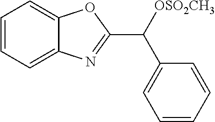

### Step A. Benzooxazol-2-yl-phenyl-methanol

2-Amino-phenol (10.2 g, 93.4 mmol) and hydroxy-phenyl-acetic acid (12.2 g, 80.1 mmol) were heated to reflux in xylene (300 mL) using Dean-Stark apparatus for four days during which a quantitative amount of water was collected. The resulting mixture was then cooled to 10° C. and the precipitates were collected, washed with EtOH and dried to yield a residue. The residue was recrystallized with 1:1 H₂O:EtOH (60 mL) to yield the title compound as a pink crystalline solid.

MS (ESI) mass calculated for C₁₄H₁₁NO₂, 225.25, m/z measured, 226.3 [M+H]⁺

¹H NMR (CDCl₃): 7.72-7.65 (m, 1H), 7.57-7.51 (m, 2H), 7.50-7.44 (m, 1H), 7.42-7.28 (m, 5H), 6.04 (s, 1H), 4.04 (br s, 1H).

### Step B. Methanesulfonic acid benzooxazol-2-yl-phenyl-methyl ester

The title compound was prepared according to the process described in Example I-G, B with appropriate reagent substitutions, reacting benzooxazol-2-yl-phenyl-methanol (630 mg, 2.8 mmol) to yield the title compound.

### Example I-S

### Methanesulfonic acid benzothiazol-2-yl-phenyl-methyl ester

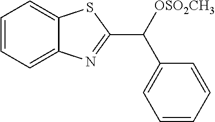

### Step A. Benzothiazol-2-yl-phenyl-methanol

Benzothiazole (5.0 g, 37 mmol) was dissolved in THF (250 mL) and cooled to −78° C. n-BuLi (2.5M in Hexane, 17.8 mL, 44.4 mmol) was added at −78° C. over 30 minutes. The resulting mixture was stirred for 1.5 h at −78° C. Then benzaldehyde (4.7 g, 44.4 mmol) was added slowly at −78° C. and the resulting mixture stirred for one more hour at −78° C. EtOH (15 ml) was then added to the resulting mixture at −78° C. The resulting mixture was brought to room temperature and stirred for 0.5 h, then diluted with water (200.0 mL) and extracted with DCM (3×150 mL). The combined organic phase was dried, filtered and concentrated to yield a residue. The residue was crystallized in a boiling mixture of DCM (100 mL) and hexane (150 mL) to yield the title compound.

MS (ESI) mass calculated for C₁₄H₁₁NOS, 241.31, m/z measured, 242.4 [M+H]⁺

¹H NMR (CDCl₃): 7.98 (d, J=8.2, 1H), 7.83 (d, J=8.0, 1H), 7.56-7.50 (m, 2H), 7.49-7.43 (m, 1H), 7.42-7.31 (m, 4H), 6.14 (s, 1H), 4.00 (br s, 1H).

### Step B. Methanesulfonic acid benzothiazol-2-yl-phenyl-methyl ester

The title compound was prepared according to the process described in Example I-G, B with appropriate reagent substitutions, reacting benzothiazol-2-yl-phenyl-methanol (603 mg, 2.5 mmol) to yield the title compound.

### Example I-T

### 3-(Chloro-phenyl-methyl)-pyridine

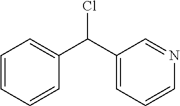

### Step A. Phenyl-pyridin-3-yl-methanol

A solution of 3-benzoylpyridine (5 g, 27.3 mmol) in methanol (55 mL) was cooled in an ice bath. Sodium borohydride (1.24 g, 32.7 mmol) was added to the resulting mixture in three portions over 1 h, and the mixture was then stirred overnight. The mixture was poured into ice water, and the resulting mixture was extracted into ethyl acetate. The ethyl acetate solution was washed with brine, dried over Na₂SO₄, filtered and concentrated. The resulting residue was purified by flash chromatography to yield phenyl-pyridin-3-yl-methanol

MS (ESI+APCI): Calculated for C₁₂H₁₁NO, 185.08; m/z measured 186.1 [M+H]⁺.

¹H NMR (400 MHz, CDCl₃): 8.57-8.56 (m, 1H), 8.44 (dd, J=4.82, 1.64 Hz, 1H), 7.72-7.69 (m, 1H), 7.38-7.28 (m 5H), 7.25-7.23 (m, 1H), 5.98 (s, 1H), 3.13 (br s, 1H).

### Step B. 3-(Chloro-phenyl-methyl)-pyridine

A solution of phenyl-pyridin-3-yl-methanol (4.36 g, 23.6 mmol) and thionyl chloride (2.22 mL, 30.6 mmol) in DCM (59 mL) was stirred at room temperature overnight and then neutralized with 1M NaOH. The organic and aqueous portions were separated, and the aqueous portion was extracted with DCM. The combined organic portions were washed with brine, dried over Na₂SO₄, filtered, and concentrated to yield the title compound, which was used in subsequent reaction steps without further purification.

### Example I-U

### 4-(Chloro-phenyl-methyl)-pyridine

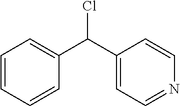

### Step A. Phenyl-pyridin-4-yl-methanol

A solution of 4-benzoylpyridine (15 g, 82 mmol) in methanol (164 mL) was cooled in an ice bath, and sodium borohydride (3.7 g, 98 mmol) was added in three aliquots over 1 hour. The resulting mixture was stirred at room temperature for 48 h, then poured into ice water and extracted into ethyl acetate. The organic portion was washed with brine, dried over magnesium sulfate, filtered and concentrated. The resulting residue was triturated with DCM to yield phenyl-pyridin-4-yl-methanol

MS (ESI+APCI): Calculated for C₁₂H₁₁NO, 185.08; m/z measured 186.1 [M+H]⁺.

¹H NMR (400 MHz, CDCl₃): 8.48-8.43 (m, 2H), 7.37-7.29 (m, 9H), 5.79 (s, 1H).

### Step B. 4-(Chloro-phenyl-methyl)-pyridine

A solution of phenyl-pyridin-4-yl-methanol (5.0 g, 27 mmol) and thionyl chloride (2.5 mL, 35 mmol) in DCM (67.5 mL) was stirred at room temperature overnight and then neutralized with 1M NaOH. The organic and aqueous portions were separated, and the aqueous portion was extracted with DCM. The combined organic portions were washed with brine, dried over Na₂SO₄, filtered, and concentrated to yield the title compound, which was used in subsequent reaction steps without further purification.

MS (ESI+APCI): mass calcd. for C₁₂H₁₀ClN, 203.05; m/z found, 204.1 [M+H]⁺.

Representative compounds of formula (II) of the present invention were prepared as described in the Examples which follow herein.

### Example 1

### 2-Ethyl-N-(4-{4-[(3-ethyl-[1,2,4]-oxadiazol-5-yl)-phenyl-methyl]-piperazin-1-yl}-3-fluoro-phenyl)-butyramide (Compound #1)

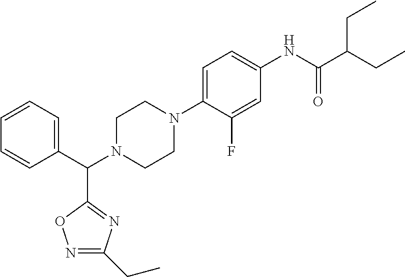

### Step A. [4-(2-fluoro-4-nitro-phenyl)-piperazin-1-yl]-phenyl-acetic acid methyl ester

To a mixture of bromo-phenyl-acetic acid methyl ester (10 mmol) and K₂CO₃ (20 mmol) in DMF (30 mL) was added 1,2-difluoro-4-nitro-benzene (1.6 g, 10 mmol). The reaction mixture was stirred at 50° C. for 3 h. To the resulting mixture was added H₂O (500 mL). After H₂O was decanted out, the crude product was obtained isolated as a semi-solid collected.

### Step B: 4-{4-[(3-ethyl-[1,2,4]-oxadiazol-5-yl)-phenyl-methyl]-piperazin-1-yl}-3-fluoro-phenylamine

To mixture of [4-(2-fluoro-4-nitro-phenyl)-piperazin-1-yl]-phenyl-acetic acid methyl ester (1 mmol) and NaOEt (1.5 mmol, 21% wt in EtOH), in EtOH (20 mL) was added N-hydroxy-propionamidine (1.5 mmol). The resulting mixture was heated at 80° C. for 4 h. After concentration, PTLC of the residue (20% EtOAc/hexanes) yielded 1-[(3-ethyl-[1,2,4]-oxadiazol-5-yl)-phenyl-methyl]-4-(2-fluoro-4-nitro-phenyl)-piperazine as a residue. The residue was re-dissolved into EtOH/EtOAc (5/5 mL). SnCl₂2H₂O (1 g) was then added. The resulting mixture was heated at 100° C. for 16 h. After being cooled down, ice-H₂O (10 mL) was added to the resulting mixture, followed by adding NaHCO₃ until pH=9. The resulting mixture was extracted by EtOAc (3×20 mL). The organic layer was collected, dried (Na₂SO₄), filtered, and concentrated to yield the title compound.

### Step C. 2-ethyl-N-(4-{4-[(3-ethyl-[1,2,4]-oxadiazol-5-yl)-phenyl-methyl]-piperazin-1-yl}-3-fluoro-phenyl)-butyramide

To a mixture of 4-{4-[(3-ethyl-[1,2,4]-oxadiazol-5-yl)-phenyl-methyl]-piperazin-1-yl}-3-fluoro-phenylamine prepared as in step B above (total amount prepared in Step B was carried over directly into this step) and TEA (0.5 mmol) in CH₂Cl₂ (5 mL) was added 2-ethyl-butyryl chloride (0.5 mmol). The resulting mixture was stirred at room temperature for 16 h. H₂O (10 mL) was added and the organic layer was separated. After concentration, PTLC yielded the title compound.

MS (ESI): mass calculated for C₂₇H₃₄FN₅O₂, 479.3; m/z measured, 480.4 [M+H]⁺

¹H NMR (CDCl₃): 7.56-7.32 (m, 6H), 7.16-7.11 (m, 2H), 6.88 (t, J=9.0, 1H), 4.90 (s, 1H), 3.15-3.03 (m, 4H), 2.84-2.68 (m, 4H), 2.64-2.57 (m, 2H), 2.05-1.95 (m, 1H), 1.78-1.67 (m, 2H), 1.62-1.52 (m, 2H), 1.37-1.20 (d, J=7.6, 3H), 0.96 (t, J=7.6, 6H).

### Example 2

### 2-Ethyl-N-(3-fluoro-4-{4-[(3-methyl-[1,2,4]-oxadiazol-5-yl)-phenyl-methyl]-piperazin-1-yl}phenyl)-butyramide (Compound #2)

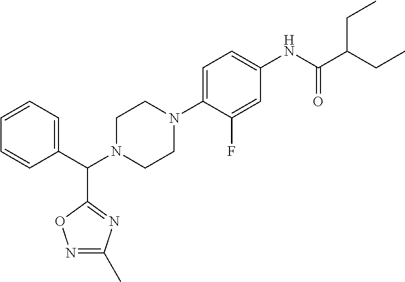

The title compound was prepared according to the process outlined in Example 1 above, with the appropriate substituent changes.

MS (ESI): mass calculated for C₂₆H₃₂FN₅O₂, 465.3; m/z measured, 466.4 [M+H]⁺

¹H NMR (CDCl₃): 7.55-7.52 (m, 2H), 7.49-7.45 (dd, J=14.0, 2.4, 1H), 7.42-7.34 (m, 3H), 7.15-7.05 (m, 2H), 6.89 (t, J=9.0, 1H), 4.92 (s, 1H), 3.15-3.05 (br, 4H), 2.77-2.70 (m, 2H), 2.65-2.57 (m, 2H), 2.44 (s, 3H), 2.04-1.97 (m, 1H), 1.78-1.67 (m, 2H), 1.64-1.53 (m, 2H), 0.97 (t, J=7.4, 6H).

### Example 3

### N-[4-(4-Benzhydryl-piperazin-1-yl)-3-fluoro-phenyl]-2-ethyl-butyramide (Compound #4)

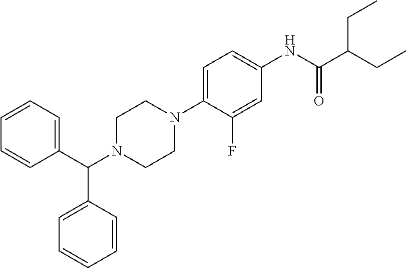

A solution of 2-ethyl-N-(3-fluoro-4-piperazin-1-yl-phenyl)butyramide (0.10 g, 0.34 mmol), chlorodiphenylmethane (0.12 mL, 0.68 mmol), and Na₂CO₃ (0.44 g, 0.41 mmol) in DMF (2 mL) was heated at 80° C. for 18 h. The resulting mixture was diluted with EtOAc (20 mL) and washed with H₂O (3×10 mL). The organic layer was dried (Na₂SO₃) and concentrated. The resulting residue was purified by SiO₂ column chromatography (EtOAc:Hex) to yield the title compound.

MS (ESI): mass calculated For C₂₄H₃₂FN₃O, 459.27; m/z measured, 460.4 [M+H]⁺

¹H NMR (CDCl₃): 7.44-7.37 (m, 6H), 7.30-7.28 (m, 3H), 7.21-7.19 (m, 2H), 7.09-7.08 (m, 1H), 6.90-6.86 (m, 1H), 4.28 (s, 1H), 3.06-3.03 (m, 4H), 2.56 (bs, 4H), 2.02-1.98 (m, 1H), 1.74-1.68 (m, 2H), 1.56-1.50 (m, 2H), 0.96 (t, J=7.4 Hz, 6H).

### Example 4

### N-(4-{4-[Bis-(4-fluoro-phenyl)-methyl]-piperazin-1-yl}-3-fluoro-phenyl)-2-ethyl-butyramide (Compound #12)

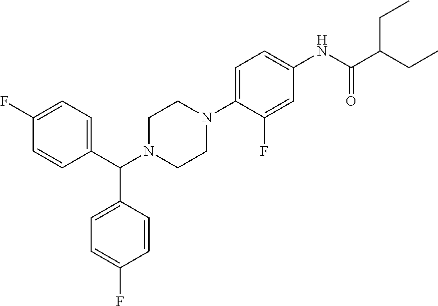

The title compound was prepared according to the process outlined in Example 3 herein, with the appropriate substituent changes.

MS (ESI): mass calculated For C₂₉H₃₂F₃N₃O, 495.58; m/z measured, 496.7 [M+H]⁺

¹H NMR (CDCl₃): 7.45-7.37 (m, 5H), 7.12-7.11 (m, 1H), 7.06 (s, 1H), 7.00-6.97 (m, 4H), 6.89 (t, J=9.1 Hz, 1H), 4.28 (s, 1H), 3.07-3.05 (m, 4H), 2.59 (bs, 4H), 1.99-1.98 (m, 1H), 1.73-1.68 (m, 2H), 1.60-1.53 (m, 2H), 0.96 (t, J=7.4 Hz, 6H).

### Example 5

### N-(4-{4-[(4-Chloro-phenyl)-phenyl-methyl]-piperazin-1-yl}-3-fluoro-phenyl)-2-ethyl-butyramide (Compound #13)

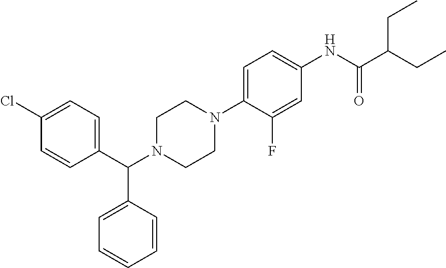

The title compound was prepared according to the process outlined in Example 3 herein, with the appropriate substituent changes.

MS (ESI): mass calculated For C₂₉H₃₃ClFN₃O, 493.23; m/z measured, 494.4 [M+H]⁺

¹H NMR (CDCl₃): 7.44-7.38 (m, 5H), 7.31-7.26 (m, 3H), 7.22-7.21 (m, 1H), 7.12 (dd, J=8.6, 1.7 Hz), 7.07 (s, 1H), 6.89 (t, J=9.1 Hz, 1H), 4.28 (s, 1H), 3.08-3.06 (m, 4H), 2.56 (bs, 4H), 2.06-1.98 (m, 1H), 1.74-1.68 (m, 2H), 1.60-1.53 (m, 2H), 0.96 (t, J=7.4 Hz, 6H).

### Example 6

### Tetrahydro-furan-3-carboxylic acid [4-(4-benzhydryl-piperazin-1-yl)-3-fluoro-phenyl]-amide (Compound #14)

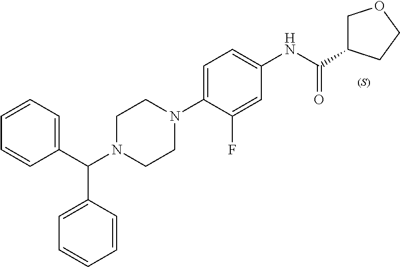

The title compound was prepared according to the process outlined in Example 3 herein, with the appropriate substituent changes.

MS (ESI): mass calculated For C₂₈H₃₀FN₃O₂, 459.23; m/z measured, 560.4 [M+H]⁺

¹H NMR (CDCl₃): 7.46-7.38 (m, 6H), 7.30-7.28 (m, 3H), 7.21-7.19 (m, 2H), 7.10-7.08 (m, 1H), 6.90-6.87 (m, 1H), 4.30 (s, 1H), 4.06-4.01 (m, 2H), 3.95-3.92 (m, 1H), 3.86-3.84 (m, 1H), 3.07-3.02 (m, 5H), 2.58 (bs, 3H), 2.27-2.24 (, 2H), 1.63 (s, 1H).

### Example 7

### 2-Ethyl-N-{3-fluoro-4-[4-(oxazol-2-yl-phenyl-methyl)-piperazin-1-yl]-phenyl}-butyramide (Compound #20)

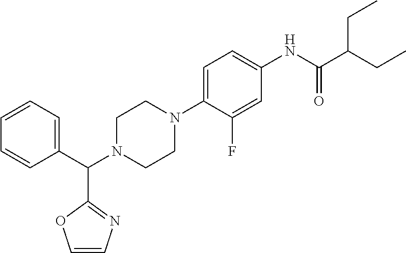

### STEP A. 1-(2-fluoro-4-nitro-phenyl)-4-(oxazol-2-yl-phenyl-methyl)-piperazine

To the solution of oxazol-2-yl-phenyl-methanone (1.7 g, 10 mmol) in THF (50 mL) was added TiCl₄ (13 mL, 1.0 M in CH₂Cl₂). The resulting mixture was stirred at room temperature for 0.5 h. 1-(2-Fluoro-4-nitro-phenyl)-piperazine (2.3 g, 10 mmol) in THF (10 mL) was then added. The resulting mixture was stirred at room temperature for 1 h. NaBH₃CN (13 mL, 1.0 M in THF) was then added. The resulting mixture was stirred at room temperature for 16 h. Saturated NaHCO₃ (50 mL) was then added. The organic layer was separated, and the aqueous layer was extracted by EtOAc (2×30 mL). The combined organic layers were concentrated to yield the title compound.

### STEP B. 3-fluoro-4-[4-(oxazol-2-yl-phenyl-methyl)-piperazin-1-yl]-phenylamine

(2-Fluoro-4-nitro-phenyl)-4-(oxazol-2-yl-phenyl-methyl)-piperazine prepared as in STEP A above (total amount prepared in Step A was carried over directly into this step) was re-dissolved in EtOH/EtOAc (50/50 mL). SnCl₂2H₂O (10 g) was added and the resulting mixture was heated at 100° C. for 16 h. After being cooled down, ice-H₂O (100 mL) was added, followed by addition of NaHCO₃ until pH=9. The resulting mixture was extracted by EtOAc (3×200 mL). The organic layer was collected, dried (Na₂SO₄), filtered, and concentrated to yield the title compound.

### Step C. 2-Ethyl-N-{3-fluoro-4-[4-(oxazol-2-yl-phenyl-methyl)-piperazin-1-yl]-phenyl}-butyramide

T a mixture of fluoro-4-[4-(oxazol-2-yl-phenyl-methyl)-piperazin-1-yl]-phenylamine prepared as in STEP B above (total amount prepared in Step B was carried over directly into this step) TEA (10.0 mmol) in CH₂Cl₂ (50 mL) was added 2-ethyl-butyryl chloride (10.0 mmol). The resulting mixture was stirred at room temperature for 16 h. H₂O (10 mL) was added, the organic layer was separated. After concentration, PTLC yield the title compound.

MS (ESI): mass calculated for C₂₆H₃₁FN₄O₂, 450.2; m/z measured, 451.4 [M+H]⁺

¹H NMR (CDCl₃): 7.57 (s, 1H), 7.57-7.51 (m, 2H), 7.48-7.25 (m, 5H), 7.15-7.07 (m, 2H), 6.87 (t, J=9.0, 1H), 4.68 (s, 1H), 3.14-3.04 (m, 4H), 2.75-2.68 (m, 2H), 2.58-2.51 (m, 2H), 2.05-1.96 (m, 1H), 1.76-1.65 (m, 2H), 1.61-1.51 (m, 2H), 0.97 (t, J=7.4, 6H).

### Example 8

### 2-Ethyl-N-{3-fluoro-4-[4-(phenyl-pyridin-3-yl-methyl)-piperazin-1-yl]-phenyl}-butyramide (Compound #24)

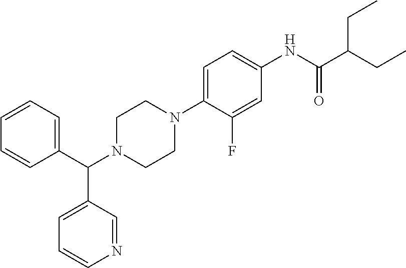

The title compound was prepared according to the process outlined in Example 3 herein, with the appropriate substituent changes.

MS (ESI): mass calculated For C₂₈H₃₃FN₄O, 460.26; m/z measured, 461.4 [M+H]⁺

¹H NMR (CDCl₃): 8.52-8.51 (m, 1H), 7.46-7.21 (m, 7H), 6.89 (t, J=9.1 Hz, 1H), 4.31 (s, 1H), 3.08 (bs, 4H), 2.57 (bs, 4H), 2.05-1.98 (m, 1H), 1.76-1.67 (m, 2H), 1.58-1.52 (m, 2H), 0.94 (t, J=7.4 Hz, 6H).

### Example 9

### 2-Ethyl-N-{3-fluoro-4-[4-(phenyl-thiophen-2-yl-methyl)-piperazin-1-yl]-phenyl}-butyramide (Compound #27)

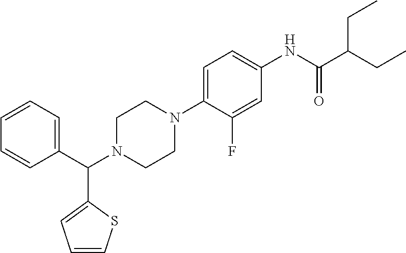

The title compound was prepared according to the process outlined in Example 7 above, with the appropriate substituent changes.

MS (ESI): mass calculated for C₂₇H₃₂FN₃OS, 465.2; m/z measured, 466.4 [M+H]⁺

¹H NMR (CDCl₃): 7.52-7.44 (m, 1H), 7.36-7.23 (m, 4H), 7.22-7.16 (m, 2H), 6.98-6.94 (m, 1H), 6.72-6.68 (m, 1H), 6.46-6.38 (m, 2H), 5.60 (s, 1H), 3.90-3.20 (m, 4H), 2.85-2.80 (m, 2H), 2.79-2.74 (m, 2H), 2.55-2.45 (m, 1H), 1.72-1.61 (m, 2H), 1.55-1.43 (m, 2H), 0.93-0.85 (m, 6H).

### Example 10

### 2-Ethyl-N-{3-fluoro-4-[4-(phenyl-pyridin-2-yl-methyl)-piperazin-1-yl]-phenyl}-butyramide (Compound #33)

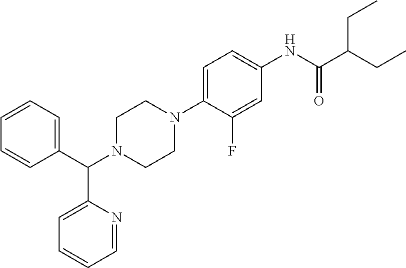

The title compound was prepared according to the process outlined in Example 7 above, with the appropriate substituent changes.

MS (ESI): mass calculated for C₂₈H₃₃FN₄O, 460.3; m/z measured, 461.4 [M+H]⁺

¹H NMR (CDCl₃): 8.53-8.48 (m, 1H), 7.65-7.56 (m, 2H), 7.55-7.47 (m, 2H), 7.44-7.37 (m, 1H), 7.32-7.24 (m, 2H), 7.24-7.18 (m, 1H), 7.17-7.08 (m, 2H), 6.88-6.83 (m, 1H), 4.48 (s, 1H), 3.13-3.05 (m, 4H), 2.65-2.53 (m, 4H), 2.05-1.96 (m, 1H), 1.75-1.65 (m, 2H), 1.58-1.47 (m, 2H), 0.97-0.86 (m, 6H).

### Example 11

### 2-Ethyl-N-{3-fluoro-4-[4-(phenyl-thiazol-2-yl-methyl)-piperazin-1-yl]-phenyl}-butyramide (Compound #39)

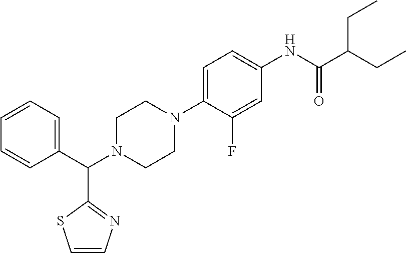

The title compound was prepared according to the process outlined in Example 7 above, with the appropriate substituent changes.

MS (ESI): mass calculated for C₂₆H₃₁FN₄OS, 466.2; m/z measured, 467.8 [M+H]⁺

¹H NMR (CDCl₃): 7.72-7.68 (m, 1H), 7.63-7.55 (s, 1H), 7.52-7.38 (m, 3H), 7.38-7.25 (m, 4H), 7.18-7.12 (m, 1H), 6.88 (t, J=9.0, 1H), 4.90 (s, 1H), 3.15-3.05 (m, 4H), 2.77-2.58 (m, 4H), 2.07-2.02 (m, 1H), 1.75-1.65 (m, 2H), 1.60-1.48 (m, 2H), 0.96 (t, J=7.4, 6H),

### Example 12

### 2-Ethyl-N-{3-fluoro-4-[4-(phenyl-pyrimidin-2-yl-methyl)-piperazin-1-yl]-phenyl}-butyramide (Compound #40)

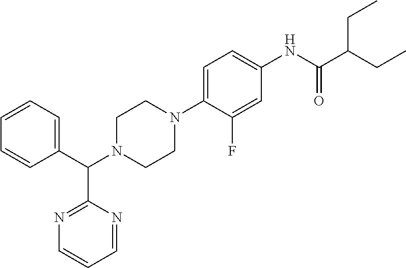

The title compound was prepared according to the process outlined in Example 7 above, with the appropriate substituent changes.

MS (ESI): mass calculated for C₂₇H₃₂FN₅O, 461.2; m/z measured, 462.3 [M+H]⁺

¹H NMR (CDCl₃): 8.75 (d, J=4.9, 2H), 7.65-7.61 (m, 2H), 7.48-7.43 (m, 2H), 7.37-7.08 (m, 6H), 6.88 (t, J=9.0, 1H), 4.67 (s, 1H), 3.18-3.08 (br, 4H), 2.75-2.57 (m, 4H), 2.05-1.97 (m, 1H), 1.78-1.68 (m, 2H), 1.61-1.50 (m, 2H), 0.96 (t, J=7.4, 6H),

### Example 13

### N-(1-Ethyl-propyl)-3-fluoro-4-[4-(oxazol-2-yl-phenyl-methyl)-piperazin-1-yl]-benzamide (Compound #46)

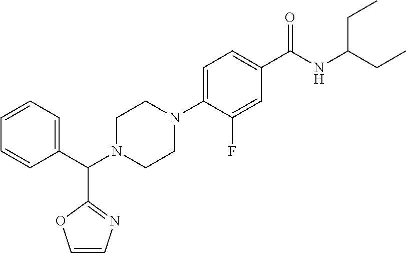

### STEP A. 1-(oxazol-2-yl-phenyl-methyl)-piperazine trifluoro-acetic acid salt

To a mixture of oxazol-2-yl-phenyl-methanone (1.7 g, 10 mmol) in THF (50 mL) was added TiCl₄ (13 mL, 1.0 M in CH₂Cl₂). The resulting mixture was stirred at room temperature for 0.5 h. piperazine-1-carboxylic acid tert-butyl ester (10 mmol) in THF (10 mL) was then added. The resulting mixture was stirred at room temperature for 1 h. NaBH₃CN (13 mL, 1.0 M in THF) was then added. The resulting mixture was stirred at room temperature for 16 h. Saturated NaHCO₃ (50 mL) was then added. The organic layer was separated, and the aqueous layer was extracted by EtOAc (2×30 mL). The combined organic layers were concentrated and re-dissolved in CH₂Cl₂ (50 mL. CF₃COOH (10 mL) was then added and the resulting mixture was stirred at room temperature for 16 h. The resulting mixture was concentrated to yield the title compound.

### STEP B. 4-bromo-N-(1-ethyl-propyl)-3-fluoro-benzamide

To a resulting mixture of 4-bromo-3-fluoro-benzoic acid (10.5 g, 5 mmol) and PS-carboimidide (6 mmol) in CH₂Cl₂ (100 mL) was added 1-ethyl-propylamine (435.0 mg, 5 mmol). The resulting mixture was stirred at room temperature for 16 h. After filtration, the filtrate was concentrated to yield the title compound.

### STEP C. N-(1-ethyl-propyl)-3-fluoro-4-[4-(oxazol-2-yl-phenyl-methyl)-piperazin-1-yl]-benzamide

A mixture of 1-(oxazol-2-yl-phenyl-methyl)piperazine; trifluoro-acetic acid salt prepared as in STEP A above (1 mmol), 4-bromo-N-(1-ethyl-propyl)-3-fluoro-benzamide (1 mmol) prepared as in STEP B above, Cs₂CO₃ (3 mmol), Pd₂ dba₃ (0.01 mmol), and Binap (0.04 mmol), in xylene (3 ml) was heated at 100° C. for 16 h. After being cooled down, preparative thin layer chromatography yielded the title compound.

MS (ESI): mass calculated for C₂₆H₃₁FN₄O₂, 450.2; m/z measured, 451.9 [M+H]⁺

¹H NMR (CDCl₃): 7.68 (s, 1H), 7.65-7.61 (m, 2H), 7.50-7.43 (m, 2H), 7.42-7.31 (m, 4H), 7.12 (s, 1H), 6.92 (t, J=8.4, 1H), 5.67 (d, J=8.8, 1H), 4.80 (s, 1H), 4.05-3.95 (m, 1H), 3.27-3.18 (m, 4H), 2.77-2.72 (m, 2H), 2.60-2.53 (m, 2H), 1.72-1.62 (m, 2H), 1.53-1.45 (m, 2H), 0.96 (t, J=7.4, 6H),

### Example 14

### N-Cyclopentyl-3-fluoro-4-[4-(oxazol-2-yl-phenyl-methyl)-piperazin-1-yl]-benzamide (Compound #49)

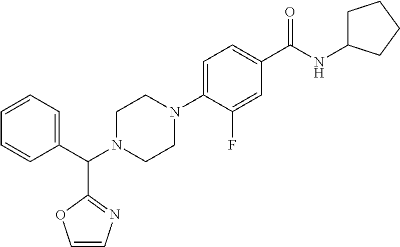

The title compound was prepared according to the process outlined in Example 13 above, with the appropriate substituent changes.

MS (ESI): mass calculated for C₂₆H₂₉FN₄O₂, 448.2; m/z measured, 449.2 [M+H]⁺

¹H NMR (CDCl₃): 7.68 (s, 1H), 7.57-7.52 (m, 2H), 7.48-7.43 (m, 2H), 7.40-7.31 (m, 4H), 7.12 (s, 1H), 6.92-6.87 (m, 1H), 6.03 (d, J=7.3, 1H), 4.80 (s, 1H), 4.92-4.85 (m, 1H), 3.27-3.18 (m, 4H), 2.77-2.70 (m, 2H), 2.60-2.53 (m, 2H), 2.12-2.05 (m, 2H), 1.78-1.62 (m, 4H), 1.52-1.42 (m, 2H),

### Example 15

### 2-[4-(4-Benzhydryl-piperazin-1-yl)-3-fluoro-phenyl]-N,N-diethyl-acetamide (Compound #51)

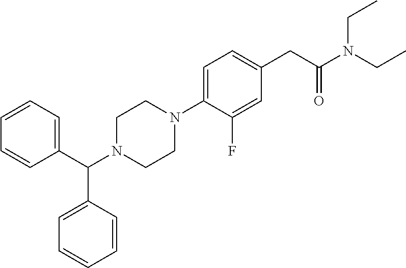

### Step A. N,N-Diethyl-2-(3-fluoro-4-hydroxy-phenyl)-acetamide

A solution of 3-fluoro-4-hydroxy-phenyl acetic acid (2.0 g, 11.7 mmol), diethylamine (1.3 mL, 13.0 mmol), and EDC (2.7 g, 14.0 mmol) in DCM (100 mL) was stirred for 15 h. The resulting mixture was diluted with 1N NaOH (50 ml) and washed with DCM. The aqueous layer was neutralized with 3N HCl and basified with 1N NaHCO₃. The desired product was extracted out of the aqueous layer using EtOAc to yield the title compound as a white powder.

### Step B. Trifluoro-methanesulfonic acid 4-diethylcarbamoylmethyl-2-fluoro-phenyl ester

A solution of N,N-diethyl-2-(3-fluoro-4-hydroxy-phenyl)-acetamide (0.95 g, 4.2 mmol), N-phenyltrifluoromethanesulfonimide (1.8 g, 5.1 mmol), and Et₃N (1.2 mL, 8.4 mmol) in DCM (50 mL) was refluxed for 15 h. The resulting mixture was concentrated and the residue was purified by SiO₂ column chromatography (EtOAc:Hex) to yield the title compound.

### Step C. 2-[4-(4-Benzhydryl-piperazin-1-yl)-3-fluoro-phenyl]-N,N-diethyl-acetamide

A solution of trifluoro-methanesulfonic acid 4-diethylcarbamoylmethyl-2-fluoro-phenyl ester (0.20 g, 0.56 mmol), 1-benzhydryl-piperazine (0.11 g, 0.62 mmol), Pd₂(dba)₃ (0.01 g, 0.0075 mmol), XPhos (0.01 g, 0.015 mmol), and sodium tert-butoxide (0.11 g, 1.05 mmol) in toluene (2 mL) was heated by microwave irradiation at 120° C. for 20 minutes. The resulting mixture was cooled and then filtered through dichotomous earth and washed with DCM (10 mL). The organic liquid was concentrated and the residue was purified by SiO₂ column chromatography (2M NH₃ in MeOH:DCM) to yield the title compound.

MS (ESI): mass calculated For C₂₉H₃₄FN₃O, 459.60; m/z measured, 460.5 [M+H]⁺

¹H NMR (CDCl₃): 7.45 (d, J=7.2 Hz, 3H), 7.30-7.28 (m, 4H), 7.21-7.18 (m, 2H), 7.05-6.88 (m, 4H), 4.30 (s, 1H), 3.70 (s, 1H), 3.60 (s, 1H), 3.40-3.38 (m, 2H), 3.32-3.29 (m, 2H), 3.10-3.08 (m, 4H), 2.58 (s, 4H), 1.15-1.10 (m, 6H).

### Example 16

### N-{4-[4-(Cyclopropyl-phenyl-methyl)-piperazin-1-yl]-3-fluoro-phenyl}-2-ethyl-butyramide (Compound #53)

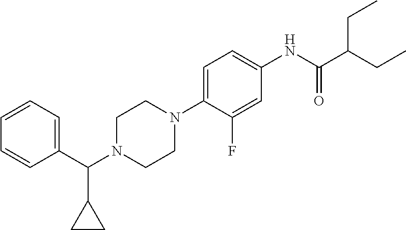

The title compound was prepared according to the process outlined in Example 7 above, with the appropriate substituent changes.

MS (ESI): mass calculated for C₂₆H₃₄FN₃O, 423.3; m/z measured, 424.6 [M+H]⁺

¹H NMR (CDCl₃): 7.48-7.25 (m, 7H), 7.14-7.08 (m, 1H), 6.92-6.83 (m, 1H), 3.12-2.84 (m, 6H), 2.58-2.50 (m, 2H), 2.30 (d, J=9.4, 1H), 2.05-1.96 (m, 1H), 1.75-1.65 (m, 2H), 1.61-1.50 (m, 2H), 1.12-1.02 (m, 1H), 0.96 (t, J=6.4, 6H), 0.83-0.75 (m, 1H), 0.60-0.30 (m, 2H), 0.10-0.01 (m, 1H)

### Example 17

### Cyclopentanecarboxylic acid {3-fluoro-4-[4-(oxazol-2-yl-phenyl-methyl)-piperazin-1-yl]-phenyl}amide (Compound #54)

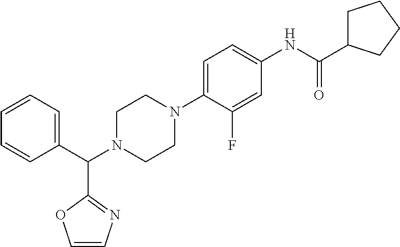

The title compound was prepared according to the process outlined in Example 7 above, with the appropriate substituent changes.

MS (ESI): mass calculated for C₂₆H₂₉FN₄O₂, 448.2; m/z measured, 449.3 [M+H]⁺

¹H NMR (CDCl₃): 7.65 (s, 1H), 7.60-7.50 (m, 2H), 7.47-7.25 (m, 5H), 7.14-7.06 (m, 2H), 6.88 (t, J=9.0, 1H), 4.76 (s, 1H), 3.15-3.02 (m, 4H), 2.78-2.47 (m, 5H), 2.00-1.72 (m, 6H), 1.71-1.52 (m, 2H).

### Example 18

### N-{4-[4-(Di-pyridin-2-yl-methyl)-piperazin-1-yl]-3-fluoro-phenyl}-2-ethyl-butyramide (Compound #55)

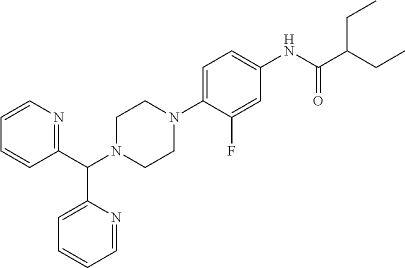

The title compound was prepared according to the process outlined in Example 7 above, with the appropriate substituent changes.

MS (ESI): mass calculated for C₂₇H₃₂FN₅O, 461.3; m/z measured, 462.3 [M+H]⁺

¹H NMR (CDCl₃): 8.58-8.56 (m, 2H), 7.71-7.65 (m, 4H), 7.46-7.42 (m, 1H), 7.22 (s, 1H), 7.18-7.10 (m, 3H), 6.88 (t, J=9.0, 1H), 4.73 (s, 1H), 3.15-3.10 (m, 4H), 2.68-2.62 (m, 4H), 2.06-2.02 (m, 1H), 1.78-1.67 (m, 2H), 1.62-1.52 (m, 2H), 0.98-0.92 (m, 6H).

### Example 19

### {3-Fluoro-4-[4-(oxazol-2-yl-phenyl-methyl)-piperazin-1-yl]-phenyl}-(3-methyl-pyridin-2-yl)-amine (Compound #57)

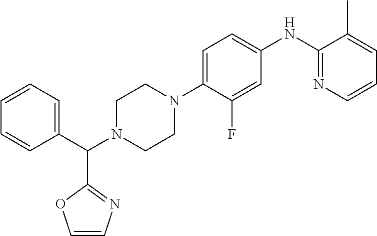

A mixture of fluoro-4-[4-(oxazol-2-yl-phenyl-methyl)-piperazin-1-yl]-phenylamine prepared as in Example 7, Step B above (100 mg) and 2-bromo-6-methyl-pyridine (1 mmol) in n-BuOH (5 mL) was heated at about 130° C. for 16 h. The resulting mixture was cooled and then subjected to preparative TLC to yield the title compound.

MS (ESI): mass calculated for C₂₆H₂₆FN₅O, 443.2; m/z measured, 444.2 [M+H]⁺

¹H NMR (CDCl₃): 7.67 (s, 1H), 7.57-7.53 (m, 2H), 7.42-7.21 (m, 4H), 7.18-7.11 (m, 2H), 6.98-6.88 (m, 2H), 6.63-6.61 (m, 2H), 6.50-6.45 (br, 1H), 4.80 (s, 1H), 3.50 (m, 1H), 3.15-3.08 (m, 4H), 2.86-2.70 (m, 2H), 2.62-2.53 (m, 2H), 2.43 (s, 3H).

### Example 20

### {3-Fluoro-4-[4-(oxazol-2-yl-phenyl-methyl)-piperazin-1-yl]-phenyl}-(1-methyl-1H-imidazol-2-yl)-amine (Compound #58)

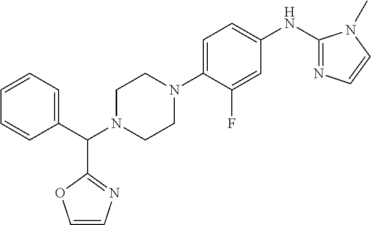

The title compound was prepared according to the processes outlined in Example 19 and above, with the appropriate substituent changes.

MS (ESI): mass calculated for C₂₆H₂₆FN₅O, 443.2; m/z measured, 444.2 [M+H]⁺

¹H NMR (CDCl₃): 7.67 (s, 1H), 7.57-7.53 (m, 2H), 7.42-7.21 (m, 4H), 7.18-7.11 (m, 2H), 6.98-6.88 (m, 2H), 6.63-6.61 (m, 2H), 6.50-6.45 (br, 1H), 4.80 (s, 1H), 3.50 (m, 1H), 3.15-3.08 (m, 4H), 2.86-2.70 (m, 2H), 2.62-2.53 (m, 2H), 2.43 (s, 3H).

### Example 21

### 2-Ethyl-N-{3-fluoro-4-[4-(oxazol-2-yl-phenyl-methyl)-piperazin-1-yl]-phenyl}-butyramide (Compound #59)

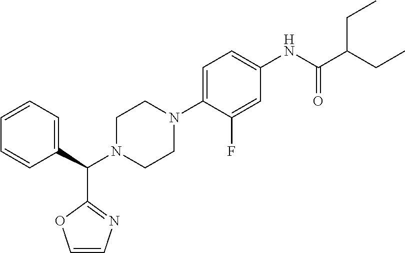

The title compound was prepared according to the process outlined in Example 7 above, with the appropriate substituent changes and separation by chiral chromatography (IPA/Hexanes).

MS (ESI): mass calculated for C₂₄H₂₅FN₆O, 432.2; m/z measured, 433.2 [M+H]⁺

¹H NMR (CDCl₃): 7.65 (s, 1H), 7.57-7.52 (m, 2H), 7.40-7.28 (m, 3H), 7.30 (s, 1H), 6.90-6.82 (m, 2H), 6.74 (d, J=1.3, 1H), 6.66-6.55 (m, 2H), 6.18-6.10 (br, 1H), 4.77 (s, 1H), 3.45 (s, 3H), 3.07-3.00 (m, 4H), 2.74-2.66 (m, 2H), 2.58-2.50 (m, 2H).

### Example 22

### 2-Ethyl-N-{3-fluoro-4-[4-(oxazol-2-yl-phenyl-methyl)-piperazin-1-yl]-phenyl}-butyramide (Compound #60)

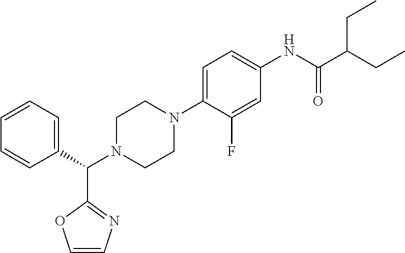

The title compound was prepared according to the process outlined in Example 7 above, with the appropriate substituent changes and separation by chiral chromatography (IPA/Hexanes).

MS (ESI): mass calculated for C₂₆H₃₁FN₄O₂, 450.2; m/z measured, 451.4 [M+H]⁺

¹H NMR (CDCl₃): 7.57 (s, 1H), 7.57-7.51 (m, 2H), 7.48-7.25 (m, 5H), 7.15-7.07 (m, 2H), 6.87 (t, J=9.0, 1H), 4.68 (s, 1H), 3.14-3.04 (m, 4H), 2.75-2.68 (m, 2H), 2.58-2.51 (m, 2H), 2.05-1.96 (m, 1H), 1.76-1.65 (m, 2H), 1.61-1.51 (m, 2H), 0.97 (t, J=7.4, 6H).

### Example 23

### {3-Fluoro-4-[4-(oxazol-2-yl-phenyl-methyl)-piperazin-1-yl]-phenyl}-(4-methyl-pyrimidin-2-yl)-amine (Compound #61)

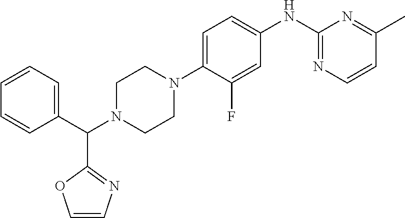

The title compound was prepared according to the process outlined in Example 19 above, with the appropriate substituent changes.

MS (ESI): mass calculated for C₂₅H₂₅FN₆O, 444.2; m/z measured, 445.2 [M+H]⁺

¹H NMR (CDCl₃): 8.24 (d, J=5.0, 1H), 7.64-7.47 (m, 3H), 7.38-7.21 (m, 3H), 7.18-7.04 (m, 3H), 6.92-6.85 (m, 1H), 6.59 (d, J=5.0, 1H), 4.77 (s, 1H), 3.15-3.08 (m, 4H), 2.86-2.67 (m, 2H), 2.58-2.51 (m, 2H), 2.40 (s, 3H).

### Example 24

### {3-Fluoro-4-[4-(oxazol-2-yl-phenyl-methyl)-piperazin-1-yl]-phenyl}-(6-methyl-pyridin-2-yl)-amine (Compound #62)

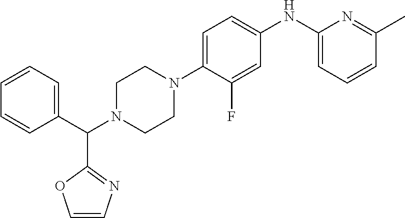

The title compound was prepared according to the process outlined in Example 19 above, with the appropriate substituent changes.

MS (ESI): mass calculated for C₂₆H₂₆FN₅O, 443.2; m/z measured, 444.2 [M+H]⁺

¹H NMR (CDCl₃): 8.12-8.06 (m, 1H), 7.80-7.25 (m, 8H), 7.16-7.05 (m, 2H), 6.89 (t, J=9.0, 1H), 6.72-6.68 (m, 1H), 6.03 (s, 1H), 4.68 (s, 1H), 3.18-3.04 (m, 4H), 2.75-2.68 (m, 2H), 2.58-2.51 (m, 2H), 2.20 (s, 3H)

### Example 25

### {3-Fluoro-4-[4-(oxazol-2-yl-phenyl-methyl)-piperazin-1-yl]-phenyl}-o-tolyl-amine (Compound #63)

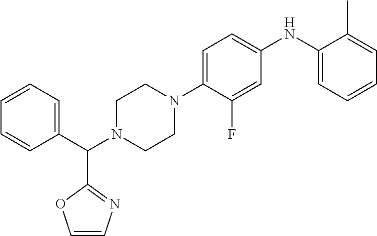

A mixture of fluoro-4-[4-(oxazol-2-yl-phenyl-methyl)-piperazin-1-yl]-phenylamine prepared as in STEP B of Example 7 above (100 mg), 1-bromo-2-methyl-benzene (1 mmol), Cs₂CO₃ (3 mmol), Pd₂ dba₃ (0.01 mmol), and BINAP (0.04 mmol), in xylene (3 ml) was heated at 130° C. for 16 h. After being cooled down, PTLC yielded the title compound.

MS (ESI): mass calculated for C₂₇H₂₇FN₄O, 442.2; m/z measured, 443.2 [M+H]⁺

¹H NMR (CDCl₃): 7.62 (s, 1H), 7.56-7.41 (m, 2H), 7.38-7.25 (m, 3H), 7.18-7.06 (m, 4H), 6.92-6.82 (m, 2H), 6.72-6.63 (m, 2H), 5.24 (s, 1H), 4.77 (s, 1H), 3.15-3.08 (m, 4H), 2.86-2.67 (m, 2H), 2.58-2.51 (m, 2H), 2.24 (s, 3H).

### Example 26

### 2-Ethyl-N-(3-fluoro-4-{4-[(1-methyl-1H-imidazol-2-yl)-phenyl-methyl]-piperazin-1-yl}-phenyl)-butyramide (Compound #64)

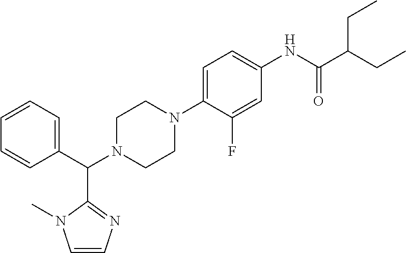

The title compound was prepared according to the process outlined in Example 7 above, with the appropriate substituent changes.

MS (ESI): mass calculated for C₂₇H₃₄FN₅O, 463.3; m/z measured, 464.3 [M+H]⁺

¹H NMR (CDCl₃): 7.56-7.52 (m, 2H), 7.46 (dd, J=14.1, 2.4, 1H), 7.38-7.34 (m, 2H), 7.30-7.25 (m, 1H), 7.25-7.12 (m, 2H), 7.01 (s, 1H), 6.90 (t, J=9.0, 1H), 6.77 (s, 1H), 4.73 (s, 1H), 3.68 (s, 3H), 3.15-3.07 (m, 4H), 2.87-2.62 (m, 2H), 2.58-2.51 (m, 2H), 2.05-1.96 (m, 1H), 1.76-1.63 (m, 2H), 1.60-1.51 (m, 2H), 0.98-0.92 (m, 6H).

### Example 27

### N-{4-[4-(Cyclopropyl-oxazol-2-yl-methyl)-piperazin-1-yl]-3-fluoro-phenyl}-2-ethyl-butyramide (Compound #66)

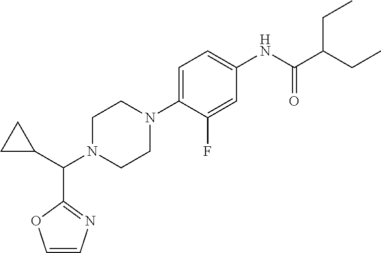

### STEP A: cyclopropyl-oxazol-2-yl-methanone

To a mixture of 2-tributylstannanyl-oxazole (5 mmol, 1.1 mL), and PdCl₂(PPh₃)₂ (35 mg) in toluene (5 mL) at 60° C. was added dropwise cyclopropanecarbonyl chloride (10 mmol). After finishing adding, the temperature was raised to 100° C. After 15 min at this temperature, the resulting mixture was cooled down and purified by PTLC (20% EtOAc/Hexanes) to yield the title compound.

### STEP B: N-{4-[4-(Cyclopropyl-oxazol-2-yl-methyl)-piperazin-1-yl]-3-fluoro-phenyl}-2-ethyl-butyramide

The title product was prepared according to the procedure described in Example 7 above, with appropriate substituent changes.

MS (ESI): mass calculated for C₂₃H₃₁FN₄O₂, 414.2; m/z measured, 415.3 [M+H]⁺

¹H NMR (CDCl₃): 7.68 (s, 1H), 7.46 (dd, J=4.0, 2.4, 1H), 7.25 (s, 1H), 7.15-7.08 (m, 2H), 6.89 (t, J=9.0, 1H), 3.15-3.05 (m, 4H), 2.96-2.89 (m, 3H), 2.71-2.64 (m, 2H), 2.05-1.97 (m, 1H), 1.77-1.68 (m, 2H), 1.62-1.52 (m, 2H), 1.40-1.32 (m, 1H), 0.98-0.93 (m, 6H), 0.84-0.75 (m, 1H), 0.58-0.51 (m 1H), 0.48-0.41 (m, 1H), 0.30-0.21 (m, 1H)

### Example 28

### N-{3-Fluoro-4-[4-(oxazol-2-yl-phenyl-methyl)-piperazin-1-yl]-phenyl}-isobutyramide (Compound #72)

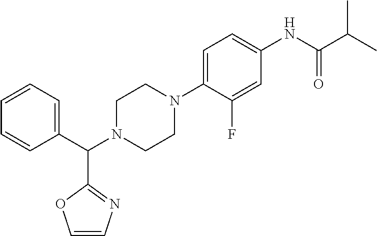

3-Fluoro-4-[4-(oxazol-2-yl-phenyl-methyl)-piperazin-1-yl]-phenylamine (52 mg, 0.15 mmol), isobutyryl chloride (16 mg, 0.15 mmol) and DIPEA (19.5 mg, 0.15 mmol) were stirred in DCM (5.0 mL) for 18 h. The resulting mixture was diluted with additional DCM (20.0 mL) and washed with 1N NaOH (5.0 mL) and water (2×20 mL). The organic phase was dried (Na₂SO₄), filtered and concentrated to dryness to yield a residue (55 mg). Chromatography of the residue (SiO₂, 0-7% acetone/DCM, gradient) yielded the title compound.

MS (ESI) mass calculated for C₂₄H₂₇FN₄O₂, 422.50; m/z measured, 423.5 [M+H]⁺

¹H NMR (CDCl₃): 7.63 (s, 1H), 7.55-7.48 (m, 2H), 7.41 (dd, J=14.0, 2.2, 1H), 7.37-7.32 (m, 2H), 7.32-7.27 (m, 1H), 7.18 (s, 1H), 7.10-7.05 (m, 2H), 6.85 (t, J=9.0, 1H), 4.77 (s, 1H), 3.11-3.04 (m, 4H), 2.73-2.65 (m, 2H), 2.57-2.42 (m, 3H), 1.27-1.16 (m, 6H).

### Example 29

### Cyclobutanecarboxylic acid {3-fluoro-4-[4-(oxazol-2-yl-phenyl-methyl)-piperazin-1-yl]-phenyl}-amide (Compound #73)

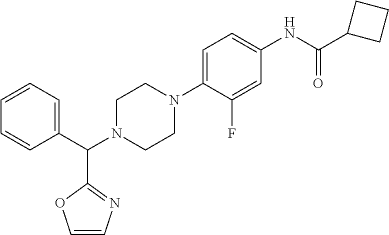

3-Fluoro-4-[4-(oxazol-2-yl-phenyl-methyl)-piperazin-1-yl]-phenylamine (67 mg, 0.19 mmol), cyclobutanecarbonyl chloride (22.6 mg, 0.19 mmol) and DIPEA (27 mg, 0.19 mmol) were stirred in DCM (5.0 mL) for 18 h. Work up of the reaction and chromatography as described in Example 28 above yielded the title compound.

MS (ESI) mass calculated for C₂₅H₂₇FN₄O₂, 434.51; m/z measured, 435.5 [M+H]⁺

¹H NMR (CDCl₃): 7.63 (s, 1H), 7.54-7.49 (m, 2H), 7.41 (dd, J=16.3, 2.2, 1H), 7.38-7.32 (m, 2H), 7.10-7.00 (m, 3H), 6.85 (t, J=9.0, 1H), 4.77 (s, 1H), 3.15-3.03 (m, 5H), 2.73-2.65 (m, 2H), 2.56-2.48 (m, 2H), 2.40-2.31 (m, 2H), 2.24-2.17 (m, 2H), 2.03-1.85 (m, 2H).

### Example 30

### N-{3-Methyl-4-[4-(oxazol-2-yl-phenyl-methyl)-piperazin-1-yl]-phenyl}-isobutyramide (Compound #74)

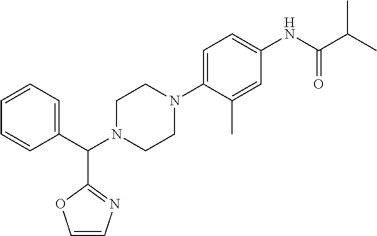

The title compound was prepared according to the process described in Example 28 above reacting 3-methyl-4-[4-(oxazol-2-yl-phenyl-methyl)-piperazin-1-yl]-phenylamine (53 mg, 0.15 mmol) to yield the title compound.

MS (ESI) mass calculated for C₂₅H₃₀N₄O₂, 418.53; m/z measured, 419.6 [M+H]⁺

¹H NMR (CDCl₃): 7.64 (s, 1H), 7.56-7.51 (m, 2H), 7.38-7.27 (m, 5H), 7.09 (s, 1H), 7.02 (s, 1H), 6.96 (d, J=8.5, 1H), 4.75 (s, 1H), 2.93-2.87 (m, 4H), 2.71-2.61 (m, 2H), 2.52-2.41 (m, 3H), 2.24 (s, 3H), 1.25-1.20 (m, 6H).

### Example 31

### Cyclobutanecarboxylic acid {3-methyl-4-[4-(oxazol-2-yl-phenyl-methyl)-piperazin-1-yl]-phenyl}-amide (Compound #76)

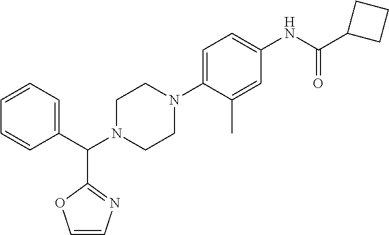

The title compound was prepared according to the process described in Example 29 above, reacting 3-methyl-4-[4-(oxazol-2-yl-phenyl-methyl)-piperazin-1-yl]-phenylamine (60 mg, 0.17 mmol) to yield the title compound.

MS (ESI) mass calculated for C₂₆H₃₀N₄O₂, 430.55; m/z measured, 431.6 [M+H]⁺

¹H NMR (CDCl₃): 7.63 (s, 1H), 7.56-7.50 (m, 2H), 7.39-7.27 (m, 5H), 7.11-7.02 (m, 2H), 6.97-6.92 (m, 1H), 4.74 (s, 1H), 3.18-3.04 (m, 1H), 2.95-2.75 (m, 4H), 2.69-2.30 (m, 5H), 2.30-2.22 (m, 3H), 2.20-2.14 (m, 2H), 2.06-1.82 (m, 2H).

### Example 32

### N-{4-[4-(Cyclobutyl-phenyl-methyl)-piperazin-1-yl]-3-fluoro-phenyl}-2-ethyl-butyramide (Compound #78)

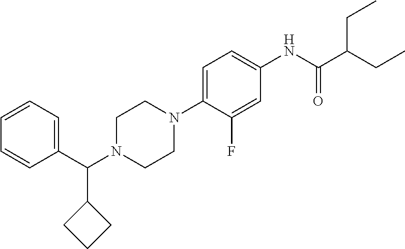

The title compound was prepared according to the process outlined in Example 7 herein, with the appropriate substituent changes.

MS (ESI): mass calculated for C₂₇H₃₆FN₃O, 437.59; m/z measured, 438.3 [M+H]⁺

¹H NMR (CDCl₃): 7.43 (dd, J=2.5, 14.0, 1H), 7.34-7.29 (m, 2H), 7.27-7.22 (m, 2H), 7.14-7.07 (m, 2H), 6.86 (t, J=9.1, 1H), 3.20 (d, J=9.6, 1H), 3.00 (t, J=4.7, 4H), 2.86-2.76 (m, 1H), 2.65-2.58 (m, 2H), 2.58-2.50 (m, 2H), 2.27-2.13 (m, 1H), 2.08-1.92 (m, 2H), 1.89-1.64 (m, 5H), 1.62-1.44 (m, 4H), 0.96 (t, J=7.4, 6H).

### Example 33

### Cyclopropanecarboxylic acid {3-methyl-4-[4-(oxazol-2-yl-phenyl-methyl)-piperazin-1-yl]-phenyl}amide (Compound #81)

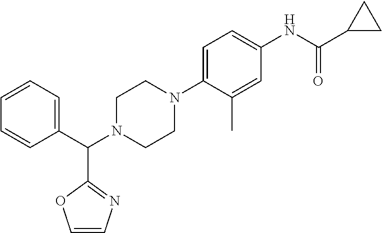

A mixture of 3-methyl-4-[4-(oxazol-2-yl-phenyl-methyl)-piperazin-1-yl]-phenylamine (66 mg, 0.19 mmol), cyclopropanecarboxylic acid (17.2 mg, 0.20 mmol) and HATU (80 mg, 0.21 mmol) in DMF (4.0 mL) was stirred at room temperature for 18 h. The resulting mixture was diluted with water and extracted with ethyl acetate. The organic phase was dried (Na₂SO₄), filtered and concentrated to dryness to yield a residue. The residue was purified on a reversed phase acidic HPLC to yield title compound as its corresponding trifluoroacetic acid salt.

MS (ESI) mass calculated for C₂₅H₂₈N₄O₂, 416.52; m/z measured, 417.3 [M+H]⁺

¹H NMR (CD₃OD): 8.04 (d, J=0.8, 1H), 7.67-7.63 (m, 2H), 7.55-7.50 (m, 3H), 7.41-7.34 (m, 3H), 7.05 (d, J=8.5, 1H), 5.78 (s, 1H), 3.30-3.07 (m, 8H), 2.28 (s, 3H), 1.75-1.68 (m, 1H), 0.94-0.89 (m, 2H), 0.87-0.80 (m, 2H).

### Example 34

### 2-Ethyl-N-(3-fluoro-4-{4-[(5-isopropyl-[1,2,4]-oxadiazol-3-yl)-phenyl-methyl]-piperazin-1-yl}-phenyl)-butyramide (Compound #82)

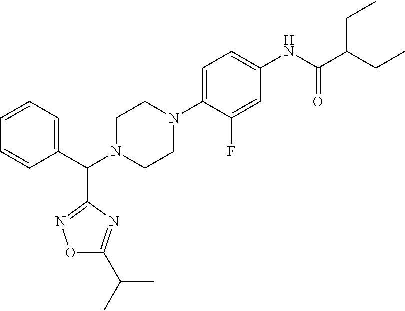

### STEP A: 4-(2-Fluoro-4-nitro-phenyl)-piperazin-1-yl]-phenyl-acetonitrile

To a heterogeneous mixture of the product of 1-(2-fluoro-4-nitro-phenyl)-piperazine (3.00 g, commercially available) in CH₃CN (50 mL) was added benzaldehyde (1.35 mL) followed after 45 min by TMSCN (1.95 mL). The resulting mixture was stirred at room tempertaure for 22 h and then quenched by saturated aqueous NH₄Cl solution. The aqueous layer was extracted with EtOAc and the combined organic layers were washed with brine, dried over Na₂SO₄, filtered and concentrated in vacuo to yield the title compound as an orange solid.

MS (electrospray): exact mass calculated for C₁₈H₁₇FN₄O₂, 340.13; measured m/z 341.5 [M+H]⁺

### STEP B: 2-[4-(2-Fluoro-4-nitro-phenyl)-piperazin-1-yl]-N-hydroxy-2-phenyl-acetamidine

To a heterogeneous mixture of the product of Step B (4.53 g) in EtOH (133 mL) was added NH₂OH.HCl (4.62 g) followed by Na₂CO₃ (7.05 g). The resulting mixture was stirred at 80° C. for 20 h and then concentrated in vacuo. The residue was chromatographed on SiO₂ (5% EtOAc/Hexanes to 90% EtOAc/Hexanes) to yield the title compound.

MS (electrospray): exact mass calculated for C₁₈H₂₀FN₅O₃, 373.16; measured m/z 374.3 [M+H]⁺.

### STEP C: 1-(2-Fluoro-4-nitro-phenyl)-4-[(5-isopropyl-[1,2,4]-oxadiazol-3-yl)-phenyl-methyl]-piperazine

To a solution of the product of Step B (228 mg) in THF (4 mL) was added DIPEA (0.177 mL) followed by isobutyryl chloride (0.065 mL). After 20 min at room temperature, the resulting mixture was heated at 155° C. for 20 min in the microwave. The resulting mixture was concentrated in vacuo and chromatography on SiO₂ (Hexanes to 20% EtOAc/Hexanes) to yield the title compound.

MS (electrospray): exact mass calculated for C₂₂H₂₄FN₅O₃, 425.19; measured m/z 426.5 [M+H]⁺.

### STEP D: 3-Fluoro-4-{4-[(5-isopropyl-[1,2,4]-oxadiazol-3-yl)-phenyl-methyl]-piperazin-1-yl}phenylamine

To a solution of the product of Step C (114 mg) in EtOH (5 mL) was added SnCl₂.2H₂O (303 mg) and the resulting mixture heated at reflux for 3 h. The resulting mixture was then treated with 1 N NaOH and the aqueous layer extracted with EtOAc and CH₂Cl₂. The combined organic layers were dried over Na₂SO₄, filtered and concentrated in vacuo to yield title compound.

MS (electrospray): exact mass calculated for C₂₂H₂₆FN₅O, 395.21; measured m/z 396.5 [M+H]⁺.

### STEP E: 2-Ethyl-N-(3-fluoro-4-{4-[(5-isopropyl-[1,2,4]-oxadiazol-3-yl)-phenyl-methyl]-piperazin-1-yl}-phenyl)-butyramide

To a solution of the product of Step D (95 mg) in CH₂Cl₂ (5 mL) was added TEA (0.035 mL) followed by 2-ethylbutyryl chloride (0.033 mL). After aging for 1 h. The resulting mixture was concentrated in vacuo and chromatography on SiO₂ (Hexanes to 30% EtOAc/Hexanes) yield the title compound.

MS (electrospray): exact mass calculated for C₂₈H₃₆FN₅O₂, 493.29; measured m/z 494.6 [M+H]⁺

¹H NMR (500 MHz, CDCl₃): 7.57-7.52 (m, 2H), 7.43 (dd, J=14.02, 2.39 Hz, 1H), 7.38-7.27 (m, 3H), 7.12-7.02 (m, 2H), 6.90-6.83 (m, 1H), 4.75 (s, 1H), 3.22 (sept., J=6.99 Hz, 1H), 3.14-3.03 (m, 4H), 2.75-2.65 (m, 2H), 2.64-2.53 (m, 2H), 2.02-1.94 (m, 1H), 1.75-1.64 (m, 2H), 1.59-1.49 (m, 2H), 1.41-1.36 (m, 6H), 0.94 (t, J=7.41 Hz, 6H).

### Example 35

### N-{4-[4-(Cyclopentyl-phenyl-methyl)-piperazin-1-yl]-3-fluoro-phenyl}-2-ethyl-butyramide (Compound #83)

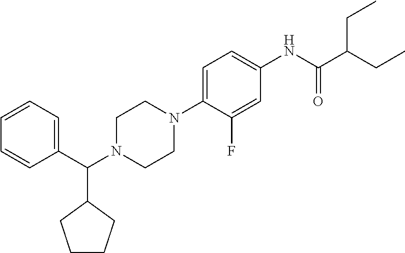

The title compound was prepared according to the process outlined in Example 7 herein, with the appropriate substituent changes.

MS (ESI): mass calculated for C₂₈H₃₈FN₃O, 451.62; m/z measured, 452.3 [M+H]⁺

¹H NMR (CDCl₃): 7.41 (dd, J=2.5, 14.3, 1H), 7.36-7.31 (m, 2H), 7.28-7.23 (m, 1H), 7.22-7.15 (m, 3H), 7.14-7.08 (m, 1H), 7.86 (t, J=9.1, 1H), 3.29 (d, J=10.4, 1H), 3.11-2.95 (m, 4H), 2.74-2.46 (m, 5H), 2.05-1.95 (m, 1H), 1.96-1.87 (m, 1H), 1.67-1.37 (m, 10H), 1.01-0.86 (m, 7H).

### Example 36

### N-(4-{4-[Cyclopropyl-(4-methoxy-phenyl)-methyl]-piperazin-1-yl}-3-fluoro-phenyl)-2-ethyl-butyramide (Compound #84)

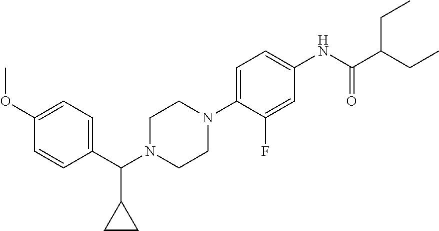

The title compound was prepared according to the process outlined in Example 7 herein, with the appropriate substituent changes.

MS (ESI): mass calculated for C₂₇H₃₆FN₃O₂, 453.59; m/z measured, 454.3 [M+H]⁺

¹H NMR (CDCl₃): 7.46 (dd, J=2.2, 14.0, 1H), 7.32-7.21 (m, 2H), 7.14-7.03 (m, 2H), 6.94-6.83 (m, 3H), 3.83 (s, 3H), 3.13-2.99 (m, 4H), 2.97-2.89 (m, 2H), 2.65-2.51 (m, 2H), 2.31 (d, J=9.6, 1H), 2.04-1.96 (m, 1H), 1.79-1.64 (m, 2H), 1.62-1.50 (m, 2H), 1.14-1.03 (m, 1H), 0.96 (t, J=7.4, 6H), 0.84-0.73 (m, 1H), 0.51-0.43 (m, 1H), 0.43-0.33 (m 1H), 0.08-0.00 (m, 1H).

### Example 37

### 2-Ethyl-N-{3-methyl-4-[4-(oxazol-2-yl-phenyl-methyl)-piperazin-1-yl]-phenyl}-butyramide (Compound #85)

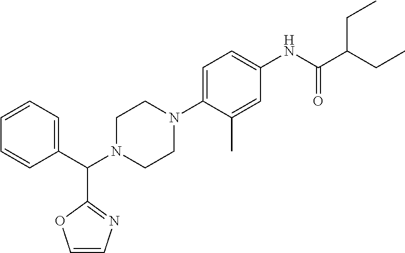

A solution of 3-methyl-4-[4-(oxazol-2-yl-phenyl-methyl)-piperazin-1-yl]-phenylamine (83 mg, 0.24 mmol) in THF (4.0 mL) was cooled to 0° C. A 1N solution of NaHCO₃ (0.26 mL) and 2-ethyl-butyryl chloride (34 mg, 0.25 mmol) were added drop-wise side by side. The resulting mixture was stirred at 0° C. for 0.5 h and then 18 h at room temperature. The resulting mixture was diluted with DCM and washed with water. The resulting mixture was concentrated to dryness to yield a residue. The residue was purified on reversed phase HPLC (acidic) to yield the title compound as its corresponding trifluoroacetic acid salt.

MS (ESI) mass calculated for C₂₇H₃₄N₄O₂, 446.60; m/z measured, 447.3 [M+H]⁺

¹H NMR (CD₃OD): 7.64 (s, 1H), 7.56-7.51 (m, 2H), 7.39-7.27 (m, 4H), 7.08 (s, 1H), 7.04-6.90 (m, 2H), 4.75 (s, 1H), 2.96-2.78 (m, 4H), 2.72-2.42 (m, 4H), 2.31 (s, 0.8H), 2.24 (s, 2.2H), 2.02-1.92 (m, 1H), 1.77-1.65 (m, 4H), 1.62-1.47 (m, 6H).

### Example 38

### N-{3-Cyano-4-[4-(oxazol-2-yl-phenyl-methyl)-piperazin-1-yl]-phenyl}-2-ethyl-butyramide (Compound #86)

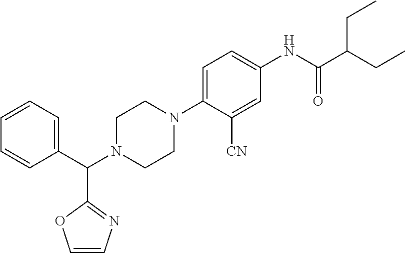

The title compound was prepared according to the process described in Example 37, reacting 5-amino-2-[4-(oxazol-2-yl-phenyl-methyl)-piperazin-1-yl]-benzonitrile (61 mg, 0.17 mmol) and 2-ethyl-butyryl chloride (24.5 mg, 0.18 mmol) and purifying the isolated residue on reversed phase HPLC (acidic) to yield the title compound as its corresponding trifluoroacetic acid salt.

MS (ESI) mass calculated for C₂₇H₃₁N₅O₂, 457.57; m/z measured, 458.6 [M+H]⁺

¹H NMR (CD₃OD): 8.03 (d, J=0.8, 1H), 7.99 (d, J=2.5, 1H), 7.75 (dd, J=8.9, 2.5, 1H), 7.68-7.62 (m, 2H), 7.55-7.50 (m, 3H), 7.35 (s, 1H), 7.20 (d, J=8.9, 1H), 5.80 (s, 1H), 3.46-3.31 (m, 8H), 2.26-2.15 (m, 1H), 1.72-1.46 (m, 4H), 0.94 (t, J=7.4, 6H).

### Example 39

### N-{3-Cyano-4-[4-(oxazol-2-yl-phenyl-methyl)-piperazin-1-yl]-phenyl}-isobutyramide (Compound #87)

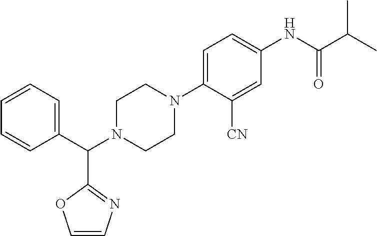

The title compound was prepared according to the process described in Example 37, reacting 5-amino-2-[4-(oxazol-2-yl-phenyl-methyl)-piperazin-1-yl]-benzonitrile (62 mg, 0.17 mmol) and isobutyryl chloride (19.5 mg, 0.18 mmol) and purifying the isolated residue on reversed phase HPLC (acidic) to yield the title compound as its corresponding trifluoroacetic acid salt.

MS (ESI) mass calculated for C₂₅H₂₇N₅O₂, 429.51; m/z measured, 430.5 [M+H]⁺

¹H NMR (CD₃OD): 8.03 (d, J=0.7, 1H), 7.96 (d, J=2.5, 1H), 7.74 (dd, J=8.9, 2.5, 1H), 7.68-7.62 (m, 2H), 7.57-7.50 (m, 3H), 7.36 (br s, 1H), 7.19 (d, J=9.0, 1H), 5.87-5.81 (m, 1H), 3.47-3.32 (m, 8H), 2.65-2.56 (m, 1H), 1.21-1.15 (m, 6H).

### Example 40

### Tetrahydro-furan-3-carboxylic acid {3-cyano-4-[4-(oxazol-2-yl-phenyl-methyl)-piperazin-1-yl]-phenyl}-amide (Compound #90)

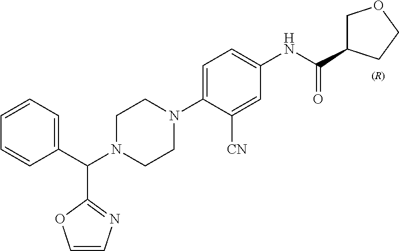

The title compound was prepared according to the procedure s described in Example 33 which follows herein; reacing 5-amino-2-[4-(oxazol-2-yl-phenyl-methyl)-piperazin-1-yl]-benzonitrile (100 mg, 0.28 mmol), and (R)-tetrahydro-furan-3-carboxylic acid acid (39.0 mg, 0.34 mmol) to yield a residue which was purified by reversed phase HPLC (acidic) to yield the title compound as its corresponding trifluoroacetic acid salt.

MS (ESI) mass calculated for C₂₆H₂₇FN₅O₃, 457.54; m/z measured, 457.2 [M+H]⁺

¹H NMR (CD₃OD): 8.02 (s, 1H), 7.95 (dd, J=2.5, 1H), 7.73 (dd, J=8.9, 2.5, 1H), 7.65-7.60 (m, 2H), 7.52-7.47 (m, 3H), 7.33 (s, 1H), 7.18 (d, 8.9, 1H), 5.67 (s, 1H), 4.0 (t, J=8.2, 1H), 3.94-3.85 (m, 2H), 3.84-3.77 (m, 1H), 3.40-3.33 (m, 4H), 3.29-3.20 (m, 4H), 3.19-3.11 (m, 1H), 2.21-2.14 (m, 2H)

### Example 41

### N-{3-Fluoro-4-[4-(oxazol-2-yl-phenyl-methyl)-piperazin-1-yl]-phenyl}-2-methyl-butyramide (Compound #91)

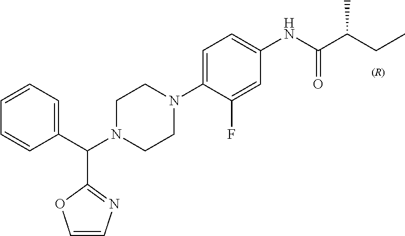

The title compound was prepared according to the process described in Example 33, reacting 3-fluoro-4-[4-(oxazol-2-yl-phenyl-methyl)-piperazin-1-yl]-phenylamine (78 mg, 0.22 mmol), and (s)-(+)-2-methylbutyric acid (28.0 mg, 0.27 mmol), and purifying the isolated residue on reversed phase HPLC (acidic) to yield the title compound as its corresponding trifluoroacetic acid salt.

MS (ESI) mass calculated for C₂₅H₂₉FN₄O₂, 436.52; m/z measured, 437.5 [M+H]⁺

¹H NMR (CD₃OD): 8.04 (d, J=0.8, 1H), 7.67-7.62 (m, 2H), 7.56-7.49 (m, 4H), 7.36 (d, J=0.7, 1H), 7.25-7.21 (m, 1H), 7.02 (t, J=9.0, 1H), 5.82 (s, 1H), 3.41-3.31 (m, 8H), 2.40-2.33 (m, 1H), 1.74-1.62 (m, 1H), 1.54-1.40 (m, 1H), 1.15 (d, J=6.8, 3H), 0.93 (t, J=7.4, 3H).

### Example 42

### Tetrahydro-furan-3-carboxylic acid {3-fluoro-4-[4-(oxazol-2-yl-phenyl-methyl)-piperazin-1-yl]-phenyl}-amide (Compound #92)

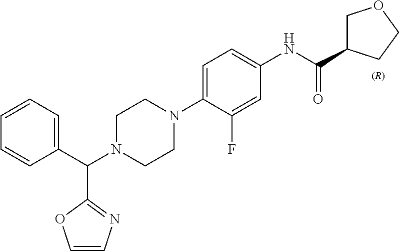

The title compound was prepared according to the process described in Example 33, reacting 3-fluoro-4-[4-(oxazol-2-yl-phenyl-methyl)-piperazin-1-yl]-phenylamine (106 mg, 0.3 mmol), and (R)-tetrahydro-furan-3-carboxylic acid (47.0 mg, 0.4 mmol), and purifying the isolated residue on reversed phase HPLC (acidic) to yield the title compound as its corresponding trifluoroacetic acid salt.

MS (ESI) mass calculated for C₂₅H₂₇FN₄O₃, 450.51; m/z measured, 451.5 [M+H]⁺

¹H NMR (CD₃OD): 8.04 (d, J=0.8, 1H), 7.67-7.62 (m, 2H), 7.56-7.49 (m, 4H), 7.37 (d, J=0.7, 1H), 7.25-7.21 (m, 1H), 7.03 (t, J=9.0, 1H), 5.89 (s, 1H), 3.99 (t, J=8.3 1H), 3.94-3.77 (m, 3H), 3.51-3.31 (m, 8H), 3.19-3.10 (m, 1H), 2.21-2.12 (m, 2H).

### Example 43

### N-{3-Cyano-4-[4-(oxazol-2-yl-phenyl-methyl)-piperazin-1-yl]-phenyl}-2-methyl-butyramide (Compound #93)

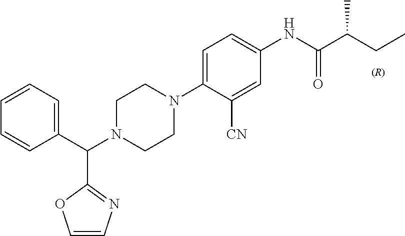

The title compound was prepared according to the process described in Example 33, reacting 5-amino-2-[4-(oxazol-2-yl-phenyl-methyl)-piperazin-1-yl]-benzonitrile (72 mg, 0.20 mmol), and (s)-(+)-2-methylbutyric acid (25.0 mg, 0.24 mmol), and purifying the isolated residue on reversed phase HPLC (acidic) to yield the title compound as its corresponding trifluoroacetic acid salt.

MS (ESI) mass calculated for C₂₆H₂₉N₅O₂, 443.54; m/z measured, 444.6 [M+H]⁺

¹H NMR (CD₃OD): 8.01 (d, J=0.8, 1H), 7.96 (d, J=2.5, 1H), 7.74 (dd, J=8.9, 2.5, 1H), 7.66-7.61 (m, 2H), 7.53-7.48 (m, 3H), 7.33 (d, J=0.7, 1H), 7.18 (d, J=8.9, 1H), 5.66 (s, 1H), 3.40-3.33 (m, 4H), 3.28-3.20 (m, 4H), 2.44-2.34 (m, 1H), 1.75-1.65 (m, 1H), 1.53-1.43 (m, 1H), 1.16 (d, J=6.8, 3H), 0.93 (t, J=7.4, 3H).

### Example 44

### Cyclobutanecarboxylic acid {3-cyano-4-[4-(oxazol-2-yl-phenyl-methyl)-piperazin-1-yl]-phenyl}-amide (Compound #95)

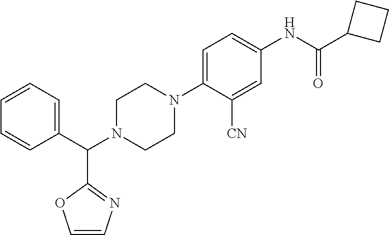

The title compound was prepared according to the process described in Example 33, reacting 5-amino-2-[4-(oxazol-2-yl-phenyl-methyl)-piperazin-1-yl]-benzonitrile (78 mg, 0.22 mmol), and cyclobutanecarboxylic acid (26.0 mg, 0.26 mmol), and purifying the isolated residue on reversed phase HPLC (acidic) to yield the title compound as its corresponding trifluoroacetic acid salt.

MS (ESI) mass calculated for C₂₆H₂₇N₅O₂, 441.54; m/z measured, 442.5 [M+H]⁺

¹H NMR (CD₃OD): 8.03 (d, J=0.7, 1H), 7.96 (d, J=2.5, 1H), 7.73 (dd, J=8.9, 2.5, 1H), 7.67-7.62 (m, 2H), 7.54-7.51 (m, 3H), 7.36 (d, J=0.5, 1H), 7.18 (d, J=8.9, 1H), 5.82 (s, 1H), 3.45-3.32 (m, 8H), 3.28-3.20 (m, 1H), 2.38-2.27 (m, 2H), 2.24-2.15 (m, 2H), 2.07-1.98 (m, 1H), 1.93-1.85 (m, 1H).

### Example 45

### N-{3-Cyano-4-[4-(oxazol-2-yl-phenyl-methyl)-piperazin-1-yl]-phenyl}-2-methyl-benzamide (Compound #96)

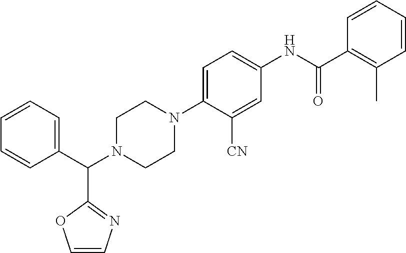

The title compound was prepared according to the process described in Example 33, reacting 5-amino-2-[4-(oxazol-2-yl-phenyl-methyl)-piperazin-1-yl]-benzonitrile (74 mg, 0.21 mmol), and 2-methyl-benzoic acid (34.0 mg, 0.25 mmol), and purifying the isolated residue on reversed phase HPLC (acidic) to yield the title compound as its corresponding trifluoroacetic acid salt.

MS (ESI) mass calculated for C₂₉H₂₇N₅O₂, 477.56; m/z measured, 478.5 [M+H]⁺

¹H NMR (CD₃OD): 8.04 (d, J=2.5, 1H), 8.02 (d, J=0.7, 1H), 7.87 (dd, J=8.9, 2.5, 1H), 7.65-7.60 (m, 2H), 7.53-7.43 (m, 4H), 7.41-7.35 (m, 1H), 7.34-7.25 (m, 3H), 7.22 (d, J=8.9, 1H), 5.61 (s, 1H), 3.42-3.34 (m, 4H), 3.26-3.18 (m, 4H), 2.44 (s, 3H).

### Example 46

### N-{3-Cyano-4-[4-(oxazol-2-yl-phenyl-methyl)-piperazin-1-yl]-phenyl}-butyramide (Compound #98)

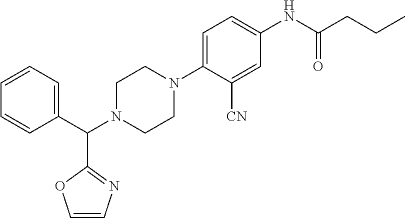

The title compound was prepared according to the process described in Example 33, reacting 5-amino-2-[4-(oxazol-2-yl-phenyl-methyl)-piperazin-1-yl]-benzonitrile (74 mg, 0.21 mmol), and butyric acid (18.5.0 mg, 0.25 mmol), and purifying the isolated residue on reversed phase HPLC (acidic) to yield the title compound as its corresponding trifluoroacetic acid salt.

MS (ESI) mass calculated for C₂₅H₂₇N₅O₂, 429.53; m/z measured, 430.5 [M+H]⁺

¹H NMR (CD₃OD): 8.03 (d, J=0.8, 1H), 7.97 (d, J=2.5, 1H), 7.71 (dd, J=8.9, 2.5, 1H), 7.67-7.62 (m, 2H), 7.54-7.49 (m, 3H), 7.52 (t, J=3.3, 3H), 7.35 (d, J=0.64, 1H), 7.18 (d, J=8.9, 1H), 5.81 (s, 1H), 3.49-3.32 (m, 8H), 2.33 (t, J=7.3, 2H), 1.76-1.65 (m, 2H), 0.98 (t, J=7.4, 3H).

### Example 47

### N-{3-Fluoro-4-[4-(oxazol-2-yl-phenyl-methyl)-piperazin-1-yl]-phenyl}-butyramide (Compound #100)

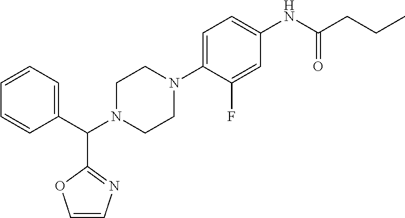

The title compound was prepared according to the process described in Example 33, reacting 3-fluoro-4-[4-(oxazol-2-yl-phenyl-methyl)-piperazin-1-yl]-phenylamine (70 mg, 0.2 mmol), and butyric acid (18.5.0 mg, 0.25 mmol) and purifying the isolated residue on reversed phase HPLC (acidic) to yield the title compound as its corresponding trifluoroacetic acid salt.

MS (ESI) mass calculated for C₂₄H₂₇FN₄O₂, 422.50; m/z measured, 423.5 [M+H]⁺

¹H NMR (CD₃OD): 8.04 (d, J=0.8, 1H), 7.67-7.63 (m, 2H), 7.56-7.48 (m, 4H), 7.34-7.35 (m, 1H), 7.24-7.19 (m, 1H), 7.05-6.99 (m, 1H), 5.87 (s, 1H), 3.49-3.32 (m, 8H), 2.32 (t, J=7.3, 2H), 1.76-1.65 (m, 2H), 0.98 (t, J=7.4, 3H).

### Example 48

### Cyclopropanecarboxylic acid {3-cyano-4-[4-(oxazol-2-yl-phenyl-methyl)-piperazin-1-yl]-phenyl}-amide (Compound #101)

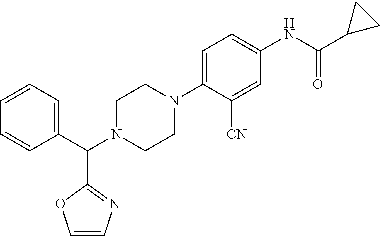

The title compound was prepared according to the process described in Example 33, reacting 5-amino-2-[4-(oxazol-2-yl-phenyl-methyl)-piperazin-1-yl]-benzonitrile (80 mg, 0.22 mmol), and cyclopropanecarboxylic acid (23.0 mg, 0.27 mmol), and purifying the isolated residue on reversed phase HPLC (acidic) to yield the title compound as its corresponding trifluoroacetic acid salt.

MS (ESI) mass calculated for C₂₅H₂₅N₅O₂, 427.5; m/z measured, 428.5 [M+H]⁺

¹H NMR (CD₃OD): 8.04 (s, 1H), 7.94 (d, J=2.5, 1H), 7.75-7.70 (m, 1H), 7.69-7.63 (m, 2H), 7.58-7.50 (m, 3H), 7.36 (s, 1H), 7.19 (d, J=8.9, 1H), 5.84 (br s, 1H), 3.46-3.32 (m, 8H), 1.77-1.69 (m, 1H), 0.97-0.92 (m, 2H), 0.90-0.84 (m, 2H).

### Example 49

### N-(4-{4-[(4-Chloro-phenyl)-cyclopropyl-methyl]-piperazin-1-yl}-3-fluoro-phenyl)-2-ethyl-butyramide (Compound #102)

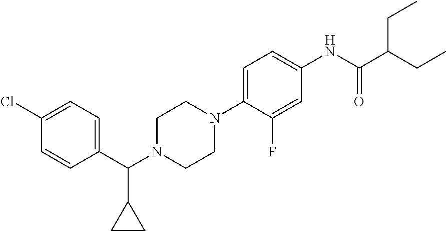

The title compound was prepared according to the process outlined in Example 7 herein, with the appropriate substituent changes.

MS (ESI): mass calculated for C₂₆H₃₃ClFN₃O, 458.01; m/z measured, 458.3 [M+H]⁺

¹H NMR (CDCl₃): 7.46 (dd, J=2.5, 14.3, 1H), 7.31 (s, 3H), 7.17 (bs, 1H), 7.15-7.09 (m, 1H), 6.89 (t, J=9.1, 1H), 3.12-2.98 (m, 4H), 2.95-2.87 (m, 2H), 2.59-2.49 (m, 2H), 2.30 (d, J=9.3, 1H), 2.05-1.96 (m, 1H), 1.84 (bs, 1H), 1.77-1.66 (m, 2H), 1.62-1.50 (m, 2H), 0.96 (t, J=7.4, 7H), 0.53-0.43 (m, 1H), 0.45-0.34 (m, 1H), 0.06-0.00 (m, 2H).

### Example 50

### N-{3-Cyano-4-[4-(oxazol-2-yl-phenyl-methyl)-piperazin-1-yl]-phenyl}-acetamide (Compound #103)

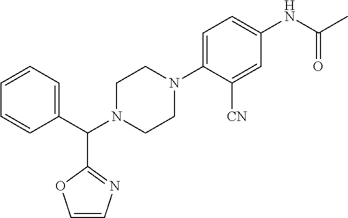

The title compound was prepared according to the process described in Example 33, reacting 5-amino-2-[4-(oxazol-2-yl-phenyl-methyl)-piperazin-1-yl]-benzonitrile (60 mg, 0.17 mmol), and acetic acid (12.0 mg, 0.20 mmol), and purifying the isolated residue on reversedphase HPLC (acidic) to yield the title compound as its corresponding trifluoroacetic acid salt.

MS (ESI) mass calculated for C₂₃H₂₃N₅O₂, 401.46; m/z measured, 402.5 [M+H]⁺

¹H NMR (CD₃OD): 8.00 (br s, 1H), 7.93 (d, J=2.5, 1H), 7.70 (dd, J=2.5, 1H), 7.64-7.59 (m, 2H), 7.51-7.46 (m, 3H), 7.32 (br s, 1H), 7.17 (d, J=9.0 1H), 5.55 (s, 1H), 3.37-3.32 (m, 4H), 3.21-3.14 (m, 4H), 2.11 (s, 3H).

### Example 51

### N-{3-Fluoro-4-[4-(oxazol-2-yl-phenyl-methyl)-piperazin-1-yl]-phenyl}-acetamide (Compound #104)

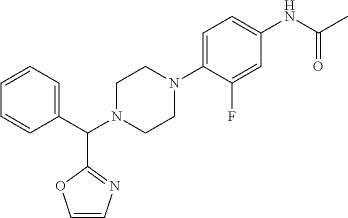

The title compound was prepared according to the process described in Example 33, reacting 3-fluoro-4-[4-(oxazol-2-yl-phenyl-methyl)-piperazin-1-yl]-phenylamine (65 mg, 0.19 mmol), and acetic acid (13.0 mg, 0.22 mmol), and the purifying the isolated residue on reversed phase HPLC (acidic) to yield the title compound as its corresponding trifluoroacetic acid salt.

MS (ESI) mass calculated for C₂₂H₂₃FN₄O₂, 394.44; m/z measured, 395.5 [M+H]⁺

¹H NMR (CD₃OD): 8.04 (d, J=0.8, 1H), 7.67-7.64 (m, 2H), 7.57-7.53 (m, 3H), 7.52-7.47 (m, 1H), 7.37 (m, 1H), 7.22-7.16 (m, 1H), 7.03 (t, J=9.0, 1H), 5.90 (s, 1H), 3.55-3.33 (m, 8H), 2.09 (s, 3H).

### Example 52

### N-(4-{4-[Cyclobutyl-(4-fluoro-phenyl)-methyl]-piperazin-1-yl}-3-fluoro-phenyl)-2-ethyl-butyramide (Compound #106)

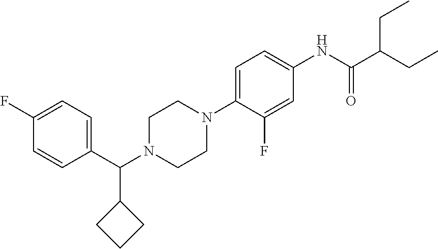

The title compound was prepared according to the process outlined in Example 7 herein, with the appropriate substituent changes.

MS (ESI): mass calculated for C₂₇H₃₅F₂N₃O, 455.58; m/z measured, 457.3 [M+H]⁺

¹H NMR (CDCl₃): 7.44 (dd, J=1.6, 14.0, 1H), 7.26-7.18 (m, 2H), 7.15-7.06 (m, 2H), 7.04-6.93 (m, 2H), 6.86 (t, J=9.1, 1H), 3.20 (d, J=9.1, 1H), 3.06-2.94 (m, 4H), 2.83-2.72 (m, 1H), 2.66-2.45 (m, 4H), 2.28-2.16 (m, 1H), 2.02-1.91 (m, 2H), 1.89-1.77 (m, 1H), 1.77-1.65 (m, 3H), 1.63-1.39 (m, 4H), 0.96 (t, J=7.4, 6H).

### Example 53

### Cyclohexanecarboxylic acid {3-fluoro-4-[4-(oxazol-2-yl-phenyl-methyl)-piperazin-1-yl]-phenyl}-amide (Compound #107)

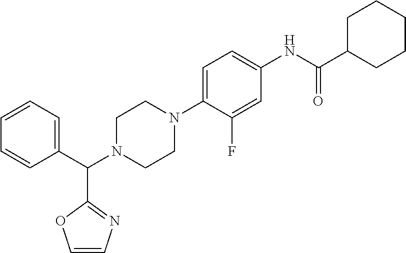

The title compound was prepared according to the process described in Example 33, reacting 3-fluoro-4-[4-(oxazol-2-yl-phenyl-methyl)-piperazin-1-yl]-phenylamine (65 mg, 0.19 mmol), and cyclohexanecarboxylic acid (30.0 mg, 0.23 mmol), and purifying the isolated residue on reversed phase HPLC (acidic) to yield the title compound as its corresponding trifluoroacetic acid salt.

MS (ESI) mass calculated for C₂₇H₃₁FN₄O₂, 462.56; m/z measured, 463.6 [M+H]⁺

¹H NMR (CD₃OD): 8.04 (d, J=0.8, 1H), 7.67-7.64 (m, 2H), 7.56-7.47 (m, 4H), 7.36 (d, J=0.6, 1H), 7.24-7.20 (m, 1H), 7.02 (t, J=9.0, 1H), 5.80 (s, 1H), 3.43-3.32 (m, 8H), 2.30-2.28 (m, 1H), 1.88-1.77 (m, 4H), 1.76-1.68 (m, 1H), 1.55-1.44 (m, 2H), 1.42-1.19 (m, 3H).

### Example 54

### 4,4-Difluoro-cyclohexanecarboxylic acid {3-fluoro-4-[4-(oxazol-2-yl-phenyl-methyl)-piperazin-1-yl]-phenyl}-amide (Compound #108)

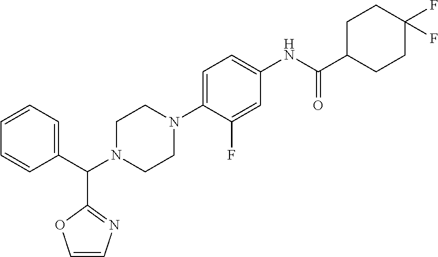

The title compound was prepared according to the process described in Example 33, reacting 3-fluoro-4-[4-(oxazol-2-yl-phenyl-methyl)-piperazin-1-yl]-phenylamine (70 mg, 0.20 mmol), and 4,4-difluoro-cyclohexanecarboxylic acid (42.0 mg, 0.25 mmol), and purifying the isolated residue on reversed phase HPLC (acidic) to yield the title compound as its corresponding trifluoroacetic acid salt.

MS (ESI) mass calculated for C₂₇H₂₉F₃N₄O₂, 498.54; m/z measured, 499.6 [M+H]⁺

¹H NMR (CD₃OD): 8.02 (d, J=0.7, 1H), 7.65-7.60 (m, 2H), 7.53-7.45 (m, 4H), 7.33 (br s, 1H), 7.24-7.18 (m, 1H), 7.02 (t, J=9.0, 1H), 5.64 (br s, 1H), 3.29-3.19 (m, 8H), 2.50-2.39 (m, 1H), 2.18-2.08 (m, 2H), 1.97-1.75 (m, 6H).

### Example 55

### 4,4-Difluoro-cyclohexanecarboxylic acid {3-cyano-4-[4-(oxazol-2-yl-phenyl-methyl)-piperazin-1-yl]-phenyl}-amide (Compound #109)

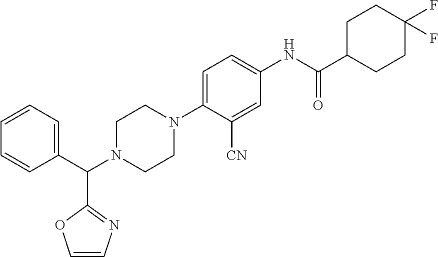

The title compound was prepared according to the process described in Example 33, reacting 5-amino-2-[4-(oxazol-2-yl-phenyl-methyl)-piperazin-1-yl]-benzonitrile (71 mg, 0.20 mmol), and 4,4-difluoro-cyclohexanecarboxylic acid (42.0 mg, 0.25 mmol), and purifying the isolated residue on reversed phase HPLC (acidic) to yield the title compound as its corresponding trifluoroacetic acid salt.

MS (ESI) mass calculated for C₂₈H₂₉F₂N₅O₂, 505.56; m/z measured, 506.6 [M+H]⁺

¹H NMR (CD₃OD): 8.02 (d, J=0.8, 1H), 7.95-7.92 (m, 1H), 7.74-7.70 (m, 1H), 7.65-7.61 (m, 2H), 7.53-7.47 (m, 3H), 7.33 (br s, 1H), 7.18 (d, J=9.0 1H), 5.63 (br s, 1H), 3.40-3.33 (m, 4H), 3.27-3.30 (m, 4H), 2.50-2.40 (m, 1H), 2.19-2.09 (m, 2H), 1.99-1.92 (m, 2H), 1.90-1.76 (m, 4H).

### Example 56

### N-{3-Cyano-4-[4-(oxazol-2-yl-phenyl-methyl)-piperazin-1-yl]-phenyl}-4,4,4-trifluoro-2-methyl-butyramide (Compound #110)

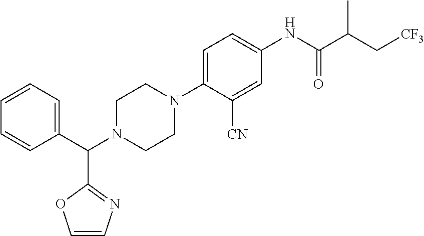

The title compound was prepared according to the process described in Example 33, reacting 5-amino-2-[4-(oxazol-2-yl-phenyl-methyl)-piperazin-1-yl]-benzonitrile (78 mg, 0.22 mmol), and 4,4,4-trifluoro-2-methyl-butyric acid (43.0 mg, 0.28 mmol), and purifying the isolated residue on reversed phase HPLC (acidic) to yield the title compound as its corresponding trifluoroacetic acid salt.

MS (ESI) mass calculated for C₂₆H₂₆F₃N₅O₂, 497.51; m/z measured, 498.5 [M+H]⁺

¹H NMR (CD₃OD): 8.00 (s, 1H), 7.94-7.91 (m, 1H), 7.74-7.69 (m, 1H), 7.64-7.59 (m, 2H), 7.52-7.45 (m, 3H), 7.31 (br s, 1H), 7.18 (d, J=9.0 1H), 5.55 (s, 1H), 3.39-3.33 (m, 4H), 3.21-3.14 (m, 4H), 2.90-2.79 (m, 1H), 2.77-2.61 (m, 1H), 2.33-2.21 (m, 1H), 1.29 (d, J=7.0, 3H).

### Example 57

### 4,4,4-Trifluoro-N-{3-fluoro-4-[4-(oxazol-2-yl-phenyl-methyl)-piperazin-1-yl]-phenyl}-2-methyl-butyramide (Compound #111)

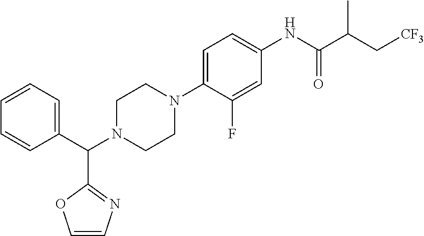

The title compound was prepared according to the process described in Example 33, reacting 3-fluoro-4-[4-(oxazol-2-yl-phenyl-methyl)-piperazin-1-yl]-phenylamine (106 mg, 0.30 mmol), and 4,4,4-trifluoro-2-methyl-butyric acid (63.0 mg, 0.40 mmol), and purifying the isolated residue on reversed phase HPLC (acidic) to yield the title compound as its corresponding trifluoroacetic acid salt.

MS (ESI) mass calculated for C₂₅H₂₆F₄N₄O₂, 490.49; m/z measured, 491.5 [M+H]⁺

¹H NMR (CD₃OD): 8.04 (s, 1H), 7.68-7.62 (m, 2H), 7.56-7.47 (m, 4H), 7.36 (s, 1H), 7.22 (dd, J=8.7, 1.7, 1H), 7.03 (t, J=9.1, 1H), 5.84 (br s, 1H), 3.44-3.31 (m, 8H), 2.89-2.78 (m, 1H), 2.75-2.62 (m, 1H), 2.31-2.17 (m, 1H), 1.27 (d, J=7.0, 3H).

### Example 58

### Cyclohexanecarboxylic acid {3-cyano-4-[4-(oxazol-2-yl-phenyl-methyl)-piperazin-1-yl]-phenyl}-amide (Compound #112)

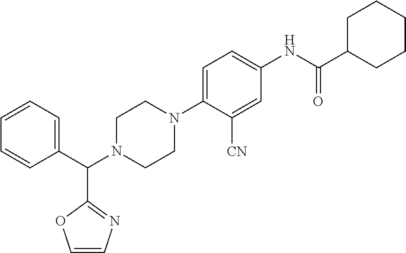

The title compound was prepared according to the process described in Example 33, reacting 2-[4-(4-amino-2-cyano-phenyl)-piperazin-1-yl]-2-phenyl-acetimidic acid methyl ester (66 mg, 0.19 mmol), and cyclohexanecarboxylic acid (31.0 mg, 0.24 mmol), and purifying the isolated residue on reversedphase HPLC (acidic) to yield the title compound as its corresponding trifluoroacetic acid salt.

MS (ESI) mass calculated for C₂₈H₃₁N₅O₂, 469.58; m/z measured, 470.6 [M+H]⁺

¹H NMR (CD₃OD): 8.03 (d, J=0.8, 1H), 7.95 (d, J=2.5, 1H), 7.74-7.21 (dd, J=9.0, 2.5, 1H), 7.66-7.63 (m, 2H), 7.55-7.50 (m, 3H), 7.35 (d, J=0.7, 1H), 7.18 (d, J=9.0, 1H), 5.79 (br s, 1H), 3.42-3.33 (m, 8H), 2.37-2.29 (m, 1H), 1.93-1.78 (m, 4H), 1.76-1.68 (m, 1H), 1.65-1.45 (m, 2H), 1.40-1.25 (m, 3H).

### Example 59

### 3,5-Dimethyl-isoxazole-4-carboxylic acid {3-fluoro-4-[4-(oxazol-2-yl-phenyl-methyl)-piperazin-1-yl]-phenyl}-amide (Compound #113)

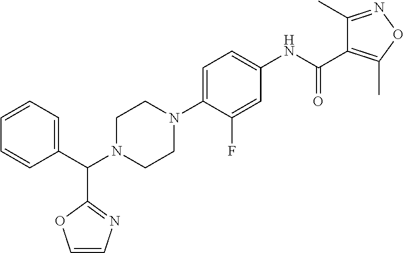

The title compound was prepared according to the process outlined in Example 7 above, with the appropriate substituent changes.

MS (ESI): mass calculated for C₂₆H₂₆FN₅O₃, 475.2; m/z measured, 476.2 [M+H]⁺

¹H NMR (CDCl₃): 7.64 (s, 1H), 7.55-7.48 (m, 2H), 7.46-7.25 (m, 5H), 7.36-7.08 (m, 2H), 6.90 (t, J=9.0, 1H), 4.68 (s, 1H), 3.15-3.05 (m, 4H), 2.76-2.68 (m, 2H), 2.63 (s, 3H), 2.58-2.52 (m, 2H), 2.47 (s, 3H).

### Example 60

### 2-Ethyl-N-(3-fluoro-4-{4-[phenyl-(tetrahydro-furan-3-yl)-methyl]-piperazin-1-yl}-phenyl)-butyramide (Compound #114)

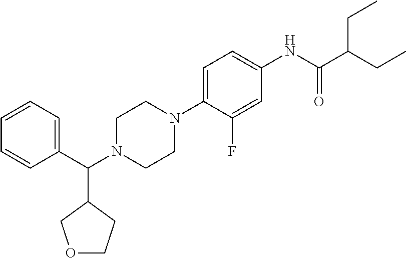

The title compound was prepared according to the process outlined in Example 27 above, with the appropriate substituent changes.

MS (ESI): mass calculated for C₂₇H₃₆FN₃O₂, 453.3; m/z measured, 454.3 [M+H]⁺.

¹H NMR (CDCl₃): 7.45-7.26 (m, 3H), 7.20-7.07 (m, 4H), 6.90-6.82 (m, 1H), 4.05-2.90 (m, 10H), 2.70-2.40 (m, 4H), 2.20-1.30 (m, 7H), 0.98-0.92 (m, 6H).

### Example 61

### N-(4-{4-[(2,2-Dichloro-3-methyl-cyclopropyl)-phenyl-methyl]-piperazin-1-yl}-3-fluoro-phenyl)-2-ethyl-butyramide (Compound #115)

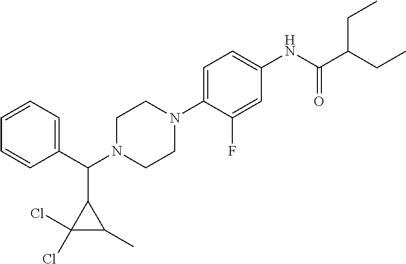

The title compound was prepared according to the process outlined in Example 7 herein, with the appropriate substituent changes.

MS (ESI): mass calculated for C₂₇H₃₄Cl₂FN₃O, 506.48; m/z measured, 506.5 [M+H]⁺

¹H NMR (CDCl₃): 7.52-7.43 (m, 1H), 7.42-7.35 (m, 2H), 7.25-7.21 (m, 2H), 7.13-7.08 (m, 1H), 7.04 (bs, 1H), 6.87 (t, J=9.1, 1H), 3.36-3.25 (m, 1H), 3.07-2.97 (m, 3H), 2.89-2.75 (m, 2H), 2.73-2.61 (m, 2H), 2.40 (s, 3H), 2.21-2.06 (m, 1H), 2.06-1.86 (m, 2H), 1.79-1.64 (m, 3H), 1.63-1.47 (m, 3H), 9.63 (t, J=7.4, 6H).

### Example 62

### N-{4-[4-(Cyclobutyl-oxazol-2-yl-methyl)-piperazin-1-yl]-3-fluoro-phenyl}-2-ethyl-butyramide (Compound #116)

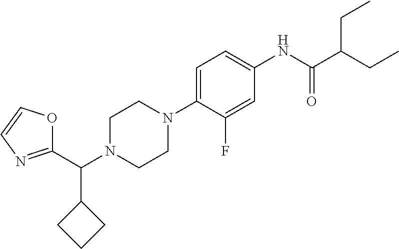

The title compound was prepared according to the process outlined in Example 27 above, with the appropriate substituent changes.

MS (ESI): mass calculated for C₂₄H₃₃FN₄O₂, 428.3; m/z measured, 429.3 [M+H]⁺

¹H NMR (CDCl₃): 7.65 (s, 1H), 7.43 (dd, J=14.1, 2.4, 1H), 7.14-7.06 (m, 3H), 6.87 (t, J=9.0, 1H), 3.77 (d, J=10.5, 1H), 3.12-2.95 (m, 5H), 2.73-2.60 (m, 4H), 2.25-2.15 (m, 1H), 2.03-1.80 (m, 5H), 1.78-1.50 (m, 5H), 0.98-0.92 (m, 6H).

### Example 63

### N-{4-[4-(Cyclopentyl-oxazol-2-yl-methyl)-piperazin-1-yl]-3-fluoro-phenyl}-2-ethyl-butyramide (Compound #117)

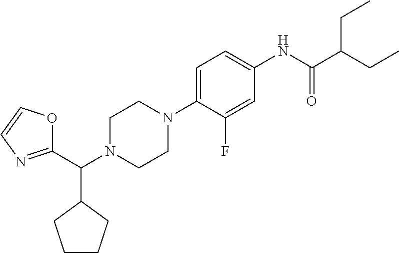

The title compound was prepared according to the process outlined in Example 27 above, with the appropriate substituent changes.

MS (ESI): mass calculated for C₂₅H₃₅FN₄O₂, 442.3; m/z measured, 443.3 [M+H]⁺

¹H NMR (CDCl₃): 7.62 (s, 1H), 7.43 (dd, J=14.1, 2.4, 1H), 7.35 (s, 1H), 7.14-7.06 (m, 2H), 6.85 (t, J=9.0, 1H), 3.52 (d, J=11.2, 1H), 3.18-2.92 (m, 4H), 2.79-2.70 (m, 2H), 2.68-2.52 (m, 3H), 2.05-1.95 (m, 1H), 1.90-1.47 (m, 11H), 1.10-0.99 (m, 1H), 0.98-0.92 (m, 6H).

### Example 64

### 2-Ethyl-N-(3-fluoro-4-{4-[(4-fluoro-phenyl)-oxazol-2-yl-methyl]-piperazin-1-yl}-phenyl)-butyramide (Compound #120)

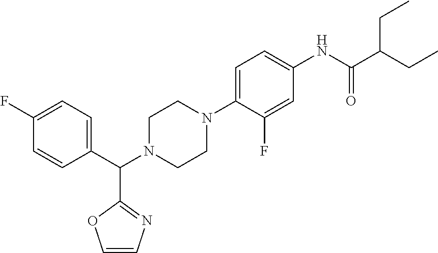

A mixture of methanesulfonic acid (3-fluoro-phenyl)-oxazol-2-yl-methyl ester (110 mg, 0.40 mmol, crude), 2-ethyl-N-(3-fluoro-4-piperazin-1-yl-phenyl)-butyramide hydrochloride (40 mg, 0.12 mmol) and DIPEA (71 mg, 0.12 mmol) in CH₃CN was heated to 60° C. for 18 h. The resulting mixture was then cooled to room temperature and diluted with water (30 mL). The diluted mixture was extracted with DCM (2×25 mL), dried (Na₂SO₄), filtered and concentrated to yield a residue. The residue was purified on reversed phase HPLC (basic) to yield the title compound.

MS (ESI) mass calculated for C₂₆H₃₀F₂N₄O₂, 468.55; m/z measured, 469.5 [M+H]⁺

¹H NMR (CDCl₃): 7.64 (d, J=0.7, 1H), 7.52-7.47 (m, 2H), 7.42-7.36 (m, 2H), 7.10-7.01 (m, 4H), 6.88-6.82 (m, 1H), 4.76 (s, 1H), 4.07-3.79 (m, 4H), 3.11-3.04 (m, 4H), 3.03-2.97 (m, 1H), 2.74-2.61 (m, 2H), 2.57-2.47 (m, 2H), 2.28-2.20 (m, 2H), 1.64-1.60 (m, 4H).

### Example 65

### N-{4-[4-(Cyclohexyl-oxazol-2-yl-methyl)-piperazin-1-yl]-3-fluoro-phenyl}-2-ethyl-butyramide (Compound #122)

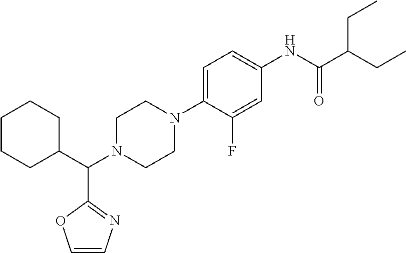

The title compound was prepared according to the process outlined in Example 27 above, with the appropriate substituent changes.

MS (ESI): mass calculated for C₂₆H₃₇FN₄O₂, 456.3; m/z measured, 457.6 [M+H]⁺

¹H NMR (CDCl₃): 7.62 (s, 1H), 7.43 (dd, J=14.1, 2.4, 1H), 7.14-7.06 (m, 3H), 6.86 (t, J=9.0, 1H), 3.52 (d, J=10.6, 1H), 3.12-2.98 (m, 4H), 2.79-2.57 (m, 4H), 2.20-1.97 (m, 3H), 1.83-1.50 (m, 7H), 1.34-1.16 (m, 4H), 1.07-0.86 (m, 8H).

### Example 66

### Cyclopentanecarboxylic acid {3-fluoro-4-[4-(oxazol-2-yl-phenyl-methyl)-piperazin-1-yl]-phenyl}-amide (Compound #126)

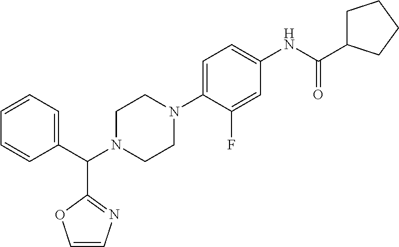

The title compound was prepared according to the process described in Example 64, reacting methanesulfonic acid (4-fluoro-phenyl)-oxazol-2-yl-methyl ester (185 mg, 0.68 mmol, crude) and cyclopentanecarboxylic acid (3-fluoro-4-piperazin-1-yl-phenyl)-amide (66 mg, 0.23 mmol) to yield the title compound.

MS (ESI) mass calculated for C₂₆H₂₈F₂N₄O₂, 466.52; m/z measured, 467.5 [M+H]⁺

¹H NMR (CDCl₃): 8.03 (s, 1H), 7.72-7.65 (m, 2H), 7.50 (dd, J=14.4, 2.3, 1H), 7.34 (s, 1H), 7.29-7.18 (m, 3H), 7.00 (t, J=9.0, 1H), 5.72 (s, 1H), 3.30-3.19 (m, 8H), 2.81-2.71 (m, 1H), 1.97-1.86 (m, 2H), 1.83-1.72 (m, 4H), 1.67-1.58 (m, 2H).

### Example 67

### Tetrahydro-furan-3-carboxylic acid (3-fluoro-4-{4-[(4-fluoro-phenyl)-oxazol-2-yl-methyl]-piperazin-1-yl}-phenyl)-amide (Compound #127)

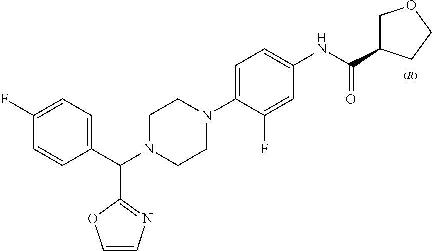

The title compound was prepared according to the process described in Example 64, reacting methanesulfonic acid (4-fluoro-phenyl)-oxazol-2-yl-methyl ester (153 mg, 0.56 mmol, crude) and R-tetrahydro-furan-3-carboxylic acid (3-fluoro-4-piperazin-1-yl-phenyl)-amide (55 mg, 0.19 mmol) and purifying the isolated residue on reversed phase HPLC (acidic) to yield the title compound.

MS (ESI) mass calculated for C₂₅H₂₆F₂N₄O₂, 468.51; m/z measured, 469.5 [M+H]⁺

¹H NMR (CD₃OD): 8.03 (s, 1H), 7.70-7.66 (m, 2H), 7.50 (dd, J=14.4, 2.4, 1H), 7.34 (s, 1H), 7.28-7.19 (m, 3H), 7.02 (t, J=9.0, 1H), 5.68 (s, 1H), 3.99 (t, J=8.2, 1H), 3.94-3.77 (m, 3H), 3.30-3.18 (m, 8H), 3.18-3.11 (m, 1H), 2.21-2.14 (m, 2H).

### Example 68

### N-(3-Cyano-4-{4-[(3-fluoro-phenyl)-oxazol-2-yl-methyl]-piperazin-1-yl}-phenyl)-2-ethyl-butyramide (Compound #131)

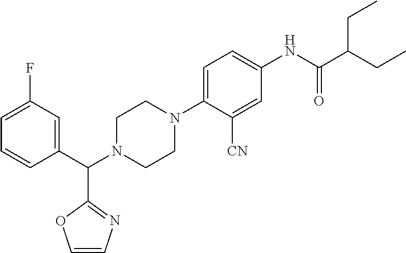

The title compound was prepared according to the process described in Example 64, reacting methanesulfonic acid (3-fluoro-phenyl)-oxazol-2-yl-methyl ester (135 mg, 0.50 mmol, crude) and N-(3-Cyano-4-piperazin-1-yl-phenyl)-2-ethyl-butyramide (51 mg, 0.17 mmol) and purifying the isolated residue on reversed phase HPLC (acidic) to yield the title compound.

MS (ESI) mass calculated for C₂₇H₃₀FN₅O₂, 475.57; m/z measured, 476.3 [M+H]⁺

¹H NMR (CD₃OD): 8.03 (s, 1H), 7.98-7.96 (m, 1H), 7.75 (dd, J=8.9, 2.5, 1H), 7.55-7.48 (m, 1H), 7.47-7.42 (m, 2H), 7.33 (s, 1H), 7.27-7.16 (m, 2H), 5.61 (s, 1H), 3.40-3.32 (m, 4H), 3.21-3.14 (m, 4H), 2.25-2.14 (m, 1H), 1.70-1.48 (m, 4H), 0.92 (t, J=7.4, 6H).

### Example 69

### 2-Ethyl-N-(3-fluoro-4-{4-[(3-fluoro-phenyl)-oxazol-2-yl-methyl]-piperazin-1-yl}-phenyl)-butyramide (Compound #132)

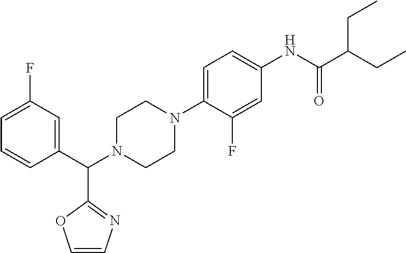

A mixture of 2-[chloro-(3-fluoro-phenyl)-methyl]-oxazole (106 mg, 0.5 mmol), 2-ethyl-N-(3-fluoro-4-piperazin-1-yl-phenyl)butyramide (147 mg, 0.5 mmol), and Cs₂CO₃ (204 mg,).63 mmol) in CH₃CN was heated to 60° C. for 18 h. The resulting mixture was then cooled to room temperature and diluted with water (30 mL). The diluted mixture was extracted with DCM (2×25 mL), dried (Na₂SO₄), filtered and concentrated to a residue. Chromatography of the residue (SiO₂, 0-5% acetone/DCM, gradient) yielded title compound.

MS (ESI) mass calculated for C₂₆H₃₀F₂N₄O₂, 468.55; m/z measured, 469.5 [M+H]⁺

¹H NMR (CDCl₃): 7.66 (s, 1H), 7.50-7.39 (m, 2H), 7.35-7.22 (m, 3H), 7.15-7.09 (m, 2H), 7.03-6.95 (m, 1H), 6.85 (t, J=9.0, 1H), 4.79 (s, 1H), 3.12-3.02 (m, 4H), 2.74-2.64 (m, 2H), 2.58-2.49 (m, 2H), 2.00-1.98 (m, 1H), 1.75-1.62 (m, 2H), 1.59-1.46 (m, 2H), 0.92 (t, J=7.4, 6H).

### Example 70

### 2-Ethyl-N-(3-fluoro-4-{4-[(3-fluoro-phenyl)-(5-methyl-[1,3,4]-oxadiazol-2-yl)-methyl]-piperazin-1-yl}-phenyl)-butyramide (Compound #134)

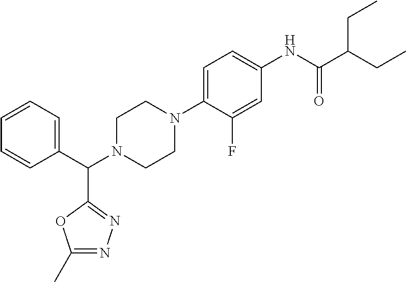

### STEP A: 2-Ethyl-N-{3-fluoro-4-[4-(hydrazinocarbonyl-phenyl-methyl)-piperazin-1-yl]-phenyl}-butyramide

To a solution of {4-[4-(2-ethyl-butyrylamino)-2-fluoro-phenyl]-piperazin-1-yl}-phenyl-acetic acid methyl ester (998 mg) in MeON (8 mL) was added NH₂NH₂.H₂O (1.1 mL) and the resulting mixture was heated at reflux for 3 days. The resulting mixture was then dried over Na₂SO₄, filtered and concentrated in vacuo to yield the title compound as a white solid.

MS (electrospray): exact mass calculated for C₂₄H₃₂FN₅O₂, 441.25; measured m/z 442.6 [M+H]⁺.

### STEP B: 2-Ethyl-N-(3-fluoro-4-{4-[(3-fluoro-phenyl)-(5-methyl-[1,3,4]-oxadiazol-2-yl)-methyl]-piperazin-1-yl}-phenyl)-butyramide

To a heterogeneous mixture of the product prepared in STEP A (204 mg) in CH₃CN (25 mL) was added TEA (0.077 mL) followed by acetyl chloride (0.036 mL). After 30 min Burgess reagent (275 mg) was added and the resulting mixture heated at reflux for 14 h. The resulting mixture was concentrated in vacuo and chromatography on SiO₂ (50% EtOAc/Hexanes) to yield the title compound.

MS (electrospray): exact mass calculated for C₂₆H₃₂FN₅O₂, 465.25; measured m/z 466.3 [M+H]⁺, 488.3 [M+Na]⁺

¹H NMR (500 MHz, CDCl₃): 7.53-7.48 (m, 2H), 7.44 (dd, J=13.99, 2.32 Hz, 1H), 7.40-7.32 (m, 3H), 7.23 (s, 1H), 7.13 (dd, J=8.63, 1.97 Hz, 1H), 6.89-6.83 (m, 1H), 4.89 (s, 1H), 3.12-3.04 (m, 4H), 2.75-2.68 (m, 2H), 2.60-2.51 (m, 5H), 2.04-1.97 (m, 1H), 1.74-1.65 (m, 2H), 1.59-1.50 (m, 2H), 0.94 (t, J=7.41 Hz, 6H).

### Example 71

### N-{4-[4-(Cyano-furan-2-yl-methyl)-piperazin-1-yl]-3-fluoro-phenyl}-2-ethyl-butyramide (Compound #135)

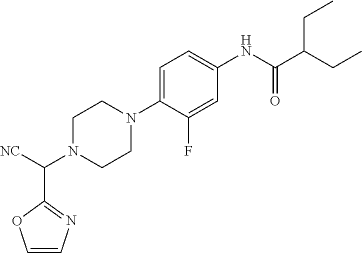

### STEP A: 4-(2-fluoro-4-methylamino-phenyl)-piperazine-1-carboxylic acid tert-butyl ester

4-(2-Fluoro-4-nitro-phenyl)-piperazine-1-carboxylic acid tert-butyl ester (10 mmol) was dissolved into EtOH/EtOAc (50/50 mL). SnCl₂2H₂O (10 g) was then added. The resulting mixture was heated at 100° C. for 16 h. After being cooled down, ice-H₂O (100 mL) was added followed by addition of NaHCO₃ until pH=9. The resulting mixture was extracted by EtOAc (3×200 mL). The organic layer was collected, dried (Na₂SO₄), filtered, and concentrated to yield the crude title compound.

### STEP B: 2-ethyl-N-(3-fluoro-4-piperazin-1-yl-phenyl)-butyramide

To 4-(2-fluoro-4-methylamino-phenyl)piperazine-1-carboxylic acid tert-butyl ester prepared as in STEP A above and TEA (10.0 mmol) in CH₂Cl₂ (50 mL) was added 2-ethyl-butyryl chloride (10.0 mmol). The resulting mixture was stirred at room temperature for 16 h. H₂O (10 mL) was then added, the organic layer was separated. After concentration, the semi-solid was collected and re-dissolved into CF₃COOH/CH₂Cl₂ (10/50 mL). The resulting mixture was stirred at room temperature for 16 h, then concentrated to yield the title compound.

### STEP C: N-{4-[4-(cyano-furan-2-yl-methyl)-piperazin-1-yl]-3-fluoro-phenyl}-2-ethyl-butyramide

A mixture of 2-ethyl-N-(3-fluoro-4-piperazin-1-yl-phenyl)-butyramide prepared as in STEP B above (0.5 mmol), TMSCN (0.75 mmol), I₂ (13 mg), and furan-2-carbaldehyde (0.5 mmol) in CH₃CN (1 mL) was stirred at room temperature for 16 h. After concentration, PTLC (20% EtOAc/CH₂Cl₂) yielded the title compound.

MS (ESI): mass calculated for C₂₂H₂₇FN₄O₂, 398.2; m/z measured, 399.3 [M+H]⁺

¹H NMR (CDCl₃): 7.50-7.40 (m, 2H), 7.17-7.10 (m, 2H), 6.87 (t, J=9.0, 1H), 6.62-6.58 (m, 1H), 6.43-6.41 (m, 1H), 4.93 (s, 1H), 3.20-3.03 (m, 4H), 2.81 (t, J=4.9, 4H), 2.06-1.92 (m, 1H), 1.78-1.64 (m, 2H), 1.62-1.50 (m, 2H), 0.94 (t, J=7.4, 6H)

### Example 72

### N-(4-{4-[Cyano-(2-cyano-phenyl)-methyl]-piperazin-1-yl}-3-fluoro-phenyl)-2-ethyl-butyramide (Compound #136)

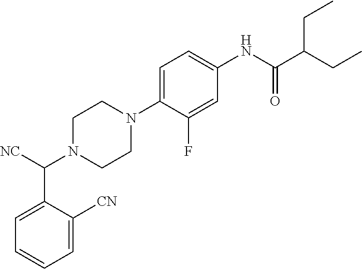

The title compound was prepared according to the process outlined in Example 71 above, with the appropriate substituent changes.

MS (ESI): mass calculated for C₂₅H₂₈FN₅O, 433.2; m/z measured, 434.3 [M+H]⁺

¹H NMR (CDCl₃): 7.80-7.76 (m, 2H), 7.72-7.66 (m, 1H), 7.56-7.43 (m, 2H), 6.12-6.07 (m, 2H), 6.85 (t, J=9.0, 1H), 5.12 (s, 1H), 3.20-3.10 (m, 2H), 3.08-2.98 (m, 2H), 2.94-2.84 (m, 2H), 2.77-2.67 (m, 2H), 2.05-1.96 (m, 1H), 1.78-1.50 (m, 4H), 0.94 (t, J=7.4, 6H),

### Example 73

### N-(4-{4-[Cyano-(2-methoxy-phenyl)-methyl]-piperazin-1-yl}-3-fluoro-phenyl)-2-ethyl-butyramide (Compound #137)

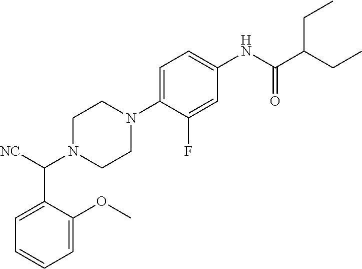

The title compound was prepared according to the process outlined in Example 71 above, with the appropriate substituent changes.

MS (ESI): mass calculated for C₂₅H₃₁FN₄O₂, 438.2; m/z measured, 439.2 [M+H]⁺

¹H NMR (CDCl₃): 7.55-7.35 (m, 3H), 7.15-7.06 (m, 2H), 7.05-7.00 (m, 1H), 6.96 (d, J=7.5, 1H), 6.85 (t, J=9.0, 1H), 5.18 (s, 1H), 3.88 (s, 3H), 3.12-3.00 (m, 4H), 2.88-2.72 (m, 4H), 2.05-1.96 (m, 1H), 1.78-1.50 (m, 4H), 0.94 (t, J=7.4, 6H),

### Example 74

### N-{4-[4-(Cyano-pyridin-2-yl-methyl)-piperazin-1-yl]-3-fluoro-phenyl}-2-ethyl-butyramide (Compound #138)

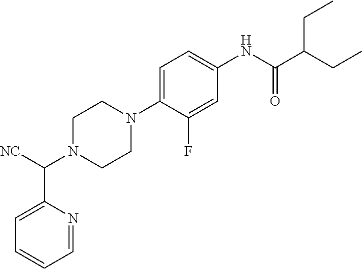

The title compound was prepared according to the process outlined in Example 71 above, with the appropriate substituent changes.

MS (ESI): mass calculated for C₂₃H₂₈FN₅O, 409.2; m/z measured, 410.2 [M+H]⁺

¹H NMR (CDCl₃): 8.73-8.64 (m, 1H), 7.84-7.75 (m, 1H), 7.73-7.59 (m, 2H), 7.50-7.42 (m, 1H), 7.36-7.31 (m, 1H), 7.22-7.16 (m, 1H), 6.85 (t, J=9.0, 1H), 5.05 (s, 1H), 3.15-3.02 (m, 4H), 2.88-2.72 (m, 4H), 2.08-1.99 (m, 1H), 1.77-1.50 (m, 4H), 0.94 (t, J=7.4, 6H),

### Example 75

### N-(3-Cyano-4-{4-[(4-fluoro-phenyl)-oxazol-2-yl-methyl]-piperazin-1-yl}-phenyl)-2-ethyl-butyramide (Compound #139)

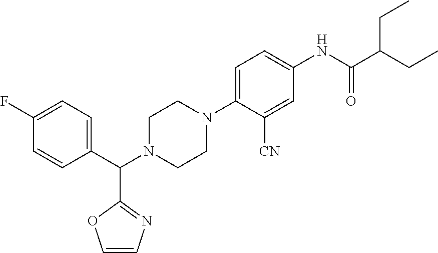

The title compound was prepared according to the process described in Example 64, with the appropriate substituent changes.

MS (ESI) mass calculated for C₂₇H₃₀FN₅O₂, 475.56; m/z measured, 476.6 [M+H]⁺

¹H NMR (CD₃OD): 7.94-7.89 (m, 2H), 7.69 (dd, J=8.9, 2.5, 1H), 7.59-7.52 (m, 2H), 7.20-7.16 (m, 1H), 7.14-7.07 (m, 3H), 4.81 (s, 1H), 3.22-3.14 (m, 4H), 2.73-2.63 (m, 2H), 2.57-2.48 (m, 2H), 2.24-2.15 (m, 1H), 1.70-1.47 (m, 4H), 0.92 (t, J=7.4, 4H).

### Example 76

### 2-Ethyl-N-(3-fluoro-4-{4-[(4-methoxy-phenyl)-oxazol-2-yl-methyl]-piperazin-1-yl}-phenyl)-butyramide (Compound #140)

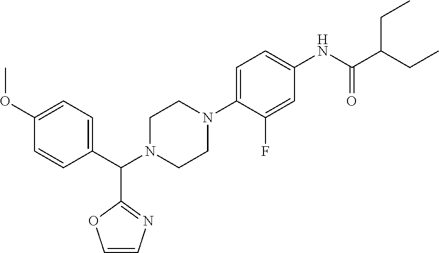

The title compound was prepared according to the process described in Example 64, with the appropriate substituent changes.

MS (ESI) mass calculated for C₂₇H₃₃FN₄O₃, 480.57; m/z measured, 481.3 [M+H]⁺

¹H NMR (CDCl₃): 7.62 (s, 1H), 7.46-7.49 (m, 2H), 7.14 (s, 1H), 7.11-7.05 (m, 2H), 6.90-6.83 (m, 3H), 4.70 (s, 1H), 3.79 (s, 3H), 3.09-3.02 (m, 4H), 2.72-2.63 (m, 2H), 2.55-2.45 (m, 2H), 2.01-1.94 (m, 1H), 1.74-1.67 (m, 2H), 1.58-1.49 (m, 2H), 0.93 (t, J=7.4, 6H).

### Example 77

### N-(3-Cyano-4-{4-[(4-methoxy-phenyl)-oxazol-2-yl-methyl]-piperazin-1-yl}-phenyl)-2-ethyl-butyramide (Compound #141)

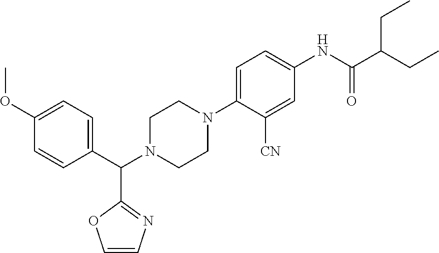

The title compound was prepared according to the process described in Example 64, with the appropriate substituent changes.

MS (ESI) mass calculated for C₂₈H₃₃N₅O₃, 487.59; m/z measured, 488.3 [M+H]⁺

¹H NMR (CDCl₃): 7.74 (d, J=2.6, 1H), 7.67 (dd, J=8.9, 2.6, 1H), 7.62 (s, 1H), 7.42 (d, J=8.7, 2H), 7.08 (s, 1H), 6.95 (d, J=8.9, 1H), 6.87 (d, J=8.7, 2H), 4.70 (s, 1H), 3.80 (s, 3H), 3.22-3.14 (m, 4H), 2.74-2.66 (m, 2H), 2.58-2.51 (m, 2H), 2.06-1.97 (m, 1H), 1.76-1.64 (m, 2H), 1.60-1.51 (m, 2H), 0.94 (t, J=7.4, 6H).

### Example 78

### N-(3-Cyano-4-{4-[(4-cyano-phenyl)-oxazol-2-yl-methyl]-piperazin-1-yl}-phenyl)-2-ethyl-butyramide (Compound #145)

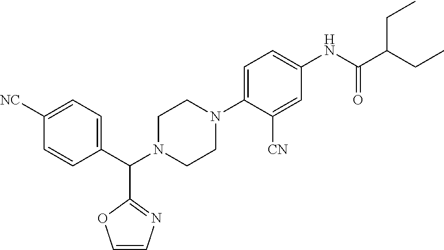

The title compound was prepared according to the process described in Example 64, with the appropriate substituent changes.

MS (ESI) mass calculated for C₂₈H₃₀N₆O₂, 482.59; m/z measured, 483.3 [M+H]⁺

¹H NMR (CDCl₃): 7.75 (d, J=2.5, 1H), 7.67-7.65 (m, 6H), 7.17 (s, 1H), 7.12 (s, 1H), 6.96 (d, J=8.9, 1H), 4.87 (s, 1H), 3.22-3.15 (m, 4H), 2.74-2.66 (m, 2H), 2.62-2.53 (m, 2H), 2.05-1.96 (m, 1H), 1.74-1.65 (m, 2H), 1.59-1.52 (m, 2H), 0.94 (t, J=7.4, 6H).

### Example 79

### N-(4-{4-[(4-Cyano-phenyl)-oxazol-2-yl-methyl]-piperazin-1-yl}-3-fluoro-phenyl)-2-ethyl-butyramide (Compound #146)

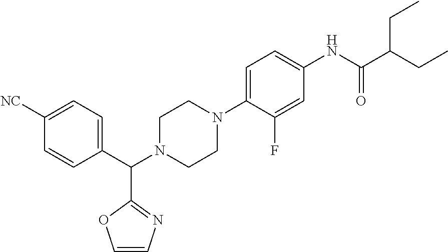

The title compound was prepared according to the process described in Example 64, with the appropriate substituent changes.

MS (ESI) mass calculated for C₂₇H₃₀FN₅O₂, 475.56; m/z measured, 476.3 [M+H]⁺

¹H NMR (CDCl₃): 7.68-7.64 (m, 5H), 7.44 (dd, J=13.4, 2.4, 1H), 7.14-7.08 (m, 2H), 7.07-7.03 (m, 1H), 6.86 (t, J=9.0, 1H), 4.87 (s, 1H), 3.11-3.04 (m, 4H), 2.71-2.63 (m, 2H), 2.58-2.50 (m, 2H), 2.03-1.94 (m, 1H), 1.75-1.64 (m, 2H), 1.56-1.52 (m, 2H), 0.94 (t, J=7.4, 6H).

### Example 80

### 2-Ethyl-N-(4-{4-[(5-ethyl-[1,3,4]-oxadiazol-2-yl)-phenyl-methyl]-piperazin-1-yl}-3-fluoro-phenyl)-butyramide (Compound #153)

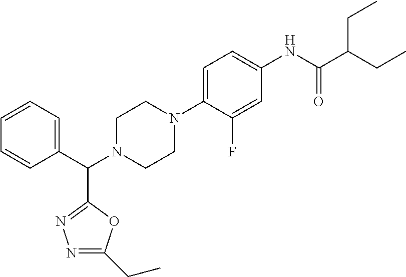

2-Ethyl-N-(4-{4-[(5-ethyl-[1,3,4]-oxadiazol-2-yl)-phenyl-methyl]-piperazin-1-yl}-3-fluoro-phenyl)-butyramide was prepared according to the procedure as described in Example 70 reacting the product prepared as in STEP A (157 mg) and propionyl chloride (0.034 mL) to yield the title compound as a colorless oil.

MS (electrospray): exact mass calculated for C₂₇H₃₄FN₅O₂, 479.27; measured m/z 480.3 [M+H]⁺

¹H NMR (500 MHz, CDCl₃): 7.53-7.47 (m, 2H), 7.43 (dd, J=14.01, 2.36 Hz, 1H), 7.40-7.30 (m, 3H), 7.19-7.09 (m, 2H), 6.90-6.83 (m, 1H), 4.91 (s, 1H), 3.12-3.03 (m, 4H), 2.90-2.83 (m, 2H), 2.76-2.67 (m, 2H), 2.61-2.51 (m, 2H), 2.03-1.95 (m, 1H), 1.76-1.65 (m, 2H), 1.60-1.50 (m, 2H), 1.36 (t, J=7.59 Hz, 3H), 0.94 (t, J=7.41 Hz, 6H).

### Example 81

### N-{3-Cyano-4-[4-(oxazol-2-yl-pyridin-2-yl-methyl)-piperazin-1-yl]-phenyl}-2-ethyl-butyramide (Compound #155)

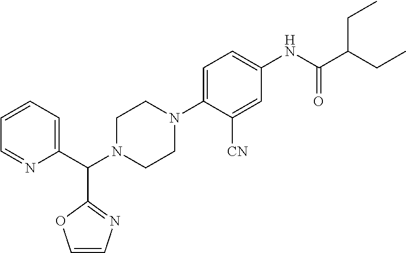

The title compound was prepared according to the process described in Example 64, with the appropriate substituent changes.

MS (ESI) mass calculated for C₂₆H₃₀N₆O₂, 458.56; m/z measured, 459.5 [M+H]⁺

¹H NMR (CDCl₃): 8.57-8.56 (m, 1H), 7.88 (d, J=2.6, 1H), 7.76-7.64 (m, 4H), 7.31 (m, 1H), 7.24-7.19 (m, 1H), 7.14-6.92 (m, 2H), 5.06 (s, 1H), 3.46-3.39 (m, 2.5H), 3.27-3.17 (m, 4H), 2.81-2.72 (m, 0.75H), 2.71-2.63 (m, 0.75H), 2.07-1.97 (m, 1H), 1.75-1.66 (m, 2H), 1.61-1.51 (m, 2H), 0.98-0.88 (m, 6H).

### Example 82

### 2-Ethyl-N-{3-fluoro-4-[4-(oxazol-2-yl-pyridin-2-yl-methyl)-piperazin-1-yl]-phenyl}-butyramide (compound #156)

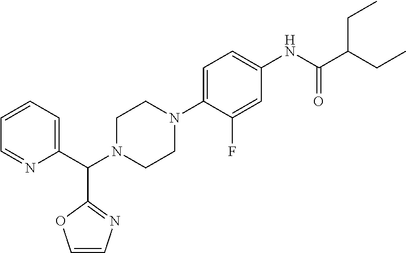

The title compound was prepared according to the process described in Example 64, with the appropriate substituent changes.

MS (ESI) mass calculated for C₂₅H₃₀FN₅O₂, 451.54; m/z measured, 452.5 [M+H]⁺

¹H NMR (CDCl₃): 8.60-8.55 (m, 1H), 7.74-7.68 (m, 1H), 7.57-7.50 (m, 1.25H), 7.45-7.39 (m, 0.75H), 7.29 (s, 1H), 7.22-7.19 (m, 1H), 7.13-7.07 (m, 2H), 6.91-6.83 (m, 2H), 5.06 (s, 1H), 3.41-3.36 (m, 2H), 3.16-3.05 (m, 3H), 2.84-2.60 (m, 3H), 2.05-1.94 (m, 1H), 1.75-1.63 (m, 2H), 1.61-1.50 (m, 2H), 0.96-0.88 (m, 6H).

### Example 83

### N-(4-{4-[4-Chloro-phenyl)-oxazol-2-yl-methyl]-piperazin-1-yl}-3-fluoro-phenyl)-2-ethyl-butyramide (Compound #157)

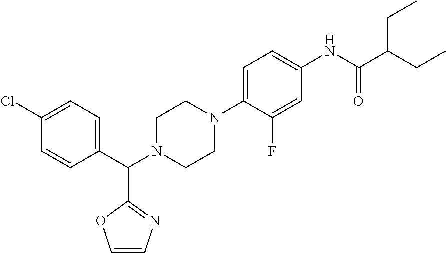

The title compound was prepared according to the process described in Example 64, with the appropriate substituent changes.

MS (ESI) mass calculated for C₂₆H₃₀ClFN₄O₂, 485.01; m/z measured, 486.3 [M+H]⁺

¹H NMR (CDCl₃): 7.64 (s, 1H), 7.49-7.39 (m, 3H), 7.35-7.29 (m, 2H), 7.17-7.06 (m, 3H), 6.89-6.82 (m, 1H), 4.75 (s, 1H), 3.11-3.02 (m, 4H), 2.72-2.63 (m, 2H), 2.56-2.46 (m, 2H), 2.01-1.94 (m, 1H), 1.74-1.63 (m, 3H), 1.59-1.49 (m, 1H), 0.93 (t, J=7.4, 6H).

### Example 84

### 2-Ethyl-N-(3-fluoro-4-{4-[(5-isopropyl-[1,3,4]-oxadiazol-2-yl)-phenyl-methyl]-piperazin-1-yl}-phenyl)-butyramide (Compound #160)

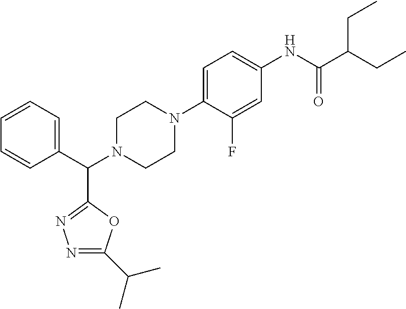

2-Ethyl-N-(3-fluoro-4-{4-[(5-isopropyl-[1,3,4]-oxadiazol-2-yl)-phenyl-methyl]-piperazin-1-yl}-phenyl)-butyramide was prepared according to the procedure as described in Example 70 reacting the product prepared as in STEP A and isobutyryl chloride (0.050 mL) to yield a colorless oil. The colorless oil was dissolved in Et₂O and treated with excess 1 M HCl in Et₂O. After 30 min the resulting mixture was concentrated in vacuo to yield the title compound as its corresponding HCl salt, as a pale yellow solid.

MS (electrospray): exact mass calculated for C₂₈H₃₆FN₅O₂, 493.29; measured m/z 494.3 [M+H]⁺

¹H NMR (600 MHz, MeOH-d₄): 7.70-7.67 (m, 2H), 7.62-7.55 (m, 4H), 7.28-7.25 (m, 1H), 7.11-7.06 (m, 1H), 6.20 (s, 1H), 3.76-3.34 (m, 8H), 3.28-3.20 (m, 1H), 2.24-2.17 (m, 1H), 1.69-1.58 (m, 2H), 1.57-1.46 (m, 2H), 1.39-1.34 (m, 6H), 0.95-0.88 (m, 6H).

### Example 85

### N-{4-[4-(Benzooxazol-2-yl-phenyl-methyl)-piperazin-1-yl]-3-fluoro-phenyl}-2-ethyl-butyramide (Compound #165)

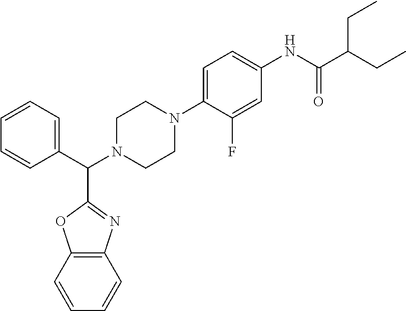

The title compound was prepared according to the process described in Example 64, with the appropriate substituent changes.

MS (ESI) mass calculated for C₃₀H₃₃FN₄O₂, 500.61; m/z measured, 501.3 [M+H]⁺

¹H NMR (CDCl₃): 7.73-7.70 (m, 1H), 7.63-7.58 (m, 2H), 7.54-7.50 (m, 1H), 7.43 (dd, J=14.0, 2.4, 1H), 7.40-7.34 (m, 2H), 7.33-7.29 (m, 3H), 7.12-7.05 (m, 2H), 6.87 (t, J=9.0 1H), 4.87 (s, 1H), 3.15-3.08 (m, 4H), 2.81-2.73 (m, 2H), 2.68-2.61 (m, 2H), 2.02-1.94 (m, 1H), 1.75-1.64 (m, 2H), 1.59-1.49 (m, 2H), 0.93 (t, J=7.4, 6H).

### Example 86

### N-{4-[4-(Benzooxazol-2-yl-phenyl-methyl)-piperazin-1-yl]-3-cyano-phenyl}-2-ethyl-butyramide (Compound #166)

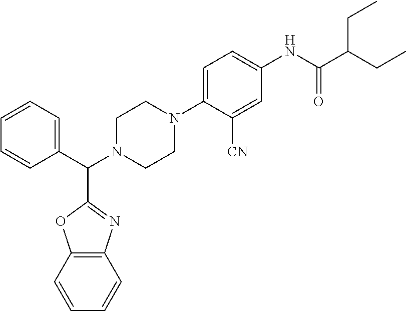

The title compound was prepared according to the process described in Example 64, with the appropriate substituent changes.

MS (ESI) mass calculated for C₃₁H₃₃N₅O₂, 507.64; m/z measured, 508.3 [M+H]⁺

¹H NMR (CDCl₃): 7.74 (d, J=2.5, 1H), 7.73-7.65 (m, 2H), 7.02-7.57 (m, 2H), 7.54-7.51 (m, 1H), 7.39-7.34 (m, 2H), 7.33-7.30 (m, 4H), 6.96 (d, J=8.9, 1H), 4.86 (s, 1H), 3.30-3.14 (m, 4H), 2.82-2.74 (m, 2H), 2.72-2.64 (m, 2H), 2.06-1.97 (m, 1H), 1.74-1.64 (m, 3H), 1.60-1.50 (m, 1H).

### Example 87

### N-{4-[4-(Benzothiazol-2-yl-phenyl-methyl)-piperazin-1-yl]-3-fluoro-phenyl}-2-ethyl-butyramide (compound #168)

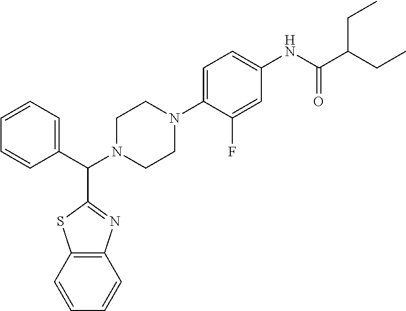

The title compound was prepared according to the process described in Example 64, with the appropriate substituent changes.

MS (ESI) mass calculated for C₃₀H₃₃FN₄OS, 516.67; m/z measured, 517.3 [M+H]⁺

¹H NMR (CDCl₃): 8.02-7.93 (dd, J=8.2, 1H), 7.09-7.81 (d, J=8.0, 1H), 7.57-7.51 (m, 2H), 7.49-7.24 (m, 6H), 7.22-7.08 (m, 2H0, 6.92-6.83 (m, 1H), 4.93 (s, 1H), 3.18-3.06 (m, 4H), 2.81-2.63 (m, 4H), 2.03-1.93 (m, 1H), 1.76-1.46 (m, 4H), 0.93 (t, J=7.4, 6H).

### Example 88

### N-{4-[4-(Benzothiazol-2-yl-phenyl-methyl)-piperazin-1-yl]-3-cyano-phenyl}-2-ethyl-butyramide (Compound #169)

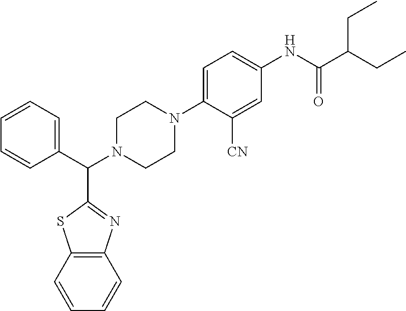

The title compound was prepared according to the process described in Example 64, with the appropriate substituent changes.

MS (ESI) mass calculated for C₃₀H₃₃N₅OS, 523.69; m/z measured, 524.3 [M+H]⁺

¹H NMR (CDCl₃): 7.96 (d, J=8.0, 1H), 7.85 (d, J=7.3, 1H), 7.75-7.66 (m, 2H), 7.57-7.50 (m, 2H), 7.45-7.40 (m, 2H), 7.38-7.32 (m, 3H), 7.10 (s, 1H), 6.98 (d, J=8.8, 1H), 4.95 (s, 1H), 3.33-3.16 (m, 4H), 2.86-2.66 (m, 4H), 2.09-2.92 (m, 1H), 1.77-1.62 (m, 2H), 1.56-1.47 (m, 2H), 0.94 (t, J=7.4, 6H).

### Example 89

### Cyclopropanecarboxylic acid 3-fluoro-4-[4-(oxazol-2-yl-phenyl-methyl)-piperazin-1-yl]-benzylamide (Compound #171)

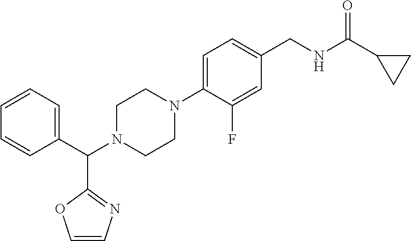

### Step A. Cyclopropanecarboxylic acid 4-chloro-3-fluoro-benzylamide

To a solution of 4-chloro-3-fluorobenzyl amine (232 mg, 1.5 mmol) in 7 mL CH₂Cl₂ was added cyclopropane carbonyl chloride (146 μL, 1.6 mmol). After a few minutes, triethylamine (222 μL, 1.6 mmol) was added and the resulting mixture was stirred for 18 h. The resulting mixture was washed (H₂O), dried (MgSO₄) and concentrated. The resulting oil was purified by PTLC to yield the title compound as a white solid.

MS (ESI): mass calculated for C₁₁H₁₁ClFNO, 227.66; m/z measured, 228.1 [M+H]⁺

¹H NMR (CDCl₃): 7.36-7.33 (m, 1H), 7.10-7.07 (m, 1H), 7.02-7.01 (m, 1H), 5.93 (bs, 1H), 4.43 (d, J=6.0, 2H), 1.39-1.34 (m, 1H), 1.03-1.01 (m, 2H), 0.80-0.76 (m, 2H).

### Step B. Cyclopropanecarboxylic acid 3-fluoro-4-piperazin-1-yl-benzylamide

A mixture of piperazine (47 mg, 0.55 mmol), cyclopropanecarboxylic acid 4-chloro-3-fluoro-benzylamide (150 mg, 0.66 mmol), tris(dibenzylideneacetone)dipalladium (126 mmol, 0.14 mmol), X-Phos (73 mg, 0.28 mmol) and sodium tert-butoxide (106 mg, 1.1 mmol) in toluene (3 mL) and CH₂Cl₂ (200 μL) was heated in a Biotage Initiator microwave for 30 min at 100° C. The solids were removed by filtration and the filtrate was concentrated and purified by reverse phase basic HPLC to yield the title compound as a white solid.

MS (ESI): mass calculated for C₁₅H₂₀FN₃O, 277.34; m/z measured, 278.3 [M+H]⁺

¹H NMR (CDCl₃): 6.97-6.95 (m, 1H), 6.90-6.87 (m, 1H), 5.93-5.89 (m, 1H), 4.36 (d, J=5.8, 2H), 3.08-3.02 (m, 8H), 2.17 (bs, 2H), 1.37-1.32 (m, 2H), 1.01-0.98 (m, 2H), 0.76-0.73 (m, 1H).

### Step C: Cyclopropanecarboxylic acid 3-fluoro-4-[4-(oxazol-2-yl-phenyl-methyl)-piperazin-1-yl]-benzylamide

A mixture of cyclopropanecarboxylic acid 3-fluoro-4-piperazin-1-yl-benzylamide (15 mg, (0.054 mmol), 2-(chloro-phenyl-methyl)-oxazole (10 mg, 0.054 mmol) and potassium carbonate (22 mg, 0.16 mmol) in ACN was heated at 60° C. for 18 h. After cooling to room temperature, the reaction was quenched with H₂O, and the resulting mixture extracted with CH₂Cl₂. The organics were washed (brine), dried (MgSO₄), and concentrated. The resulting oil was purified by reverse phase basic HPLC to yield the title compound as a white solid.

MS (ESI): mass calculated for C₂₅H₂₇FN₄O₂, 434.51; m/z measured, 435.3 [M+H]⁺

¹H NMR (CDCl₃): 7.63 (s, 1H), 7.52-7.51 (m, 2H), 7.36-7.34 (m, 2H), 7.31-7.28 (m, 1H), 7.08 (s, 1H), 6.97-6.93 (m, 2H), 6.88-6.85 (m, 1H), 5.86 (bs, 1H), 4.76 (bs, 1H), 4.35 (d, J=6.0, 2H), 3.14-3.02 (m, 4H), 2.74, 2.65, (m, 2H), 2.54-2.52 (m, 2H), 1.35-1.30 (m, 1H), 1.01-0.98 (m, 2H), 0.76-0.72 (m, 2H).

### Example 90

### 2-Ethyl-N-(3-fluoro-4-{4-[(3-methyl-isoxazol-5-yl)-phenyl-methyl]-piperazin-1-yl}-phenyl)-butyramide (Compound #230)

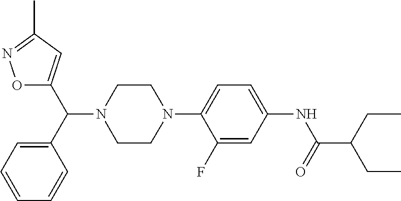

To a solution of acetone oxime (52 mg, 0.71 mmol) in dry THF (50 mL) cooled to 0° C., was added n-BuLi (1.6 M in hexane, 0.89 mL, 1.4 mmol). In a separate vessel, a solution of {4-[4-(2-ethyl-butyrylamino)-2-fluoro-phenyl]-piperazin-1-yl}-phenyl-acetic acid methyl ester (240 mg, 0.55 mmol) in THF (30 mL) was dried over 3A molecular sieves. Two hours after the initial addition of the n-BuLi to the acetone oxime, the solution of {4-[4-(2-ethyl-butyrylamino)-2-fluoro-phenyl]-piperazin-1-yl}-phenyl-acetic acid methyl ester was added to the mixture containing the acetone oxime and the resulting mixture was allowed to warm to room temperature, then stirred for 5 hours. Additional acetone oxime (104 mg), n-BuLi (1.6 M in hexane, 1.8 mL) and DMF (3 mL) were then added and the resulting mixture was allowed to stir for 12 h. The resulting mixture was then poured into a stirred solution of H₂SO₄ (173 μL), THF (5 mL) and H₂O (1 mL). After 1 h, additional H₂SO₄ (1 mL) was added and the resulting mixture was heated to reflux for 5 h. After cooling to room temperature, saturated aqueous NaHCO₃ was added until the solution reached pH 13. Ethyl acetate was added and the organic portion was washed with brine, dried (Na₂SO₄) and concentrated under reduced pressure to yield a residue. The residue was purified on the Agilent RP HPLC to yield the title compound.

MS (ESI) mass calculated for C₂₇H₃₃FN₄O₂, 464.58; m/z measured, 465.3 [M+H]⁺

¹H NMR 7.49-7.40 (m, 3H), 7.39-7.27 (m, 3H), 7.17-7.06 (m, 2H), 6.86 (dd, J=9.1, 8.9, 1H), 6.05 (s, 1H), 4.65 (s, 1H), 3.11-3.01 (m, 4H), 2.67-2.53 (m, 4H), 2.28 (s, 3H), 2.06-1.92 (m, 1H), 1.77-1.64 (m, 2H), 1.58-1.48 (m, 2H), 0.94 (t, J=7.6, 6H)

### Example 91

### 2-Ethyl-N-{3-fluoro-4-[4-([1,2,4]-oxadiazol-5-yl-phenyl-methyl)-piperazin-1-yl]-phenyl}-butyramide (Compound #231)

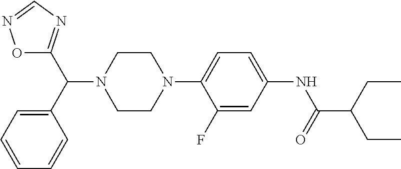

The title compound was prepared according to the process outlined in Example 1 above, with the appropriate substituent changes.

MS (ESI): mass calculated for C₂₅H₃₀FN₅O₂, 451.2; m/z measured, 452.3 [M+H]⁺

¹H NMR (CDCl₃): 8.42 (s, 1H), 7.52-7.32 (m, 5H), 7.13-7.03 (m, 2H), 6.87 (t, J=9.0, 1H), 5.01 (s, 1H), 3.15-3.02 (m, 4H), 2.78-2.52 (m, 4H), 2.05-1.94 (m, 1H), 1.77-1.50 (m, 4H), 0.94 (t, J=7.4, 6H),

### Example 92

### 2-Ethyl-N-{3-fluoro-4-[4-(2-oxo-1-phenyl-propyl)-piperazin-1-yl]-phenyl}-butyramide (Compound #232)

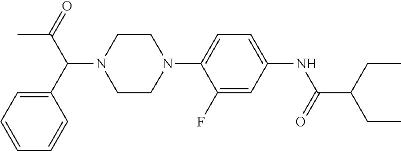

### STEP A: 1-chloro-1-phenyl-propan-2-one

To a mixture of 1-phenyl-propan-2-one (1.1 g, 8.4 mmol) in CCl₄ (3 mL) at 0° C. was added SO₂Cl₂ (0.75 mL, 9.3 mmol). The resulting mixture was stirred at room temperature for 24 h. After concentration, the title compound was isolated.

### STEP B: 2-ethyl-N-{3-fluoro-4-[4-(2-oxo-1-phenyl-propyl)-piperazin-1-yl]-phenyl}-butyramide

A mixture of 2-ethyl-N-(3-fluoro-4-piperazin-1-yl-phenyl)butyramide prepared as in STEP B of Example 71 (1 mmol), and 1-chloro-1-phenyl-propan-2-one (1 mmol) in DMA was heated at 100° C. for 0.5 h. After concentration, PTLC (20% EtOAc/CH₂Cl₂) yielded the title compound.

MS (ESI): mass calculated for C₂₅H₃₂FN₃O₂, 425.3; m/z measured, 426.3 [M+H]⁺

¹H NMR (CDCl₃): 7.43-7.22 (m, 7H), 7.08-7.02 (m, 1H), 6.80 (t, J=9.0, 1H), 4.92 (s, 1H), 3.08-2.98 (m, 4H), 2.58-2.42 (m, 4H), 2.07 (s, 3H), 2.05-1.94 (m, 1H), 1.70-1.40 (m, 4H), 0.94 (t, J=7.4, 6H),

### Example 93

### N-{3,5-Difluoro-4-[4-(oxazol-2-yl-phenyl-methyl)-piperazin-1-yl]-phenyl}-2-ethyl-butyramide (Compound #243)

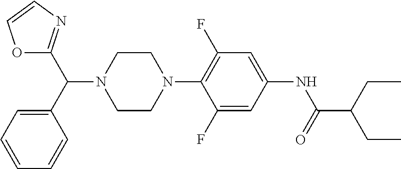

### STEP A: 4-(2,6-difluoro-4-nitro-phenyl)-piperazine-1-carboxylic acid tert-butyl ester

To a solution of N-Boc-piperazine (590 mg, 3.2 mmol) and 3,4,5-trifluoronitrobenzene (560 mg, 3.2 mmol) in acetonitrile (5 mL) was added K₂CO₃ (870 mg, 6.3 mmol) and the resulting mixture was heated to 50° C. for 18 hrs. After cooling to room temperature, water (30 mL) and a 3:1 mixture of ethyl acetate:hexane (20 mL) were added. The organic portion was extracted with water three times, extracted with brine once, dried (Na₂SO₄) and concentrated under reduced pressure to yield the title compound as a yellow solid.

¹H NMR 7.79-7.64 (m, 2H), 3.54-3.43 (m, 4H), 3.29-3.16 (m, 4H), 1.41 (s, 9H).

### STEP B: 4-(4-amino-2,6-difluoro-phenyl)-piperazine-1-carboxylic acid tert-butyl ester

To a solution of 4-(2,6-difluoro-4-nitro-phenyl)piperazine-1-carboxylic acid tert-butyl ester (2.4 mmol, 820 mg) in ethanol (50 mL) and ethyl acetate (25 mL), was added 5% Pd on carbon (20 mg). The reaction flask was evacuated and flushed with nitrogen three times, a septum was attached and a balloon of H₂ gas was inserted into the septum. After 18 h, the catalyst was filtered off through a pad of Celite® and the pad washed with ethyl acetate. The filtrate was concentrated to yield the title compound.

¹H NMR 6.14-6.04 (m, 2H), 3.47-3.38 (m, 4H), 2.97-2.87 (m, 4H), 1.39 (s, 9H).

### STEP C: 4-[4-(2-ethyl-butyrylamino)-2,6-difluoro-phenyl]-piperazine-1-carboxylic acid tert-butyl ester

To a mixture of the compound prepared as in STEP B above (1.3 mmol, 420 mg) in dichloromethane (6 mL) at 0° C. was added triethylamine (750 μL, 5.4 mmol) followed by 2-ethyl-butyryl chloride (1.6 mmol, 220 mL). After 18 h, additional dichloromethane and saturated NaHCO₃ solution were added. The organic portion was dried and concentrated under reduced pressure to yield a yellow oil which was purified by RP prep HPLC to yield the title compound.

¹H NMR 8.08 (br s, 1H), 7.93-7.82 (m, 2H), 4.32-4.19 (m, 4H), 3.85-3.74 (m, 4H), 2.79-2.68 (m, 1H), 2.50-2.35 (m, 2H), 2.34-2.22 (m, 2H), 2.20 (s, 9H), 1.65 (t, J=7.4, 6H).

### STEP D: N-(3,5-difluoro-4-piperazin-1-yl-phenyl)-2-ethyl-butyramide

To a mixture of the compound prepared as in STEP C above (290 mg, 0.70 mmol) in dichloromethane (10 mL) was added trifluoroacetic acid (3 mL). After 18 h, saturated NaHCO₃ solution was added until the pH of the solution was pH 13. After the addition of DCM, the organic portion was separated, dried (Na₂SO₄) and concentrated under reduced pressure to yield the title compound as a white solid, which was used in the next step without further purification.

### STEP E: N-{3,5-difluoro-4-[4-(oxazol-2-yl-phenyl-methyl)-piperazin-1-yl]-phenyl}-2-ethyl-butyramide

To a mixture of the compound prepared as in STEP D above (130 mg, 0.41 mmol) in DMF (1.5 mL) was added 2-(chloro-phenyl-methyl)-oxazole (96 mg, 0.5 mmol) and K₂CO₃ (170 mg, 1.2 mmol). After 48 h, the resulting mixture was diluted with water and ethyl acetate. The organic portion was extracted twice with brine, dried (Na₂SO₄) and concentrated under reduced pressure to yield a residue. The residue was purified by RP HPLC on the Agilent HP 1100 preparative HPLC to yield the title compound.

MS (ESI) mass calculated for C₂₆H₃₀F₂N₄O₂, 468.54; m/z measured, 469.3 [M+H]⁺

¹H NMR 7.63 (br s, 1H), 7.52 (br d, J=7.1, 2H), 7.38-7.32 (m, 2H), 7.32-7.26 (m, 1H), 7.14-7.04 (m, 4H), 4.76 (s, 1H), 3.27-3.10 (m, 4H), 2.70-2.56 (m, 2H), 2.52-2.37 (m, 2H), 2.04-1.90 (m, 1H), 1.77-1.62 (m, 2H), 1.59-1.46 (m, 2H), 0.93 (t, J=7.5, 6H)

### Example 94

### 2-Ethyl-N-(3-fluoro-4-{4-[phenyl-(5-trifluoromethyl-[1,2,4]-oxadiazol-3-yl)-methyl]-piperazin-1-yl}-phenyl)-butyramide (Compound #234)

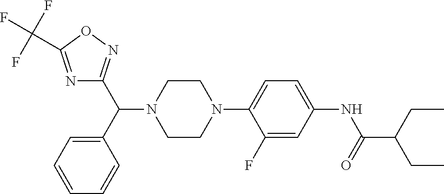

### STEP A: 1-(2-Fluoro-4-nitro-phenyl)-4-[phenyl-(5-trifluoromethyl-[1,2,4]-oxadiazol-3-yl)-methyl]-piperazine

To a solution of the product prepared as in Example 34, Step C (340 mg) in CH₂Cl₂ (8 mL) was added DIPEA (0.238 mL) followed by TFAA (0.13 mL) dropwise. After 30 min the resulting mixture was concentrated in vacuo and the chromatographed on SiO₂ (Hexanes to 20% EtOAc/Hexanes) to yield the title compound.

MS (electrospray): exact mass calculated for C₂₀H₁₇F₄N₅O₃, 451.13; measured m/z 452.2 [M+H]⁺.

### STEP B: 2-Ethyl-N-(3-fluoro-4-{4-[phenyl-(5-trifluoromethyl-[1,2,4]-oxadiazol-3-yl)-methyl]-piperazin-1-yl}-phenyl)-butyramide

2-Ethyl-N-(3-fluoro-4-{4-[phenyl-(5-trifluoromethyl-[1,2,4]-oxadiazol-3-yl)-methyl]-piperazin-1-yl}-phenyl)-butyramide was prepared according to the procedure as described in Example 34, Steps E and F reacting the product prepared as in Step A (426 mg) above to yield the title compound as a yellow solid.

MS (electrospray): exact mass calculated for C₂₆H₂₉F₄N₅O₂, 519.23; measured m/z 520.3 [M+H]⁺

¹H NMR (400 MHz, MeOH-d₄): 7.82-7.75 (m, 2H), 7.62-7.54 (m, 4H), 7.27 (dd, J=8.70, 1.51 Hz, 1H), 7.13-7.04 (m, 1H), 6.26 (s, 1H), 3.70-3.33 (m, 8H), 2.25-2.16 (m, 1H), 1.71-1.46 (m, 4H), 0.97-0.86 (m, 6H).

### Example 95

### 2-Ethyl-N-(3-fluoro-4-{4-[(5-fluoromethyl-[1,2,4]-oxadiazol-3-yl)-phenyl-methyl]-piperazin-1-yl}-phenyl)-butyramide (Compound #235)

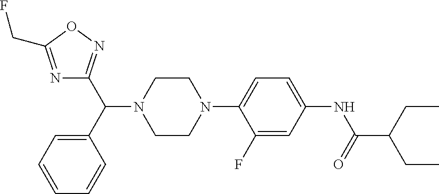

### STEP A: 1-[(5-Fluoromethyl-[1,2,4]-oxadiazol-3-yl)-phenyl-methyl]-4-(2-fluoro-4-nitro-phenyl)-piperazine

To a solution of the product prepared as in Example 34, Step C (342 mg) in CH₂Cl₂ (5 mL) was added DIPEA (0.24 mL) followed by 2-fluoroacetyl chloride (0.076 mL). After 10 min the resulting mixture was concentrated in vacuo and the residue chromatographed on SiO₂ (Hexanes to 50% EtOAc/Hexanes) to yield an orange viscous oil. The oil was dissolved in ^{t}BuOH (4 mL) and treated with NaOAc (34 mg) in H₂O (0.1 mL) and heated at 85° C. for 14 h. The resulting mixture was concentrated in vacuo and the residue chromatographed on SiO₂ (Hexanes to 30% EtOAc/Hexanes) to yield the title compound.

MS (electrospray): exact mass calculated for C₂₀H₁₉F₂N₅O₃, 415.15; measured m/z 416.2 [M+H]⁺.

### STEP B: 2-Ethyl-N-(3-fluoro-4-{4-[(5-fluoromethyl-[1,2,4]-oxadiazol-3-yl)-phenyl-methyl]-piperazin-1-yl}-phenyl)-butyramide

2-Ethyl-N-(3-fluoro-4-{4-[(5-fluoromethyl-[1,2,4]-oxadiazol-3-yl)-phenyl-methyl]-piperazin-1-yl}-phenyl)-butyramide was prepared according to the procedure as described in Example 34, Steps E and F reacting f the product prepared as in Step A (68 mg) above to yield the title compound as a colorless oil.

MS (electrospray): exact mass calculated for C₂₆H₃₁F₂N₅O₂, 483.24; measured m/z 484.3 [M+H]⁺

¹H NMR (500 MHz, CDCl₃): 7.57-7.51 (m, 2H), 7.44 (dd, J=14.02, 2.38 Hz, 1H), 7.39-7.29 (m, 3H), 7.13-7.04 (m, 2H), 6.91-6.83 (m, 1H), 5.63-5.45 (m, 2H), 4.82 (s, 1H), 3.15-3.03 (m, 4H), 2.76-2.67 (m, 2H), 2.64-2.54 (m, 2H), 2.03-1.95 (m, 1H), 1.78-1.43 (m, 4H), 0.98-0.90 (m, 6H).

### Example 96

### 2-Ethyl-N-(4-{4-[(5-ethyl-[1,2,4]-oxadiazol-3-yl)-phenyl-methyl]-piperazin-1-yl}-3-fluoro-phenyl)-butyramide (Compound #236)

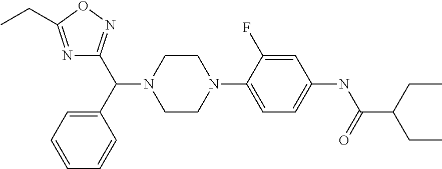

2-Ethyl-N-(4-{4-[(5-ethyl-[1,2,4]-oxadiazol-3-yl)-phenyl-methyl]-piperazin-1-yl}-3-fluoro-phenyl)-butyramide was prepared according to the procedure as described in Example 34 reacting the product prepared as in Example 34, Step C (337 mg) and propionyl chloride (0.086 mL) to yield the title compound as a white solid.

MS (electrospray): exact mass calculated for C₂₇H₃₄FN₅O₂, 479.27; measured m/z 480.3 [M+H]⁺

¹H NMR (400 MHz, CDCl₃): 7.59-7.52 (m, 2H), 7.44 (dd, J=14.04, 2.39 Hz, 1H), 7.39-7.27 (m, 3H), 7.16-7.05 (m, 2H), 6.91-6.82 (m, 1H), 4.74 (s, 1H), 3.16-3.03 (m, 4H), 2.91 (q, J=7.63 Hz, 2H), 2.76-2.52 (m, 4H), 2.04-1.93 (m, 1H), 1.77-1.62 (m, 2H), 1.61-1.47 (m, 2H), 1.37 (t, J=7.63 Hz, 3H), 0.93 (t, J=7.41 Hz, 6H).

### Example 97

### N-{3-Bromo-4-[4-(oxazol-2-yl-phenyl-methyl)-piperazin-1-yl]-phenyl}-2-ethyl-butyramide (Compound #237)

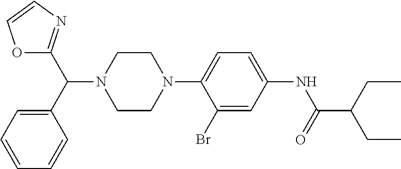

### STEP A: 4-(2-Bromo-4-nitro-phenyl)-piperazine-1-carboxylic acid tert-butyl ester

The title compound was prepared according to the procedure outlined in Example 93, STEP A above, substituting the appropriate materials as necessary.

¹H NMR 8.48 (d, J=2.6, 1H), 8.16 (dd, J=8.9, 2.6, 1H), 7.04 (d, J=8.9, 1H), 3.72-3.58 (m, 4H), 3.20-3.06 (m, 4H), 1.49 (s, 9H).

### STEP B: 1-(2-Bromo-4-nitro-phenyl)-piperazine

The title compound was prepared according to the procedure outlined in Example 93, STEP D above, substituting the appropriate materials as necessary.

¹H NMR 8.36-8.32 (m, 1H), 8.10-8.05 (m, 1H), 7.09-7.04 (m, 1H), 3.33-3.20 (m, 8H).

### STEP C: 1-(2-Bromo-4-nitro-phenyl)-4-(oxazol-2-yl-phenyl-methyl)-piperazine

The title compound was prepared according to the procedure outlined in Example 93, STEP E above, substituting the appropriate materials as necessary.

¹H NMR 8.44 (d, J=2.6, 1H), 8.15 (dd, J=8.9, 2.6, 1H), 7.67 (br s, 1H), 7.57-7.52 (m, 2H), 7.41-7.36 (m, 2H), 7.35-7.31 (m, 1H), 7.13 (br s, 1H), 7.04 (d, J=9.0, 1H), 4.81 (s, 1H), 3.28-3.22 (m, 4H), 2.83-2.70 (m, 2H), 2.64-2.52 (m, 2H).

### STEP D: 3-bromo-4-[4-(oxazol-2-yl-phenyl-methyl)-piperazin-1-yl]-phenylamine

To a refluxing solution of 1-(2-bromo-4-nitro-phenyl)-4-(oxazol-2-yl-phenyl-methyl)-piperazine (100 mg, 0.2 mmol) in ethanol (20 mL) was added SnCl₂ (305 mg, 1.35 mmol). After 30 min, the reaction flask was removed from the oil bath, and allowed to cool to room temperature. To the resulting mixture was added ethyl acetate and a solution of 1N NaOH (25 mL). The aqueous portion was extracted with ethyl acetate and the organic portions were combined, dried (Na₂SO₄) and concentrated under reduced pressure to the title compound, which was used in the next step without further purification.

¹H NMR 7.64 (d, J=0.7, 1H), 7.58-7.53 (m, 2H), 7.40-7.34 (m, 2H), 7.33-7.28 (m, 1H), 7.09 (d, J=0.7, 1H), 6.93 (d, J=2.7, 1H), 6.90 (d, J=8.5, 1H), 6.60 (dd, J=8.5, 2.7, 1H), 4.76 (s, 1H), 3.02-2.94 (m, 4H), 2.78-2.62 (m, 2H), 2.59-2.43 (m, 2H).

### STEP E: N-{3-Bromo-4-[4-(oxazol-2-yl-phenyl-methyl)-piperazin-1-yl]-phenyl}-2-ethyl-butyramide

The title compound was prepared according to the procedure outlined in Example 93, STEP C, substituting the appropriate materials as necessary.

MS (ESI) mass calculated for C₂₆H₃₁BrN₄O₂, 511.45; m/z measured, 511.2 [M+H]⁺

¹H NMR 7.77 (d, J=2.4, 1H), 7.64 (s, 1H), 7.55-7.51 (m, 2H), 7.46 (dd, J=8.6, 2.4, 1H), 7.41-7.28 (m, 3H), 7.24 (br s, 1H), 7.08 (s, 1H), 6.99 (d, J=8.7, 1H), 4.75 (s, 1H), 3.09-2.97 (m, 4H), 2.77-2.62 (m, 2H), 2.58-2.45 (m, 2H), 2.07-1.92 (m, 1H), 1.82-1.61 (m, 2H), 1.59-1.45 (m, 2H), 0.93 (t, J=7.4, 6H)

### Example 98

### 2-Ethyl-N-(3-fluoro-4-{4-[phenyl-(5-pyridin-3-yl-[1,2,4]-oxadiazol-3-yl)-methyl]-piperazin-1-yl}-phenyl)-butyramide (Compound #238)

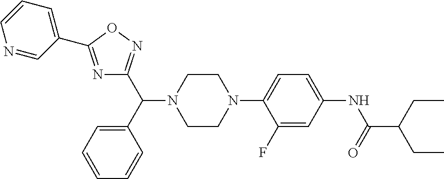

2-Ethyl-N-(3-fluoro-4-{-4-[phenyl-(5-pyridin-3-yl-[1,2,4]-oxadiazol-3-yl)-methyl]-piperazin-1-yl}-phenyl)-butyramide was prepared according to the procedure as described in Example 95 reacting the product prepared as in Example 34, Step C (302 mg) and nicotinoyl chloride (158 mg) to yield the title compound as a white solid.

MS (electrospray): exact mass calculated for C₃₀H₃₃FN₆O₂, 528.26; measured m/z 529.3 [M+H]⁺

¹H NMR (500 MHz, CDCl₃): 9.38 (d, J=1.47 Hz, 1H), 8.81 (dd, J=4.87, 1.68 Hz, 1H), 8.42 (td, J=8.00, 1.93 Hz, 1H), 7.64-7.59 (m, 2H), 7.49-7.41 (m, 2H), 7.41-7.35 (m, 2H), 7.35-7.29 (m, 1H), 7.12-7.03 (m, 2H), 6.91-6.84 (m, 1H), 4.86 (s, 1H), 3.16-3.07 (m, 4H), 2.81-2.72 (m, 2H), 2.72-2.61 (m, 2H), 2.03-1.94 (m, 1H), 1.75-1.64 (m, 2H), 1.62-1.49 (m, 2H), 0.96-0.91 (m, 6H).

### Example 99

### 2-Ethyl-N-(3-fluoro-4-{4-[(5-methyl-[1,2,4]-oxadiazol-3-yl)-phenyl-methyl]-piperazin-1-yl}-phenyl)-butyramide (Compound #239)

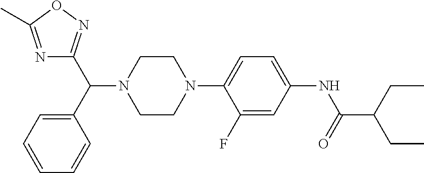

2-Ethyl-N-(3-fluoro-4-{4-[(5-methyl-[1,2,4]-oxadiazol-3-yl)-phenyl-methyl]-piperazin-1-yl}-phenyl)-butyramide was prepared according to the procedure as described in Example 95 reacting the product prepared as in Example 34, Step C (301 mg) and acetyl chloride (0.063 mL) to yield the title compound as a white solid.

MS (electrospray): exact mass calculated for C₂₆H₃₂FN₅O₂, 465.25; measured m/z 466.3 [M+H]⁺

¹H NMR (500 MHz, CDCl₃): 7.57-7.52 (m, 2H), 7.44 (dd, J=14.02, 2.38 Hz, 1H), 7.38-7.28 (m, 3H), 7.12-7.03 (m, 2H), 6.90-6.83 (m, 1H), 4.73 (s, 1H), 3.14-3.04 (m, 4H), 2.75-2.65 (m, 2H), 2.64-2.54 (m, 5H), 2.02-1.94 (m, 1H), 1.77-1.64 (m, 2H), 1.62-1.49 (m, 2H), 0.99-0.90 (m, 6H).

### Example 100

### 2-Ethyl-N-(3-fluoro-4-{4-[phenyl-(5-phenyl-[1,2,4]-oxadiazol-3-yl)-methyl]-piperazin-1-yl}-phenyl)-butyramide (Compound #240)

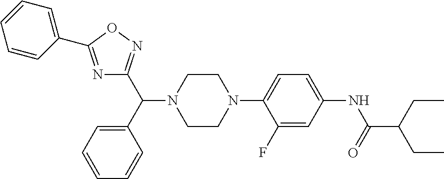

2-Ethyl-N-(3-fluoro-4-{4-[phenyl-(5-phenyl-[1,2,4]-oxadiazol-3-yl)-methyl]-piperazin-1-yl}-phenyl)-butyramide was prepared according to the procedure as described in Example 95 reacting the product prepared as in Example 34, Step C (301 mg) and benzoyl chloride (0.103 mL) to yield the title compound as a white solid.

MS (electrospray): exact mass calculated for C₃₁H₃₄FN₅O₂, 527.27; measured m/z 528.3 [M+H]⁺

¹H NMR (500 MHz, CDCl₃): 8.18-8.12 (m, 2H), 7.65-7.60 (m, 2H), 7.60-7.55 (m, 1H), 7.54-7.48 (m, 2H), 7.43 (dd, J=14.02, 2.38 Hz, 1H), 7.40-7.28 (m, 3H), 7.12-7.03 (m, 2H), 6.91-6.84 (m, 1H), 4.84 (s, 1H), 3.16-3.07 (m, 4H), 2.81-2.72 (m, 2H), 2.72-2.62 (m, 2H), 2.04-1.94 (m, 1H), 1.76-1.63 (m, 2H), 1.60-1.49 (m, 2H), 0.93 (t, J=7.41 Hz, 6H).

### Example 101

### 2-Ethyl-N-(3-fluoro-4-{4-[3-isopropyl-[1,2,4]-oxadiazol-5-yl)-phenyl-methyl]-piperazin-1-yl}-phenyl)-butyramide (Compound #241)

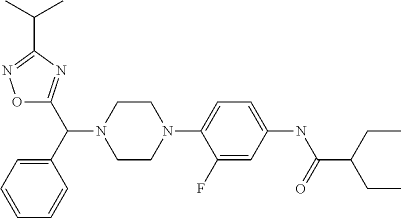

To a heterogeneous mixture of {4-[4-(2-ethyl-butyrylamino)-2-fluoro-phenyl]-piperazin-1-yl}-phenyl-acetic acid methyl ester (337 mg) in toluene (5 mL) was added K₂CO₃ (232 mg) followed by N-hydroxy-isobutyramidine (172 mg). The resulting mixture was heated at vigorous reflux for 3 days. The resulting mixture was filtered and concentrated in vacuo. The resulting residue was chromatographed on SiO₂ (Hexanes to 25% EtOAc/Hexanes) to yield the title compound as a white solid.

MS (electrospray): exact mass calculated for C₂₈H₃₆FN₅O₂, 493.29; measured m/z 494.3 [M+H]⁺

¹H NMR (500 MHz, CDCl₃): 7.56-7.50 (m, 2H), 7.44 (dd, J=14.02, 2.36 Hz, 1H), 7.41-7.30 (m, 3H), 7.13-7.05 (m, 2H), 6.92-6.82 (m, 1H), 4.90 (s, 1H), 3.17-3.01 (m, 5H), 2.77-2.65 (m, 2H), 2.63-2.51 (m, 2H), 2.04-1.93 (m, 1H), 1.76-1.64 (m, 2H), 1.61-1.50 (m, 2H), 1.40-1.31 (m, 6H), 0.98-0.89 (m, 6H).

### Example 102

### 2-Ethyl-N-(3-fluoro-4-{4-[phenyl-(3-phenyl-[1,2,4]-oxadiazol-5-yl)-methyl]-piperazin-1-yl}-phenyl)-butyramide (Compound #242)

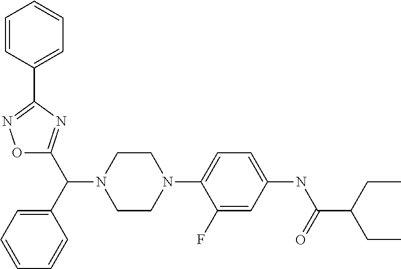

To a heterogeneous mixture of {4-[4-(2-ethyl-butyrylamino)-2-fluoro-phenyl]-piperazin-1-yl}-phenyl-acetic acid methyl ester (377 mg) in toluene (3 mL) was added K₂CO₃ (150 mg) followed by N-hydroxy-benzamidine (150 mg). The resulting mixture was heated in the microwave at 180° C. for 4 h and then filtered and concentrated in vacuo. The resulting residue was chromatographed on SiO₂ (Hexanes to 25% EtOAc/Hexanes) to yield the title compound as a pale yellow foam.

MS (electrospray): exact mass calculated for C₃₁H₃₄FN₅O₂, 527.27; measured m/z 528.3 [M+H]⁺

¹H NMR (500 MHz, CDCl₃): 8.14-8.07 (m, 2H), 7.61-7.55 (m, 2H), 7.52-7.32 (m, 7H), 7.13-7.03 (m, 2H), 6.91-6.84 (m, 1H), 5.01 (s, 1H), 3.16-3.05 (m, 4H), 2.84-2.74 (m, 2H), 2.70-2.59 (m, 2H), 2.03-1.94 (m, 1H), 1.77-1.64 (m, 2H), 1.63-1.49 (m, 2H), 0.94 (t, J=7.41 Hz, 6H).

### Example 103

### 2-({4-[4-(2-Ethyl-butyrylamino)-2-fluoro-phenyl]-piperazin-1-yl}-phenyl-methyl)-4,5-dihydro-oxazole-4-carboxylic acid methyl ester (Compound #179)

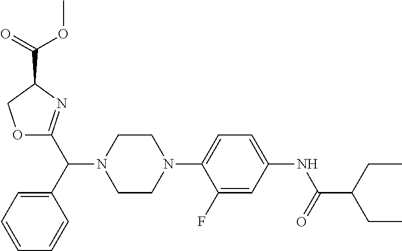

### STEP A: [4-(2-fluoro-4-nitro-phenyl)-piperazin-1-yl]-phenyl-acetic acid

To a solution of 1-(2-fluoro-4-nitro-phenyl)-piperazine (6.8 g, 30 mmol) and K₂CO₃ (6.3 g, 45 mmol) in DMF (20 mL) was added bromo-phenyl-acetic acid (7.2 g, 34 mmol). After 12 h, the pH of the solution was adjusted to pH 4 using 1 N HCl solution and then ethyl acetate was added. The aqueous portion was extracted three times with ethyl acetate. The organic extracts were dried (Na₂SO₄) and concentrated under reduced pressure to yield a yellow oil. The yellow oil was recrystallized from dichloromethane to yield the title compound as a yellow solid.

MS (ESI) Calculated for C₁₈H₁₈FN₃O₄, 359.35; m/z measured, 360.2 [M+H]⁺

### STEP B: 2-{2-[4-(2-Fluoro-4-nitro-phenyl)-piperazin-1-yl]-2-phenyl-acetylamino}-3-hydroxy-propionic acid methyl ester

To a solution of [4-(2-fluoro-4-nitro-phenyl)-piperazin-1-yl]-phenyl-acetic acid (5 g, 14 mmol) and triethylamine (7.8 mL, 55 mmol) in DMF (15 mL) was added (2S)-2-amino-3-hydroxy-propionic acid methyl ester hydrochloride (4.3 g, 27 mmol) followed by HATU (10.6 g, 28 mmol). A slight exotherm was observed and the resulting mixture was cooled to 0° C. for 1 h. After 12 h, saturated NaHCO₃ solution and ethyl acetate were added. The organic portion was dried (Na₂SO₄) and concentrated under reduced pressure to yield a residue, which was purified by silica gel chromatography followed by RP HPLC to yield the title compound.

MS (ESI) Calculated for C₂₂H₂₅FN₄O₆, 460.46; m/z measured, 461.2 [M+H]⁺

¹H NMR 8.04 (d, J=8.1, 0.5H), 7.98 (d, J=8.2, 0.5H), 7.91-7.82 (m, 1H), 7.81-7.72 (m, 1H), 7.34-7.21 (m, 5H), 6.85-6.74 (m, 1H), 4.65-4.53 (m, 1H), 3.95 (s, 0.5H), 3.93-3.87 (m, 1H), 3.85 (s, 0.5H), 3.84-3.79 (m, 0.5H), 3.77-3.72 (m, 0.5H), 3.70 (s, 1.5H), 3.68 (s, 1.5H), 3.31-3.20 (m, 4H), 2.69-2.61 (m, 1H), 2.59-2.45 (m, 4H)

### STEP C: 2-{[4-(2-fluoro-4-nitro-phenyl)-piperazin-1-yl]-phenyl-methyl}-4,5-dihydro-oxazole-4-carboxylic acid methyl ester

To a solution of the compound prepared as in STEP B above (33 mg, 0.07 mmol) in THF (15 mL) was added Burgess Reagent (20 mg, 0.09 mmol). The resulting mixture was heated to reflux for 2.5 h. After cooling to room temperature, the resulting mixture was concentrated under reduced pressure to yield a residue, which was purified on the Agilent RP HPLC to yield the title compound.

¹H NMR 8.68 (dd, J=8.9, 2.3, 1H), 8.60 (dd, J=13.1, 2.6, 1H), 8.26-8.19 (m, 2H), 8.13-8.02 (m, 3H), 7.60 (dd, J=8.9, 8.8, 1H), 5.53 (dd, J=10.5, 7.6, 0.5H), 5.44 (dd, J=10.5, 7.6, 0.5H), 5.33-5.20 (m, 1H), 5.18-5.10 (m, 1H), 5.05 (s, 0.5H), 5.02 (s, 0.5H), 4.53 (s, 1.5H), 4.48 (s, 1.5H), 4.10-4.03 (m, 4H), 3.51-3.32 (m, 4H).

### STEP D: 2-{[4-(4-Amino-2-fluoro-phenyl)-piperazin-1-yl]-phenyl-methyl}-4,5-dihydro-oxazole-4-carboxylic acid methyl ester

The title compound was prepared according to the procedure outlined in Example 93, STEP B, substituting the appropriate materials as necessary.

MS (ESI) Calculated for C₂₂H₂₅FN₄O₃, 412.46; m/z measured, 413.3 [M+H]⁺

¹H NMR 7.55-7.47 (m, 2H), 7.39-7.27 (m, 3H), 6.78 (dt, J=8.7, 2.5, 1H), 6.44-6.35 (m, 2H), 4.79 (dd, J=10.5, 7.7, 0.5H), 4.69 (dd, J=10.5, 7.8, 0.5H), 4.59-4.47 (m, 1H), 4.46-4.35 (m, 1H), 4.29 (d, J=3.7, 1H), 3.80 (s, 1.5H), 3.75 (s, 1.5H), 3.06-2.96 (m, 4H), 2.75-2.55 (m, 4H)

### STEP E: 2-({4-[4-(2-Ethyl-butyrylamino)-2-fluoro-phenyl]-piperazin-1-yl}-phenyl-methyl)-4,5-dihydro-oxazole-4-carboxylic acid methyl ester

The title compound was prepared according to the process outlined in Example 93, STEP C above, substituting the appropriate materials as necessary.

MS (ESI) Calculated for C₂₈H₃₅FN₄O₄, 510.60; m/z measured, 511.3 [M+H]⁺

¹H NMR 7.55-7.49 (m, 2H), 7.49-7.46 (m, 0.5H), 7.45-7.42 (m, 0.5H), 7.40-7.31 (m, 3H), 7.15-7.08 (m, 2H), 6.83 (dt, J=9.1, 2.0, 1H), 4.81 (dd, J=10.5, 7.7, 0.5H), 4.71 (dd, J=10.5, 7.7, 0.5H), 4.61-4.49 (m, 1H), 4.48-4.39 (m, 1H), 4.32 (d, J=5.7, 1H), 3.82 (s, 1.5H), 3.77 (s, 1.5H), 3.16-3.06 (m, 4H), 2.81-2.57 (m, 4H), 2.08-1.94 (m, 1H), 1.82-1.62 (m, 2H), 1.61-1.49 (m, 1H), 0.95 (t, J=7.4, 6H)

### Example 104

### 2-Ethyl-N-[4-(4-{[5-(1-ethyl-propyl)-[1,3,4]oxadiazol-2-yl]-phenyl-methyl}-piperazin-1-yl)-3-fluoro-phenyl]-butyramide (Compound #163)

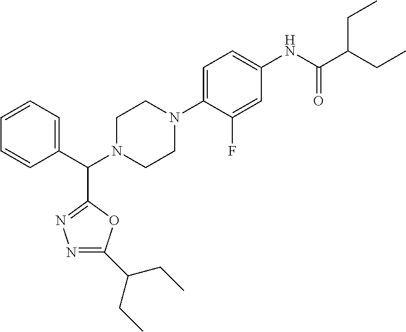

2-Ethyl-N-[4-(4-{[5-(1-ethyl-propyl)-[1,3,4]oxadiazol-2-yl]-phenyl-methyl}-piperazin-1-yl)-3-fluoro-phenyl]-butyramide was prepared according to the procedure as described in Example 70 above and 2-ethylbutyryl chloride (0.069 mL) to yield a colorless oil. The colorless oil was dissolved in Et₂O and treated with excess 1 M HCl in Et₂O. After 30 min the resulting mixture was concentrated in vacuo to yield the title compound as its corresponding HCl salt, as a white solid.

MS (electrospray): exact mass calculated for C₃₀H₄₀FN₅O₂, 521.32; measured m/z 522.4 [M+H]⁺

¹H NMR (600 MHz, MeOH-d₄): 7.65-7.55 (m, 6H), 7.27 (dd, J=8.72, 1.56 Hz, 1H), 7.12-7.06 (m, 1H), 6.21 (s, 1H), 3.88-3.35 (m, 8H), 2.96-2.89 (m, 1H), 2.24-2.16 (m, 1H), 1.82-1.58 (m, 6H), 1.57-1.47 (m, 2H), 0.94-0.78 (m, 12H).

### Example 105

### N-[4-(4-Benzhydryl-piperazin-1-yl)-3-cyano-phenyl]-2-dimethylamino-acetamide (Compound #300)

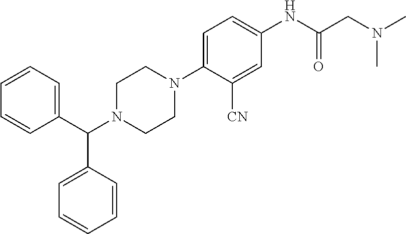

### Step A: 4-(2-Cyano-4-nitro-phenyl)-piperazine-1-carboxylic acid tert-butyl ester

piperazine-1-carboxylic acid tert-butyl ester (14 g, 75 mmol), 2-fluoro-5-nitrobenzonitrile (12.5 g, 75 mmol), and K₂CO₃ (31 g, 225 mmol) were combined in DMF (37.5 mL) and the resulting mixture heated to 90° C. for 18 h. The resulting mixture was then allowed to cool and filtered. The filter cake was washed with copious amounts of ethyl acetate, and the filtrate was concentrated to yield 4-(2-cyano-4-nitro-phenyl)-piperazine-1-carboxylic acid tert-butyl ester as a dark orange solid.

¹H NMR (400 MHz, CDCl₃): 8.45 (d, J=2.7 Hz, 1H), 8.29 (dd, J=9.29, 2.72 Hz, 1H), 6.99 (d, J=9.31 Hz, 1H), 3.66 (m, 4H), 3.46 (m, 4H), 1.48 (s, 9H).

### Step B: 5-Nitro-2-piperazin-1-yl-benzonitrile

4-(2-Cyano-4-nitro-phenyl)-piperazine-1-carboxylic acid tert-butyl ester (10 g, 30 mmol) was dissolved in DCM (230 mL). Trifluoroacetic acid (20 mL) was added to the resulitng mixture, which was then stirred at room temperature for 5 h. Saturated, aqueous sodium bicarbonate was added until aqueous layer was at neutral pH. Layers were separated, and the aqueous layer was extracted with ethyl acetate. The combined organic layers were dried over Na₂SO₄ and concentrated to yield 5-nitro-2-piperazin-1-yl-benzonitrile.

### Step C: 2-(4-Benzhydryl-piperazin-1-yl)-5-nitro-benzonitrile

5-A solution of nitro-2-piperazin-1-yl-benzonitrile (1.46 g, 6.32 mmol), diphenylchloromethane (1.35 mL, 7.58 mmol) and DIPEA (3.3 mL, 18.9 mmol) in acetonitrile (4.2 mL) was heated at 70° C. for 22 h. The resulting mixture was allowed to cool, then filtered to yield 2-(4-benzhydryl-piperazin-1-yl)-5-nitro-benzonitrile.

MS (ESI+APCI): mass calcd. for C₂₄H₂₂N₄O₂, 398.17; m/z found, 399.2 [M+H]⁺

¹H NMR (400 MHz, CDCl₃): 8.40 (d, J=2.72 Hz, 1H), 8.24 (dd, J=9.36, 2.75 Hz, 1H), 7.45-7.43 (m, 4H), 7.32-7.28 (m, 2H), 7.23-7.19 (m, 2H), 6.98 (d, J=9.39, 1H), 4.31 (s, 1H), 3.55-3.53 (m, 4H), 2.63-2.60 (m, 4H).

### Step D: 5-Amino-2-(4-benzhydryl-piperazin-1-yl)-benzonitrile

2-(4-Benzhydryl-piperazin-1-yl)-5-nitro-benzonitrile was suspended in acetone/H₂O (5/1, 45.6 mL). Ammonium chloride (3.7 g, 69 mmol) and zinc (1.5 g, 23 mmol) were added to resulitng mixture. The mixture was stirred for 2 h, then filtered through a pad of CELITE®. The filter cake was washed with copious amounts of ethyl acetate. The resulting filtrate was dried over Na₂SO₄ and filtered. Concentration in vacuo yielded 5-amino-2-(4-benzhydryl-piperazin-1-yl)benzonitrile, which was used in the next step without further purification.

### Step E. N-[4-(4-Benzhydryl-piperazin-1-yl)-3-cyano-phenyl]-2-dimethylamino-acetamide

N,N-dimethylglycine (33.4 mg, 0.32 mmol) was suspended in DMF (2 mL). DIPEA (0.141 mL, 0.81 mmol) and HATU (123 mg, 0.324 mmol) were added to the resulting mixture, which was then allowed to stir for 1 minute. 5-Amino-2-(4-benzhydryl-piperazin-1-yl)-benzonitrile (100 mg, 0.27 mmol) in DMF (1 mL) was added, and the resulting mixture was allowed to stir overnight. Water was added to the resulitng mixture, and the product was extracted into ethyl acetate. The organic layer was dried over Na₂SO₄ and concentrated. The resulting residue was purified by reverse phase HPLC (acidic) to yield the title compound, N-[4-(4-Benzhydryl-piperazin-1-yl)-3-cyano-phenyl]-2-dimethylamino-acetamide, as its corresponding TFA salt.

MS (ESI+APCI): Calculated for C₂₈H₃₁N₅O, 453.25; m/z measured 454.2 [M+H]⁺.

¹H NMR (400 MHz, CDCl₃): 10.82 (s, 1H), 7.72-7.69 (m, 5H), 7.64-7.37 (m, 7H), 6.87 (d, J=8.85 Hz, 1H), 4.92 (s, 1H), 4.05 (br S, 2H), 3.58-3.16 (m, 8H), 2.97 (br S, 6H).

### Example 106

### N-{3-Cyano-4-[4-(diphenylmethyl)piperazin-1-yl]phenyl}-3,5-dimethylisoxazole-4-carboxamide (Compound #302)

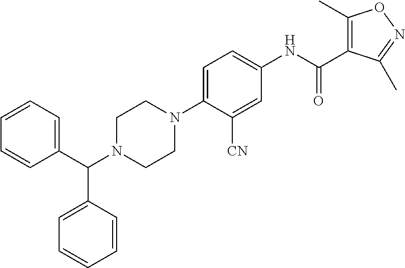

5-Amino-2-(4-benzhydryl-piperazin-1-yl)-benzonitrile (108 mg, 0.29 mmol) was dissolved in THF (2 mL), and triethylamine (0.123 mL, 0.88 mmol) was added. The resulitng mixture was cooled in an ice bath. 3,5-dimethyl-isoxazole-4-carbonyl chloride (61 mg, 0.38 mmol) was added, and the resulting mixture was allowed to stir at room temperature overnight. The resulting mixture was concentrated, redissolved in ethyl acetate, washed with H₂O, and dried over Na₂SO₄. The resulting residue was purified by column chromatography followed by trituration with methanol to yield the title compound.

MS (ESI+APCI): Calculated for C₃₀H₂₉N₅O₂, 491.23; m/z measured 492.2 [M+H]⁺.

¹H NMR (400 MHz, CDCl₃): 7.73 (d, J=2.60 Hz, 1H), 7.65 (dd, J=8.93, 2.64 Hz, 1H), 7.46-7.44 (m, 4H), 7.31-7.27 (m, 4H), 7.22-7.17 (m, 3H), 7.01 (d, J=8.93 Hz, 1H), 4.31 (s, 1H), 3.23-3.20 (m, 4H), 2.66 (s, 3H), 2.62-2.60 (m, 4H), 2.49 (s, 3H).

### Example 107

### N-[4-(4-Benzhydryl-piperazin-1-yl)-3-cyano-phenyl]-2-ethyl-butyramide (Compound #304)

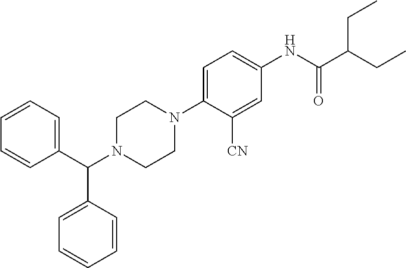

N-[4-(4-Benzhydryl-piperazin-1-yl)-3-cyano-phenyl]-2-ethyl-butyramide was prepared as described in Example 106, reacting the product prepared in Example 105, Step D (100 mg, 0.27 mmol) with 2-ethylbutyryl chloride (484, 0.35 mmol) to yield the title compound.

MS (ESI+APCI): Calculated for C₃₀H₃₄N₄O, 466.27; m/z measured 467.3 [M+H]⁺.

¹H NMR (400 MHz, d₆-DMSO): 10.021 (s, 1H), 7.99 (d, J=2.54 Hz, 1H), 7.71 (dd, J=8.98, 2.55 Hz, 1H), 7.48-7.45 (m, 4H), 7.33-7.30 (m, 4H), 7.22-7.15 (m, 3H), 4.39 (s, 1H), 3.11-3.09 (m, 4H), 2.51-2.46 (m, 4H [Note: overlaps with DMSO protons at 2.5 ppm]), 2.19-2.13 (m, 1H), 1.60-1.39 (m, 4H), 0.83 (t, J=7.40, 7.40 Hz, 1H).

### Example 108

### N-(3-Cyano-4-{4-[(2-methylphenyl)(phenyl)methyl]piperazin-1-yl}phenyl)-3,5-dimethylisoxazole-4-carboxamide (Compound #301)

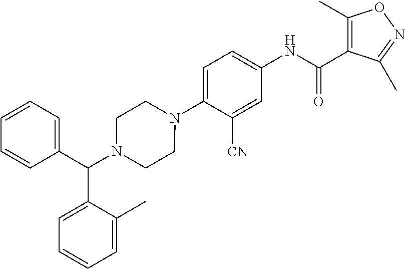

### Step A: 5-Nitro-2-[4-(phenyl-o-tolyl-methyl)-piperazin-1-yl]-benzonitrile

A solution of 2-methylbenzhydrol (585 mg, 2.95 mmol) and triethylamine (617 μL, 4.42 mmol) dissolved in DCM (9.8 mL) was cooled to 0° C., and methanesulfonyl chloride (253 μL, 3.25 mmol) was added. After stirring for 20 minutes at 0° C., the resulting mixture was allowed to stir at room temperature for 2 hours. The resulitng mixture was diluted with water, extracted into DCM, and concentrated. The resulting material was dissolved in acetonitrile (10 mL), and DIPEA (2.6 mL, 14.7 mmol) and 5-Amino-2-(4-benzhydryl-piperazin-1-yl)-10 benzonitrile (750 mg, 3.25 mmol) were added. The resulting mixture was heated at 60° C. for 12 hours, diluted with water and extracted into DCM. The combined organics were washed with brine, dried over Na₂SO₄ and concentrated to yield an oil, which was purified by column chromatography to yield 5-Nitro-2-[4-(phenyl-o-tolyl-methyl)-piperazin-1-yl]-benzonitrile.

MS (ESI): Calculated for C₂₅H₂₄N₄O₂ 412.19; m/z measured 413.2 [M+H]⁺.

¹H NMR (400 MHz, CDCl₃): 8.41 (d, J=2.72 Hz, 1H), 8.25 (dd, J=9.35, 2.74 Hz, 1H), 7.81 (d, J=7.58 Hz, 1H), 7.41-7.39 (m, 2H), 7.30-7.18 (m, 4H [Note: overlaps with CDCl₃]), 7.14-7.07 (m, 2H), 6.94 (d, J=9.2 Hz, 1H), 4.52 (s, 1H), 3.57-3.47 (m, 4H), 2.71-2.65 (m, 2H), 2.55-2.50 (m, 2H), 2.35 (s, 3H).

### Step B: 5-Amino-2-[4-(phenyl-o-tolyl-methyl)-piperazin-1-yl]benzonitrile

5-Nitro-2-[4-(phenyl-o-tolyl-methyl)-piperazin-1-yl]-benzonitrile (260 mg, 0.63 mmol) was suspended in acetone/H₂O (5/1, 6.3 mL). Ammonium chloride (508 mg, 9.5 mmol) and zinc (413 mg, 6.32 mmol) were added to resulitng mixture. The resulitng mixture was stirred for 2 h, then filtered through a pad of CELITE®. The filter cake was washed with copious amounts of acetone. The resulting filtrate was concentrated and suspended in ethyl acetate. Aqueous work-up followed by concentration in vacuo yielded the 5-amino-2-[4-(phenyl-o-tolyl-methyl)-piperazin-1-yl]benzonitrile, which was used in the next step without further purification.

MS (ESI): Calculated for C₂₅H₂₆N₄, 382.22; m/z measured 383.2 [M+H]⁺.

### Step C: N-(3-Cyano-4-{4-[(2 methylphenyl)(phenyl)methyl]piperazin-1-yl}phenyl)-3,5-dimethylisoxazole-4-carboxamide

5-Amino-2-[4-(phenyl-o-tolyl-methyl)-piperazin-1-yl]-benzonitrile was dissolved in tetrahydryofuran (2 mL). Triethylamine (110 μL, 0.37 mmol) was added, and the resulting mixture was cooled to 0° C. 3,5-Dimethylisoxazole-4-carbonyl chloride (54.4 mg, 0.34 mmol) was added, and the resulting mixture was then allowed to stir at room temperature for 12 h. Purification by column chromatography (0 to 100% ethyl acetate in hexane) yielded the title compound, N-(3-cyano-4-{4-[(2 methylphenyl)(phenyl)methyl]piperazin-1-yl}phenyl)-3,5-dimethylisoxazole-4-carboxamide.

MS (ESI): Calculated for C₃₁H₃₁N₅O₂, 505.25; m/z measured 506.2 [M+H]⁺.

¹H NMR (400 MHz, CDCl₃): 7.82-7.80 (m, 1H), 7.74 (d, J=2.6 Hz, 1H), 7.65 (dd, J=8.92, 2.63 Hz, 1H), 7.42-7.40 (m, 2H), 7.29-7.17 (m, 5H [Note: overlap with CDCl₃ at 7.26 ppm]), 7.12-7.06 (m, 2H), 7.00 (d, J=8.80 Hz, 1H), 4.52 (s, 1H), 3.22-3.17 (m, 4H), 2.71-2.64 (m, 5H), 2.56-2.49 (m, 5H), 2.35 (s, 3H).

### Example 109

### N-{3-Cyano-4-[4-(phenyl-o-tolyl-methyl)-piperazin-1-yl]-phenyl}-2-ethyl-butyramide (Compound #305)

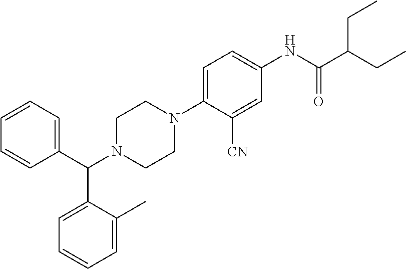

N-{3-Cyano-4-[4-(phenyl-o-tolyl-methyl)-piperazin-1-yl]-phenyl}-2-ethyl-butyramide was prepared as described a in Example 108, Step C, reacting 5-amino-2-[4-(phenyl-o-tolyl-methyl)-piperazin-1-yl]-benzonitrile (100 mg, 0.262 mmol) with 2-ethylbutyryl chloride (49 μL, 0.34 mmol) to yield the title compound.

MS (ESI+APCI): Calculated for C₃₁H₃₆N₄O, 480.29; m/z measured 481.1 [M+H]⁺.

¹H NMR (400 MHz, CDCl₃): 7.82-7.80 (m, 1H), 7.72 (d, J=2.56 Hz, 1H), 7.68 (dd, J=8.88, 2.62 Hz, 1H), 7.42-7.40 (m, 2H), 7.29-7.16 (m, 4H [Note: overlaps with CDCl₃ peak at 7.26 ppm]), 7.12-7.06 (m, 3H), 6.97 (d, J=8.90 Hz, 1H), 4.52 (s, 1H), 3.20-3.12 (m, 4H), 2.69-2.64 (m, 2H), 2.54-2.49 (m, 2H), 2.35 (s, 3H), 2.04-1.97 (m, 1H), 1.76-1.65 (m, 2H), 1.61-1.51 (m, 2H [Note: overlaps with H₂O peak from solvent]), 0.94 (t, J=7.41 Hz, 6H).

### Example 110

### N-(4-{4-[Bis(4-fluorophenyl)methyl]piperazin-1-yl}-3-cyanophenyl)-2-ethylbutanamide (Compound #307)

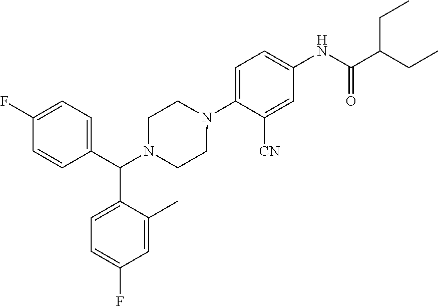

### Step A: 2-{4-[Bis-(4-fluoro-phenyl)-methyl]-piperazin-1-yl}-5-nitro-benzonitrile

Nitro-2-piperazin-1-yl-benzonitrile (2 g, 8.65 mmol), prepared as described in Example 105 Step B, was combined with chlorobis(4-fluorophenyl)methane (1.93 mL, 11 mmol) and DIPEA (4.5 mL, 26 mmol) in acetonitrile (20 mL). The resulting mixture was heated at 70° C. for 15 hours, then at reflux for 6 hours. The resulitng mixture was concentrated and purified by flash chromatography, then triturated with methanol to yield 2-{4-[bis-(4-fluoro-phenyl)-methyl]-piperazin-1-yl}-5-nitro-benzonitrile.

MS (ESI+APCI): Calculated for C₂₄H₂₀F₂N₄O₂, 434.16; m/z found 435.2, [M+H]⁺.

¹H NMR (400 MHz, CDCl₃): 8.41 (d, J=2.71 Hz, 1H), 8.26 (dd, J=9.34, 2.73 Hz, 1H), 7.40-7.35 (m, 4H), 7.03-6.97 (m, 4H), 6.95 (d, J=9.37 Hz, 1H), 4.31 (s, 1H), 3.54-3.52 (m, 4H), 2.61-2.58 (m, 4H).

### Step B: 5-Amino-2-{4-[bis-(4-fluoro-phenyl)-methyl]-piperazin-1-yl}-benzonitrile

2-{4-[Bis-(4-fluoro-phenyl)-methyl]-piperazin-1-yl}-5-nitro-benzonitrile (776 mg, 1.8 mmol) was suspended in acetone/H₂O (5/1, 9.0 mL). Ammonium chloride (1.4 g, 26.8 mmol) and then zinc (1.1 g, 17.9 mmol) were added and the resulting mixture was allowed to stir for 2 h. The resulitng mixture was then filtered through a pad of CELITE®, the filtrate was dried over Na₂SO₄ and then concentrated to yield 5-amino-2-{4-[bis-(4-fluoro-phenyl)-methyl]-piperazin-1-yl}-benzonitrile.

MS (ESI+APCI): Calculated for C₂₄H₂₂F₂N₄, 404.18; m/z measured 405.2 [M+H]⁺.

### Step C: N-(4-{-4-[Bis(4-fluorophenyl)methyl]piperazin-1-yl}-3-cyanophenyl)-2-ethylbutanamide

5-Amino-2-{4-[bis-(4-fluoro-phenyl)-methyl]-piperazin-1-yl}-benzonitrile (100 mg, 0.25 mmol) and triethylamine (104 μL, 0.75 mmol) were combined in THF (2 mL) and cooled in an ice bath. 2-Ethylbutyryl chloride (44.5 μL, 0.33 mmol) was added, and the resulting mixture was allowed to stir at room temperature for 12 hours. Aqueous work-up and purification by flash chromatography (0 to 100% ethyl acetate in hexane) yielded N-(4-{4-[bis(4-fluorophenyl)methyl]piperazin-1-yl}-3-cyanophenyl)-2-ethylbutanamide.

MS (ESI+APCI): Calculated for C₃₀H₃₂F₂N₄O, 502.25; m/z measured 503.2 [M+H]⁺.

¹H NMR (400 MHz, CDCl₃): 7.72-7.67 (m, 2H), 7.38-7.35 (m, 4H), 7.13 (br s, 1H), 7.00-6.96 (m, 5H), 4.30 (s, 1H), 3.18-3.16 (m, 4H), 2.58-2.56 (m, 4H), 2.05-1.98 (m, 1H), 1.76-1.65 (m, 2H), 1.61-1.51 (m, 2H, [Note: overlap with H₂O peak from solvent]), 0.942 (t, J=7.41 Hz, 6H).

### Example 111

### 3,5-Dimethyl-isoxazole-4-carboxylic acid (4-{4-[bis-(4-fluoro-phenyl)methyl]-piperazin-1-yl}-3-cyano-phenyl)-amide (Compound #309)

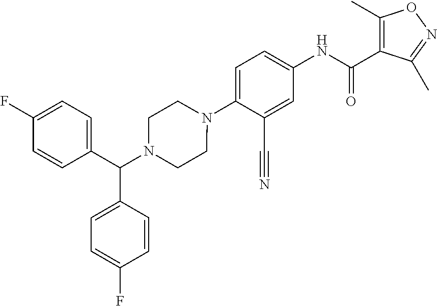

3,5-Dimethyl-isoxazole-4-carboxylic acid (4-{4-[bis-(4-fluoro-phenyl)-methyl]-piperazin-1-yl}-3-cyano-phenyl)-amide was prepared as described in Example 110 Step C, reacting 5-Amino-2-{-4-[bis-(4-fluoro-phenyl)-methyl]-piperazin-1-yl}-benzonitrile (104 mg, 0.26 mmol) with 3,5 dimethylisoxazole-4-carbonyl chloride (53.6 mg, 0.34 mmol) in the presence of triethylamine (109 μL, 0.78 mmol) to yield the title compound.

MS (ESI+APCI): Calculated for C₃₀H₂₇F₂N₅O₂, 527.21; m/z measured 528.2 [M+H]⁺.

¹H NMR (400 MHz, CDCl₃): 7.75 (d, J=2.6 Hz, 1H), 7.65 (dd, J=8.92, 2.64 Hz, 1H), 7.40-7.35 (m, 4H), 7.2 (br s, 1H), 7.02-6.96 (m, 5H), 4.31 (s, 1H), 3.22 (m, 4H), 2.67 (s, 3H), 2.59-2.57 (m, 4H), 2.50 (s, 3H).

### Example 112

### N-(4-{4-[(4-Chloro-phenyl)-phenyl-methyl]-piperazin-1-yl}-3-cyano-phenyl)-2-ethyl-butyramide (Compound #311)

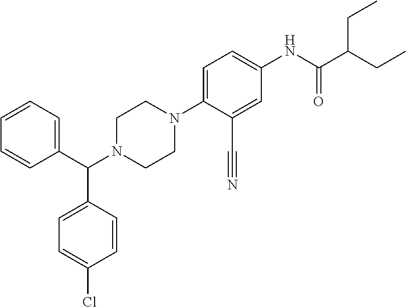

### Step A. 2-{4-[(4-Chloro-phenyl)-phenyl-methyl]-piperazin-1-yl}-5-nitro-benzonitrile

5-Amino-2-(4-benzhydryl-piperazin-1-yl)-benzonitrile (2 g, 8.65 mmol), prepared as described in Example 105, Step D, was combined with 4-chlorobenzhydryl chloride (1.99 mL, 10.4 mmol) and DIPEA (4.5 mL, 26 mmol) in acetonitrile (20 mL) and heated at 85-90° C. for 20 h. The resulitng mixture was allowed to cool and then concentrated. The resulting residue was purified by flash chromatography (0 to 100% ethyl acetate in hexane) to yield 2-{4-[(4-chloro-phenyl)-phenyl-methyl]-piperazin-1-yl}-5-nitro-benzonitrile.

MS (ESI+APCI): Calculated for C₂₄H₂₁ClN₄O₂, 432.14; m/z measured 433.1 [M+H]⁺.

¹H NMR (400 MHz, CDCl₃): 8.41 (d, J=2.71 Hz, 1H), 8.25 (dd, J=9.35, 2.75 Hz, 1H), 7.41-7.37 (m, 4H), 7.32-7.20 (m, 5H [Note: overlaps with CDCl₃ at 7.26 ppm]), 6.94 (d, J=9.38 Hz, 1H), 4.30 (s, 1H), 3.55-3.52 (m, 4H), 2.62-2.60 (m, 4H).

### Step B. 5-Amino-2-{4-[4-chloro-phenyl)-phenyl-methyl]-piperazin-1-yl}-benzonitrile

2-{4-[(4-Chloro-phenyl)-phenyl-methyl]-piperazin-1-yl}-5-nitro-benzonitrile was suspended in acetone/H₂O (5/1, 7.0 mL). Ammonium chloride (1.12 g, 21 mmol) and then zinc (0.92 g, 14 mmol) were added and the resulitng mixture was stirred at room temperature for 3 hours. The resulitng mixture was then filtered through CELITE®, and the filter cake was washed with ethyl acetate. The resulting filtrate was dried over Na₂SO₄, then concentrated in vacuo to yield 5-amino-2-{4-[(4-chloro-phenyl)-phenyl-methyl]-piperazin-1-yl}-benzonitrile.

MS (ESI+APCI): Calculated for C₂₄H₂₃ClN₄, 402.16; m/z measured 403.2 [M+H]⁺.

### Step C. N-(4-{4-[(4-Chloro-phenyl)-phenyl-methyl]-piperazin-1-yl}-3-cyano-phenyl)-2-ethyl-butyramide

A solution of 5-Amino-2-{4-[(4-chloro-phenyl)-phenyl-methyl]-piperazin-1-yl}-benzonitrile and triethylamine (105 μL, 0.75 mmol) in THF (2 mL) was cooled in an ice bath. 2-Ethylbutyryl chloride (55 μL, 0.40 mmol) was added, and the resulting mixture was allowed to stir at room temperature for 12 hours. The resulitng mixture was then concentrated, taken up in sufficient ethyl acetate to solubilize, washed with water and brine, and dried over Na₂SO₄. The resulting residue was purified by flash chromatography (0 to 100% EtOAc in hexane) to yield the title compound, N-(4-{4-[(4-chloro-phenyl)-phenyl-methyl]-piperazin-1-yl}-3-cyano-phenyl)-2-ethyl-butyramide.

MS (ESI+APCI): Calculated for C₃₀H₃₃ClN₄O, 500.23; m/z measured 501.2 [M+H]⁺.

¹H NMR (400 MHz, d₆-DMSO): 10.02 (s, 1H), 7.99 (d, J=2.53 Hz, 1H), 7.71 (dd, J=8.98, 2.54 Hz, 1H), 7.52-7.43 (m, 4H), 7.39-7.36 (m, 2H), 7.34-7.31 (m, 2H), 7.24-7.20 (m, 1H), 7.16 (d, J=9.00 Hz, 1H), 4.45 (s, 1H), 3.11-3.09 (m, 4H), 2.51-2.13 (m, 4H [Note: overlaps with DMSO peak at 2.50 ppm]), 2.20-2.13 (m, 1H), 1.60-1.39 (m, 4H), 0.83 (t, J=7.39 Hz, 6H).

### Example 113

### 3,5-Dimethyl-isoxazole-4-carboxylic acid (4-{4-[(4-chloro-phenyl)-phenyl-methyl]-piperazin-1-yl}-3-cyano-phenyl)-amide (Compound #312)

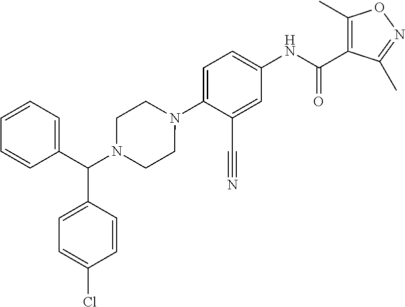

3,5-Dimethyl-isoxazole-4-carboxylic acid (4-{4-[(4-chloro-phenyl)-phenyl-methyl]-piperazin-1-yl}-3-cyano-phenyl)-amide was prepared as described in Example 112, Step C with the appropriate reagent changes (41.2 mg, 31%).

MS (ESI+APCI): Calculated for C₃₀H₂₈ClN₅O₂, 525.19; m/z measured 526.2 [M+H]⁺.

¹H NMR (400 MHz, CDCl₃): 7.74 (d, J=2.60 Hz, 1H), 7.66 (dd, J=8.92, 2.64 Hz, 1H), 7.41-7.37 (m, 4H), 7.31-7.18 (m, 6H [Note: overlaps with CDCl₃ peak at 7.26 ppm]), 7.01 (d, J=8.93 Hz, 1H), 4.30 (s, 1H), 3.22-3.20 (m, 4H), 2.67 (s, 3H), 2.61-2.59 (m, 4H), 2.49 (s, 3H).

### Example 114

### N-{3-Cyano-4-[4-(phenyl-pyridin-4-yl-methyl)-piperazin-1-yl]-phenyl}-2-ethyl-butyramide (Compound #315)

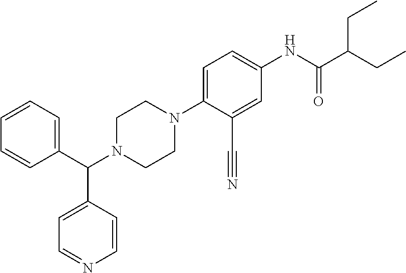

### Step A. 5-Nitro-2-[4-(phenyl-pyridin-4-yl-methyl)-piperazin-1-yl]-benzonitrile

A solution of nitro-2-piperazin-1-yl-benzonitrile (1.81 g, 7.80 mmol), prepared as described in Example 105, Step B, 4-(chloro-phenyl-methyl)-5 pyridine (Example I-U) (2.1 g, 10.2 mmol), and DIPEA (4.2 mL, 23.4 mmol) was heated in a sealed tube at 90° C. for 7 days and then allowed to cool. The resulitng mixture was concentrated in vacuo. The resulting residue was purified by flash chromatography (0 to 100% ethyl acetate in hexane) to yield 5-nitro-2-[4-(phenyl-pyridin-4-yl-methyl)-piperazin-1-yl]-benzonitrile.

MS (ESI+APCI): Calculated for C₂₃H₂₁N₅O₂, 399.17; m/z measured 400.2 [M+H]⁺.

### Step B. 5-Amino-2-[4-(phenyl-pyridin-4-yl-methyl)-piperazin-1-yl]-benzonitrile

5-Nitro-2-[4-(phenyl-pyridin-4-yl-methyl)-piperazin-1-yl]-benzonitrile (1.49 g, 3.7 mmol) was suspended in acetone/water (5/1, 18.6 mL). Ammonium chloride (3.0 g, 55.9 mmol), and then zinc (2.4 g, 37.3 mmol) were added, and the resulting mixture was allowed to stir at room temperature for 3 hours. An additional portion of Zinc (1.2 g, 18.6 mmol) was added, and the resulting mixture was allowed to stir for another hour. The resulitng mixture was then filtered through CELITE®, and the filter cake was washed with ethyl acetate. The filtrate was dried over Na₂SO₄ and concentrated to yield 5-amino-2-[4-(phenyl-pyridin-4-yl-methyl)-piperazin-1-yl]-benzonitrile, which was used in the next step without further purification.

MS (ESI+APCI): Calculated for C₂₃H₂₃N₅, 369.20; m/z measured 370.2 [M+H]⁺.

¹H NMR (400 MHz, CDCl₃): 8.60-8.59 (m, 2H), 7.53-7.52 (m, 2H), 7.36-7.21 (m, 5H [Note: overlaps with CDCl₃ peak at 7.26 ppm]), 6.91-6.81 (m, 3H), 4.34 (s, 1H), 3.63 (br s, 2H), 3.08-3.05 (m, 4H), 2.59-2.54 (m, 4H).

### Step C. N-{3-Cyano-4-[4-(phenyl-pyridin-4-yl-methyl)-piperazin-1-yl]-phenyl}-2-ethyl-butyramide

A solution of 5-Amino-2-[4-(phenyl-pyridin-4-yl-methyl)-piperazin-1-yl]-benzonitrile (100 mg, 0.27 mmol) and triethylamine (113 μL, 0.81 mmol) in THF (2 mL) was cooled in an ice bath. 2-Ethylbutyryl chloride (48 μL, 0.352 mmol) was added. The resulting mixture was allowed to warm and stirred at room temperature overnight. The resulting mixture was then concentration, and the resoidue purified via flash chromatography (0 to 100% ethyl acetate in hexane) to yield N-{3-cyano-4-[4-(phenyl-pyridin-4-yl-methyl)-piperazin-1-yl]-phenyl}-2-ethyl-butyramide.

MS (ESI+APCI): Calculated for C₂₉H₃₃N₅O, 467.27; m/z measured 468.3 [M+H]⁺.

¹H NMR (400 MHz, CDCl₃): 8.52-8.51 (m, 2H), 7.74-7.69 (m, 2H), 7.41-7.38 (m, 4H), 7.33-7.23 (m, 3H, [Note: overlaps with CDCl₃ at 7.26 ppm]), 6.98 (d, 8.8 Hz, 1H), 4.31 (s, 1H), 3.21-3.17 (m, 4H), 2.61-2.59 (m, 4H), 2.05-1.98 (m, 1H), 1.76-1.51 (m, 4H [Note: overlaps with H₂O peak]), 0.94 (t, J=7.41 Hz, 6H).

### Example 115

### Cyclopropanecarboxylic acid {3-cyano-4-[4-(phenyl-pyridin-4-yl-methyl)-piperazin-1-yl]-phenyl}-amide (Compound #322)

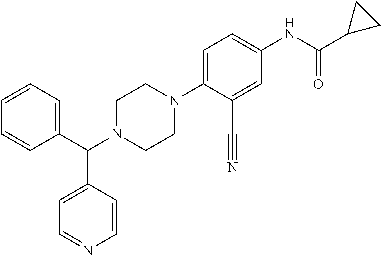

Cyclopropanecarboxylic acid {3-cyano-4-[4-(phenyl-pyridin-4-yl-methyl)-piperazin-1-yl]-phenyl}-amide was prepared as described in Example 114, Step C with the appropriate reagent substitutions. Following purification by flash chromatography (0-100% ethyl acetate in hexanes), trituration with methanol yielded the title compound.

MS (ESI+APCI): Calculated for C₂₇H₂₇N₅O, 437.22; m/z measured 438.2 [M+H]⁺.

¹H NMR (400 MHz, CDCl₃): 8.53-8.51 (m, 2H), 7.71-7.62 (m, 3H), 7.41-7.37 (m, 4H), 7.33-7.29 (m, 2H), 7.26-7.22 (m, 1H), 6.96 (d, J=8.89 Hz, 1H), 4.31 (s, 1H), 3.22-3.14 (m, 4H), 2.61-2.58 (m, 4H), 1.52-1.45 (m, 1H), 1.09-1.06 (m, 2H), 0.88-0.83 (m, 2H).

### Example 116

### N-{3-Cyano-4-[4-(phenyl-pyridin-3-yl-methyl)-piperazin-1-yl]-phenyl}-2-ethyl-butyramide (Compound #317)

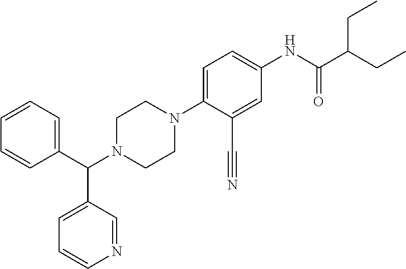

### Step A. 5-Nitro-2-[4-(phenyl-pyridin-3-yl-methyl)-piperazin-1-yl]-benzonitrile

A solution of nitro-2-piperazin-1-yl-benzonitrile (5.14 g, 22.1 mmol), prepared as described in Example 105, Step B, 3-(Chloro-phenyl-methyl)-pyridine (6.31 g, 30.9 mmol) (prepared as described in Example I-T), and DIPEA (11.8 mL, 66.39 mmol) was heated in a sealed tube at 90° C. for 24 hours and then allowed to cool. The resulitng mixture was concentrated, and the resulting residue was triturated with methanol to yield 5-nitro-2-[4-(phenyl-pyridin-3-yl-methyl)-piperazin-1-yl]-benzonitrile.

MS (ESI+APCI): Calculated for C₂₃H₂₁N₅O₂, 399.17; m/z measured 400.2 [M+H]⁺.

¹H NMR (400 MHz, CDCl₃): 8.72 (d, J=1.99 Hz, 1H), 8.48 (dd, J=4.79, 1.64 Hz, 1H), 8.42 (d, J=2.71 Hz, 1H), 8.26 (dd, J=9.34, 2.74 Hz, 1H), 7.75 (dt, J=7.90, 1.93 Hz, 1H), 7.43-7.40 (m, 2H), 7.35-7.30 (m, 2H), 7.27-7.23 (m, 3H [Note: overlaps with CDCl₃ peak at 7.26 ppm]), 6.95 (d, J=9.37 Hz, 1H), 4.38 (s, 1H), 3.54 (t, J=4.88 Hz, 4H), 2.68-2.58 (m, 4H).

### Step B. 5-Amino-2-[4-(phenyl-pyridin-3-yl-methyl)-piperazin-1-yl]-benzonitrile

5-Nitro-2-[4-(phenyl-pyridin-3-yl-methyl)-piperazin-1-yl]-benzonitrile (6.14 g, 15.4 mmol) was suspended in acetone/H₂O (5/1, 76.8 mL), and the resulting solution was cooled in an ice bath. Ammonium chloride (12.3 g, 230.5 mmol) was added, and then Zinc (15.1 g, 230.5 mmol) was added in three portions. The resulting mixture was allowed to stir at room temperature for 1.5 h and then filtered through CELITE®. The filter pad was washed with copious amounts of ethyl acetate, and the resulting filtrate was dried over Na₂SO₄, filtered, then concentrated to yield 5-amino-2-[4-(phenyl-pyridin-3-yl-methyl)-piperazin-1-yl]-benzonitrile.

MS (ESI+APCI): Calculated for C₂₃H₂₃N₅, 369.20; m/z measured 370.2 [M+H]⁺.

¹H NMR (400 MHz, CDCl₃): 8.79 (d, J=1.87 Hz, 1H) 8.51 (dd, J=4.95, 1.59 Hz, 1H), 7.84 (dt, J=7.90, 1.74 Hz, 1H), 7.38-7.35 (m, 2H), 7.32-7.20 (m, 4H), 6.91-6.81 (m, 3H), 4.39 (s, 1H), 3.63 (br s, 2H), 3.09-3.05 (m, 4H), 2.62-2.52 (m, 4H).

### Step C. N-{3-Cyano-4-[4-(phenyl-pyridin-3-yl-methyl)-piperazin-1-yl]-phenyl}-2-ethyl-butyramide

A solution of 5-Amino-2-[4-(phenyl-pyridin-3-yl-methyl)-piperazin-1-yl]-benzonitrile (100 mg, 0.27 mmol), triethyl amine (113 μL, 0.81 mmol) and 2-ethylbutyryl chloride (48 μL, 0.352 mmol) in THF (2 mL) was stirred at room temperature overnight. The resulting mixture was concentrated and purified by reverse phase HPLC. Fractions containing desired product were combined and concentrated to remove acetonitrile. The remaining aqueous solution was neutralized (1M NaOH), and the solid that precipitated was collected via filtration. Removal of residual water under high vacuum at 45° C. for 12 hours yielded the title compound, N-{3-cyano-4-[4-(phenyl-pyridin-3-yl-methyl)-piperazin-1-yl]-phenyl}-2-ethyl-butyramide.

MS (ESI+APCI): Calculated for C₂₉H₃₃N₅O, 467.27; m/z measured 468.3 [M+H]⁺.

¹H NMR (400 MHz, CDCl₃): 10.03 (s, 1H), 8.67-8.66 (m, 1H), 8.44 (dd, J=4.74, 1.63 Hz, 1H), 7.99 (d, J=2.53 Hz, 1H), 7.85 (dt, J=7.92, 1.89 Hz, 1H), 7.49-7.47 (m, 2H), 7.37-7.33 (m, 3H), 7.26-7.22 (m, 1H), 7.17 (d, J=9.0 Hz, 1H), 4.53 (s, 1H), 3.12-3.10 (m, 4H), 2.56-2.43 (m, 4H, [overlaps with DMSO at 2.5 ppm]), 2.20-2.13 (m, 1H), 1.60-1.38 (m, 4H), 0.83 (t, J=7.40 Hz, 6H).

### Example 117

### Cyclopropanecarboxylic acid {3-cyano-4-[4-(phenyl-pyridin-3-yl-methyl)-piperazin-1-yl]-phenyl}-amide (Compound #321)

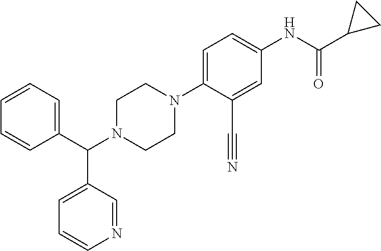

A solution of 5-amino-2-[4-(phenyl-pyridin-3-yl-methyl)-piperazin-1-yl]-benzonitrile (152.5 mg, 0.41 mmol), triethylamine (173 μL, 1.24 mmol) in THF (2 mL) was cooled in an ice bath. Cyclopropyl carbonyl chloride (49 μL, 0.54 mmol) was added dropwise, and the resulting mixture was allowed to warm and then stirred at room temperature overnight. The resulitng mixture was concentrated and purified by flash chromatography (0 to 100% ethyl acetate in hexane), then triturated with DCM and hexane to yield the title compound.

MS (ESI+APCI): Calculated for C₂₇H₂₇N₅O, 437.22; m/z measured 438.2 [M+H]⁺.

¹H NMR (400 MHz, CDCl₃): 8.70 (d, J=1.83 Hz, 1H), 8.45 (dd, J=4.75, 1.51 Hz, 1H), 7.75 (td, J=7.88, 1.82 Hz, 1H), 7.70-7.69 (m, 1H), 7.65-7.62 (m, 2H), 7.42-7.39 (m, 2H), 7.32-7.28 (m, 2H), 7.24-7.19 (m, 2H), 6.95 (d, J=8.91 Hz), 4.36 (s, 1H), 3.18-3.15 (m, 4H), 2.64-2.55 (m, 4H), 1.51-1.45 (m, 1H), 1.09-1.05 (m, 2H), 0.87-0.83 (m, 2H).

### Example 118

### Neuropeptide Y2 Radioligand Binding and pKb Assays

KAN-Ts endogenously expressing Y2 receptors were used for the radioligand binding assay. Cells were grown to confluence on 150 cm² tissue culture plates, washed with phosphate-buffered saline (PBS), and scraped into 50 mL tubes. After centrifugation, the supernatant was aspirated, and the pellets frozen and stored at −80° C. Thawed pellets were homogenized with a polytron tissue grinder for 15 sec in 20 mM Tris-HCl, 5 mM EDTA. The homogenate was centrifuged at 800×g for 5 min and the collected supernatant was recentrifuged at 25000×g for 25 min. The resulting pellet was resuspended in binding buffer (20 mM HEPES, 120 mM NaCl, 0.22 mM KH₂PO₄, 1.3 mM CaCl₂, 0.8 mM MgSO₄). Membranes were incubated with [¹²⁵I]PYY (80 pM) in the presence or absence of test compound for 1 h at rt. The reaction was stopped by filtration through GF/C filter plates pre-soaked in 0.3% polyethylenimine and subsequently washed with Tris 50 mM, 5 mM EDTA buffer. Plates were dried for 1 h in a 55° C. oven, scintillation fluid was added and the radioactivity was counted in a Packard TopCount. Specific binding to the NPY receptor subtypes was determined by radioactivity that was bound in the presence of 1 mM NPY. Each binding experiment was repeated three to eight times, each in duplicate. IC₅₀ values (i.e. concentration of unlabelled peptide or antagonist required to compete for 50% of specific binding to the radioligand) were calculated using the GraphPad Prism software (GraphPad Software Inc., San Diego Calif.) with a fit to a sigmoidal dose response curve. Apparent K, values were calculated as Kᵢ=IC₅₀/(1+C/K_{D}), where C is concentration of the radioligand.

### Example 119

### Neuropeptide Y2 pK_{B} Assay

The assay was performed using the fluorimetric imaging plate reader (FLIPR) format as described in Dautzenberg, F. M., Biochemical Pharmacology 2005, 69, 1493.

KAN-Ts cells stably expressing chimeric G proteins were seeded at a density of 100,000 cells into poly-d-lysine coated 384-well blackwall, clear-bottom microtiter plates (Corning, N.Y.). One day later, the medium was removed and 50 μl loading medium DMEM high glucose, without serum, supplemented with 10 mM HEPES-acid, 0.1% BSA, 5 mM probenecid and 2 μM Fluo-3AM was added. Cells were loaded for 1 h at 37° C., washed twice with 50 μl assay buffer (5 mM HEPES-acid, 140 mM NaCl, 1 mM MgCl₂, 5 mM KCl, 10 mM glucose) and then 30 μl assay buffer was added. Cells were further pre-incubated at room temperature before adding agonists or agonists plus antagonists in 20 μl assay buffer and then measured on a T-channel fluorometric imaging plate reader (FLIPR, Molecular Devices, Sunnyvale, Calif.). Antagonistic potency values were converted to apparent pK_{B} values using a modified Cheng-Prusoff correction. Apparent pK_{B} was calculated as pK_{B}=−log IC₅₀/1+[conc agonist/EC₅₀].

Representative compounds of the present invention were tested for NPY Y₂ radioligand binding and pK_{B} activity, as described in Examples 118 and 119 above, with results as listed in Table 3 below.

TABLE 3
NPY Y2 Binding and pK_{B}

| | | |
|---|---|---|
| Example No. | IC₅₀ (μM) | pK_{B} |

| 1 | 0.027 | | 7.45 |
|---|---|---|---|
| 2 | 0.027 | | 7.4 |
| 4 | 0.033 | | 7.1 |
| 12 | 0.33 | | |
| 13 | 0.45 | | |
| 14 | 0.08 | | |
| 20 | 0.04 | | 7.3 |
| 24 | 0.04 | | 7.1 |
| 27 | 10 | | |
| 33 | 0.018 | | 7.7 |
| 39 | 0.028 | | 7.2 |
| 40 | 0.052 | | 7.0 |
| 46 | 1.2 | | |
| 49 | >10 | | |
| 51 | 1.55 | | |
| 53 | 0.078 | | |
| 54 | 0.095 | | 5 |
| 55 | 0.054 | | |
| 57 | >10 | | |
| 58 | >10 | | |
| 59 | 0.096 | | 6.7 |
| 60 | 0.034 | | 7.2 |
| 61 | >10 | | |
| 62 | 4.7 | | |
| 63 | 0.71 | | |
| 64 | 0.17 | | |
| 66 | 1.5 | | |
| 72 | 0.32 | | |
| 73 | 0.18 | | |
| 74 | 2.6 | | |
| 76 | 1.4 | | |
| 78 | 0.053 | | |
| 81 | 1.7 | | |
| 82 | 0.022 | | 7.3 |
| 83 | 0.2 | | |
| 84 | 0.29 | | |
| 85 | 0.075 | | 6.9 |
| 86 | 0.005 | | |
| 87 | 0.054 | | |
| 90 | 0.038 | | 7.5 |
| 91 | 0.24 | | 6.8 |
| 92 | 0.27 | | |
| 93 | 0.032 | | |
| 95 | 0.1 | | |
| 96 | 0.15 | | |
| 98 | 0.18 | | |
| 100 | 1.3 | | |
| 101 | 0.17 | | |
| 102 | 0.72 | | |
| 103 | >10 | | |
| 104 | >10 | | |
| 106 | 0.25 | | |
| 107 | 1.2 | | |
| 108 | >10 | | |
| 109 | 3.6 | | |
| 110 | 0.029 | | 7.0 |
| 111 | 0.36 | | |
| 112 | 0.43 | | |
| 113 | 0.14 | | 6.7 |
| 114 | 0.46 | | |
| 115 | 0.45 | | |
| 116 | 0.41 | | |
| 117 | 0.31 | | |
| 120 | 0.77 | | |
| 122 | 0.43 | | |
| 126 | 0.34 | | |
| 127 | >5 | | |
| 131 | 0.007 | | 7.9 |
| 132 | 0.045 | | 7.2 |
| 134 | 0.052 | | 7.2 |
| 135 | >10 | | |
| 136 | 9.8 | | |
| 137 | 1.2 | | |
| 138 | >10 | | |
| 139 | 0.021 | | 7.6 |
| 140 | 0.32 | | |
| 141 | 0.029 | | 7.3 |
| 145 | 0.033 | | 7.2 |
| 146 | 0.26 | | |
| 153 | 0.039 | | 7.4 |
| 155 | 0.15 | | |
| 156 | 0.79 | | |
| 157 | 0.16 | | |
| 160 | 0.043 | | 7.0 |
| 163 | 0.02 | | 7.0 |
| 165 | 0.018 | | 7.0 |
| 166 | 0.007 | | 7.1 |
| 168 | 0.058 | | 7.15 |
| 169 | 0.039 | | 7.3 |
| 171 | >10 | | |
| 179 | 0.041 | | |
| 230 | 0.012 | | 7.6 |
| 231 | 0.22 | | |
| 232 | 0.38 | | |
| 234 | 0.12 | | |
| 235 | 0.03 | | 7.2 |
| 236 | 0.023 | | 7.8 |
| 237 | 0.016 | | 7.2 |
| 238 | 0.025 | | 7.5 |
| 239 | 0.013 | | 7.9 |
| 240 | 0.039; | | 6.9 |
| 241 | 0.035 | | |
| 242 | 0.071 | | |
| 243 | 0.05 | | 6.9 |
| 280 | 0.43 | | |
| 281 | 0.065 | | |
| 300 | Ki = 0.25 | μM | |
| 301 | Ki = 0.14 | μM | |
| 302 | Ki = 0.014 | μM | 6.3 |
| 304 | Ki = 0.0047 | μM | 6.3 |
| 305 | Ki = 0.036 | μM | 6.5 |
| 307 | Ki = 0.15 | μM | |
| 309 | Ki = 0.74 | μM | |
| 311 | Ki = 0.063 | μM | |
| 312 | Ki = 0.084 | μM | |
| 315 | Ki = 0.0044 | μM | 7.3 |
| 317 | Ki = 0.0043 | μM | 7.5 |
| 321 | Ki = 0.011 | μM | 7.5 |
| 322 | Ki = 0.034 | μM | |

### Example 119

### Prophetic Example, Pharmaceutical Composition

As a specific embodiment of an oral composition, 100 mg of the Compound #20 (prepared as in Example 7) is formulated with sufficient finely divided lactose to provide a total amount of 580 to 590 mg to fill a size 0 hard gel capsule.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

What is claimed:

1. A compound of formula (II) 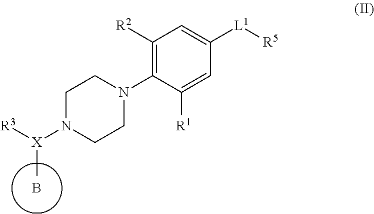
wherein
R¹ and R² are each independently selected from the group consisting of hydrogen, halogen, C₁₋₄alkyl, —C₁₋₄alkyl-OH, —C₁₋₄alkyl-O—C₁₋₄alkyl, —C₁₋₄alkoxy, —S—C₁₋₄alkyl, —SO—C₁₋₄alkyl, —SO₂—C₁₋₄alkyl, cyano, nitro, —NR^{A}R^{B}, —CH₂—NR^{A}R^{B}, —C(O)—NR^{A}R^{B} and —C(O)H; wherein R^{A} and R^{B} are each independently selected from the group consisting of hydrogen and C₁₋₄alkyl;
provided that at least one of R¹ or R² is other than hydrogen;
L¹ is selected from the group consisting of —NR^{J}—, —NR^{J}—C(O)—, —C(O)—NR^{J}—, —(CH₂)ₐ—C(O)—NR^{J}—, —(CH₂)ₐ—NR^{J}—C(O)— and —C(O)O—; wherein R^{J} is selected from the group consisting of hydrogen and C₁₋₄alkyl; and wherein a is an integer from 1 to 3;
R⁵ is selected from the group consisting of C₁₋₈alkyl, C₃₋₈cycloalkyl, aryl, heteroaryl, and heterocycloalkyl; wherein the C₁₋₈alkyl, C₃₋₈cycloalkyl, aryl, heteroaryl or heterocycloalkyl is optionally substituted with one or more substituents independently selected from the group consisting of halogen, C₁₋₄alkyl, halogenated C₁₋₄alkyl, C₁₋₄alkoxy, halogenated C₁₋₄alkoxy, hydroxy, cyano, nitro and NR^{K}R^{L}; wherein R^{K} and R^{L} are each independently selected from the group consisting of hydrogen and C₁₋₄alkyl;
X is selected from the group consisting of CH and CR¹⁰; wherein R¹⁰ is selected from the group consisting of —C₁₋₄alkyl;
R³ is selected from the group consisting of cyano, C₁₋₄alkyl, C₂₋₄alkenyl, C₃₋₈cycloalkyl, aryl, C₁₋₄aralkyl, and 5 to 6 membered heteroaryl; wherein the aryl or heteroaryl, whether alone or as part of a substituent group is optionally substituted with one or more substituents independently selected from the group consisting of halogen, hydroxy, C₁₋₄alkyl, halogenated C₁₋₄alkyl, C₁₋₄alkoxy, halogenated C₁₋₄alkoxy, cyano, nitro, NR^{E}R^{F} and —C(O)—NR^{E}R^{F}; wherein R^{E} and R^{F} are each independently selected from the group consisting of hydrogen and C₁₋₄alkyl; 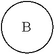
is selected from the group consisting of cycloalkyl, aryl, heteroaryl, heterocycloalkyl, —C(O)—C₁₋₄alkyl, —C(O)-aryl, and —C(O)-aryl; wherein the cycloalkyl, aryl, heteroaryl or heterocycloalkyl is optionally substituted with one or more substituents independently selected from the group consisting of halogen, C₁₋₈alkyl, halogenated C₁₋₄alkyl, C₁₋₄alkoxy, cyano, oxo, —C(O)OH, —C(O)O—C₁₋₄alkyl, —C(O)—NR^{C}R^{D}, —C(O)—NR^{E}—NR^{C}R^{D}, C₃₋₈cycloalkyl, aryl, heteroaryl and heterocycloalkyl;
wherein R^{C} and R^{D} are each independently selected from the group consisting of hydrogen and C₁₋₄alkyl; alternatively, R^{C} and R^{D} are taken together with the nitrogen atom to which they are bound to form a 4 to 8 membered saturated ring structure; and wherein R^{E} is selected from the group consisting of hydrogen and C₁₋₄alkyl;
provided that when R¹ is fluoro, R² is hydrogen, X is CH, R³ is phenyl, L¹ is —CH₂—C(O)—N(CH₃)— and R⁵ is ethyl, then 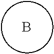
is not isopropyl-carbonyl;
provided further that when R¹ is fluoro, R² is hydrogen, X is CH, R³ is phenyl, L¹ is —NH—C(O)— and R⁵ is isopropyl, then 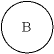
is not phenyl-carbonyl;
provided further that when R¹ is nitro or amino, R² is hydrogen, X is CH, R³ is phenyl or 4-fluoro-phenyl, L¹ is —C(O)O— and R⁵ is methyl; then 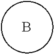
is other than phenyl or 4-fluoro-phenyl;
provided further that when R¹ fluoro, R² is hydrogen, X is CH, R³ is phenyl, L¹ is —NH—C(O)— and R⁵ is isopropyl, then 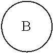
is other than 1-pyrrolidinyl;
or an enantiomer or pharmaceutically acceptable salt thereof.

2. A compound as in claim 1, wherein
R¹ and R² are each independently selected from the group consisting of hydrogen, halogen, C₁₋₄alkyl, —C₁₋₄alkyl-OH, —C₁₋₄alkoxy, —S—C₁₋₄alkyl, —SO—C₁₋₄alkyl, —SO₂—C₁₋₄alkyl, cyano, nitro and —NR^{A}R^{B}; wherein R^{A} and R^{B} are each independently selected from the group consisting of hydrogen and C₁₋₂alkyl;
provided that at least one of R¹ or R² is other than hydrogen;
L¹ is selected from the group consisting of —NR^{J}—, —NR^{J}—C(O)—, —(CH₂)ₐ—NR^{J}—C(O)—, —C(O)—NR^{J}— and —(CH₂)ₐ—C(O)—NR^{J}—; wherein R^{J} is selected from the group consisting of hydrogen and C₁₋₄alkyl; and wherein a is an integer from 1 to 3;
R⁵ is selected from the group consisting of C₁₋₈alkyl, C₃₋₈cycloalkyl, aryl, heteroaryl, and heterocycloalkyl; wherein the C₁₋₈alkyl, C₃₋₈cycloalkyl, aryl, heteroaryl or heterocycloalkyl is optionally substituted with one or more substituents independently selected from the group consisting of halogen, C₁₋₄alkyl, fluorinated C₁₋₄alkyl, C₁₋₄alkoxy, fluorinated C₁₋₄alkoxy, hydroxy, cyano, nitro and NR^{K}R^{L}; wherein R^{K} and R^{L} are each independently selected from the group consisting of hydrogen and C₁₋₄alkyl;
X is selected from the group consisting of CH and CR¹⁰; wherein R¹⁰ is selected from the group consisting of —C₁₋₂alkyl;
R³ is selected from the group consisting of cyano, C₁₋₄alkyl, C₃₋₈cycloalkyl, aryl and 5 to 6 membered heteroaryl; wherein the aryl or heteroaryl, whether alone or as part of a substituent group is optionally substituted with one or more substituents independently selected from the group consisting of halogen, hydroxy, C₁₋₄alkyl, halogenated C₁₋₄alkyl, C₁₋₄alkoxy, halogenated C₁₋₄alkoxy, cyano, nitro, NR^{E}R^{F} and —C(O)—NR^{E}R^{F}; wherein R^{E} and R^{F} are each independently selected from the group consisting of hydrogen and C₁₋₄alkyl; 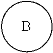
is selected from the group consisting of cycloalkyl, aryl, heteroaryl, heterocycloalkyl, —C(O)—C₁₋₄alkyl, —C(O)-aryl, and —C(O)-aryl; wherein the cycloalkyl, aryl, heteroaryl or heterocycloalkyl is optionally substituted with one or more substituents independently selected from the group consisting of halogen, C₁₋₆alkyl, fluorinated C₁₋₄alkyl, C₁₋₄alkoxy, cyano, —C(O)O—C₁₋₄alkyl, C₃₋₈cycloalkyl, aryl, heteroaryl and heterocycloalkyl;
provided that when R¹ is fluoro, R² is hydrogen, X is CH, R³ is phenyl, L¹ is —CH₂—C(O)—N(CH₃)— and R⁵ is ethyl, then 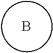
is not isopropyl-carbonyl;
provided further that when R¹ is fluoro, R² is hydrogen, X is CH, R³ is phenyl, L¹ is —NH—C(O)— and R⁵ is isopropyl, then 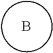
is not phenyl-carbonyl;
provided further that when R¹ fluoro, R² is hydrogen, X is CH, R³ is phenyl, L¹ is —NH—C(O)— and R⁵ is isopropyl, then 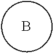
is other than 1-pyrrolidinyl;
or an enantiomer or pharmaceutically acceptable salt thereof.

3. A compound as in claim 2, wherein
R¹ and R² are each independently selected from the group consisting of hydrogen, halogen, C₁₋₄alkyl and cyano; provided that at least one of R¹ or R² is other than hydrogen;
L¹ is selected from the group consisting of —NR^{J}—, —NR^{J}—C(O)—, —(CH₂)ₐ—NR^{J}—C(O)—, —C(O)—NR^{J}— and —(CH₂)ₐ—C(O)—NR^{J}—; wherein R^{J} is selected from the group consisting of hydrogen and C₁₋₂alkyl; and wherein a is an integer from 1 to 2;
R⁵ is selected from the group consisting of C₁₋₆alkyl, C₃₋₈cycloalkyl, aryl, heteroaryl and heterocycloalkyl; wherein the C₃₋₈cycloalkyl, aryl, heteroaryl or heterocycloalkyl is optionally substituted with one to three substituents independently selected from the group consisting of halogen, C₁₋₄alkyl and NR^{K}R^{L}; wherein R^{K} and R^{L} are each independently selected from the group consisting of hydrogen and C₁₋₂alkyl;
X is CH;
R³ is selected from the group consisting of cyano, C₃₋₈cycloalkyl, aryl and 5 to 6 membered heteroaryl; wherein the aryl is optionally substituted with a substituent selected from the group consisting of halogen, C₁₋₄alkoxy, fluorinated C₁₋₄alkoxy and cyano; 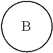
is selected from the group consisting of cycloalkyl, aryl, heteroaryl, heterocycloalkyl and —C(O)—C₁₋₄alkyl; wherein the cycloalkyl, aryl, heteroaryl and heterocycloalkyl is optionally substituted with one to two substituents independently selected from halogen, cyano, C₁₋₄alkyl, fluorinated C₁₋₆alkyl, C₁₋₄alkoxy, —C(O)O—C₁₋₄alkyl, phenyl and 5 to 6 membered heteroaryl;
provided that when R¹ is fluoro, R² is hydrogen, X is CH, R³ is phenyl, L¹ is —CH₂—C(O)—N(CH₃)— and R⁵ is ethyl, then 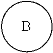
is not isopropyl-carbonyl;
provided further that when R¹ fluoro, R² is hydrogen, X is CH, R¹³ is phenyl, L¹ is —NH—C(O)— and R⁵ is isopropyl, then 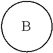
is other than 1-pyrrolidinyl;
or an enantiomer or pharmaceutically acceptable salt thereof.

4. A compound as in claim 3, wherein
R¹ is selected from the group consisting of fluoro, bromo, methyl and cyano; and R² is selected from the group consisting of hydrogen and fluoro;
L¹ is selected from the group consisting of —NH—, —NH—C(O)—, —CH₂—NH—C(O)—, —C(O)—NH— and —CH₂—C(O)—N(ethyl)-;
R⁵ is selected from the group consisting of methyl, ethyl, 2-n-propyl, isopropyl, n-butyl, 3-n-pentyl, 1-(1-(R)-methyl-n-propyl), 1-(1-methyl-3,3,3-trifluoro-n-propyl), dimethylamino-methyl-, cyclopropyl, cyclobutyl, cylopentyl, cyclohexyl, 4,4-difluoro-cyclohexyl, 2-methyl-phenyl, 3-tetrahydrofuranyl, 3-(S)-tetrahydrofuranyl, 3-(R)-tetrahydrofuranyl, 2-(3-methyl-pyridyl), 2-(6-methyl-pyridyl), 2-(1-methyl-imidazolyl), 2-(4-methyl-pyrimidinyl) and 4-(3,5-dimethyl-isoxazolyl);
X is CH;
R³ is selected from the group consisting of cyano, cyclopropyl, cyclohexyl, phenyl, (R)-phenyl, (S)-phenyl, 4-fluorophenyl, 3-fluorophenyl, 4-chlorophenyl, 4-methoxy-phenyl, 4-cyano-phenyl, 4-trifluoromethoxyphenyl, 2-pyridyl and 2-oxazolyl; 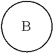
is selected from the group consisting of methylcarbonyl-, cyclopropyl, cyclobutyl, cyclopentyl, 1-(2,2-dichloro-3-methyl-cyclopropyl), phenyl, 4-fluorophenyl, 4-chlorophenyl, 2-methylphenyl, 2-methoxy-phenyl, 2-cyano-phenyl, 3-tetrahydrofuranyl, 2-furyl, 2-pyridyl, 3-pyridyl, 4-pyridyl, 2-thienyl, 2-thiazolyl, 2-pyrimidinyl, 2-(1-methyl-imidazolyl), 2-benzoxazolyl, 2-benzthiazolyl, 5-(3-methyl-isoxazolyl), 2-(4,5-dihydro-4-methoxycarbonyl-oxazolyl), 2-oxazolyl, 2-(5-methyl-[1,3,4]-oxadiazolyl), 2-(5-ethyl-[1,3,4]-oxadiazolyl), 2-(5-isopropyl-[1,3,4]-oxadiazolyl), 2-(5-(3-n-pentyl)-[1,3,4]-oxadiazolyl), 5-(3-methyl-[1,2,4]-oxadiazolyl), 5-(3-ethyl-[1,2,4]-oxadiazolyl), 5-(3-isopropyl-[1,2,4]-oxadiazolyl), 3-(5-isopropyl-[1,2,4]-oxadiazolyl), 3-(5-methyl-[1,2,4]-oxadiazolyl), 3-(5-fluoromethyl-[1,2,4]-oxadiazolyl), 3-(5-trifluoromethyl-[1,2,4]-oxadiazolyl), 3-(5-ethyl-[1,2,4]-oxadiazolyl), 3-(5-phenyl-[1,2,4]-oxadiazolyl), and 3-(5-(3-pyridyl)[1,2,4]-oxadiazolyl);
or an enantiomer or pharmaceutically acceptable salt thereof.

5. A compound as in claim 3 wherein
R¹ is selected from the group consisting of fluoro, bromo, methyl and cyano; and R² is selected from the group consisting of hydrogen and fluoro;
L¹ is selected from the group consisting of —NH—, —NH—C(O)—, —CH₂—NH—C(O)—, —C(O)—NH— and —CH₂—C(O)—N(ethyl)-;
R⁵ is selected from the group consisting of methyl, ethyl, 2-n-propyl, isopropyl, n-butyl, 3-n-pentyl, 1-(1-(R)-methyl-n-propyl), 1-(1-methyl-3,3,3-trifluoro-n-propyl), dimethylamino-methyl-, cyclopropyl, cyclobutyl, cylopentyl, cyclohexyl, 4,4-difluoro-cyclohexyl, 2-methyl-phenyl, 3-tetrahydrofuranyl, 3-(S)-tetrahydrofuranyl, 3-(R)-tetrahydrofuranyl, 2-(3-methyl-pyridyl), 2-(6-methyl-pyridyl), 2-(1-methyl-imidazolyl), 2-(4-methyl-pyrimidinyl) and 4-(3,5-dimethyl-isoxazolyl);
X is CH;
R³ is selected from the group consisting of cyano, cyclopropyl, cyclohexyl, phenyl, (R)-phenyl, (S)-phenyl, 4-fluorophenyl, 3-fluorophenyl, 4-chlorophenyl, 4-methoxy-phenyl, 4-cyano-phenyl, 4-trifluoromethoxyphenyl, 2-pyridyl, 3-pyridyl, 4-pyridyl and 2-oxazolyl; 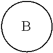
is selected from the group consisting of methylcarbonyl-, cyclopropyl, cyclobutyl, cyclopentyl, 1-(2,2-dichloro-3-methyl-cyclopropyl), phenyl, 4-fluorophenyl, 4-chlorophenyl, 2-methylphenyl, 2-methoxy-phenyl, 2-cyano-phenyl, 3-tetrahydrofuranyl, 2-furyl, 2-pyridyl, 3-pyridyl, 4-pyridyl, 2-thienyl, 2-thiazolyl, 2-pyrimidinyl, 2-(1-methyl-imidazolyl), 2-benzoxazolyl, 2-benzthiazolyl, 5-(3-methyl-isoxazolyl), 2-(4,5-dihydro-4-methoxycarbonyl-oxazolyl), 2-oxazolyl, 2-(5-methyl-[1,3,4]-oxadiazolyl), 2-(5-ethyl-[1,3,4]-oxadiazolyl), 2-(5-isopropyl-[1,3,4]-oxadiazolyl), 2-(5-(3-n-pentyl)-[1,3,4]-oxadiazolyl), 5-(3-methyl-[1,2,4]-oxadiazolyl), 5-(3-ethyl-[1,2,4]-oxadiazolyl), 5-(3-isopropyl-[1,2,4]-oxadiazolyl), 3-(5-isopropyl-[1,2,4]-oxadiazolyl), 3-(5-methyl-[1,2,4]-oxadiazolyl), 3-(5-fluoromethyl-[1,2,4]-oxadiazolyl), 3-(5-trifluoromethyl-[1,2,4]-oxadiazolyl), 3-(5-ethyl-[1,2,4]-oxadiazolyl), 3-(5-phenyl-[1,2,4]-oxadiazolyl), and 3-(5-(3-pyridyl)[1,2,4]-oxadiazolyl);
or an enantiomer or pharmaceutically acceptable salt thereof.

6. A compound as in claim 4, wherein
R¹ is selected from the group consisting of fluoro, bromo, methyl and cyano; and R² is selected from the group consisting of hydrogen and fluoro;
L¹ is —NH—C(O)—;
R⁵ is selected from the group consisting of isopropyl, 3-n-pentyl, 1-(1-(R)-methyl-n-propyl), 1-(1-methyl-3,3,3-trifluoro-n-propyl), cyclopropyl, cyclobutyl, 3-tetrahydrofuranyl, 3-(S)-tetrahydrofuranyl and 3-(R)-tetrahydrofuranyl;
X is CH;
R³ is selected from the group consisting of phenyl, (R)-phenyl, (S)-phenyl, 4-fluorophenyl, 3-fluorophenyl, 4-methoxy-phenyl, 4-cyano-phenyl, 4-trifluoromethoxyphenyl, and 2-pyridyl; 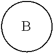
is selected from the group consisting of cyclopropyl, cyclobutyl, phenyl, 2-pyridyl, 3-pyridyl, 2-thiazolyl, 2-pyrimidinyl, 2-benzoxazolyl, 2-benzthiazolyl, 5-(3-methyl-isoxazolyl), 2-(4,5-dihydro-4-methoxycarbonyl-oxazolyl), 2-oxazolyl, 2-(5-methyl-[1,3,4]-oxadiazolyl), 2-(5-ethyl-[1,3,4]-oxadiazolyl), 2-(5-isopropyl-[1,3,4]-oxadiazolyl), 2-(5-(3-n-pentyl)-[1,3,4]-oxadiazolyl), 5-(3-methyl-[1,2,4]-oxadiazolyl), 5-(3-ethyl-[1,2,4]-oxadiazolyl), 5-(3-isopropyl-[1,2,4]-oxadiazolyl), 3-(5-isopropyl-[1,2,4]-oxadiazolyl), 3-(5-methyl-[1,2,4]-oxadiazolyl), 3-(5-fluoromethyl-[1,2,4]-oxadiazolyl), 3-(5-ethyl-[1,2,4]-oxadiazolyl), 3-(5-phenyl-[1,2,4]-oxadiazolyl), and 3-(5-(3-pyridyl)-[1,2,4]-oxadiazolyl);
or an enantiomer or pharmaceutically acceptable salt thereof.

7. A compound as in claim 4, wherein
R¹ is selected from the group consisting of fluoro, bromo and cyano;
R² is hydrogen;
L¹ is —NH—C(O)—;
R⁵ is selected from the group consisting of 3-n-pentyl, 1-(1-(R)-methyl-n-propyl), 1-(1-methyl-3,3,3-trifluoro-n-propyl) and 3-(R)-tetrahydrofuranyl;
X is CH;
R³ is selected from the group consisting of phenyl, (S)-phenyl, 4-fluorophenyl, 3-fluorophenyl, 4-methoxy-phenyl and 4-cyano-phenyl; 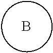
is selected from the group consisting of phenyl, 2-pyridyl, 3-pyridyl, 2-thiazolyl, 2-pyrimidinyl, 2-benzoxazolyl, 2-benzthiazolyl, 5-(3-methyl-isoxazolyl), 2-(4,5-dihydro-4-methoxycarbonyl-oxazolyl), 2-oxazolyl, 2-(5-methyl-[1,3,4]-oxadiazolyl), 2-(5-ethyl-[1,3,4]-oxadiazolyl), 2-(5-isopropyl-[1,3,4]-oxadiazolyl), 2-(5-(3-n-pentyl)[1,3,4]-oxadiazolyl), 5-(3-methyl-[1,2,4]-oxadiazolyl), 5-(3-ethyl-[1,2,4]-oxadiazolyl), 5-(3-isopropyl-[1,2,4]-oxadiazolyl), 3-(5-isopropyl-[1,2,4]-oxadiazolyl), 3-(5-methyl-[1,2,4]-oxadiazolyl), 3-(5-fluoromethyl-[1,2,4]-oxadiazolyl), 3-(5-ethyl-[1,2,4]-oxadiazolyl), 3-(5-phenyl-[1,2,4]-oxadiazolyl), and 3-(5-(3-pyridyl)[1,2,4]-oxadiazolyl);
or an enantiomer or pharmaceutically acceptable salt thereof.

8. A compound as in claim 4, wherein
R¹ is selected from the group consisting of fluoro, bromo and cyano;
R² is hydrogen;
L¹ is —NH—C(O)—;
R⁵ is 3-n-pentyl;
X is CH;
R³ is selected from the group consisting of phenyl and 3-fluorophenyl; 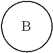
is selected from the group consisting of 2-pyridyl, 2-benzoxazolyl, 5-(3-methyl-isoxazolyl), 2-oxazolyl, 2-(5-(3-n-pentyl)-[1,3,4]-oxadiazolyl), and 3-(5-methyl-[1,2,4]-oxadiazolyl);
or an enantiomer or pharmaceutically acceptable salt thereof.

9. A compound as in claim 4, wherein
R¹ is selected from the group consisting of fluoro, bromo, methyl and cyano; and R² is selected from the group consisting of hydrogen and fluoro;
L¹ is —NH—C(O)—;
R⁵ is selected from the group consisting of 3-n-pentyl, 1-(1-(R)-methyl-n-propyl), 1-(1-methyl-3,3,3-trifluoro-n-propyl), cyclopropyl, 3-(R)-tetrahydrofuranyl and 4-(3,5-dimethyl-isoxazolyl);
X is CH;
R³ is selected from the group consisting of phenyl, (R)-phenyl, (S)-phenyl, 4-fluorophenyl, 3-fluorophenyl, 4-methoxy-phenyl and 4-cyano-phenyl; 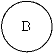
is selected from the group consisting of phenyl, 2-methyl-phenyl, 2-pyridyl, 3-pyridyl, 4-pyridyl, 2-thiazolyl, 2-pyrimidinyl, 2-benzoxazolyl, 2-benzthiazolyl, 5-(3-methyl-isoxazolyl), 2-oxazolyl, 2-(5-methyl-[1,3,4]-oxadiazolyl), 2-(5-ethyl-[1,3,4]-oxadiazolyl), 2-(5-isopropyl-[1,3,4]-oxadiazolyl), 2-(5-(3-n-pentyl)[1,3,4]-oxadiazolyl), 5-(3-methyl-[1,2,4]-oxadiazolyl), 5-(3-ethyl-[1,2,4]-oxadiazolyl), 3-(5-isopropyl-[1,2,4]-oxadiazolyl), 3-(5-methyl-[1,2,4]-oxadiazolyl), 3-(5-fluoromethyl-[1,2,4]-oxadiazolyl), 3-(5-ethyl-[1,2,4]-oxadiazolyl), 3-(5-phenyl-[1,2,4]-oxadiazolyl), and 3-(5-(3-pyridyl)-[1,2,4]-oxadiazolyl);
or an enantiomer or pharmaceutically acceptable salt thereof.

10. A compound as in claim 4, wherein
R¹ is cyano;
R² is hydrogen;
L¹ is —NH—C(O)—;
R⁵ is selected from the group consisting of 3-n-pentyl, cyclopropyl, and 4-(3,5-dimethyl-isoxazolyl);
X is CH;
R³ is phenyl; 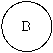
is selected from the group consisting of phenyl, 2-methyl-phenyl, 4-chlorophenyl, 3-pyridyl and 4-pyridyl
or an enantiomer or pharmaceutically acceptable salt thereof.

11. A pharmaceutical composition comprising a pharmaceutically acceptable carrier and a compound of claim 1.

12. A pharmaceutical composition made by mixing a compound of claim 1 and a pharmaceutically acceptable carrier.

13. A process for making a pharmaceutical composition comprising mixing a compound of claim 1 and a pharmaceutically acceptable carrier.

14. A method of treating a disorder mediated by the NPY Y2 receptor, comprising administering to a subject in need thereof, a therapeutically effective amount of a compound as in claim 1.

15. A method as in claim 14, wherein the disorder mediated by the NPY Y2 receptor is selected from the group consisting of anxiolytic disorders, depression; pain, injured mammalian nerve tissue; conditions responsive to treatment with a neurotrophic factor; neurological disorders; bone loss; cardiovascular diseases; sleep-wake state disorders, substance abuse and addiction related disorders; obesity; obesity-related disorders, disorders responsive to modulation of endocrine function, inovulation and infertility.

16. A method as in claim 14, wherein the disorder mediated by the NPY Y2 receptor is selected from the group consisting of substance abuse and addiction related disorders.

17. A method as in claim 16, wherein the substance of abuse or addiction is alcohol.

18. A method of treating a disorder selected from the group consisting of anxiolytic disorders, depression; pain, injured mammalian nerve tissue; conditions responsive to treatment with a neurotrophic factor; neurological disorders; bone loss; cardiovascular diseases; sleep-wake state disorders, substance abuse and addiction related disorders; obesity; obesity-related disorders, disorders responsive to modulation of endocrine function, inovulation and infertility; comprising administering to a subject in need thereof, a therapeutically effective amount of a compound as in claim 1.

19. The use of a compound as in claim 1, for the preparation of a medicament for treating: (a) anxiolytic disorders, (b) depression; (c) pain, (d) injured mammalian nerve tissue; (d) conditions responsive to treatment with a neurotrophic factor; (e) neurological disorders; (f) bone loss; (g) cardiovascular diseases; (h) sleep-wake state disorders, (i) substance abuse and addiction related disorders; (j) obesity; (k) obesity-related disorders, (l) disorders responsive to modulation of endocrine function (more particularly, disorders responsive to modulation of the pituitary and/or hypothalamic gland); (m) inovulation; and (n) infertility; in a subject in need thereof.
